SEQUENCE LISTING (709 sequences)

SEQ ID NO: 1 - protein, 638 aa, Eptesicus fuscus
MDKFSKDIESSDDEFYFENEEKSEKCNSDESEFSEDASGDDEQIAGPSGTTERKKSLALPKDLAESTDSDSDIEFIKAKRRRTIVYSSESDGDIGDIIEKSGIRPSESYVSRGKQEKEKWTSTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 2 - DNA, 1869 bp, Eptesicus fuscus
  source 1..1869: mol_type = genomic DNA
atggacaagttttccaaggacattgaaagctctgacgatgaattttacttcgagaacgaggagaaaagcgagaagtgtaattccgatgagtccgagtttagcgaggacgctagcggcgacgacgagcagatcgctggacccagcgggaccacggagcgcaaaaagagcctggctctgcctaaagacttggccgagagtaccgacagcgactccgatatcgagttcatcaaggccaaacgcaggcgcacaatcgtgtactcttccgagagcgacggcgacatcggcgatattatcgagaaaagcgggatccggccttccgaaagctacgtgtctcggggcaagcaggagaaggaaaagtggacaagcacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtg

SEQ ID NO: 3 - DNA, 200 bp, Eptesicus fuscus
  source 1..200: mol_type = genomic DNA
ccttttgcactcggatgtcgagtgtgactcgacacggttagcatcggtagcagctcgtatgtcgagccacactcgacacgtagtttcaccgaggggggaagggggatttttgtctatttttccagtatcttttcttgttttcattagcatgaaaggacaagtaaaatgtaaatgccgtctcaactgatgccaccacctaa

SEQ ID NO: 4 - DNA, 200 bp, Eptesicus fuscus
  source 1..200: mol_type = genomic DNA
tgaaaaattatagagattaaaattactctttgaatgtatcaataatttgaaatataaaaaaatccaaataaataagtttgtatgaaaagaaactccagttttttattctactgccgcgctttgtaaaatctggggtatttaaaaaattaaatcccgagtagaataaaggaatcgagaaaaaagcaagcgagtgcaaaggg

SEQ ID NO: 5 - DNA, 4104 bp, synthetic construct
  source 1..4104: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctggaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 6 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 508 aa, synthetic construct
MRNFPVPYSNELIYSTIARAGVYQGIVSPKQLLDEVYGNRKVVATLGLPSHLGVIARHLHQTGRYAVQQLIYEHTLFPLYAPFVGKERRDEAIRLMEYQAQGAVHLMLGVAASRVKSDNRFRYCPDCVALQLNRYGEAFWQRDWYLPALPYCPKHGALVFFDRAVDDHRHQFWALGHTELLSDYPKDSLSQLTALAAYIAPLLDAPRAQELSPSLEQWTLFYQRLAQDLGLTKSKHIRHDLVAERVRQTFSDEALEKLDLKLAENKDTCWLKSIFRKHRKAFSYLQHSIVWQALLPKLTVIEALQQASALTEHSITTRPVSQSVQPNSEDLSVKHKDWQQLVHKYQGIKAARQSLEGGVLYAWLYRHDRDWLVHWNQQHQQERLAPAPRVDWNQRDRIAVRQLLRIIKRLDSSLDHPRATSSWLLKQTPNGTSLAKNLQKLPLVALCLKRYSESVEDYQIRRISQAFIKLKQEDVELRRWRLLRSATLSKERITEEAQRFLEMVYGEE

SEQ ID NO: 8 - DNA, 19 bp, synthetic construct
  source 1..19: mol_type = other DNA
tggccggcctgaccactgg

SEQ ID NO: 9 - DNA, 19 bp, synthetic construct
  source 1..19: mol_type = other DNA
tgaaggcctggccggcctg

SEQ ID NO: 10 - DNA, 19 bp, synthetic construct
  source 1..19: mol_type = other DNA
tgagcactgaaggcctggc

SEQ ID NO: 11 - DNA, 19 bp, synthetic construct
  source 1..19: mol_type = other DNA
tccactgagcactgaaggc

SEQ ID NO: 12 - DNA, 19 bp, synthetic construct
  source 1..19: mol_type = other DNA
tggtttccactgagcactg

SEQ ID NO: 13 - DNA, 19 bp, synthetic construct
  source 1..19: mol_type = other DNA
tggggaaaatgacccaaca

SEQ ID NO: 14 - DNA, 19 bp, synthetic construct
  source 1..19: mol_type = other DNA
taggacagtggggaaaatg

SEQ ID NO: 15 - DNA, 19 bp, synthetic construct
  source 1..19: mol_type = other DNA
tccagggacacggtgctag

SEQ ID NO: 16 - DNA, 19 bp, synthetic construct
  source 1..19: mol_type = other DNA
tcagagccaggagtcctgg

SEQ ID NO: 17 - DNA, 19 bp, synthetic construct
  source 1..19: mol_type = other DNA
tccttcagagccaggagtc

SEQ ID NO: 18 - DNA, 19 bp, synthetic construct
  source 1..19: mol_type = other DNA
tcctccttcagagccagga

SEQ ID NO: 19 - DNA, 19 bp, synthetic construct
  source 1..19: mol_type = other DNA
tccagcccctcctccttca

SEQ ID NO: 20 - DNA, 19 bp, synthetic construct
  source 1..19: mol_type = other DNA
tccgagcttgacccttgga

SEQ ID NO: 21 - DNA, 19 bp, synthetic construct
  source 1..19: mol_type = other DNA
tggtttccgagcttgaccc

SEQ ID NO: 22 - DNA, 19 bp, synthetic construct
  source 1..19: mol_type = other DNA
tggggtggtttccgagctt

SEQ ID NO: 23 - DNA, 19 bp, synthetic construct
  source 1..19: mol_type = other DNA
tctgctggggtggtttccg

SEQ ID NO: 24 - DNA, 19 bp, synthetic construct
  source 1..19: mol_type = other DNA
tgcagagtatctgctgggg

SEQ ID NO: 25 - DNA, 14 bp, synthetic construct
  source 1..14: mol_type = other DNA
ccaatcccctcagt

SEQ ID NO: 26 - DNA, 15 bp, synthetic construct
  source 1..15: mol_type = other DNA
cagtgctcagtggaa

SEQ ID NO: 27 - DNA, 18 bp, synthetic construct
  source 1..18: mol_type = other DNA
gaaacatccggcgactca

SEQ ID NO: 28 - DNA, 19 bp, synthetic construct
  source 1..19: mol_type = other DNA
tcgcccctcaaatcttaca

SEQ ID NO: 29 - DNA, 19 bp, synthetic construct
  source 1..19: mol_type = other DNA
tcaaatcttacagctgctc

SEQ ID NO: 30 - DNA, 19 bp, synthetic construct
  source 1..19: mol_type = other DNA
tcttacagctgctcactcc

SEQ ID NO: 31 - DNA, 19 bp, synthetic construct
  source 1..19: mol_type = other DNA
tacagctgctcactcccct

SEQ ID NO: 32 - DNA, 19 bp, synthetic construct
  source 1..19: mol_type = other DNA
tgctcactcccctgcaggg

SEQ ID NO: 33 - DNA, 19 bp, synthetic construct
  source 1..19: mol_type = other DNA
tcccctgcagggcaacgcc

SEQ ID NO: 34 - DNA, 19 bp, synthetic construct
  source 1..19: mol_type = other DNA
tgcagggcaacgcccaggg

SEQ ID NO: 35 - DNA, 19 bp, synthetic construct
  source 1..19: mol_type = other DNA
tctcgattatgggcgggat

SEQ ID NO: 36 - DNA, 19 bp, synthetic construct
  source 1..19: mol_type = other DNA
tcgcttctcgattatgggc

SEQ ID NO: 37 - DNA, 19 bp, synthetic construct
  source 1..19: mol_type = other DNA
tgtcgagtcgcttctcgat

SEQ ID NO: 38 - DNA, 19 bp, synthetic construct
  source 1..19: mol_type = other DNA
tccatgtcgagtcgcttct

SEQ ID NO: 39 - DNA, 19 bp, synthetic construct
  source 1..19: mol_type = other DNA
tcgcctccatgtcgagtcg

SEQ ID NO: 40 - DNA, 19 bp, synthetic construct
  source 1..19: mol_type = other DNA
tcgtcatcgcctccatgtc

SEQ ID NO: 41 - DNA, 19 bp, synthetic construct
  source 1..19: mol_type = other DNA
tgatctcgtcatcgcctcc

SEQ ID NO: 42 - DNA, 14 bp, synthetic construct
  source 1..14: mol_type = other DNA
gcttcagcttccta

SEQ ID NO: 43 - DNA, 14 bp, synthetic construct
  source 1..14: mol_type = other DNA
ctgtgatcatgcca

SEQ ID NO: 44 - DNA, 16 bp, synthetic construct
  source 1..16: mol_type = other DNA
acagtggtacacacct

SEQ ID NO: 45 - DNA, 15 bp, synthetic construct
  source 1..15: mol_type = other DNA
ccaccccccactaag

SEQ ID NO: 46 - DNA, 14 bp, synthetic construct
  source 1..14: mol_type = other DNA
cattggccgggcac

SEQ ID NO: 47 - DNA, 16 bp, synthetic construct
  source 1..16: mol_type = other DNA
gcttgaacccaggaga

SEQ ID NO: 48 - DNA, 17 bp, synthetic construct
  source 1..17: mol_type = other DNA
acacccgatccactggg

SEQ ID NO: 49 - DNA, 14 bp, synthetic construct
  source 1..14: mol_type = other DNA
gctgcatcaacccc

SEQ ID NO: 50 - DNA, 16 bp, synthetic construct
  source 1..16: mol_type = other DNA
gccacaaacagaaata

SEQ ID NO: 51 - DNA, 15 bp, synthetic construct
  source 1..15: mol_type = other DNA
ggtggctcatgcctg

SEQ ID NO: 52 - DNA, 16 bp, synthetic construct
  source 1..16: mol_type = other DNA
gatttgcacagctcat

SEQ ID NO: 53 - DNA, 15 bp, synthetic construct
  source 1..15: mol_type = other DNA
aagctctgaggagca

SEQ ID NO: 54 - DNA, 13 bp, synthetic construct
  source 1..13: mol_type = other DNA
ccctagctgtccc

SEQ ID NO: 55 - DNA, 14 bp, synthetic construct
  source 1..14: mol_type = other DNA
gcctagcatgctag

SEQ ID NO: 56 - DNA, 15 bp, synthetic construct
  source 1..15: mol_type = other DNA
atgggcttcacggat

SEQ ID NO: 57 - DNA, 14 bp, synthetic construct
  source 1..14: mol_type = other DNA
gaaactatgcctgc

SEQ ID NO: 58 - DNA, 14 bp, synthetic construct
  source 1..14: mol_type = other DNA
gcaccattgctccc

SEQ ID NO: 59 - DNA, 14 bp, synthetic construct
  source 1..14: mol_type = other DNA
gacatgcaactcag

SEQ ID NO: 60 - DNA, 14 bp, synthetic construct
  source 1..14: mol_type = other DNA
acaccactaggggt

SEQ ID NO: 61 - DNA, 14 bp, synthetic construct
  source 1..14: mol_type = other DNA
gtctgctagacagg

SEQ ID NO: 62 - DNA, 15 bp, synthetic construct
  source 1..15: mol_type = other DNA
ggcctagacaggctg

SEQ ID NO: 63 - DNA, 15 bp, synthetic construct
  source 1..15: mol_type = other DNA
gaggcattcttatcg

SEQ ID NO: 64 - DNA, 15 bp, synthetic construct
  source 1..15: mol_type = other DNA
gcctggaaacgttcc

SEQ ID NO: 65 - DNA, 14 bp, synthetic construct
  source 1..14: mol_type = other DNA
gtgctctgacaata

SEQ ID NO: 66 - DNA, 14 bp, synthetic construct
  source 1..14: mol_type = other DNA
gttttgcagcctcc

SEQ ID NO: 67 - DNA, 15 bp, synthetic construct
  source 1..15: mol_type = other DNA
acagctgtggaacgt

SEQ ID NO: 68 - DNA, 15 bp, synthetic construct
  source 1..15: mol_type = other DNA
ggctctcttcctcct

SEQ ID NO: 69 - DNA, 15 bp, synthetic construct
  source 1..15: mol_type = other DNA
ctatcccaaaactct

SEQ ID NO: 70 - DNA, 14 bp, synthetic construct
  source 1..14: mol_type = other DNA
gaaaaactatgtat

SEQ ID NO: 71 - DNA, 15 bp, synthetic construct
  source 1..15: mol_type = other DNA
aggcaggctggttga

SEQ ID NO: 72 - DNA, 14 bp, synthetic construct
  source 1..14: mol_type = other DNA
caatacaaccacgc

SEQ ID NO: 73 - DNA, 15 bp, synthetic construct
  source 1..15: mol_type = other DNA
atgacggactcaact

SEQ ID NO: 74 - DNA, 14 bp, synthetic construct
  source 1..14: mol_type = other DNA
cacaacatttgtaa

SEQ ID NO: 75 - DNA, 14 bp, synthetic construct
  source 1..14: mol_type = other DNA
atttccagtgcaca

SEQ ID NO: 76 - DNA, 20 bp, synthetic construct
  source 1..20: mol_type = other DNA
gtttagctcacccgtgagcc

SEQ ID NO: 77 - DNA, 20 bp, synthetic construct
  source 1..20: mol_type = other DNA
cccaatattattgttctctg

SEQ ID NO: 78 - DNA, 20 bp, synthetic construct
  source 1..20: mol_type = other DNA
ggggtgggataggggatacg

SEQ ID NO: 79 - DNA, 20 bp, synthetic construct
  source 1..20: mol_type = other DNA
ggatccccctctacatttaa

SEQ ID NO: 80 - DNA, 20 bp, synthetic construct
  source 1..20: mol_type = other DNA
gtgatcttgtacaaatcatt

SEQ ID NO: 81 - DNA, 20 bp, synthetic construct
  source 1..20: mol_type = other DNA
ctacacagaatctgttagaa

SEQ ID NO: 82 - DNA, 20 bp, synthetic construct
  source 1..20: mol_type = other DNA
taagctagagaatagatctc

SEQ ID NO: 83 - DNA, 20 bp, synthetic construct
  source 1..20: mol_type = other DNA
tcaatacacttaatgattta

SEQ ID NO: 84 - DNA, 25 bp, synthetic construct
  source 1..25: mol_type = other DNA
caccgggagccacgaaaacagatcc

SEQ ID NO: 85 - DNA, 25 bp, synthetic construct
  source 1..25: mol_type = other DNA
caccgcgaaaacagatccagggaca

SEQ ID NO: 86 - DNA, 25 bp, synthetic construct
  source 1..25: mol_type = other DNA
caccgagatccagggacacggtgct

SEQ ID NO: 87 - DNA, 25 bp, synthetic construct
  source 1..25: mol_type = other DNA
caccggacacggtgctaggacagtg

SEQ ID NO: 88 - DNA, 25 bp, synthetic construct
  source 1..25: mol_type = other DNA
caccggaaaatgacccaacagcctc

SEQ ID NO: 89 - DNA, 25 bp, synthetic construct
  source 1..25: mol_type = other DNA
caccggcctggccggcctgaccact

SEQ ID NO: 90 - DNA, 25 bp, synthetic construct
  source 1..25: mol_type = other DNA
caccgctgagcactgaaggcctggc

SEQ ID NO: 91 - DNA, 25 bp, synthetic construct
  source 1..25: mol_type = other DNA
caccgtggtttccactgagcactga

SEQ ID NO: 92 - DNA, 25 bp, synthetic construct
  source 1..25: mol_type = other DNA
caccggatagccaggagtcctttcg

SEQ ID NO: 93 - DNA, 25 bp, synthetic construct
  source 1..25: mol_type = other DNA
caccggcgcttccagtgctcagact

SEQ ID NO: 94 - DNA, 25 bp, synthetic construct
  source 1..25: mol_type = other DNA
caccgcagtgctcagactagggaag

SEQ ID NO: 95 - DNA, 25 bp, synthetic construct
  source 1..25: mol_type = other DNA
caccggcccctcctccttcagagcc

SEQ ID NO: 96 - DNA, 25 bp, synthetic construct
  source 1..25: mol_type = other DNA
caccgtccttcagagccaggagtcc

SEQ ID NO: 97 - DNA, 25 bp, synthetic construct
  source 1..25: mol_type = other DNA
caccgtggtttccgagcttgaccct

SEQ ID NO: 98 - DNA, 25 bp, synthetic construct
  source 1..25: mol_type = other DNA
caccgctgcagagtatctgctgggg

SEQ ID NO: 99 - DNA, 25 bp, synthetic construct
  source 1..25: mol_type = other DNA
caccgcgttcctgcagagtatctgc

SEQ ID NO: 100 - DNA, 25 bp, synthetic construct
  source 1..25: mol_type = other DNA
aaacggatctgttttcgtggctccc

SEQ ID NO: 101 - DNA, 25 bp, synthetic construct
  source 1..25: mol_type = other DNA
aaactgtccctggatctgttttcgc

SEQ ID NO: 102 - DNA, 25 bp, synthetic construct
  source 1..25: mol_type = other DNA
aaacagcaccgtgtccctggatctc

SEQ ID NO: 103 - DNA, 25 bp, synthetic construct
  source 1..25: mol_type = other DNA
aaaccactgtcctagcaccgtgtcc

SEQ ID NO: 104 - DNA, 25 bp, synthetic construct
  source 1..25: mol_type = other DNA
aaacgaggctgttgggtcattttcc

SEQ ID NO: 105 - DNA, 25 bp, synthetic construct
  source 1..25: mol_type = other DNA
aaacagtggtcaggccggccaggcc

SEQ ID NO: 106 - DNA, 25 bp, synthetic construct
  source 1..25: mol_type = other DNA
aaacgccaggccttcagtgctcagc

SEQ ID NO: 107 - DNA, 25 bp, synthetic construct
  source 1..25: mol_type = other DNA
aaactcagtgctcagtggaaaccac

SEQ ID NO: 108 - DNA, 25 bp, synthetic construct
  source 1..25: mol_type = other DNA
aaaccgaaaggactcctggctatcc

SEQ ID NO: 109 - DNA, 25 bp, synthetic construct
  source 1..25: mol_type = other DNA
aaacagtctgagcactggaagcgcc

SEQ ID NO: 110 - DNA, 25 bp, synthetic construct
  source 1..25: mol_type = other DNA
aaaccttccctagtctgagcactgc

SEQ ID NO: 111 - DNA, 25 bp, synthetic construct
  source 1..25: mol_type = other DNA
aaacggctctgaaggaggaggggcc

SEQ ID NO: 112 - DNA, 25 bp, synthetic construct
  source 1..25: mol_type = other DNA
aaacggactcctggctctgaaggac

SEQ ID NO: 113 - DNA, 25 bp, synthetic construct
  source 1..25: mol_type = other DNA
aaacagggtcaagctcggaaaccac

SEQ ID NO: 114 - DNA, 25 bp, synthetic construct
  source 1..25: mol_type = other DNA
aaacccccagcagatactctgcagc

SEQ ID NO: 115 - DNA, 25 bp, synthetic construct
  source 1..25: mol_type = other DNA
aaacgcagatactctgcaggaacgc

SEQ ID NO: 116 - DNA, 20 bp, synthetic construct
  source 1..20: mol_type = other DNA
tcccctcccagaaagacctg

SEQ ID NO: 117 - DNA, 20 bp, synthetic construct
  source 1..20: mol_type = other DNA
tgggctccaagcaatcctgg

SEQ ID NO: 118 - DNA, 20 bp, synthetic construct
  source 1..20: mol_type = other DNA
gtggctcaggaggtacctgg

SEQ ID NO: 119 - DNA, 20 bp, synthetic construct
  source 1..20: mol_type = other DNA
gagccacgaaaacagatcca

SEQ ID NO: 120 - DNA, 20 bp, synthetic construct
  source 1..20: mol_type = other DNA
aagtgaacggggaagggagg

SEQ ID NO: 121 - DNA, 20 bp, synthetic construct
  source 1..20: mol_type = other DNA
gacaaaagccgaagtccagg

SEQ ID NO: 122 - DNA, 20 bp, synthetic construct
  source 1..20: mol_type = other DNA
gtggttgataaacccacgtg

SEQ ID NO: 123 - DNA, 20 bp, synthetic construct
  source 1..20: mol_type = other DNA
tgggaacagccacagcaggg

SEQ ID NO: 124 - DNA, 20 bp, synthetic construct
  source 1..20: mol_type = other DNA
gcaggggaacggggatgcag

SEQ ID NO: 125 - DNA, 20 bp, synthetic construct
  source 1..20: mol_type = other DNA
gagatggtggacgaggaagg

SEQ ID NO: 126 - DNA, 20 bp, synthetic construct
  source 1..20: mol_type = other DNA
gagatggctccaggaaatgg

SEQ ID NO: 127 - DNA, 20 bp, synthetic construct
  source 1..20: mol_type = other DNA
taaggaatctgcctaacagg

SEQ ID NO: 128 - DNA, 20 bp, synthetic construct
  source 1..20: mol_type = other DNA
tcaggagactaggaaggagg

SEQ ID NO: 129 - DNA, 20 bp, synthetic construct
  source 1..20: mol_type = other DNA
tataaggtggtcccagctcg

SEQ ID NO: 130 - DNA, 20 bp, synthetic construct
  source 1..20: mol_type = other DNA
ctggaagatgccatgacagg

SEQ ID NO: 131 - DNA, 20 bp, synthetic construct
  source 1..20: mol_type = other DNA
gcacagactagagaggtaag

SEQ ID NO: 132 - DNA, 20 bp, synthetic construct
  source 1..20: mol_type = other DNA
acagactagagaggtaaggg

SEQ ID NO: 133 - DNA, 20 bp, synthetic construct
  source 1..20: mol_type = other DNA
gagaggtgacccgaatccac

SEQ ID NO: 134 - DNA, 20 bp, synthetic construct
  source 1..20: mol_type = other DNA
gcacaggccccagaaggaga

SEQ ID NO: 135 - DNA, 20 bp, synthetic construct
  source 1..20: mol_type = other DNA
ccggagaggacccagacacg

SEQ ID NO: 136 - DNA, 20 bp, synthetic construct
  source 1..20: mol_type = other DNA
gagaggacccagacacgggg

SEQ ID NO: 137 - DNA, 20 bp, synthetic construct
  source 1..20: mol_type = other DNA
gcaacacagcagagagcaag

SEQ ID NO: 138 - DNA, 20 bp, synthetic construct
  source 1..20: mol_type = other DNA
gaagagggagtggaggaaga

SEQ ID NO: 139 - DNA, 20 bp, synthetic construct
  source 1..20: mol_type = other DNA
aagacggaacctgaaggagg

SEQ ID NO: 140 - DNA, 20 bp, synthetic construct
  source 1..20: mol_type = other DNA
agaaagcggcacaggcccag

SEQ ID NO: 141 - DNA, 20 bp, synthetic construct
  source 1..20: mol_type = other DNA
gggaaacagtgggccagagg

SEQ ID NO: 142 - DNA, 20 bp, synthetic construct
  source 1..20: mol_type = other DNA
gtccggactcaggagagaga

SEQ ID NO: 143 - DNA, 20 bp, synthetic construct
  source 1..20: mol_type = other DNA
ggcacagcaagggcactcgg

SEQ ID NO: 144 - DNA, 19 bp, synthetic construct
  source 1..19: mol_type = other DNA
gaagaggggaagtcgaggg

SEQ ID NO: 145 - DNA, 20 bp, synthetic construct
  source 1..20: mol_type = other DNA
gggaatggtaaggaggcctg

SEQ ID NO: 146 - DNA, 20 bp, synthetic construct
  source 1..20: mol_type = other DNA
gcagagtggtcagcacagag

SEQ ID NO: 147 - DNA, 20 bp, synthetic construct
  source 1..20: mol_type = other DNA
gcacagagtggctaagccca

SEQ ID NO: 148 - DNA, 20 bp, synthetic construct
  source 1..20: mol_type = other DNA
gacggggtgtcagcataggg

SEQ ID NO: 149 - DNA, 20 bp, synthetic construct
  source 1..20: mol_type = other DNA
gcccagggccaggaacgacg

SEQ ID NO: 150 - DNA, 20 bp, synthetic construct
  source 1..20: mol_type = other DNA
ggtggagtccagcacggcgc

SEQ ID NO: 151 - DNA, 20 bp, synthetic construct
  source 1..20: mol_type = other DNA
acaggccgccaggaactcgg

SEQ ID NO: 152 - DNA, 20 bp, synthetic construct
  source 1..20: mol_type = other DNA
actaggaagtgtgtagcacc

SEQ ID NO: 153 - DNA, 20 bp, synthetic construct
  source 1..20: mol_type = other DNA
atgaatagcagactgccccg

SEQ ID NO: 154 - DNA, 20 bp, synthetic construct
  source 1..20: mol_type = other DNA
acacccctaaaagcacagtg

SEQ ID NO: 155 - DNA, 20 bp, synthetic construct
  source 1..20: mol_type = other DNA
caaggagttccagcaggtgg

SEQ ID NO: 156 - DNA, 20 bp, synthetic construct
  source 1..20: mol_type = other DNA
aaggagttccagcaggtggg

SEQ ID NO: 157 - DNA, 20 bp, synthetic construct
  source 1..20: mol_type = other DNA
tggaaagaggagggaagagg

SEQ ID NO: 158 - DNA, 20 bp, synthetic construct
  source 1..20: mol_type = other DNA
tcgaattcctaactgccccg

SEQ ID NO: 159 - DNA, 20 bp, synthetic construct
  source 1..20: mol_type = other DNA
gacctgcccagcacaccctg

SEQ ID NO: 160 - DNA, 20 bp, synthetic construct
  source 1..20: mol_type = other DNA
ggagcagctgcggcagtggg

SEQ ID NO: 161 - DNA, 20 bp, synthetic construct
  source 1..20: mol_type = other DNA
gggagggagagcttggcagg

SEQ ID NO: 162 - DNA, 20 bp, synthetic construct
  source 1..20: mol_type = other DNA
gttacgtggccaagaagcag

SEQ ID NO: 163 - DNA, 20 bp, synthetic construct
  source 1..20: mol_type = other DNA
gctgaacagagaagagctgg

SEQ ID NO: 164 - DNA, 20 bp, synthetic construct
  source 1..20: mol_type = other DNA
tctgagggtggagggactgg

SEQ ID NO: 165 - DNA, 20 bp, synthetic construct
  source 1..20: mol_type = other DNA
ggagaggtgagggacttggg

SEQ ID NO: 166 - DNA, 20 bp, synthetic construct
  source 1..20: mol_type = other DNA
gtgaaccaggcagacaacga

SEQ ID NO: 167 - DNA, 20 bp, synthetic construct
  source 1..20: mol_type = other DNA
caggtacctcctgagccacg

SEQ ID NO: 168 - DNA, 20 bp, synthetic construct
  source 1..20: mol_type = other DNA
gggggagtaggggcatgcag

SEQ ID NO: 169 - DNA, 20 bp, synthetic construct
  source 1..20: mol_type = other DNA
gcaaatggccagcaagggtg

SEQ ID NO: 170 - DNA, 25 bp, synthetic construct
  source 1..25: mol_type = other DNA
caccgaatcgagaagcgactcgaca

SEQ ID NO: 171 - DNA, 25 bp, synthetic construct
  source 1..25: mol_type = other DNA
caccggtccctgggcgttgccctgc

SEQ ID NO: 172 - DNA, 25 bp, synthetic construct
  source 1..25: mol_type = other DNA
caccgccctgggcgttgccctgcag

SEQ ID NO: 173 - DNA, 24 bp, synthetic construct
  source 1..24: mol_type = other DNA
caccgccgtgggaagataaactaa

SEQ ID NO: 174 - DNA, 25 bp, synthetic construct
  source 1..25: mol_type = other DNA
caccgtcccctgcagggcaacgccc

SEQ ID NO: 175 - DNA, 25 bp, synthetic construct
  source 1..25: mol_type = other DNA
caccggtcgagtcgcttctcgatta

SEQ ID NO: 176 - DNA, 25 bp, synthetic construct
  source 1..25: mol_type = other DNA
caccgctgctgcctcccgtcttgta

SEQ ID NO: 177 - DNA, 25 bp, synthetic construct
  source 1..25: mol_type = other DNA
caccggagtgccgcaatacctttat

SEQ ID NO: 178 - DNA, 25 bp, synthetic construct
  source 1..25: mol_type = other DNA
caccgacactttggtggtgcagcaa

SEQ ID NO: 179 - DNA, 25 bp, synthetic construct
  source 1..25: mol_type = other DNA
caccgtctcaaatggtataaaactc

SEQ ID NO: 180 - DNA, 25 bp, synthetic construct
  source 1..25: mol_type = other DNA
caccgaatcccgcccataatcgaga

SEQ ID NO: 181 - DNA, 25 bp, synthetic construct
  source 1..25: mol_type = other DNA
caccgtcccgcccataatcgagaag

SEQ ID NO: 182 - DNA, 25 bp, synthetic construct
  source 1..25: mol_type = other DNA
caccgcccataatcgagaagcgact

SEQ ID NO: 183 - DNA, 25 bp, synthetic construct
  source 1..25: mol_type = other DNA
caccggagaagcgactcgacatgga

SEQ ID NO: 184 - DNA, 25 bp, synthetic construct
  source 1..25: mol_type = other DNA
caccggaagcgactcgacatggagg

SEQ ID NO: 185 - DNA, 25 bp, synthetic construct
  source 1..25: mol_type = other DNA
caccggcgactcgacatggaggcga

SEQ ID NO: 186 - DNA, 25 bp, synthetic construct
  source 1..25: mol_type = other DNA
aaactgtcgagtcgcttctcgattc

SEQ ID NO: 187 - DNA, 25 bp, synthetic construct
  source 1..25: mol_type = other DNA
aaacgcagggcaacgcccagggacc

SEQ ID NO: 188 - DNA, 25 bp, synthetic construct
  source 1..25: mol_type = other DNA
aaacctgcagggcaacgcccagggc

SEQ ID NO: 189 - DNA, 25 bp, synthetic construct
  source 1..25: mol_type = other DNA
aaacattagtttatcttcccacggc

SEQ ID NO: 190 - DNA, 25 bp, synthetic construct
  source 1..25: mol_type = other DNA
aaacgggcgttgccctgcaggggac

SEQ ID NO: 191 - DNA, 25 bp, synthetic construct
  source 1..25: mol_type = other DNA
aaactaatcgagaagcgactcgacc

SEQ ID NO: 192 - DNA, 25 bp, synthetic construct
  source 1..25: mol_type = other DNA
aaactacaagacgggaggcagcagc

SEQ ID NO: 193 - DNA, 25 bp, synthetic construct
  source 1..25: mol_type = other DNA
aaacataaaggtattgcggcactcc

SEQ ID NO: 194 - DNA, 25 bp, synthetic construct
  source 1..25: mol_type = other DNA
aaacttgctgcaccaccaaagtgtc

SEQ ID NO: 195 - DNA, 25 bp, synthetic construct
  source 1..25: mol_type = other DNA
aaacgagttttataccatttgagac

SEQ ID NO: 196 - DNA, 25 bp, synthetic construct
  source 1..25: mol_type = other DNA
aaactctcgattatgggcgggattc

SEQ ID NO: 197 - DNA, 25 bp, synthetic construct
  source 1..25: mol_type = other DNA
aaaccttctcgattatgggcgggac

SEQ ID NO: 198 - DNA, 25 bp, synthetic construct
  source 1..25: mol_type = other DNA
aaacagtcgcttctcgattatgggc

SEQ ID NO: 199 - DNA, 25 bp, synthetic construct
  source 1..25: mol_type = other DNA
aaactccatgtcgagtcgcttctcc

SEQ ID NO: 200 - DNA, 25 bp, synthetic construct
  source 1..25: mol_type = other DNA
aaaccctccatgtcgagtcgcttcc

SEQ ID NO: 201 - DNA, 25 bp, synthetic construct
  source 1..25: mol_type = other DNA
aaactcgcctccatgtcgagtcgcc

SEQ ID NO: 202 - DNA, 25 bp, synthetic construct
  source 1..25: mol_type = other DNA
caccgacagggttaatgtgaagtcc

SEQ ID NO: 203 - DNA, 25 bp, synthetic construct
  source 1..25: mol_type = other DNA
caccgtccccctctacatttaaagt

SEQ ID NO: 204 - DNA, 25 bp, synthetic construct
  source 1..25: mol_type = other DNA
caccgcatttaaagttggtttaagt

SEQ ID NO: 205 - DNA, 25 bp, synthetic construct
  source 1..25: mol_type = other DNA
caccgttagaaaatataaagaataa

SEQ ID NO: 206 - DNA, 25 bp, synthetic construct
  source 1..25: mol_type = other DNA
caccgtaaatgcttactggtttgaa

SEQ ID NO: 207 - DNA, 25 bp, synthetic construct
  source 1..25: mol_type = other DNA
caccgtcctgggtccagaaaaagat

SEQ ID NO: 208 - DNA, 25 bp, synthetic construct
  source 1..25: mol_type = other DNA
caccgttgggtggtgagcatctgtg

SEQ ID NO: 209 - DNA, 25 bp, synthetic construct
  source 1..25: mol_type = other DNA
caccgcggggagagtggagaaaaag

SEQ ID NO: 210 - DNA, 25 bp, synthetic construct
  source 1..25: mol_type = other DNA
caccggttaaaactctttagacaac

SEQ ID NO: 211 - DNA, 25 bp, synthetic construct
  source 1..25: mol_type = other DNA
caccggaaaatccccactaagatcc

SEQ ID NO: 212 - DNA, 25 bp, synthetic construct
  source 1..25: mol_type = other DNA
aaacggacttcacattaaccctgtc

SEQ ID NO: 213 - DNA, 25 bp, synthetic construct
  source 1..25: mol_type = other DNA
aaacactttaaatgtagagggggac

SEQ ID NO: 214 - DNA, 25 bp, synthetic construct
  source 1..25: mol_type = other DNA
aaacacttaaaccaactttaaatgc

SEQ ID NO: 215 - DNA, 25 bp, synthetic construct
  source 1..25: mol_type = other DNA
aaacttattctttatattttctaac

SEQ ID NO: 216 - DNA, 25 bp, synthetic construct
  source 1..25: mol_type = other DNA
aaacttcaaaccagtaagcatttac

SEQ ID NO: 217 - DNA, 25 bp, synthetic construct
  source 1..25: mol_type = other DNA
aaacatctttttctggacccaggac

SEQ ID NO: 218 - DNA, 25 bp, synthetic construct
  source 1..25: mol_type = other DNA
aaaccacagatgctcaccacccaac

SEQ ID NO: 219 - DNA, 25 bp, synthetic construct
  source 1..25: mol_type = other DNA
aaacctttttctccactctccccgc

SEQ ID NO: 220 - DNA, 25 bp, synthetic construct
  source 1..25: mol_type = other DNA
aaacgttgtctaaagagttttaacc

SEQ ID NO: 221 - DNA, 25 bp, synthetic construct
  source 1..25: mol_type = other DNA
aaacggatcttagtggggattttcc

SEQ ID NO: 222 - DNA, 20 bp, synthetic construct
  source 1..20: mol_type = other DNA
agtagcagtaatgaagctgg

SEQ ID NO: 223 - DNA, 20 bp, synthetic construct
  source 1..20: mol_type = other DNA
atacccagacgagaaagctg

SEQ ID NO: 224 - DNA, 20 bp, synthetic construct
  source 1..20: mol_type = other DNA
tacccagacgagaaagctga

SEQ ID NO: 225 - DNA, 20 bp, synthetic construct
  source 1..20: mol_type = other DNA
ggtggtgagcatctgtgtgg

SEQ ID NO: 226 - DNA, 20 bp, synthetic construct
  source 1..20: mol_type = other DNA
aaatgagaagaagaggcaca

SEQ ID NO: 227 - DNA, 20 bp, synthetic construct
  source 1..20: mol_type = other DNA
cttgtggcctgggagagctg

SEQ ID NO: 228 - DNA, 20 bp, synthetic construct
  source 1..20: mol_type = other DNA
gctgtagaaggagacagagc

SEQ ID NO: 229 - DNA, 20 bp, synthetic construct
  source 1..20: mol_type = other DNA
gagctggttgggaagacatg

SEQ ID NO: 230 - DNA, 20 bp, synthetic construct
  source 1..20: mol_type = other DNA
ctggttgggaagacatgggg

SEQ ID NO: 231 - DNA, 20 bp, synthetic construct
  source 1..20: mol_type = other DNA
cgtgaggatgggaaggaggg

SEQ ID NO: 232 - DNA, 20 bp, synthetic construct
  source 1..20: mol_type = other DNA
atgcagagtcagcagaactg

SEQ ID NO: 233 - DNA, 20 bp, synthetic construct
  source 1..20: mol_type = other DNA
aagacatcaagcacagaagg

SEQ ID NO: 234 - DNA, 20 bp, synthetic construct
  source 1..20: mol_type = other DNA
tcaagcacagaaggaggagg

SEQ ID NO: 235 - DNA, 20 bp, synthetic construct
  source 1..20: mol_type = other DNA
aaccgtcaataggcaaaggg

SEQ ID NO: 236 - DNA, 20 bp, synthetic construct
  source 1..20: mol_type = other DNA
ccgtatttcagactgaatgg

SEQ ID NO: 237 - DNA, 20 bp, synthetic construct
  source 1..20: mol_type = other DNA
gagaggacaggtgctacagg

SEQ ID NO: 238 - DNA, 20 bp, synthetic construct
  source 1..20: mol_type = other DNA
aaccaaggaagggcaggagg

SEQ ID NO: 239 - DNA, 20 bp, synthetic construct
  source 1..20: mol_type = other DNA
gacctctgggtggagacaga

SEQ ID NO: 240 - DNA, 20 bp, synthetic construct
  source 1..20: mol_type = other DNA
cagatgaccatgacaagcag

SEQ ID NO: 241 - DNA, 20 bp, synthetic construct
  source 1..20: mol_type = other DNA
aacaccagtgagtagagcgg

SEQ ID NO: 242 - DNA, 20 bp, synthetic construct
  source 1..20: mol_type = other DNA
aggaccttgaagcacagaga

SEQ ID NO: 243 - DNA, 20 bp, synthetic construct
  source 1..20: mol_type = other DNA
tacagaggcagactaaccca

SEQ ID NO: 244 - DNA, 20 bp, synthetic construct
  source 1..20: mol_type = other DNA
acagaggcagactaacccag

SEQ ID NO: 245 - DNA, 20 bp, synthetic construct
  source 1..20: mol_type = other DNA
taaatgacgtgctagacctg

SEQ ID NO: 246 - DNA, 20 bp, synthetic construct
  source 1..20: mol_type = other DNA
agtaaccactcaggacaggg

SEQ ID NO: 247 - DNA, 20 bp, synthetic construct
  source 1..20: mol_type = other DNA
accacaaaacagaaacacca

SEQ ID NO: 248 - DNA, 20 bp, synthetic construct
  source 1..20: mol_type = other DNA
gtttgaagacaagcctgagg

SEQ ID NO: 249 - DNA, 20 bp, synthetic construct
  source 1..20: mol_type = other DNA
gctgaaccccaaaagacagg

SEQ ID NO: 250 - DNA, 20 bp, synthetic construct
  source 1..20: mol_type = other DNA
gcagctgagacacacaccag

SEQ ID NO: 251 - DNA, 20 bp, synthetic construct
  source 1..20: mol_type = other DNA
aggacaccccaaagaagctg

SEQ ID NO: 252 - DNA, 20 bp, synthetic construct
  source 1..20: mol_type = other DNA
ggacaccccaaagaagctga

SEQ ID NO: 253 - DNA, 20 bp, synthetic construct
  source 1..20: mol_type = other DNA
ccagtgcaatggacagaaga

SEQ ID NO: 254 - DNA, 20 bp, synthetic construct
  source 1..20: mol_type = other DNA
agaagagggagcctgcaagt

SEQ ID NO: 255 - DNA, 20 bp, synthetic construct
  source 1..20: mol_type = other DNA
gtgtttgggccctagagcga

SEQ ID NO: 256 - DNA, 20 bp, synthetic construct
  source 1..20: mol_type = other DNA
catgtgcctggtgcaatgca

SEQ ID NO: 257 - DNA, 20 bp, synthetic construct
  source 1..20: mol_type = other DNA
tacaaagaggaagataagtg

SEQ ID NO: 258 - DNA, 20 bp, synthetic construct
  source 1..20: mol_type = other DNA
gtcacagaatacaccactag

SEQ ID NO: 259 - DNA, 20 bp, synthetic construct
  source 1..20: mol_type = other DNA
gggttaccctggacatggaa

SEQ ID NO: 260 - DNA, 20 bp, synthetic construct
  source 1..20: mol_type = other DNA
catggaagggtattcactcg

SEQ ID NO: 261 - DNA, 20 bp, synthetic construct
  source 1..20: mol_type = other DNA
agagtggcctagacaggctg

SEQ ID NO: 262 - DNA, 20 bp, synthetic construct
  source 1..20: mol_type = other DNA
catgctggacagctcggcag

SEQ ID NO: 263 - DNA, 20 bp, synthetic construct
  source 1..20: mol_type = other DNA
agtgaaagaagagaaaattc

SEQ ID NO: 264 - DNA, 20 bp, synthetic construct
  source 1..20: mol_type = other DNA
tggtaagtctaagaaaccta

SEQ ID NO: 265 - DNA, 20 bp, synthetic construct
  source 1..20: mol_type = other DNA
cccacagcctaaccacccta

SEQ ID NO: 266 - DNA, 20 bp, synthetic construct
  source 1..20: mol_type = other DNA
aatatttcaaagccctaggg

SEQ ID NO: 267 - DNA, 20 bp, synthetic construct
  source 1..20: mol_type = other DNA
gcactcggaacagggtctgg

SEQ ID NO: 268 - DNA, 20 bp, synthetic construct
  source 1..20: mol_type = other DNA
agataggagctccaacagtg

SEQ ID NO: 269 - DNA, 20 bp, synthetic construct
  source 1..20: mol_type = other DNA
aagttagagcagccaggaaa

SEQ ID NO: 270 - DNA, 20 bp, synthetic construct
  source 1..20: mol_type = other DNA
tagagcagccaggaaaggga

SEQ ID NO: 271 - DNA, 20 bp, synthetic construct
  source 1..20: mol_type = other DNA
tgaatacccttccatgtcca

SEQ ID NO: 272 - DNA, 20 bp, synthetic construct
  source 1..20: mol_type = other DNA
cctgcattgcaccaggcaca

SEQ ID NO: 273 - DNA, 20 bp, synthetic construct
  source 1..20: mol_type = other DNA
tctagggcccaaacacacct

SEQ ID NO: 274 - DNA, 20 bp, synthetic construct
  source 1..20: mol_type = other DNA
tccctccatctatcaaaagg

SEQ ID NO: 275 - DNA, 20 bp, synthetic construct
  source 1..20: mol_type = other DNA
agccctgagacagaagcagg

SEQ ID NO: 276 - DNA, 20 bp, synthetic construct
  source 1..20: mol_type = other DNA
gccctgagacagaagcaggt

SEQ ID NO: 277 - DNA, 20 bp, synthetic construct
  source 1..20: mol_type = other DNA
aggagatgcagtgatacgca

SEQ ID NO: 278 - DNA, 20 bp, synthetic construct
  source 1..20: mol_type = other DNA
acaataccaagggtatccgg

SEQ ID NO: 279 - DNA, 20 bp, synthetic construct
  source 1..20: mol_type = other DNA
tgataaagaaaacaaagtga

SEQ ID NO: 280 - DNA, 20 bp, synthetic construct
  source 1..20: mol_type = other DNA
aaagaaaacaaagtgaggga

SEQ ID NO: 281 - DNA, 20 bp, synthetic construct
  source 1..20: mol_type = other DNA
gtggcaagtggagaaattga

SEQ ID NO: 282 - DNA, 20 bp, synthetic construct
  source 1..20: mol_type = other DNA
caagtggagaaattgaggga

SEQ ID NO: 283 - DNA, 20 bp, synthetic construct
  source 1..20: mol_type = other DNA
gtggtgatgattgcagctgg

SEQ ID NO: 284 - DNA, 20 bp, synthetic construct
  source 1..20: mol_type = other DNA
ctatgtgcctgacacacagg

SEQ ID NO: 285 - DNA, 20 bp, synthetic construct
  source 1..20: mol_type = other DNA
gggttggaccaggaaagagg

SEQ ID NO: 286 - DNA, 20 bp, synthetic construct
  source 1..20: mol_type = other DNA
gatgcctggaaaaggaaaga

SEQ ID NO: 287 - DNA, 20 bp, synthetic construct
  source 1..20: mol_type = other DNA
tagtatgcacctgcaagagg

SEQ ID NO: 288 - DNA, 20 bp, synthetic construct
  source 1..20: mol_type = other DNA
tatgcacctgcaagaggcgg

SEQ ID NO: 289 - DNA, 20 bp, synthetic construct
  source 1..20: mol_type = other DNA
aggggaagaagagaagcaga

SEQ ID NO: 290 - DNA, 20 bp, synthetic construct
  source 1..20: mol_type = other DNA
gctgaatcaagagacaagcg

SEQ ID NO: 291 - DNA, 20 bp, synthetic construct
  source 1..20: mol_type = other DNA
aagcaaataaatctcctggg

SEQ ID NO: 292 - DNA, 20 bp, synthetic construct
  source 1..20: mol_type = other DNA
agatgagtgctagagactgg

SEQ ID NO: 293 - DNA, 20 bp, synthetic construct
  source 1..20: mol_type = other DNA
ctgatggttgagcacagcag

SEQ ID NO: 294 - DNA, 20 bp, synthetic construct
  source 1..20: mol_type = other DNA
caaatggccagcaagggtgg

SEQ ID NO: 295 - DNA, 20 bp, synthetic construct
  source 1..20: mol_type = other DNA
gcagaacctgaggatatgga

SEQ ID NO: 296 - DNA, 20 bp, synthetic construct
  source 1..20: mol_type = other DNA
aatacacagaatgaaaatag

SEQ ID NO: 297 - DNA, 20 bp, synthetic construct
  source 1..20: mol_type = other DNA
ctggtgactagaataggcag

SEQ ID NO: 298 - DNA, 20 bp, synthetic construct
  source 1..20: mol_type = other DNA
tggtgactagaataggcagt

SEQ ID NO: 299 - DNA, 20 bp, synthetic construct
  source 1..20: mol_type = other DNA
taaaagaatgtgaaaagatg

SEQ ID NO: 300 - DNA, 20 bp, synthetic construct
  source 1..20: mol_type = other DNA
tcaggagttcaagaccaccc

SEQ ID NO: 301 - DNA, 20 bp, synthetic construct
  source 1..20: mol_type = other DNA
tgtagtcccagttatgcagg

SEQ ID NO: 302 - DNA, 20 bp, synthetic construct
  source 1..20: mol_type = other DNA
gggttcacaccacaaatgca

SEQ ID NO: 303 - DNA, 20 bp, synthetic construct
  source 1..20: mol_type = other DNA
ggcaaatggccagcaagggt

SEQ ID NO: 304 - DNA, 20 bp, synthetic construct
  source 1..20: mol_type = other DNA
agaaaccaatcccaaagcaa

SEQ ID NO: 305 - DNA, 20 bp, synthetic construct
  source 1..20: mol_type = other DNA
gccaaggacaccaaaaccca

SEQ ID NO: 306 - DNA, 20 bp, synthetic construct
  source 1..20: mol_type = other DNA
agtggtgataaggcaacagt

SEQ ID NO: 307 - DNA, 20 bp, synthetic construct
  source 1..20: mol_type = other DNA
cctgagacagaagtattaag

SEQ ID NO: 308 - DNA, 20 bp, synthetic construct
  source 1..20: mol_type = other DNA
aaggtcacacaatgaatagg

SEQ ID NO: 309 - DNA, 20 bp, synthetic construct
  source 1..20: mol_type = other DNA
caccatactagggaagaaga

SEQ ID NO: 310 - DNA, 20 bp, synthetic construct
  source 1..20: mol_type = other DNA
caataccctgcccttagtgg

SEQ ID NO: 311 - DNA, 20 bp, synthetic construct
  source 1..20: mol_type = other DNA
aataccctgcccttagtggg

SEQ ID NO: 312 - DNA, 20 bp, synthetic construct
  source 1..20: mol_type = other DNA
ttagtggggggtggagtggg

SEQ ID NO: 313 - DNA, 20 bp, synthetic construct
  source 1..20: mol_type = other DNA
gtggggggtggagtgggggg

SEQ ID NO: 314 - DNA, 20 bp, synthetic construct
  source 1..20: mol_type = other DNA
ggggggtggagtggggggtg

SEQ ID NO: 315 - DNA, 20 bp, synthetic construct
  source 1..20: mol_type = other DNA
ggggtggagtggggggtggg

SEQ ID NO: 316 - DNA, 20 bp, synthetic construct
  source 1..20: mol_type = other DNA
gggtggagtggggggtgggg

SEQ ID NO: 317 - DNA, 20 bp, synthetic construct
  source 1..20: mol_type = other DNA
gggggtggggaaagacatcg

SEQ ID NO: 318 - DNA, 20 bp, synthetic construct
  source 1..20: mol_type = other DNA
gcagctgtgaattctgatag

SEQ ID NO: 319 - DNA, 20 bp, synthetic construct
  source 1..20: mol_type = other DNA
gagatcagagaaaccagatg

SEQ ID NO: 320 - DNA, 20 bp, synthetic construct
  source 1..20: mol_type = other DNA
tctatactgattgcagccag

SEQ ID NO: 321 - protein, 9 aa, synthetic construct
PAAKRVKLD

SEQ ID NO: 322 - protein, 16 aa, synthetic construct
KRPAATKKAGQAKKKK

SEQ ID NO: 323 - protein, 7 aa, synthetic construct
PKKKRKV

SEQ ID NO: 324 - protein, 24 aa, synthetic construct
DPKKKRKVDPKKKRKVDPKKKRKV

SEQ ID NO: 325 - DNA, 333 bp, synthetic construct
  source 1..333: mol_type = other DNA
ggcggatctggcggtagtgctgagtattgtctgagttacgaaacggaaatactcacggttgagtatgggcttcttccaattggcaaaatcgttgaaaagcgcatagagtgtacggtgtattccgtcgataacaacggtaatatctacacccagccggtagctcagtggcacgaccgaggcgaacaggaagtgttcgagtattgcttggaagatggctcccttatccgcgccactaaagaccataagtttatgacggttgacgggcagatgctgcctatagacgaaatatttgagagagagctggacttgatgagagtcgataatctgccaaat

SEQ ID NO: 326 - DNA, 180 bp, synthetic construct
  source 1..180: mol_type = other DNA
ggcggatctggcggtagtgggggttccggatccataaagatagctactaggaaatatcttggcaaacaaaacgtctatgacataggagttgagcgagatcacaattttgctttgaagaatgggttcatcgcgtctaattgcttcaacgctagcggcgggtcaggaggctctggtggaagc

SEQ ID NO: 327 - DNA, 20 bp, synthetic construct
  source 1..20: mol_type = other DNA
aatcgagaagcgactcgaca

SEQ ID NO: 328 - DNA, 20 bp, synthetic construct
  source 1..20: mol_type = other DNA
tgccctgcaggggagtgagc

SEQ ID NO: 329 - DNA, 20 bp, synthetic construct
  source 1..20: mol_type = other DNA
gaagcgactcgacatggagg

SEQ ID NO: 330 - DNA, 20 bp, synthetic construct
  source 1..20: mol_type = other DNA
cctgcaggggagtgagcagc

SEQ ID NO: 331 - protein, 638 aa, synthetic construct; identical to SEQ ID NO: 1

SEQ ID NO: 332 - protein, 603 aa, synthetic construct
MASGDDEQIAGPSGTTERKKSLALPKDLAESTDSDSDIEFIKAKRRRTIVYSSESDGDIGDIIEKSGIRPSESYVSRGKQEKEKWTSTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 333 - DNA, 1809 bp, synthetic construct
  source 1..1809: mol_type = other DNA
atggctagcggcgacgacgagcagatcgctggacccagcgggaccacggagcgcaaaaagagcctggctctgcctaaagacttggccgagagtaccgacagcgactccgatatcgagttcatcaaggccaaacgcaggcgcacaatcgtgtactcttccgagagcgacggcgacatcggcgatattatcgagaaaagcgggatccggccttccgaaagctacgtgtctcggggcaagcaggagaaggaaaagtggacaagcacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtgggggagtgcttcaagaagtaccacacaatgaaaaactacagggat

SEQ ID NO: 334 - protein, 592 aa, synthetic construct
MSGTTERKKSLALPKDLAESTDSDSDIEFIKAKRRRTIVYSSESDGDIGDIIEKSGIRPSESYVSRGKQEKEKWTSTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 335 - DNA, 1776 bp, synthetic construct
  source 1..1776: mol_type = other DNA
atgagcgggaccacggagcgcaaaaagagcctggctctgcctaaagacttggccgagagtaccgacagcgactccgatatcgagttcatcaaggccaaacgcaggcgcacaatcgtgtactcttccgagagcgacggcgacatcggcgatattatcgagaaaagcgggatccggccttccgaaagctacgtgtctcggggcaagcaggagaaggaaaagtggacaagcacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtgggggagtgcttcaagaagtaccacacaatgaaaaactacagggat

SEQ ID NO: 336 - protein, 522 aa, synthetic construct
MEKWTSTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 337 - DNA, 1569 bp, synthetic construct
  source 1..1569: mol_type = other DNA
atggaaaagtggacaagcacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtgggggagtgcttcaagaagtaccacacaatgaaaaactacagggattaa

SEQ ID NO: 338 - protein, 519 aa, synthetic construct
MTSTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 339 - DNA, 1560 bp, synthetic construct
  source 1..1560: mol_type = other DNA
atgacaagcacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtgggggagtgcttcaagaagtaccacacaatgaaaaactacagggattaa

SEQ ID NO: 340 - protein, 517 aa, synthetic construct
MTSVNDKEPSRIPFSTGQLHVGPQVPSGCATPIDFFQLFFTETLIKNITDETNEYARHKISQKELSQRSTWNNWKDVTIEEMKAFLGVILNMGVLNHPNLQSYWSMDFESHIPFFRSVFKRERFLQIFWMLHLKNDQKSSKDLRTRTEKVNCFLSYLEMKFRERFCPGREIAVDEAVVGFKGKIHFITYNPKKPTKWGIRLYVLSDSKCGYVHSFVPYYGGITSETLVRPDLPFTSRIVLELHERLKNSVPGSQGYHFFTDRYYTSVTLAKELFKEKTHLTGTIMPNRKDNPPVIKHPKLMKGEIVAFRDENVMLLAWKDKRIVTMLSTWDTSETESVERRVRGGGKEIVLKPKVVTNYTKFMGGVDIADHYTGTYCFMRKTLKWWRKLFFWGLEVSVVNSYILYKECQKRKNEKPITHVKFIRKLVHDLVGEFRDGTLTSRGRLLSTNLEQRLDGKLHIITPHPNKKHKDCVVCSNRKIKGGRRETIYICETCECKPGLHVGECFKKYHTMKNYRD

SEQ ID NO: 341 - DNA, 1554 bp, synthetic construct
  source 1..1554: mol_type = other DNA
atgacctctgtgaacgacaaagagccttccagaatccccttcagcaccggccagctgcatgtgggcccccaggtgcccagcggctgcgccactcctatcgacttcttccagctgttttttactgagaccctgatcaagaacatcaccgatgagacaaatgagtacgccaggcacaagatctctcagaaggagctgagccagcgcagtacatggaacaactggaaggacgtgaccatcgaagagatgaaggccttcctgggcgtgatcctgaatatgggagtgctgaaccatcctaatctgcagtcctattggtccatggatttcgagtcccacattccattcttcaggtccgtgttcaagcgcgagcgtttcctgcagatcttctggatgctgcacctgaaaaatgaccagaagagctccaaggacctgcggacacggactgagaaggtgaattgtttcctgtcctacctggagatgaaattcagggagaggttttgtcccggccgggaaattgccgtggatgaggccgtggtgggcttcaagggcaagatccacttcatcacctacaacccaaagaagccaacaaagtggggcatccggctgtatgtcctgagtgactccaagtgtggctacgtgcacagctttgtgccctattatggcggcatcacctccgagaccctggtgaggcccgacctgcctttcacctctagaattgtgctggagctgcatgagcggctgaagaactctgtgcctggcagccagggctaccattttttcaccgacaggtactatacatccgttaccctggccaaggaactgttcaaagaaaaaacccacctgaccggcactatcatgcccaaccgcaaggacaacccccctgtgatcaaacatcccaaactgatgaagggcgagatcgtggccttcagagacgagaacgtcatgctgctggcttggaaagataagcggatcgtgactatgctgtctacatgggatacctccgagacagagagcgttgaacggcgggtgaggggtggaggcaaggagatcgtgctgaagccaaaggtggtgaccaactacaccaagttcatgggcggagtggatattgcagaccattacaccggcacctactgtttcatgcggaagaccctgaagtggtggcggaagctgttcttctgggggctggaggtcagcgtggtgaactcctacatcctctacaaggagtgccagaagaggaagaacgagaaaccaatcacacacgtgaagtttatcaggaagctggtgcacgacctggtgggagagttccgcgacggcaccctcaccagtcggggccggctgctgagtacaaacctggagcagaggctggacggaaagctgcacattatcactccccatccaaataagaagcacaaggactgcgtggtctgcagcaaccggaagattaaaggagggcggcgggaaaccatttacatttgtgagacctgcgaatgcaagcctggcctgcacgtgggggagtgcttcaagaagtaccacacaatgaaaaactacagggattaa

SEQ ID NO: 342 - DNA, 20 bp, synthetic construct
  source 1..20: mol_type = other DNA
aatcgagaagcgactcgaca

SEQ ID NO: 343 - DNA, 20 bp, synthetic construct
  source 1..20: mol_type = other DNA
gtccctgggcgttgccctgc

SEQ ID NO: 344 - DNA, 20 bp, synthetic construct
  source 1..20: mol_type = other DNA
ccctgggcgttgccctgcag

SEQ ID NO: 345 - DNA, 20 bp, synthetic construct
  source 1..20: mol_type = other DNA
gagtgagcagctgtaagatt

SEQ ID NO: 346 - DNA, 20 bp, synthetic construct
  source 1..20: mol_type = other DNA
caggggagtgagcagctgta

SEQ ID NO: 347 - DNA, 20 bp, synthetic construct
  source 1..20: mol_type = other DNA
cctgcaggggagtgagcagc

SEQ ID NO: 348 - DNA, 20 bp, synthetic construct
  source 1..20: mol_type = other DNA
tgccctgcaggggagtgagc

SEQ ID NO: 349 - DNA, 20 bp, synthetic construct
  source 1..20: mol_type = other DNA
cgttgccctgcaggggagtg

SEQ ID NO: 350 - DNA, 20 bp, synthetic construct
  source 1..20: mol_type = other DNA
tgggcgttgccctgcagggg

SEQ ID NO: 351 - DNA, 20 bp, synthetic construct
  source 1..20: mol_type = other DNA
ttggtccctgggcgttgccc

SEQ ID NO: 352 - DNA, 20 bp, synthetic construct
  source 1..20: mol_type = other DNA
aagaatcccgcccataatcg

SEQ ID NO: 353 - DNA, 20 bp, synthetic construct
  source 1..20: mol_type = other DNA
aatcccgcccataatcgaga

SEQ ID NO: 354 - DNA, 20 bp, synthetic construct
  source 1..20: mol_type = other DNA
tcccgcccataatcgagaag

SEQ ID NO: 355 - DNA, 20 bp, synthetic construct
  source 1..20: mol_type = other DNA
cccataatcgagaagcgact

SEQ ID NO: 356 - DNA, 20 bp, synthetic construct
  source 1..20: mol_type = other DNA
gagaagcgactcgacatgga

SEQ ID NO: 357 - DNA, 20 bp, synthetic construct
  source 1..20: mol_type = other DNA
gaagcgactcgacatggagg

SEQ ID NO: 358 - DNA, 20 bp, synthetic construct
  source 1..20: mol_type = other DNA
gcgactcgacatggaggcga

SEQ ID NO: 359 - DNA, 20 bp, synthetic construct
  source 1..20: mol_type = other DNA
ccgtgggaagataaactaat

SEQ ID NO: 360 - DNA, 20 bp, synthetic construct
  source 1..20: mol_type = other DNA
tcccctgcagggcaacgccc

SEQ ID NO: 361 - DNA, 20 bp, synthetic construct
  source 1..20: mol_type = other DNA
gtcgagtcgcttctcgatta

SEQ ID NO: 362 - DNA, 20 bp, synthetic construct
  source 1..20: mol_type = other DNA
cgacaccaactctagtccgt

SEQ ID NO: 363 - DNA, 20 bp, synthetic construct
  source 1..20: mol_type = other DNA
cagctgctcactcccctgca

SEQ ID NO: 364 - DNA, 20 bp, synthetic construct
  source 1..20: mol_type = other DNA
agtcgcttctcgattatggg

SEQ ID NO: 365 - DNA, 23 bp, synthetic construct
  source 1..23: mol_type = other DNA
acccttggaaggacctggctggg

SEQ ID NO: 366 - DNA, 20 bp, synthetic construct
  source 1..20: mol_type = other DNA
tccgagcttgacccttggaa

SEQ ID NO: 367 - DNA, 23 bp, synthetic construct
  source 1..23: mol_type = other DNA
ggagccacgaaaacagatccagg

SEQ ID NO: 368 - DNA, 20 bp, synthetic construct
  source 1..20: mol_type = other DNA
tggtttccgagcttgaccct

SEQ ID NO: 369 - DNA, 23 bp, synthetic construct
  source 1..23: mol_type = other DNA
ggagccacgaaaacagatccagg

SEQ ID NO: 370 - DNA, 23 bp, synthetic construct
  source 1..23: mol_type = other DNA
agatccagggacacggtgctagg

SEQ ID NO: 371 - DNA, 23 bp, synthetic construct
  source 1..23: mol_type = other DNA
gacacggtgctaggacagtgggg

SEQ ID NO: 372 - DNA, 23 bp, synthetic construct
  source 1..23: mol_type = other DNA
gaaaatgacccaacagcctctgg

SEQ ID NO: 373 - DNA, 23 bp, synthetic construct
  source 1..23: mol_type = other DNA
gcctggccggcctgaccactggg

SEQ ID NO: 374 - DNA, 23 bp, synthetic construct
  source 1..23: mol_type = other DNA
ctgagcactgaaggcctggccgg

SEQ ID NO: 375 - DNA, 23 bp, synthetic construct
  source 1..23: mol_type = other DNA
tggtttccactgagcactgaagg

SEQ ID NO: 376 - DNA, 23 bp, synthetic construct
  source 1..23: mol_type = other DNA
ggtgctttcctgaggaccgatag

SEQ ID NO: 377 - DNA, 23 bp, synthetic construct
  source 1..23: mol_type = other DNA
gcgcttccagtgctcagactagg

SEQ ID NO: 378 - DNA, 23 bp, synthetic construct
  source 1..23: mol_type = other DNA
cagtgctcagactagggaagagg

SEQ ID NO: 379 - DNA, 23 bp, synthetic construct
  source 1..23: mol_type = other DNA
gcccctcctccttcagagccagg

SEQ ID NO: 380 - DNA, 23 bp, synthetic construct
  source 1..23: mol_type = other DNA
tccttcagagccaggagtcctgg

SEQ ID NO: 381 - DNA, 23 bp, synthetic construct
  source 1..23: mol_type = other DNA
ccaagggtcaagctcggaaacca

SEQ ID NO: 382 - DNA, 23 bp, synthetic construct
  source 1..23: mol_type = other DNA
ctgcagagtatctgctggggtgg

SEQ ID NO: 383 - DNA, 23 bp, synthetic construct
  source 1..23: mol_type = other DNA
cgttcctgcagagtatctgctgg

SEQ ID NO: 384 - DNA, 20 bp, synthetic construct
  source 1..20: mol_type = other DNA
gtggggaaaatgacccaaca

SEQ ID NO: 385 - DNA, 20 bp, synthetic construct
  source 1..20: mol_type = other DNA
gaaggcctggccggcctgac

SEQ ID NO: 386 - DNA, 20 bp, synthetic construct
  source 1..20: mol_type = other DNA
actcctggctctgaaggagg

SEQ ID NO: 387 - DNA, 20 bp, synthetic construct
  source 1..20: mol_type = other DNA
gggctgggggccaggactcc

SEQ ID NO: 388 - DNA, 20 bp, synthetic construct
  source 1..20: mol_type = other DNA
gtccttccaagggtcaagct

SEQ ID NO: 389 - DNA, 20 bp, synthetic construct
  source 1..20: mol_type = other DNA
tcaagctcggaaaccacccc

SEQ ID NO: 390 - DNA, 23 bp, synthetic construct
  source 1..23: mol_type = other DNA
attgtcttcactaaacccgttgg

SEQ ID NO: 391 - DNA, 23 bp, synthetic construct
  source 1..23: mol_type = other DNA
taaacccgttgggaatacaatgg

SEQ ID NO: 392 - DNA, 23 bp, synthetic construct
  source 1..23: mol_type = other DNA
ttgtcttcactaaacccgttggg

SEQ ID NO: 393 - DNA, 23 bp, synthetic construct
  source 1..23: mol_type = other DNA
tgattcataggagtctattaagg

SEQ ID NO: 394 - DNA, 23 bp, synthetic construct
  source 1..23: mol_type = other DNA
ttacatatgcttcgagtttgtgg

SEQ ID NO: 395 - DNA, 23 bp, synthetic construct
  source 1..23: mol_type = other DNA
actcttaaggtaggactaattgg

SEQ ID NO: 396 - DNA, 23 bp, synthetic construct
  source 1..23: mol_type = other DNA
tatgtgtgcaatagcgttaaagg

SEQ ID NO: 397 - DNA, 23 bp, synthetic construct
  source 1..23: mol_type = other DNA
cgttgggaatacaatggcttagg

SEQ ID NO: 398 - DNA, 23 bp, synthetic construct
  source 1..23: mol_type = other DNA
tcacaatggaactctgcctttgg

SEQ ID NO: 399 - DNA, 23 bp, synthetic construct
  source 1..23: mol_type = other DNA
gaccacaaatcaatgcccaaagg

SEQ ID NO: 400 - DNA, 23 bp, synthetic construct
  source 1..23: mol_type = other DNA
ctaagccattgtattcccaacgg

SEQ ID NO: 401 - DNA, 23 bp, synthetic construct
  source 1..23: mol_type = other DNA
agcattctggagtgtcacaatgg

SEQ ID NO: 402 - DNA, 23 bp, synthetic construct
  source 1..23: mol_type = other DNA
caatagcccactttaatactagg

SEQ ID NO: 403 - DNA, 23 bp, synthetic construct
  source 1..23: mol_type = other DNA
ctttatccaagtgaatcctttgg

SEQ ID NO: 404 - DNA, 23 bp, synthetic construct
  source 1..23: mol_type = other DNA
ggcattgatttgtggtcatttgg

SEQ ID NO: 405 - DNA, 23 bp, synthetic construct
  source 1..23: mol_type = other DNA
taagccattgtattcccaacggg

SEQ ID NO: 406 - DNA, 23 bp, synthetic construct
  source 1..23: mol_type = other DNA
aatacaatcactcttaaggtagg

SEQ ID NO: 407 - DNA, 23 bp, synthetic construct
  source 1..23: mol_type = other DNA
gaagtacctttcactattttggg

SEQ ID NO: 408 - DNA, 23 bp, synthetic construct
  source 1..23: mol_type = other DNA
caagcaacaaatgacttctaagg

SEQ ID NO: 409 - DNA, 23 bp, synthetic construct
  source 1..23: mol_type = other DNA
tttgaatacaatcactcttaagg

SEQ ID NO: 410 - DNA, 23 bp, synthetic construct
  source 1..23: mol_type = other DNA
acaaacggactacgtaaacttgg

SEQ ID NO: 411 - DNA, 23 bp, synthetic construct
  source 1..23: mol_type = other DNA
acaagatgtgaacacgacgatgg

SEQ ID NO: 412 - DNA, 23 bp, synthetic construct
  source 1..23: mol_type = other DNA
gttgcaccgttgattccttcagg

SEQ ID NO: 413 - DNA, 23 bp, synthetic construct
  source 1..23: mol_type = other DNA
agtaatattgaattagggcgtgg

SEQ ID NO: 414 - DNA, 23 bp, synthetic construct
  source 1..23: mol_type = other DNA
cctgatgttggctcgacattagg

SEQ ID NO: 415 - DNA, 23 bp, synthetic construct
  source 1..23: mol_type = other DNA
ctttgttgggtcttagcttaagg

SEQ ID NO: 416 - DNA, 23 bp, synthetic construct
  source 1..23: mol_type = other DNA
tcggaacagctccttcctgaagg

SEQ ID NO: 417 - DNA, 23 bp, synthetic construct
  source 1..23: mol_type = other DNA
agtagtttctgaggtcatgttgg

SEQ ID NO: 418 - DNA, 23 bp, synthetic construct
  source 1..23: mol_type = other DNA
cttgaaaatacgatgatgtgagg

SEQ ID NO: 419 - DNA, 23 bp, synthetic construct
  source 1..23: mol_type = other DNA
gcattaatctagagagagggagg

SEQ ID NO: 420 - DNA, 23 bp, synthetic construct
  source 1..23: mol_type = other DNA
gggtcatgttagaattcatgtgg

SEQ ID NO: 421 - DNA, 23 bp, synthetic construct
  source 1..23: mol_type = other DNA
tgatgcattaatctagagagagg

SEQ ID NO: 422 - DNA, 23 bp, synthetic construct
  source 1..23: mol_type = other DNA
acatcatcgtattttcaagttgg

SEQ ID NO: 423 - DNA, 23 bp, synthetic construct
  source 1..23: mol_type = other DNA
ctagctgacaaacatgtgagtgg

SEQ ID NO: 424 - DNA, 23 bp, synthetic construct
  source 1..23: mol_type = other DNA
aacatgacccaagtgagtccagg

SEQ ID NO: 425 - DNA, 23 bp, synthetic construct
  source 1..23: mol_type = other DNA
gattccgtatttgctttgttggg

SEQ ID NO: 426 - DNA, 23 bp, synthetic construct
  source 1..23: mol_type = other DNA
tacgatgatgtgaggaaataagg

SEQ ID NO: 427 - DNA, 23 bp, synthetic construct
  source 1..23: mol_type = other DNA
gtaatatgtctaagtactgatgg

SEQ ID NO: 428 - DNA, 23 bp, synthetic construct
  source 1..23: mol_type = other DNA
gtaaagtgagctggttcattagg

SEQ ID NO: 429 - DNA, 23 bp, synthetic construct
  source 1..23: mol_type = other DNA
actagagtccttaagaagggggg

SEQ ID NO: 430 - DNA, 23 bp, synthetic construct
  source 1..23: mol_type = other DNA
ggaagacttttctctatacgagg

SEQ ID NO: 431 - DNA, 23 bp, synthetic construct
  source 1..23: mol_type = other DNA
gcattcctttcatccatggcagg

SEQ ID NO: 432 - DNA, 23 bp, synthetic construct
  source 1..23: mol_type = other DNA
gacatatggttataaaaatcagg

SEQ ID NO: 433 - DNA, 23 bp, synthetic construct
  source 1..23: mol_type = other DNA
ggagtgcagtccctgacatatgg

SEQ ID NO: 434 - DNA, 23 bp, synthetic construct
  source 1..23: mol_type = other DNA
gtgggttagggtggttaactggg

SEQ ID NO: 435 - DNA, 23 bp, synthetic construct
  source 1..23: mol_type = other DNA
aggtgcaaaaaggttgctgtggg

SEQ ID NO: 436 - DNA, 23 bp, synthetic construct
  source 1..23: mol_type = other DNA
cgtgacaaggcaaagtggcgtgg

SEQ ID NO: 437 - DNA, 23 bp, synthetic construct
  source 1..23: mol_type = other DNA
gaaggactgcccctgacgtcagg

SEQ ID NO: 438 - DNA, 23 bp, synthetic construct
  source 1..23: mol_type = other DNA
ctgcccctgacgtcaggagttgg

SEQ ID NO: 439 - DNA, 23 bp, synthetic construct
  source 1..23: mol_type = other DNA
tgtgggttagggtggttaactgg

SEQ ID NO: 440 - DNA, 23 bp, synthetic construct
  source 1..23: mol_type = other DNA
acccttttagagttttctgctgg

SEQ ID NO: 441 - DNA, 23 bp, synthetic construct
  source 1..23: mol_type = other DNA
aacttcctgccatggatgaaagg

SEQ ID NO: 442 - DNA, 23 bp, synthetic construct
  source 1..23: mol_type = other DNA
gcaaaaaggttgctgtgggttgg

SEQ ID NO: 443 - DNA, 23 bp, synthetic construct
  source 1..23: mol_type = other DNA
aatttgggggtagataggcatgg

SEQ ID NO: 444 - DNA, 23 bp, synthetic construct
  source 1..23: mol_type = other DNA
agaaaactctaaaagggtatagg

SEQ ID NO: 445 - DNA, 23 bp, synthetic construct
  source 1..23: mol_type = other DNA
attagcattcctttcatccatgg

SEQ ID NO: 446 - DNA, 23 bp, synthetic construct
  source 1..23: mol_type = other DNA
cccagcagaaaactctaaaaggg

SEQ ID NO: 447 - DNA, 23 bp, synthetic construct
  source 1..23: mol_type = other DNA
caggtgcaaaaaggttgctgtgg

SEQ ID NO: 448 - DNA, 23 bp, synthetic construct
  source 1..23: mol_type = other DNA
gcaagagatgaaattccatatgg

SEQ ID NO: 449 - DNA, 23 bp, synthetic construct
  source 1..23: mol_type = other DNA
gggctgttctaacgaagtctggg

SEQ ID NO: 450 - DNA, 23 bp, synthetic construct
  source 1..23: mol_type = other DNA
tgtccattcagcgaccctagagg

SEQ ID NO: 451 - DNA, 23 bp, synthetic construct
  source 1..23: mol_type = other DNA
ggctgttctaacgaagtctgggg

SEQ ID NO: 452 - DNA, 23 bp, synthetic construct
  source 1..23: mol_type = other DNA
gtccattcagcgaccctagaggg

SEQ ID NO: 453 - DNA, 23 bp, synthetic construct
  source 1..23: mol_type = other DNA
ggggctgttctaacgaagtctgg

SEQ ID NO: 454 - DNA, 23 bp, synthetic construct
  source 1..23: mol_type = other DNA
ggctgaatcagcatgcgaaaggg

SEQ ID NO: 455 - DNA, 23 bp, synthetic construct
  source 1..23: mol_type = other DNA
ttccaatggggggcatagcctgg

SEQ ID NO: 456 - DNA, 23 bp, synthetic construct
  source 1..23: mol_type = other DNA
taccctctagggtcgctgaatgg

SEQ ID NO: 457 - DNA, 23 bp, synthetic construct
  source 1..23: mol_type = other DNA
atcctcttgggccttataagagg

SEQ ID NO: 458 - DNA, 23 bp, synthetic construct
  source 1..23: mol_type = other DNA
ggccaggctatgccccccattgg

SEQ ID NO: 459 - DNA, 23 bp, synthetic construct
  source 1..23: mol_type = other DNA
ctagaggaccagaacaactctgg

SEQ ID NO: 460 - DNA, 23 bp, synthetic construct
  source 1..23: mol_type = other DNA
tccctcttataaggcccaagagg

SEQ ID NO: 461 - DNA, 23 bp, synthetic construct
  source 1..23: mol_type = other DNA
aggctgaatcagcatgcgaaagg

SEQ ID NO: 462 - DNA, 23 bp, synthetic construct
  source 1..23: mol_type = other DNA
ggaccagaacaactctggcctgg

SEQ ID NO: 463 - DNA, 23 bp, synthetic construct
  source 1..23: mol_type = other DNA
gggcttttatttggcccagcagg

SEQ ID NO: 464 - DNA, 23 bp, synthetic construct
  source 1..23: mol_type = other DNA
gtcgctgaatggacagactctgg

SEQ ID NO: 465 - DNA, 23 bp, synthetic construct
  source 1..23: mol_type = other DNA
ctcatgagttttaccctctaggg

SEQ ID NO: 466 - DNA, 23 bp, synthetic construct
  source 1..23: mol_type = other DNA
tcctcttgggccttataagaggg

SEQ ID NO: 467 - DNA, 23 bp, synthetic construct
  source 1..23: mol_type = other DNA
tcttgggccttataagagggagg

SEQ ID NO: 468 - DNA, 23 bp, synthetic construct
  source 1..23: mol_type = other DNA
tagaacagccccccacacagtgg

SEQ ID NO: 469 - DNA, 23 bp, synthetic construct
  source 1..23: mol_type = other DNA
gttacgttatgactaatctttgg

SEQ ID NO: 470 - DNA, 23 bp, synthetic construct
  source 1..23: mol_type = other DNA
tacgttatgactaatctttgggg

SEQ ID NO: 471 - DNA, 23 bp, synthetic construct
  source 1..23: mol_type = other DNA
ggaagtagtgttatgatgtatgg

SEQ ID NO: 472 - DNA, 23 bp, synthetic construct
  source 1..23: mol_type = other DNA
gttatgatgtatgggcataaagg

SEQ ID NO: 473 - DNA, 23 bp, synthetic construct
  source 1..23: mol_type = other DNA
gaagtagtgttatgatgtatggg

SEQ ID NO: 474 - DNA, 23 bp, synthetic construct
  source 1..23: mol_type = other DNA
atagctgctggcagtataactgg

SEQ ID NO: 475 - DNA, 23 bp, synthetic construct
  source 1..23: mol_type = other DNA
gcatcacaacattgacactgtgg

SEQ ID NO: 476 - DNA, 23 bp, synthetic construct
  source 1..23: mol_type = other DNA
aaggcgagtttctacaaagatgg

SEQ ID NO: 477 - DNA, 23 bp, synthetic construct
  source 1..23: mol_type = other DNA
ttacgttatgactaatctttggg

SEQ ID NO: 478 - DNA, 23 bp, synthetic construct
  source 1..23: mol_type = other DNA
caagactgattaagactgatggg

SEQ ID NO: 479 - DNA, 23 bp, synthetic construct
  source 1..23: mol_type = other DNA
agcagcaatgtattaaaggctgg

SEQ ID NO: 480 - DNA, 23 bp, synthetic construct
  source 1..23: mol_type = other DNA
ctacaggattgatgtaaacatgg

SEQ ID NO: 481 - DNA, 23 bp, synthetic construct
  source 1..23: mol_type = other DNA
tgggcataaagggttttaatggg

SEQ ID NO: 482 - DNA, 23 bp, synthetic construct
  source 1..23: mol_type = other DNA
acatcaatcctgtaggtgattgg

SEQ ID NO: 483 - DNA, 23 bp, synthetic construct
  source 1..23: mol_type = other DNA
attctagtcattatagctgctgg

SEQ ID NO: 484 - DNA, 23 bp, synthetic construct
  source 1..23: mol_type = other DNA
catcaatcctgtaggtgattggg

SEQ ID NO: 485 - DNA, 23 bp, synthetic construct
  source 1..23: mol_type = other DNA
gttataagatcaattctgagtgg

SEQ ID NO: 486 - DNA, 23 bp, synthetic construct
  source 1..23: mol_type = other DNA
ggcagactgtggatcaaaagtgg

SEQ ID NO: 487 - DNA, 23 bp, synthetic construct
  source 1..23: mol_type = other DNA
atggctgcccaatcacctacagg

SEQ ID NO: 488 - DNA, 23 bp, synthetic construct
  source 1..23: mol_type = other DNA
tcaaagcatgtacttagagttgg

SEQ ID NO: 489 - DNA, 20 bp, synthetic construct
  source 1..20: mol_type = other DNA
tcgcccctcaaatcttacag

SEQ ID NO: 490 - DNA, 20 bp, synthetic construct
  source 1..20: mol_type = other DNA
tcaaatcttacagctgctca

SEQ ID NO: 491 - DNA, 20 bp, synthetic construct
  source 1..20: mol_type = other DNA
tcttacagctgctcactccc

SEQ ID NO: 492 - DNA, 20 bp, synthetic construct
  source 1..20: mol_type = other DNA
tacagctgctcactcccctg

SEQ ID NO: 493 - DNA, 20 bp, synthetic construct
  source 1..20: mol_type = other DNA
tgctcactcccctgcagggc

SEQ ID NO: 494 - DNA, 20 bp, synthetic construct
  source 1..20: mol_type = other DNA
tcccctgcagggcaacgccc

SEQ ID NO: 495 - DNA, 20 bp, synthetic construct
  source 1..20: mol_type = other DNA
tgcagggcaacgcccaggga

SEQ ID NO: 496 - DNA, 20 bp, synthetic construct
  source 1..20: mol_type = other DNA
tctcgattatgggcgggatt

SEQ ID NO: 497 - DNA, 20 bp, synthetic construct
  source 1..20: mol_type = other DNA
tcgcttctcgattatgggcg

SEQ ID NO: 498 - DNA, 20 bp, synthetic construct
  source 1..20: mol_type = other DNA
tgtcgagtcgcttctcgatt

SEQ ID NO: 499 - DNA, 20 bp, synthetic construct
  source 1..20: mol_type = other DNA
tccatgtcgagtcgcttctc

SEQ ID NO: 500 - DNA, 20 bp, synthetic construct
  source 1..20: mol_type = other DNA
tcgcctccatgtcgagtcgc

SEQ ID NO: 501 - DNA, 20 bp, synthetic construct
  source 1..20: mol_type = other DNA
tcgtcatcgcctccatgtcg

SEQ ID NO: 502 - DNA, 20 bp, synthetic construct
  source 1..20: mol_type = other DNA
tgatctcgtcatcgcctcca

SEQ ID NO: 503 - DNA, 20 bp, synthetic construct
  source 1..20: mol_type = other DNA
tggccggcctgaccactggg

SEQ ID NO: 504 - DNA, 20 bp, synthetic construct
  source 1..20: mol_type = other DNA
tgaaggcctggccggcctga

SEQ ID NO: 505 - DNA, 20 bp, synthetic construct
  source 1..20: mol_type = other DNA
tgagcactgaaggcctggcc

SEQ ID NO: 506 - DNA, 20 bp, synthetic construct
  source 1..20: mol_type = other DNA
tccactgagcactgaaggcc

SEQ ID NO: 507 - DNA, 20 bp, synthetic construct
  source 1..20: mol_type = other DNA
tggtttccactgagcactga

SEQ ID NO: 508 - DNA, 20 bp, synthetic construct
  source 1..20: mol_type = other DNA
tggggaaaatgacccaacag

SEQ ID NO: 509 - DNA, 20 bp, synthetic construct
  source 1..20: mol_type = other DNA
taggacagtggggaaaatga

SEQ ID NO: 510 - DNA, 20 bp, synthetic construct
  source 1..20: mol_type = other DNA
tccagggacacggtgctagg

SEQ ID NO: 511 - DNA, 20 bp, synthetic construct
  source 1..20: mol_type = other DNA
tcagagccaggagtcctggc

SEQ ID NO: 512 - DNA, 20 bp, synthetic construct
  source 1..20: mol_type = other DNA
tccttcagagccaggagtcc

SEQ ID NO: 513 - DNA, 20 bp, synthetic construct
  source 1..20: mol_type = other DNA
tcctccttcagagccaggag

SEQ ID NO: 514 - DNA, 20 bp, synthetic construct
  source 1..20: mol_type = other DNA
tccagcccctcctccttcag

SEQ ID NO: 515 - DNA, 20 bp, synthetic construct
  source 1..20: mol_type = other DNA
tccgagcttgacccttggaa

SEQ ID NO: 516 - DNA, 19 bp, synthetic construct
  source 1..19: mol_type = other DNA
tggtttccgagcttgaccc

SEQ ID NO: 517 - DNA, 20 bp, synthetic construct
  source 1..20: mol_type = other DNA
tggggtggtttccgagcttg

SEQ ID NO: 518 - DNA, 20 bp, synthetic construct
  source 1..20: mol_type = other DNA
tctgctggggtggtttccga

SEQ ID NO: 519 - DNA, 20 bp, synthetic construct
  source 1..20: mol_type = other DNA
tgcagagtatctgctggggt

SEQ ID NO: 520 - DNA, 18 bp, synthetic construct
  source 1..18: mol_type = other DNA
tcttcctagtattaaagt

SEQ ID NO: 521 - DNA, 17 bp, synthetic construct
  source 1..17: mol_type = other DNA
tccttaatattaccagt

SEQ ID NO: 522 - DNA, 25 bp, synthetic construct
  source 1..25: mol_type = other DNA
taccaagctgaaatgacacaaaagt

SEQ ID NO: 523 - DNA, 25 bp, synthetic construct
  source 1..25: mol_type = other DNA
tggctgtgtcacataccagcagaat

SEQ ID NO: 524 - DNA, 22 bp, synthetic construct
  source 1..22: mol_type = other DNA
tgttaatttgaatacaatcact

SEQ ID NO: 525 - DNA, 21 bp, synthetic construct
  source 1..21: mol_type = other DNA
tgtgtcacataccagcagaat

SEQ ID NO: 526 - DNA, 16 bp, synthetic construct
  source 1..16: mol_type = other DNA
tggtaactactaattt

SEQ ID NO: 527 - DNA, 19 bp, synthetic construct
  source 1..19: mol_type = other DNA
tgtcacataccagcagaat

SEQ ID NO: 528 - DNA, 21 bp, synthetic construct
  source 1..21: mol_type = other DNA
tgtgacacagccatcaacaat

SEQ ID NO: 529 - DNA, 16 bp, synthetic construct
  source 1..16: mol_type = other DNA
tcctttgatgaacagt

SEQ ID NO: 530 - DNA, 29 bp, synthetic construct
  source 1..29: mol_type = other DNA
tgtgtgcaatagcgttaaaggaactacat

SEQ ID NO: 531 - DNA, 17 bp, synthetic construct
  source 1..17: mol_type = other DNA
tctttcaatagcccact

SEQ ID NO: 532 - DNA, 22 bp, synthetic construct
  source 1..22: mol_type = other DNA
tctcaaatgacaagagcacagt

SEQ ID NO: 533 - DNA, 18 bp, synthetic construct
  source 1..18: mol_type = other DNA
taccagttaattagcact

SEQ ID NO: 534 - DNA, 18 bp, synthetic construct
  source 1..18: mol_type = other DNA
tgttgtgacctaagccat

SEQ ID NO: 535 - DNA, 23 bp, synthetic construct
  source 1..23: mol_type = other DNA
tctcatgttttaaagtcaagaat

SEQ ID NO: 536 - DNA, 19 bp, synthetic construct
  source 1..19: mol_type = other DNA
tcctgaattcagaacagat

SEQ ID NO: 537 - DNA, 26 bp, synthetic construct
  source 1..26: mol_type = other DNA
tagcatgatgtttcatgttgtgacct

SEQ ID NO: 538 - DNA, 25 bp, synthetic construct
  source 1..25: mol_type = other DNA
tgtttcatgttgtgacctaagccat

SEQ ID NO: 539 - DNA, 18 bp, synthetic construct
  source 1..18: mol_type = other DNA
tacaacagtctatttcat

SEQ ID NO: 540 - DNA, 26 bp, synthetic construct
  source 1..26: mol_type = other DNA
tcttcctagtctcttctctacccagt

SEQ ID NO: 541 - DNA, 25 bp, synthetic construct
  source 1..25: mol_type = other DNA
tacactccagcctgggaaacagagt

SEQ ID NO: 542 - DNA, 19 bp, synthetic construct
  source 1..19: mol_type = other DNA
tcttttccttaggacggct

SEQ ID NO: 543 - DNA, 17 bp, synthetic construct
  source 1..17: mol_type = other DNA
tcgctcaggcctgtcat

SEQ ID NO: 544 - DNA, 16 bp, synthetic construct
  source 1..16: mol_type = other DNA
tccatatggaagactt

SEQ ID NO: 545 - DNA, 18 bp, synthetic construct
  source 1..18: mol_type = other DNA
tacccagttaaccaccct

SEQ ID NO: 546 - DNA, 27 bp, synthetic construct
  source 1..27: mol_type = other DNA
tggcgcatgcctgtaatcccagctact

SEQ ID NO: 547 - DNA, 26 bp, synthetic construct
  source 1..26: mol_type = other DNA
tatacgaggagaaaattagcattcct

SEQ ID NO: 548 - DNA, 23 bp, synthetic construct
  source 1..23: mol_type = other DNA
tctgcctcccaggttcacgcaat

SEQ ID NO: 549 - DNA, 22 bp, synthetic construct
  source 1..22: mol_type = other DNA
tgccttgtcacgttttcacagt

SEQ ID NO: 550 - DNA, 25 bp, synthetic construct
  source 1..25: mol_type = other DNA
tgtcaccttctgtatgtgcaaccat

SEQ ID NO: 551 - DNA, 17 bp, synthetic construct
  source 1..17: mol_type = other DNA
tctgtatgtgcaaccat

SEQ ID NO: 552 - DNA, 17 bp, synthetic construct
  source 1..17: mol_type = other DNA
tagtcaagcaacaggat

SEQ ID NO: 553 - DNA, 18 bp, synthetic construct
  source 1..18: mol_type = other DNA
tccaagataattccccat

SEQ ID NO: 554 - DNA, 16 bp, synthetic construct
  source 1..16: mol_type = other DNA
tctgcaagatcctttt

SEQ ID NO: 555 - DNA, 29 bp, synthetic construct
  source 1..29: mol_type = other DNA
tgctatgtaaggtagcaaaaaggtaacct

SEQ ID NO: 556 - DNA, 16 bp, synthetic construct
  source 1..16: mol_type = other DNA
tctctctcctcctgct

SEQ ID NO: 557 - DNA, 19 bp, synthetic construct
  source 1..19: mol_type = other DNA
tccaaatgctattctctct

SEQ ID NO: 558 - DNA, 17 bp, synthetic construct
  source 1..17: mol_type = other DNA
tgctgattcagcctcct

SEQ ID NO: 559 - DNA, 21 bp, synthetic construct
  source 1..21: mol_type = other DNA
tagaacagccccccacacagt

SEQ ID NO: 560 - DNA, 18 bp, synthetic construct
  source 1..18: mol_type = other DNA
tttagcagatgcatcagc

SEQ ID NO: 561 - DNA, 20 bp, synthetic construct
  source 1..20: mol_type = other DNA
tgaccaggggcatgtcctgg

SEQ ID NO: 562 - DNA, 21 bp, synthetic construct
  source 1..21: mol_type = other DNA
tggtccacctacctgaaaatg

SEQ ID NO: 563 - DNA, 21 bp, synthetic construct
  source 1..21: mol_type = other DNA
tgtcccacaggtattacgggc

SEQ ID NO: 564 - DNA, 21 bp, synthetic construct
  source 1..21: mol_type = other DNA
tacgggccaacctgacaatac

SEQ ID NO: 565 - DNA, 21 bp, synthetic construct
  source 1..21: mol_type = other DNA
tgagctttggggactgaaaga

SEQ ID NO: 566 - DNA, 21 bp, synthetic construct
  source 1..21: mol_type = other DNA
ctggcataatcttttccccca

SEQ ID NO: 567 - DNA, 21 bp, synthetic construct
  source 1..21: mol_type = other DNA
ccagcctcctggccatgtgca

SEQ ID NO: 568 - DNA, 21 bp, synthetic construct
  source 1..21: mol_type = other DNA
ggccatgtgcacaggggctga

SEQ ID NO: 569 - DNA, 21 bp, synthetic construct
  source 1..21: mol_type = other DNA
ctgatatgtgaaggtttagca

SEQ ID NO: 570 - DNA, 21 bp, synthetic construct
  source 1..21: mol_type = other DNA
tgaccaggcgtggtggctcac

SEQ ID NO: 571 - DNA, 16 bp, synthetic construct
  source 1..16: mol_type = other DNA
tatagacattttcact

SEQ ID NO: 572 - DNA, 21 bp, synthetic construct
  source 1..21: mol_type = other DNA
tctacatttaactatcaacct

SEQ ID NO: 573 - DNA, 17 bp, synthetic construct
  source 1..17: mol_type = other DNA
tcgtgcaaacgtttgat

SEQ ID NO: 574 - DNA, 18 bp, synthetic construct
  source 1..18: mol_type = other DNA
tacatcaatcctgtaggt

SEQ ID NO: 575 - DNA, 20 bp, synthetic construct
  source 1..20: mol_type = other DNA
tctattttagtgacccaagt

SEQ ID NO: 576 - DNA, 19 bp, synthetic construct
  source 1..19: mol_type = other DNA
tagagtcaaagcatgtact

SEQ ID NO: 577 - DNA, 18 bp, synthetic construct
  source 1..18: mol_type = other DNA
tcctacccataagctcct

SEQ ID NO: 578 - DNA, 17 bp, synthetic construct
  source 1..17: mol_type = other DNA
tccccatccccatcagt

SEQ ID NO: 579 - DNA, 29 bp, synthetic construct
  source 1..29: mol_type = other DNA
tctttaattcaagcaagactttaacaagt

SEQ ID NO: 580 - DNA, 16 bp, synthetic construct
  source 1..16: mol_type = other DNA
tgcagtcccctttctt

SEQ ID NO: 581 - DNA, 20 bp, synthetic construct
  source 1..20: mol_type = other DNA
tctgcacaaatccccaaaga

SEQ ID NO: 582 - DNA, 16 bp, synthetic construct
  source 1..16: mol_type = other DNA
tacatgctttgactct

SEQ ID NO: 583 - DNA, 29 bp, synthetic construct
  source 1..29: mol_type = other DNA
tggccagttatactgccagcagctataat

SEQ ID NO: 584 - DNA, 15 bp, synthetic construct
  source 1..15: mol_type = other DNA
tgggaagataaacta

SEQ ID NO: 585 - DNA, 18 bp, synthetic construct
  source 1..18: mol_type = other DNA
actcccctgcagggcaac

SEQ ID NO: 586 - DNA, 18 bp, synthetic construct
  source 1..18: mol_type = other DNA
cccctgcagggcaacgcc

SEQ ID NO: 587 - DNA, 18 bp, synthetic construct
  source 1..18: mol_type = other DNA
ctgcagggcaacgcccag

SEQ ID NO: 588 - DNA, 18 bp, synthetic construct
  source 1..18: mol_type = other DNA
cagggcaacgcccaggga

SEQ ID NO: 589 - DNA, 18 bp, synthetic construct
  source 1..18: mol_type = other DNA
ggcaacgcccagggacca

SEQ ID NO: 590 - DNA, 18 bp, synthetic construct
  source 1..18: mol_type = other DNA
aacgcccagggaccaagt

SEQ ID NO: 591 - DNA, 18 bp, synthetic construct
  source 1..18: mol_type = other DNA
gcccagggaccaagttag

SEQ ID NO: 592 - DNA, 18 bp, synthetic construct
  source 1..18: mol_type = other DNA
cagggaccaagttagccc

SEQ ID NO: 593 - DNA, 15 bp, synthetic construct
  source 1..15: mol_type = other DNA
gcctaggcaaaagaa

SEQ ID NO: 594 - DNA, 18 bp, synthetic construct
  source 1..18: mol_type = other DNA
cgcgaggaggaaaggagg

SEQ ID NO: 595 - DNA, 18 bp, synthetic construct
  source 1..18: mol_type = other DNA
gaggaggaaaggagggag

SEQ ID NO: 596 - DNA, 18 bp, synthetic construct
  source 1..18: mol_type = other DNA
gaggaaaggagggagggc

SEQ ID NO: 597 - protein, 176 aa, synthetic construct
LEPGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGEKPYKCPECGKSFSTHLDLIRHQRTHTGKKTS

SEQ ID NO: 598 - protein, 176 aa, synthetic construct
LEPGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGKKTS

SEQ ID NO: 599 - protein, 176 aa, synthetic construct
LEPGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGKKTS

SEQ ID NO: 600 - protein, 176 aa, synthetic construct
LEPGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGKKTS

SEQ ID NO: 601 - protein, 176 aa, synthetic construct
LEPGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGKKTS

SEQ ID NO: 602 - protein, 176 aa, synthetic construct
LEPGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGKKTS

SEQ ID NO: 603 - protein, 176 aa, synthetic construct
LEPGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGKKTS

SEQ ID NO: 604 - protein, 176 aa, synthetic construct
LEPGEKPYKCPECGKSFSSKKHLAEHQRTHTGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGKKTS

SEQ ID NO: 605 - protein, 148 aa, synthetic construct
LEPGEKPYKCPECGKSFSQSSNLVRHQRTHTGEKPYKCPECGKSFSQRANLRAHQRTHTGEKPYKCPECGKSFSQSGDLRRHQRTHTGEKPYKCPECGKSFSREDNLHTHQRTHTGEKPYKCPECGKSFSDCRDLARHQRTHTGKKTS

SEQ ID NO: 606 - protein, 176 aa, synthetic construct
LEPGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGEKPYKCPECGKSFSHTGHLLEHQRTHTGKKTS

SEQ ID NO: 607 - protein, 176 aa, synthetic construct
LEPGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGKKTS

SEQ ID NO: 608 - protein, 176 aa, synthetic construct
LEPGEKPYKCPECGKSFSDPGHLVRHQRTHTGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGKKTS

SEQ ID NO: 609 - DNA, 30 bp, synthetic construct
  source 1..30: mol_type = other DNA
taggacagtggggaaaatgacccaacagcc

SEQ ID NO: 610 - DNA, 21 bp, synthetic construct
  source 1..21: mol_type = other DNA
agagggagccacgaaaacaga

SEQ ID NO: 611 - DNA, 15 bp, synthetic construct
  source 1..15: mol_type = other DNA
gcagatagccaggag

SEQ ID NO: 612 - DNA, 18 bp, synthetic construct
  source 1..18: mol_type = other DNA
agatagccaggagtcctt

SEQ ID NO: 613 - DNA, 24 bp, synthetic construct
  source 1..24: mol_type = other DNA
cccagtggtcaggccggccaggcc

SEQ ID NO: 614 - DNA, 18 bp, synthetic construct
  source 1..18: mol_type = other DNA
ggccggccaggccttcag

SEQ ID NO: 615 - DNA, 27 bp, synthetic construct
  source 1..27: mol_type = other DNA
agtgctcagtggaaaccacgaaaggac

SEQ ID NO: 616 - protein, 21 aa, synthetic construct
TGGCCCCCAGCCCCTCCTGCC

SEQ ID NO: 617 - DNA, 27 bp, synthetic construct
  source 1..27: mol_type = other DNA
agagccaggagtcctggcccccagccc

SEQ ID NO: 618 - DNA, 24 bp, synthetic construct
  source 1..24: mol_type = other DNA
gcaggaggggctgggggccaggac

SEQ ID NO: 619 - DNA, 24 bp, synthetic construct
  source 1..24: mol_type = other DNA
atagccctgggcccacggcttcgt

SEQ ID NO: 620 - DNA, 15 bp, synthetic construct
  source 1..15: mol_type = other DNA
gaaggacctggctgg

SEQ ID NO: 621 - DNA, 27 bp, synthetic construct
  source 1..27: mol_type = other DNA
gcaggaacgaagccgtgggcccagggc

SEQ ID NO: 622 - DNA, 18 bp, synthetic construct
  source 1..18: mol_type = other DNA
ggaaaccaccccagcaga

SEQ ID NO: 623 - DNA, 33 bp, synthetic construct
  source 1..33: mol_type = other DNA
aagggtcaagctcggaaaccaccccagcagata

SEQ ID NO: 624 - protein, 288 aa, synthetic construct
LEPGEKPYKCPECGKSFSDCRDLARHQRTHTGEKPYKCPECGKSFSSPADLTRHQRTHTGEKPYKCPECGKSFSTSHSLTEHQRTHTGEKPYKCPECGKSFSDPGNLVRHQRTHTGEKPYKCPECGKSFSTTGNLTVHQRTHTGEKPYKCPECGKSFSQSSNLVRHQRTHTGEKPYKCPECGKSFSRSDKLVRHQRTHTGEKPYKCPECGKSFSHRTTLTNHQRTHTGEKPYKCPECGKSFSDPGNLVRHQRTHTGEKPYKCPECGKSFSREDNLHTHQRTHTGKKTS

SEQ ID NO: 625 - protein, 204 aa, synthetic construct
LEPGEKPYKCPECGKSFSQLAHLRAHQRTHTGEKPYKCPECGKSFSDSGNLRVHQRTHTGEKPYKCPECGKSFSQSSNLVRHQRTHTGEKPYKCPECGKSFSSKKALTEHQRTHTGEKPYKCPECGKSFSERSHLREHQRTHTGEKPYKCPECGKSFSRSDKLVRHQRTHTGEKPYKCPECGKSFSQLAHLRAHQRTHTGKKTS

SEQ ID NO: 626 - protein, 148 aa, synthetic construct
LEPGEKPYKCPECGKSFSRSDNLVRHQRTHTGEKPYKCPECGKSFSRADNLTEHQRTHTGEKPYKCPECGKSFSERSHLREHQRTHTGEKPYKCPECGKSFSTSGNLVRHQRTHTGEKPYKCPECGKSFSQSGDLRRHQRTHTGKKTS

SEQ ID NO: 627 - protein, 176 aa, synthetic construct
LEPGEKPYKCPECGKSFSTTGALTEHQRTHTGEKPYKCPECGKSFSDPGALVRHQRTHTGEKPYKCPECGKSFSQRAHLERHQRTHTGEKPYKCPECGKSFSTSHSLTEHQRTHTGEKPYKCPECGKSFSREDNLHTHQRTHTGEKPYKCPECGKSFSQLAHLRAHQRTHTGKKTS

SEQ ID NO: 628 - protein, 232 aa, synthetic construct
LEPGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSDCRDLARHQRTHTGEKPYKCPECGKSFSRADNLTEHQRTHTGEKPYKCPECGKSFSTSGHLVRHQRTHTGEKPYKCPECGKSFSHRTTLTNHQRTHTGEKPYKCPECGKSFSSKKHLAEHQRTHTGKKTS

SEQ ID NO: 629 - protein, 176 aa, synthetic construct
LEPGEKPYKCPECGKSFSRADNLTEHQRTHTGEKPYKCPECGKSFSTTGALTEHQRTHTGEKPYKCPECGKSFSDPGHLVRHQRTHTGEKPYKCPECGKSFSTSHSLTEHQRTHTGEKPYKCPECGKSFSRSDKLTEHQRTHTGEKPYKCPECGKSFSDPGHLVRHQRTHTGKKTS

SEQ ID NO: 630 - protein, 260 aa, synthetic construct
LEPGEKPYKCPECGKSFSDPGNLVRHQRTHTGEKPYKCPECGKSFSRKDNLKNHQRTHTGEKPYKCPECGKSFSQSGHLTEHQRTHTGEKPYKCPECGKSFSTSHSLTEHQRTHTGEKPYKCPECGKSFSQRANLRAHQRTHTGEKPYKCPECGKSFSRSDHLTTHQRTHTGEKPYKCPECGKSFSRADNLTEHQRTHTGEKPYKCPECGKSFSTSGELVRHQRTHTGEKPYKCPECGKSFSHRTTLTNHQRTHTGKKTS

SEQ ID NO: 631 - protein, 204 aa, synthetic construct
LEPGEKPYKCPECGKSFSDCRDLARHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSTSHSLTEHQRTHTGEKPYKCPECGKSFSSKKHLAEHQRTHTGEKPYKCPECGKSFSRSDHLTTHQRTHTGKKTS

SEQ ID NO: 632 - protein, 260 aa, synthetic construct
LEPGEKPYKCPECGKSFSSKKHLAEHQRTHTGEKPYKCPECGKSFSRADNLTEHQRTHTGEKPYKCPECGKSFSSKKHLAEHQRTHTGEKPYKCPECGKSFSDPGHLVRHQRTHTGEKPYKCPECGKSFSTKNSLTEHQRTHTGEKPYKCPECGKSFSHRTTLTNHQRTHTGEKPYKCPECGKSFSRSDHLTNHQRTHTGEKPYKCPECGKSFSDCRDLARHQRTHTGEKPYKCPECGKSFSQLAHLRAHQRTHTGKKTS

SEQ ID NO: 633 - protein, 232 aa, synthetic construct
LEPGEKPYKCPECGKSFSDPGNLVRHQRTHTGEKPYKCPECGKSFSRADNLTEHQRTHTGEKPYKCPECGKSFSDPGHLVRHQRTHTGEKPYKCPECGKSFSRSDKLVRHQRTHTGEKPYKCPECGKSFSTSGELVRHQRTHTGEKPYKCPECGKSFSRSDKLVRHQRTHTGEKPYKCPECGKSFSQRAHLERHQRTHTGEKPYKCPECGKSFSQSGDLRRHQRTHTGKKTS

SEQ ID NO: 634 - protein, 231 aa, synthetic construct
LEPGEKPYKCPECGKSFSSRRTCRAHQRTHTGEKPYKCPECGKSFSTTGALTEHQRTHTGEKPYKCPECGKSFSRSDKLTEHQRTHTGEKPYKCPECGKSFSTSHSLTEHQRTHTGEKPYKCPECGKSFSDPGHLVRHQRTHTGEKPYKCPECGKSFSRNDALTEHQRTHTGEKPYKCPECGKSFSDCRDLARHQRTHTGEKPYKCPECGKSFSQKSSLIAHQRTHTGKKT

SEQ ID NO: 635 - protein, 148 aa, synthetic construct
LEPGEKPYKCPECGKSFSRSDHLTTHQRTHTGEKPYKCPECGKSFSDPGHLVRHQRTHTGEKPYKCPECGKSFSTKNSLTEHQRTHTGEKPYKCPECGKSFSQRAHLERHQRTHTGEKPYKCPECGKSFSQSSNLVRHQRTHTGKKTS

SEQ ID NO: 636 - protein, 260 aa, synthetic construct
LEPGEKPYKCPECGKSFSDPGHLVRHQRTHTGEKPYKCPECGKSFSRADNLTEHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRKDNLKNHQRTHTGEKPYKCPECGKSFSRTDTLRDHQRTHTGEKPYKCPECGKSFSQRAHLERHQRTHTGEKPYKCPECGKSFSQSGDLRRHQRTHTGKKTS

SEQ ID NO: 637 - protein, 176 aa, synthetic construct
LEPGEKPYKCPECGKSFSQLAHLRAHQRTHTGEKPYKCPECGKSFSERSHLREHQRTHTGEKPYKCPECGKSFSSKKHLAEHQRTHTGEKPYKCPECGKSFSSKKALTEHQRTHTGEKPYKCPECGKSFSDSGNLRVHQRTHTGEKPYKCPECGKSFSQRAHLERHQRTHTGKKTS

SEQ ID NO: 638 - protein, 316 aa, synthetic construct
LEPGEKPYKCPECGKSFSQKSSLIAHQRTHTGEKPYKCPECGKSFSRADNLTEHQRTHTGEKPYKCPECGKSFSRADNLTEHQRTHTGEKPYKCPECGKSFSSKKHLAEHQRTHTGEKPYKCPECGKSFSTSHSLTEHQRTHTGEKPYKCPECGKSFSQRANLRAHQRTHTGEKPYKCPECGKSFSRSDKLTEHQRTHTGEKPYKCPECGKSFSTSGELVRHQRTHTGEKPYKCPECGKSFSQSGNLTEHQRTHTGEKPYKCPECGKSFSTSGHLVRHQRTHTGEKPYKCPECGKSFSRKDNLKNHQRTHTGKKTS

SEQ ID NO: 639 - DNA, 18 bp, synthetic construct
  source 1..18: mol_type = other DNA
ctttgatgaacagtcaca

SEQ ID NO: 640 - DNA, 18 bp, synthetic construct
  source 1..18: mol_type = other DNA
cttccaattagtcctacc

SEQ ID NO: 641 - DNA, 21 bp, synthetic construct
  source 1..21: mol_type = other DNA
atactaggaagaaatacaata

SEQ ID NO: 642 - DNA, 18 bp, synthetic construct
  source 1..18: mol_type = other DNA
gctcttgtcatttgagat

SEQ ID NO: 643 - DNA, 33 bp, synthetic construct
  source 1..33: mol_type = other DNA
ccaagctgaaatgacacaaaagttaaaacaaag

SEQ ID NO: 644 - DNA, 21 bp, synthetic construct
  source 1..21: mol_type = other DNA
cttataccagttaattagcac

SEQ ID NO: 645 - DNA, 24 bp, synthetic construct
  source 1..24: mol_type = other DNA
aacgctattgcacacatagttaca

SEQ ID NO: 646 - DNA, 21 bp, synthetic construct
  source 1..21: mol_type = other DNA
tgaattcaggaacaaagtata

SEQ ID NO: 647 - DNA, 27 bp, synthetic construct
  source 1..27: mol_type = other DNA
gctggtatgtgacacagccatcaacaa

SEQ ID NO: 648 - protein, 176 aa, synthetic construct
LEPGEKPYKCPECGKSFSSPADLTRHQRTHTGEKPYKCPECGKSFSDPGALVRHQRTHTGEKPYKCPECGKSFSSPADLTRHQRTHTGEKPYKCPECGKSFSQAGHLASHQRTHTGEKPYKCPECGKSFSQAGHLASHQRTHTGEKPYKCPECGKSFSTTGALTEHQRTHTGKKTS

SEQ ID NO: 649 - protein, 176 aa, synthetic construct
LEPGEKPYKCPECGKSFSDKKDLTRHQRTHTGEKPYKCPECGKSFSTKNSLTEHQRTHTGEKPYKCPECGKSFSHRTTLTNHQRTHTGEKPYKCPECGKSFSHKNALQNHQRTHTGEKPYKCPECGKSFSTSHSLTEHQRTHTGEKPYKCPECGKSFSTTGALTEHQRTHTGKKTS

SEQ ID NO: 650 - protein, 204 aa, synthetic construct
LEPGEKPYKCPECGKSFSQKSSLIAHQRTHTGEKPYKCPECGKSFSSPADLTRHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSQNSTLTEHQRTHTGEKPYKCPECGKSFSQKSSLIAHQRTHTGKKTS

SEQ ID NO: 651 - protein, 176 aa, synthetic construct
LEPGEKPYKCPECGKSFSTSGNLVRHQRTHTGEKPYKCPECGKSFSQAGHLASHQRTHTGEKPYKCPECGKSFSHKNALQNHQRTHTGEKPYKCPECGKSFSDPGALVRHQRTHTGEKPYKCPECGKSFSTTGALTEHQRTHTGEKPYKCPECGKSFSTSGELVRHQRTHTGKKTS

SEQ ID NO: 652 - protein, 316 aa, synthetic construct
LEPGEKPYKCPECGKSFSRKDNLKNHQRTHTGEKPYKCPECGKSFSSPADLTRHQRTHTGEKPYKCPECGKSFSQRANLRAHQRTHTGEKPYKCPECGKSFSTSGSLVRHQRTHTGEKPYKCPECGKSFSQRANLRAHQRTHTGEKPYKCPECGKSFSSPADLTRHQRTHTGEKPYKCPECGKSFSDPGNLVRHQRTHTGEKPYKCPECGKSFSTTGNLTVHQRTHTGEKPYKCPECGKSFSQAGHLASHQRTHTGEKPYKCPECGKSFSERSHLREHQRTHTGEKPYKCPECGKSFSTSHSLTEHQRTHTGKKTS

SEQ ID NO: 653 - protein, 204 aa, synthetic construct
LEPGEKPYKCPECGKSFSSKKALTEHQRTHTGEKPYKCPECGKSFSREDNLHTHQRTHTGEKPYKCPECGKSFSTTGNLTVHQRTHTGEKPYKCPECGKSFSTSGSLVRHQRTHTGEKPYKCPECGKSFSTSHSLTEHQRTHTGEKPYKCPECGKSFSQKSSLIAHQRTHTGEKPYKCPECGKSFSTTGALTEHQRTHTGKKTS

SEQ ID NO: 654 - protein, 232 aa, synthetic construct
LEPGEKPYKCPECGKSFSSPADLTRHQRTHTGEKPYKCPECGKSFSTSGSLVRHQRTHTGEKPYKCPECGKSFSQKSSLIAHQRTHTGEKPYKCPECGKSFSSKKALTEHQRTHTGEKPYKCPECGKSFSQSGDLRRHQRTHTGEKPYKCPECGKSFSHKNALQNHQRTHTGEKPYKCPECGKSFSTSGELVRHQRTHTGEKPYKCPECGKSFSDSGNLRVHQRTHTGKKTS

SEQ ID NO: 655 - protein, 204 aa, synthetic construct
LEPGEKPYKCPECGKSFSQKSSLIAHQRTHTGEKPYKCPECGKSFSHRTTLTNHQRTHTGEKPYKCPECGKSFSQSGNLTEHQRTHTGEKPYKCPECGKSFSQSSNLVRHQRTHTGEKPYKCPECGKSFSRADNLTEHQRTHTGEKPYKCPECGKSFSHKNALQNHQRTHTGEKPYKCPECGKSFSQAGHLASHQRTHTGKKTS

SEQ ID NO: 656 - protein, 260 aa, synthetic construct
LEPGEKPYKCPECGKSFSQSGNLTEHQRTHTGEKPYKCPECGKSFSQSGNLTEHQRTHTGEKPYKCPECGKSFSTSGNLTEHQRTHTGEKPYKCPECGKSFSERSHLREHQRTHTGEKPYKCPECGKSFSSKKALTEHQRTHTGEKPYKCPECGKSFSQAGHLASHQRTHTGEKPYKCPECGKSFSRRDELNVHQRTHTGEKPYKCPECGKSFSTSGHLVRHQRTHTGEKPYKCPECGKSFSTSGELVRHQRTHTGKKTS

SEQ ID NO: 657 - DNA, 24 bp, synthetic construct
  source 1..24: mol_type = other DNA
cttcctgaaagcaagagatgaaat

SEQ ID NO: 658 - DNA, 24 bp, synthetic construct
  source 1..24: mol_type = other DNA
ctgaaagcaagagatgaaattcca

SEQ ID NO: 659 - DNA, 21 bp, synthetic construct
  source 1..21: mol_type = other DNA
atacgaggagaaaattagcat

SEQ ID NO: 660 - DNA, 36 bp, synthetic construct
  source 1..36: mol_type = other DNA
catccatggcaggaagttgaagccaaaataaatctg

SEQ ID NO: 661 - DNA, 27 bp, synthetic construct
  source 1..27: mol_type = other DNA
atggcaggaagttgaagccaaaataaa

SEQ ID NO: 662 - DNA, 33 bp, synthetic construct
  source 1..33: mol_type = other DNA
gaaaagaagactcaaggaaacagagccaaacac

SEQ ID NO: 663 - DNA, 24 bp, synthetic construct
  source 1..24: mol_type = other DNA
aggaaacagagccaaacacttaca

SEQ ID NO: 664 - DNA, 60 bp, synthetic construct
  source 1..60: mol_type = other DNA
atgcagatttggacacagagtagtaaactgtgaaaacgtgacaaggcaaagtggcgtggg

SEQ ID NO: 665 - DNA, 18 bp, synthetic construct
  source 1..18: mol_type = other DNA
ggacacagagtagtaaac

SEQ ID NO: 666 - DNA, 18 bp, synthetic construct
  source 1..18: mol_type = other DNA
aaagctagcagcatggca

SEQ ID NO: 667 - DNA, 24 bp, synthetic construct
  source 1..24: mol_type = other DNA
cctcttataaggcccaagaggata

SEQ ID NO: 668 - DNA, 21 bp, synthetic construct
  source 1..21: mol_type = other DNA
caacatccttgacttaatcac

SEQ ID NO: 669 - DNA, 18 bp, synthetic construct
  source 1..18: mol_type = other DNA
ggtagcaaaaaggtaacc

SEQ ID NO: 670 - DNA, 36 bp, synthetic construct
  source 1..36: mol_type = other DNA
tggggtgcaagaggccaggccagagttgttctggtc

SEQ ID NO: 671 - DNA, 24 bp, synthetic construct
  source 1..24: mol_type = other DNA
cgcatgctgattcagcctcctgac

SEQ ID NO: 672 - DNA, 18 bp, synthetic construct
  source 1..18: mol_type = other DNA
agtcaagcaacaggatga

SEQ ID NO: 673 - DNA, 30 bp, synthetic construct
  source 1..30: mol_type = other DNA
gtcaagcaacaggatgatccaaatgctatt

SEQ ID NO: 674 - protein, 232 aa, synthetic construct
LEPGEKPYKCPECGKSFSTTGNLTVHQRTHTGEKPYKCPECGKSFSQAGHLASHQRTHTGEKPYKCPECGKSFSQLAHLRAHQRTHTGEKPYKCPECGKSFSRKDNLKNHQRTHTGEKPYKCPECGKSFSERSHLREHQRTHTGEKPYKCPECGKSFSQSSNLVRHQRTHTGEKPYKCPECGKSFSTKNSLTEHQRTHTGEKPYKCPECGKSFSTTGALTEHQRTHTGKKTS

SEQ ID NO: 675 - protein, 232 aa, synthetic construct
LEPGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSTSGNLVRHQRTHTGEKPYKCPECGKSFSQLAHLRAHQRTHTGEKPYKCPECGKSFSQSGDLRRHQRTHTGEKPYKCPECGKSFSQRANLRAHQRTHTGEKPYKCPECGKSFSRNDALTEHQRTHTGKKTS

SEQ ID NO: 676 - protein, 204 aa, synthetic construct
LEPGEKPYKCPECGKSFSTSGNLTEHQRTHTGEKPYKCPECGKSFSREDNLHTHQRTHTGEKPYKCPECGKSFSTTGNLTVHQRTHTGEKPYKCPECGKSFSQSSNLVRHQRTHTGEKPYKCPECGKSFSQRAHLERHQRTHTGEKPYKCPECGKSFSQSGHLTEHQRTHTGEKPYKCPECGKSFSQKSSLIAHQRTHTGKKTS

SEQ ID NO: 677 - protein, 344 aa, synthetic construct
LEPGEKPYKCPECGKSFSRNDALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSDCRDLARHQRTHTGEKPYKCPECGKSFSQSSNLVRHQRTHTGEKPYKCPECGKSFSTSGSLVRHQRTHTGEKPYKCPECGKSFSQSSNLVRHQRTHTGEKPYKCPECGKSFSRADNLTEHQRTHTGEKPYKCPECGKSFSRSDHLTTHQRTHTGEKPYKCPECGKSFSTSHSLTEHQRTHTGEKPYKCPECGKSFSTSGNLTEHQRTHTGKKTS

SEQ ID NO: 678 - protein, 260 aa, synthetic construct
LEPGEKPYKCPECGKSFSQRANLRAHQRTHTGEKPYKCPECGKSFSTTGNLTVHQRTHTGEKPYKCPECGKSFSQSGNLTEHQRTHTGEKPYKCPECGKSFSERSHLREHQRTHTGEKPYKCPECGKSFSQAGHLASHQRTHTGEKPYKCPECGKSFSHRTTLTNHQRTHTGEKPYKCPECGKSFSQRAHLERHQRTHTGEKPYKCPECGKSFSQSGDLRRHQRTHTGEKPYKCPECGKSFSRRDELNVHQRTHTGKKTS

SEQ ID NO: 679 - protein, 316 aa, synthetic construct
LEPGEKPYKCPECGKSFSSKKALTEHQRTHTGEKPYKCPECGKSFSQRANLRAHQRTHTGEKPYKCPECGKSFSDCRDLARHQRTHTGEKPYKCPECGKSFSQLAHLRAHQRTHTGEKPYKCPECGKSFSDSGNLRVHQRTHTGEKPYKCPECGKSFSQRAHLERHQRTHTGEKPYKCPECGKSFSQSGNLTEHQRTHTGEKPYKCPECGKSFSTHLDLIRHQRTHTGEKPYKCPECGKSFSRKDNLKNHQRTHTGEKPYKCPECGKSFSRKDNLKNHQRTHTGEKPYKCPECGKSFSQSSNLVRHQRTHTGKKTS

SEQ ID NO: 680 - protein, 232 aa, synthetic construct
LEPGEKPYKCPECGKSFSSPADLTRHQRTHTGEKPYKCPECGKSFSTTGALTEHQRTHTGEKPYKCPECGKSFSSPADLTRHQRTHTGEKPYKCPECGKSFSQSGNLTEHQRTHTGEKPYKCPECGKSFSERSHLREHQRTHTGEKPYKCPECGKSFSRADNLTEHQRTHTGEKPYKCPECGKSFSQRANLRAHQRTHTGEKPYKCPECGKSFSRSDHLTNHQRTHTGKKTS

SEQ ID NO: 681 - protein, 568 aa, synthetic construct
LEPGEKPYKCPECGKSFSRSDKLVRHQRTHTGEKPYKCPECGKSFSSRRTCRAHQRTHTGEKPYKCPECGKSFSRSDHLTTHQRTHTGEKPYKCPECGKSFSRKDNLKNHQRTHTGEKPYKCPECGKSFSQSGDLRRHQRTHTGEKPYKCPECGKSFSRKDNLKNHQRTHTGEKPYKCPECGKSFSDPGNLVRHQRTHTGEKPYKCPECGKSFSSRRTCRAHQRTHTGEKPYKCPECGKSFSQRANLRAHQRTHTGEKPYKCPECGKSFSQAGHLASHQRTHTGEKPYKCPECGKSFSRNDALTEHQRTHTGEKPYKCPECGKSFSQRANLRAHQRTHTGEKPYKCPECGKSFSHRTTLTNHQRTHTGEKPYKCPECGKSFSHRTTLTNHQRTHTGEKPYKCPECGKSFSRADNLTEHQRTHTGEKPYKCPECGKSFSSPADLTRHQRTHTGEKPYKCPECGKSFSRSDHLTTHQRTHTGEKPYKCPECGKSFSHKNALQNHQRTHTGEKPYKCPECGKSFSRADNLTEHQRTHTGEKPYKCPECGKSFSRRDELNVHQRTHTGKKTS

SEQ ID NO: 682 - protein, 176 aa, synthetic construct
LEPGEKPYKCPECGKSFSDSGNLRVHQRTHTGEKPYKCPECGKSFSQSSSLVRHQRTHTGEKPYKCPECGKSFSQSSSLVRHQRTHTGEKPYKCPECGKSFSQLAHLRAHQRTHTGEKPYKCPECGKSFSSKKALTEHQRTHTGEKPYKCPECGKSFSQRAHLERHQRTHTGKKTS

SEQ ID NO: 683 - protein, 176 aa, synthetic construct
LEPGEKPYKCPECGKSFSQSGDLRRHQRTHTGEKPYKCPECGKSFSRRDELNVHQRTHTGEKPYKCPECGKSFSERSHLREHQRTHTGEKPYKCPECGKSFSERSHLREHQRTHTGEKPYKCPECGKSFSTSGELVRHQRTHTGEKPYKCPECGKSFSQRANLRAHQRTHTGKKTS

SEQ ID NO: 684 - protein, 232 aa, synthetic construct
LEPGEKPYKCPECGKSFSQKSSLIAHQRTHTGEKPYKCPECGKSFSRSDHLTNHQRTHTGEKPYKCPECGKSFSRKDNLKNHQRTHTGEKPYKCPECGKSFSSKKHLAEHQRTHTGEKPYKCPECGKSFSRSDHLTNHQRTHTGEKPYKCPECGKSFSQKSSLIAHQRTHTGEKPYKCPECGKSFSTTGALTEHQRTHTGEKPYKCPECGKSFSTKNSLTEHQRTHTGKKTS

SEQ ID NO: 685 - protein, 204 aa, synthetic construct
LEPGEKPYKCPECGKSFSSKKALTEHQRTHTGEKPYKCPECGKSFSTTGNLTVHQRTHTGEKPYKCPECGKSFSTTGALTEHQRTHTGEKPYKCPECGKSFSQAGHLASHQRTHTGEKPYKCPECGKSFSTKNSLTEHQRTHTGEKPYKCPECGKSFSTSGNLTEHQRTHTGEKPYKCPECGKSFSQSGNLTEHQRTHTGKKTS

SEQ ID NO: 686 - protein, 176 aa, synthetic construct
LEPGEKPYKCPECGKSFSDKKDLTRHQRTHTGEKPYKCPECGKSFSQSSSLVRHQRTHTGEKPYKCPECGKSFSRKDNLKNHQRTHTGEKPYKCPECGKSFSQRANLRAHQRTHTGEKPYKCPECGKSFSERSHLREHQRTHTGEKPYKCPECGKSFSTSGHLVRHQRTHTGKKTS

SEQ ID NO: 687 - protein, 344 aa, synthetic construct
LEPGEKPYKCPECGKSFSDPGALVRHQRTHTGEKPYKCPECGKSFSRNDALTEHQRTHTGEKPYKCPECGKSFSTSGSLVRHQRTHTGEKPYKCPECGKSFSTSGSLVRHQRTHTGEKPYKCPECGKSFSQLAHLRAHQRTHTGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSQLAHLRAHQRTHTGEKPYKCPECGKSFSQSGDLRRHQRTHTGEKPYKCPECGKSFSTSGHLVRHQRTHTGEKPYKCPECGKSFSRSDHLTTHQRTHTGKKTS

SEQ ID NO: 688 - protein, 232 aa, synthetic construct
LEPGEKPYKCPECGKSFSDPGNLVRHQRTHTGEKPYKCPECGKSFSTKNSLTEHQRTHTGEKPYKCPECGKSFSTKNSLTEHQRTHTGEKPYKCPECGKSFSRADNLTEHQRTHTGEKPYKCPECGKSFSHKNALQNHQRTHTGEKPYKCPECGKSFSRNDALTEHQRTHTGEKPYKCPECGKSFSRRDELNVHQRTHTGEKPYKCPECGKSFSHTGHLLEHQRTHTGKKTS

SEQ ID NO: 689 - protein, 176 aa, synthetic construct
LEPGEKPYKCPECGKSFSQAGHLASHQRTHTGEKPYKCPECGKSFSQRAHLERHQRTHTGEKPYKCPECGKSFSSPADLTRHQRTHTGEKPYKCPECGKSFSQSGDLRRHQRTHTGEKPYKCPECGKSFSQSGNLTEHQRTHTGEKPYKCPECGKSFSHRTTLTNHQRTHTGKKTS

SEQ ID NO: 690 - protein, 288 aa, synthetic construct
LEPGEKPYKCPECGKSFSHKNALQNHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGEKPYKCPECGKSFSTSGNLVRHQRTHTGEKPYKCPECGKSFSRADNLTEHQRTHTGEKPYKCPECGKSFSQSGNLTEHQRTHTGEKPYKCPECGKSFSRKDNLKNHQRTHTGEKPYKCPECGKSFSDPGALVRHQRTHTGKKTS

SEQ ID NO: 691 - DNA, 18 bp, synthetic construct
  source 1..18: mol_type = other DNA
gtagaaactcgccttatg

SEQ ID NO: 692 - DNA, 21 bp, synthetic construct
  source 1..21: mol_type = other DNA
tgaatgagtcctgtccatctt

SEQ ID NO: 693 - DNA, 21 bp, synthetic construct
  source 1..21: mol_type = other DNA
aagattagaacaaatgtccag

SEQ ID NO: 694 - DNA, 18 bp, synthetic construct
  source 1..18: mol_type = other DNA
actctaagcagcaatgta

SEQ ID NO: 695 - DNA, 18 bp, synthetic construct
  source 1..18: mol_type = other DNA
tgggatagtgaaaatgtc

SEQ ID NO: 696 - DNA, 27 bp, synthetic construct
  source 1..27: mol_type = other DNA
aaaacttgggtcactaaaatagatgat

SEQ ID NO: 697 - DNA, 27 bp, synthetic construct
  source 1..27: mol_type = other DNA
aaacatggaaaaggtcaaaaacttggg

SEQ ID NO: 698 - DNA, 24 bp, synthetic construct
  source 1..24: mol_type = other DNA
aatgactagaatgaagtcctactg

SEQ ID NO: 699 - protein, 176 aa, synthetic construct
LEPGEKPYKCPECGKSFSRRDELNVHQRTHTGEKPYKCPECGKSFSTTGALTEHQRTHTGEKPYKCPECGKSFSHTGHLLEHQRTHTGEKPYKCPECGKSFSTHLDLIRHQRTHTGEKPYKCPECGKSFSQSSNLVRHQRTHTGEKPYKCPECGKSFSQSSSLVRHQRTHTGKKTS

SEQ ID NO: 700 - protein, 204 aa, synthetic construct
LEPGEKPYKCPECGKSFSTTGALTEHQRTHTGEKPYKCPECGKSFSTSGNLTEHQRTHTGEKPYKCPECGKSFSDPGALVRHQRTHTGEKPYKCPECGKSFSTKNSLTEHQRTHTGEKPYKCPECGKSFSHRTTLTNHQRTHTGEKPYKCPECGKSFSRRDELNVHQRTHTGEKPYKCPECGKSFSQAGHLASHQRTHTGKKTS

SEQ ID NO: 701 - protein, 204 aa, synthetic construct
LEPGEKPYKCPECGKSFSRADNLTEHQRTHTGEKPYKCPECGKSFSDPGALVRHQRTHTGEKPYKCPECGKSFSTTGNLTVHQRTHTGEKPYKCPECGKSFSSPADLTRHQRTHTGEKPYKCPECGKSFSQLAHLRAHQRTHTGEKPYKCPECGKSFSHKNALQNHQRTHTGEKPYKCPECGKSFSRKDNLKNHQRTHTGKKTS

SEQ ID NO: 702 - protein, 176 aa, synthetic construct
LEPGEKPYKCPECGKSFSQSSSLVRHQRTHTGEKPYKCPECGKSFSTTGNLTVHQRTHTGEKPYKCPECGKSFSERSHLREHQRTHTGEKPYKCPECGKSFSERSHLREHQRTHTGEKPYKCPECGKSFSQNSTLTEHQRTHTGEKPYKCPECGKSFSTHLDLIRHQRTHTGKKTS

SEQ ID NO: 703 - protein, 176 aa, synthetic construct
LEPGEKPYKCPECGKSFSDPGALVRHQRTHTGEKPYKCPECGKSFSTTGNLTVHQRTHTGEKPYKCPECGKSFSQSSNLVRHQRTHTGEKPYKCPECGKSFSHRTTLTNHQRTHTGEKPYKCPECGKSFSTSGNLVRHQRTHTGEKPYKCPECGKSFSRSDHLTTHQRTHTGKKTS

SEQ ID NO: 704 - protein, 260 aa, synthetic construct
LEPGEKPYKCPECGKSFSTSGNLVRHQRTHTGEKPYKCPECGKSFSTSGNLVRHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSDPGALVRHQRTHTGEKPYKCPECGKSFSRSDHLTTHQRTHTGEKPYKCPECGKSFSTHLDLIRHQRTHTGEKPYKCPECGKSFSQRANLRAHQRTHTGKKTS

SEQ ID NO: 705 - protein, 260 aa, synthetic construct
LEPGEKPYKCPECGKSFSRSDKLVRHQRTHTGEKPYKCPECGKSFSTTGALTEHQRTHTGEKPYKCPECGKSFSQRANLRAHQRTHTGEKPYKCPECGKSFSQSGNLTEHQRTHTGEKPYKCPECGKSFSTSGHLVRHQRTHTGEKPYKCPECGKSFSQRANLRAHQRTHTGEKPYKCPECGKSFSQRAHLERHQRTHTGEKPYKCPECGKSFSTSGNLTEHQRTHTGEKPYKCPECGKSFSQRANLRAHQRTHTGKKTS

SEQ ID NO: 706 - protein, 232 aa, synthetic construct
LEPGEKPYKCPECGKSFSRNDALTEHQRTHTGEKPYKCPECGKSFSQNSTLTEHQRTHTGEKPYKCPECGKSFSDPGALVRHQRTHTGEKPYKCPECGKSFSQSSNLVRHQRTHTGEKPYKCPECGKSFSTTGNLTVHQRTHTGEKPYKCPECGKSFSREDNLHTHQRTHTGEKPYKCPECGKSFSDPGNLVRHQRTHTGEKPYKCPECGKSFSTTGNLTVHQRTHTGKKTS

SEQ ID NO: 707 - DNA, 23 bp, synthetic construct
  source 1..23: mol_type = other DNA
ataacacgtgagccgtcctaagg

SEQ ID NO: 708 - protein, 26 aa, synthetic construct
AKLAGGAPAVGGGPKAADKFAATGGS

SEQ ID NO: 709 - protein, 148 aa, synthetic construct
LEPGEKPYKCPECGKSFSQNSTLTEHQRTHTGEKPYKCPECGKSFSQRANLRAHQRTHTGEKPYKCPECGKSFSTSGNLVRHQRTHTGEKPYKCPECGKSFSQSSNLVRHQRTHTGEKPYKCPECGKSFSRSDHLTTHQRTHTGKKTS

(19) United States
(12) Patent Application Publication
(10) Pub. No.: US 2025/0207111 A1
(43) Pub. Date: Jun. 26, 2025

(54) MOBILE GENETIC ELEMENTS FROM EPTESICUS FUSCUS

(71) Applicant: Saliogen Therapeutics, Inc., Lexington, MA (US)

(72) Inventors: Joseph J. HIGGINS, Lexington, MA (US); Nancy CRAIG, Lexington, MA (US); David RAY, Lexington, MA (US); Subhrangshu GUHATHAKURTA, Lexington, MA (US); Sharmishtha MUSALGAONKAR, Lexington, MA (US)

(21) Appl. No.: 18/867,104

(22) PCT Filed: May 25, 2023
(86) PCT No.: PCT/US2023/067472

§ 371 Date: Nov. 19, 2024

Related U.S. Application Data
(60) Provisional application No. 63/498,967, filed on Apr. 28, 2023.
(60) Provisional application No. 63/346,145, filed on May 26, 2022.

(51) Int. Cl. C12N 9/12; C12N 9/22; C12N 15/11; C12N 15/88

(52) U.S. Cl. CPC C12N 9/1241; C12N 9/22; C12N 15/11; C12N 15/88; C12N 2310/20

(57) ABSTRACT

Gene therapy compositions and methods related to transposition are provided, e.g., those engineered from Eptesicus fuscus.

### CROSS-REFERENCE TO RELATED APPLICATIONS

This application claims priority to and the benefit of U.S. Provisional Patent Application Nos. 63/346,145, filed on May 26, 2022, and 63/498,967 filed on Apr. 28, 2023 the entire contents of all of which are hereby incorporated by reference.

### FIELD

The present disclosure relates to recombinant mobile element systems and uses thereof. Specifically, the recombinant mobile element systems of the present disclosure are derived from Eptesicus fuscus.

### DESCRIPTION OF THE TEXT FILE SUBMITTED ELECTRONICALLY

The instant application contains a sequence listing, which has been submitted in XML format via EFS-Web. The contents of the XML copy named “SAL-018PC_126933-5018_Sequence Listing,” which was created on May 22, 2023 and is 659,511 bytes in size, the contents of which are incorporated herein by reference in their entirety.

### BACKGROUND

Mobile elements are genetic sequences that are found, with small exceptions, in all living organisms. These elements have deep evolutionary origins and diversification and have an astonishing variety of forms and shapes.

The most widely used transposon system is that of the piggyBac system, which was originally identified in moths in 1983. When combining a piggyBac transposon with a piggyBac helper enzyme, the DNA sequence from the transposon vector can be transferred to one of many specific nucleotide sequence (i.e., TTAA) sequences distributed throughout the genome.

However, current transposition systems only find use in laboratory applications. Therapeutic uses have proven elusive.

There is a need for novel and safer transposon systems for this technology to find use in medicine.

### SUMMARY

Accordingly, this disclosure describes, in part, a helper enzyme, optionally in the form of RNA, which is optionally engineered to target a single human genomic locus by introducing a DNA binding protein to yield a chimeric agent. The present disclosure provides, inter alia, a composition comprising a recombinant mobile element enzyme and a DNA binder (e.g., without limitation, dCas9, dCasX, TALEs, TniQ subdomain of TnsD TniQ subdomain of TniQ, and ZnF) that guide donor insertion to specific genomic sites.

In embodiments, the helper enzyme is an engineered form of an enzyme reconstructed from Eptesicus fuscus. In embodiments, the enzyme includes but is not limited to an engineered version that is a monomer, dimer, tetramer (or another multimer), hyperactive (Exc+), and/or has a reduced interaction with non-TTAA recognitions sites (Int−), of a helper enzyme reconstructed from Eptesicus fuscus or a predecessor thereof.

In embodiments, the helper enzyme is a recombinant molecule which has at least about 90% identity to the nucleotide sequence of SEQ ID NO: 2 or the amino acid sequence SEQ ID NO: 1. In embodiments, the helper enzyme has at least about 95%, or at least about 96%, at least about 97%, at least about 98%, at least about 99% identity to the amino acid sequence of SEQ ID NO: 1, or a nucleotide sequence encoding the same.

In embodiments, the composition comprises a gene transfer construct. In embodiments, the gene transfer construct comprises a donor (e.g., donor DNA) and can be or can comprise a vector comprising a mobile element comprising one or more end sequences recognized by the enzyme. In embodiments, the end sequences are left and right end sequences that are recombinant or synthetic sequences. In embodiments, the end sequences are from Eptesicus fuscus, or end sequences with similarity to piggyBac-like mobile elements and exhibit duplications of their presumed TTAA target sites.

In embodiments, the end sequences include at least one repeat from a nucleotide sequence having at least about 90% identity to the nucleotide sequence of SEQ ID NO: 3, and wherein the at least one repeat from the nucleotide sequence having at least about 90% identity to the nucleotide sequence of SEQ ID NO: 3 is positioned at the 5′ end of the donor. In embodiments, the end sequences can further include at least one repeat from a nucleotide sequence having at least about 90% identity to the nucleotide sequence of SEQ ID NO: 4, and wherein the at least one repeat from the nucleotide sequence having at least about 90% identity to the nucleotide sequence of SEQ ID NO: 4 is positioned at the 3′ end of the donor. In embodiments, the end sequences, which can be, e.g., from Eptesicus fuscus, are optionally flanked by a TTAA sequence.

In embodiments, the enzyme is included in the gene transfer construct. In embodiments, the composition comprises a nucleic acid binding component of a gene-editing system. In embodiments, the gene-editing system is included in the gene transfer construct.

In embodiments, the gene-editing system comprises a CRISPR/Cas enzyme (class I, class II), or their six subtypes (type I-VI) (e.g., Cas9, Cas12a, Cas12j, Cas12k), or a variant thereof. In embodiments, the gene-editing system comprises a nuclease-deficient a CRISPR/Cas enzyme (class I, class II), or their six subtypes (type I-VI) (e.g., dCas9, dCas12a, dCas12j, dCas12k). In embodiments, the gene-editing system comprises Cas9, Cas12a, Cas12j, or Cas12k, or a variant thereof. For example, the gene-editing system comprises a nuclease-deficient dCas9, dCas12a, dCas12j, or dCas12k. In embodiments, the gene-editing system comprises a TALE, ZnF, TniQ subdomain of TnsD, or TniQ subdomain of TniQ.

In embodiments, the composition has the helper enzyme and the nucleic acid binding component of the gene-editing system.

In embodiments, the composition comprises a chimeric mobile element construct comprising the helper enzyme and the nucleic acid binding component of the gene-editing system fused or linked thereto. In embodiments, the helper enzyme and the nucleic acid binding component of the gene-editing system can be fused or linked to one another via a linker (e.g., original linker AKLAGGAPAVGGGPKAADKFAATGGS (SEQ ID NO: 913), a flexible linker, or in the case of non-covalent bonding, a small peptide for covalent binding of a monobody, nanobody or single-chain variable fragment (scFv) antibody linked to a DNA binding domain (TALE, ZnF, or dCas). In embodiments, the flexible linker can be substantially comprised of glycine and serine residues, optionally wherein the flexible linker comprises (Gly₄Ser)ₙ, where n is from about 1 to about 12. In embodiments, the flexible linker is of or about 50, or about 100, or about 150, or about 200 amino acid residues. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt. In embodiments, the helper enzyme is capable of inserting a donor at a TA dinucleotide site or a TTAA tetranucleotide site in a genomic safe harbor site (GSHS) of a nucleic acid molecule.

In embodiments, the donor comprises a gene encoding a complete polypeptide. In embodiments, the donor comprises a gene which is defective or substantially absent in a disease state.

In aspects, a composition is provided comprising (a) a nucleic acid binding component of a gene-editing system, and (b) a recombinant mammalian helper enzyme, the helper enzyme having at least about 90% identity to the amino acid sequence of SEQ ID NO: 1, or a nucleotide sequence encoding the same. In embodiments, the helper enzyme has at least about 95%, or at least about 96%, at least about 97%, at least about 98%, at least about 99% identity to the amino acid sequence of SEQ ID NO: 1, or a nucleotide sequence encoding the same.

In embodiments, a mobile element construct comprises a helper enzyme (both herein called “helper”) constructed as a DNA vector or RNA vector (FIG. 1A) fused or linked to a DNA binding domain (DBD), or TALE (FIG. 1B), zinc finger (ZnF) (FIG. 1C), inactive Cas protein (dCas9, dCas12a, dCas12j, or dCas12k) programmed by a guide RNA (gRNA) (FIG. 1D), or a construct with an intein or dimerization enhancer such as SH3, biotin, avidin, or rapamycin binders (FIG. 1E).

In embodiments, a composition comprising a recombinant mammalian helper enzyme in accordance with embodiments of the present disclosure can include one or more non-viral vectors. Also, in embodiments, the recombinant mammalian helper enzyme can be disposed on the same (cis) or different vector (trans) than a donor with a transgene. Accordingly, in embodiments, the recombinant mammalian helper enzyme and the donor encompassing a transgene are in cis configuration such that they are included in the same vector. In embodiments, the recombinant mammalian helper enzyme and the donor encompassing a transgene are in trans configuration such that they are included in different vectors. In embodiments, the vector is any non-viral vector in accordance with the present disclosure.

In embodiments, the present disclosure provides a method for inserting a gene into the genome of a cell, comprising contacting a cell with the composition of the present disclosure or host cell of the present disclosure. In embodiments, the method of the present disclosure further comprising contacting the cell with a polynucleotide encoding a donor DNA. In embodiments, the donor comprises a gene encoding a complete polypeptide. In embodiments, the donor comprises a gene which is defective or substantially absent in a disease state.

In embodiments, the present disclosure provides a method for treating a disease or disorder ex vivo, comprising contacting a cell with the composition of the present disclosure or host cell of the present disclosure and administering the cell to a subject in need thereof.

In embodiments, the present disclosure provides a method for treating a disease or disorder in vivo, comprising administering the composition of the present disclosure or host cell of the present disclosure to a subject in need thereof.

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof.

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof, wherein the heterologous polynucleotide is transposable by a helper enzyme having the sequence of SEQ ID NO: 1, or a functional variant thereof.

In embodiments, there is provided a polynucleotide comprising an open reading frame encoding a helper enzyme which is at least 90% identical to SEQ ID NO: 2, or a functional variant thereof, operably linked to a heterologous promoter.

In embodiments, there is provided a polynucleotide comprising an open reading frame encoding a transposase, the amino acid sequence of which is at least 90% identical to SEQ ID NO: 1, or a functional variant thereof, operably linked to a heterologous promoter.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIG. 1A-FIG. 1E depict five illustrative bioengineered RNA helper constructs that are contained in a replication backbone (e.g., plasmid or miniplasmid) with a T7 promoter (cap dependent), beta-globin 5′-UTR, and a helper enzyme (SEQ ID NO: 1, SEQ ID NO: 2) from Eptesicus fuscus followed by a beta-globin 3′-UTR, and a poly-alanine tail (FIG. 1A). TALES (FIG. 1B, TABLE 7-TABLE 12), ZnF (FIG. 1C, TABLE 13-TABLE 17), or a dead Cas9 (dCas9) binding protein (FIG. 1D, SEQ ID NO: 5, SEQ ID NO: 6) with guide RNAs (TABLE 1-TABLE 6) were joined by a linker to the N-terminus to target the specific TTAA sites at hROSA 26, AAVS1, chromosome 4, chromosome 22, and chromosome X loci. FIG. 1E depicts a construct with a dimerization enhancer to assure activation of the two monomers.

FIG. 2A depicts an illustrative core donor construct that is contained in a replication backbone (e.g., plasmid or miniplasmid) with a promoter driving a gene of interest (GOI) with a polyA tail flanked by two insulators and ITRs. The inverted terminal repeat (ITR) recognition sequences are included at the 5′-(SEQ ID NO: 3) and 3′-ends (SEQ ID NO: 4). This construct is used for targeting genomic safe harbor sites (GSHS) or other loci.

FIG. 2B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. The inverted terminal repeat (ITR) recognition sequences are included at the 5′-(SEQ ID NO: 3) and 3′-ends (SEQ ID NO: 4). This construct is used for targeting endogenous genes in the first intron (or other introns) to repair downstream mutations.

FIG. 2C depicts an illustrative core donor construct that is contained in a replication backbone (e.g., plasmid or miniplasmid) with tandem promoters to affect expression in different tissues (e.g., without limitation, liver specific promoter, cardiac specific promoter) and a gene(s) of interest (GOI) followed by a polyA tail and flanked by ITRs. The inverted terminal repeat (ITR) recognition sequences are included at the 5′-(SEQ ID NO: 3) and 3′-ends (SEQ ID NO: 4). This construct is used to differentially promote expression of genes in different organs, tissues or cell types.

FIG. 2D depicts an illustrative core donor construct that is contained in a replication backbone (e.g., plasmid or miniplasmid) with two or more genes of interest (GOI) linked by P2A “self-cleaving” peptides and followed by WPRE and a polyA tail. The construct is flanked by ITRs. The inverted terminal repeat (ITR) recognition sequences are included at the 5′-(SEQ ID NO: 3) and 3′-ends (SEQ ID NO: 4). This construct is used for delivering multiple genes or genetic factors.

FIG. 2E depicts an illustrative core donor construct that is contained in a replication backbone (e.g., plasmid or miniplasmid) with a promoter(s) driving the expression of two or more genes as in FIG. 2D and linked to a sequence consisting of a 5′-miRNA, a sense and antisense miRNA pair, and completed with the 3′-miRNA. The construct is followed by WPRE and flanked by ITRs. The inverted terminal repeat (ITR) recognition sequences are included at the 5′-(SEQ ID NO: 3) and 3′-ends (SEQ ID NO: 4). This construct combines protein replacement and miRNA to inhibit the expression of other related proteins.

FIG. 3 depicts the TTAA site in hROSA26 (hg38 chr3: 9,396, 133-9,396,332) that is targeted by guideRNAs (TABLE 2), TALEs (TABLE 8), and ZnF (TABLE 13).

FIG. 4 depicts two TTAA sites in AAVS1 (hg38 chr19: 55, 112,851-55, 113,324) that are targeted by guideRNAs (TABLE 3) or TALEs (TABLE 9), and ZnF (TABLE 14).

FIG. 5 depicts two TTAA sites in Chromosome 4 (hg38 chr4: 30,039,534-30,793,980) that are targeted by guideRNAs (TABLE 4) or TALEs (TABLE 10), and ZnF (TABLE 15).

FIG. 6 depicts two TTAA sites in Chromosome 22 (hg38 chr22: 35,373,429-35,380,000) that are targeted by guideRNAs (TABLE 5) or TALEs (TABLE 11), and ZnF (TABLE 16).

FIG. 7 depicts two TTAA sites in Chromosome X (hg38 chrX: 134,475,809-134,476,794) that are targeted by guideRNAs (TABLE 6) or TALEs (TABLE 12), and ZnF (TABLE 17).

FIG. 8 depicts an illustrative strategy to identify hyperactive variants of synthetic Epticus fuscus transposase.

FIG. 9A and FIG. 9B depicts the excision activity of synthetic Epticus fuscus transposase (sEFT) as measured flow cytometry GFP expression in FIG. 9A and direct visualization of the transposed cells in FIG. 9B.

### DETAILED DESCRIPTION

The present invention is based, in part, on the discovery of an engineered helper enzyme capable of gene insertion that finds uses in multiple applications, including, without limitation, in gene therapy. In aspects, there is provided an engineered enzyme from Eptesicus fuscus, e.g., having an amino acid sequence of SEQ ID NO: 1 or a variant thereof, inclusive of variants generated (e.g., by random mutagenesis and/or site directed mutagenesis) (occasionally may be referred to as “engineered”, “the present EFT”, “hyperactive helper from Eptesicus fuscus”, or “hyperactive helper”). “EFT”, as used herein, refers to Eptesicus fuscus helper, as engineered herein.

The present invention is based, in part, on the discovery that a helper enzyme, e.g., a recombinant helper enzyme derived from Eptesicus fuscus, can be fused with a transcription activator-like effector proteins (TALE) DNA binding domain (DBD), a dCas9/gRNA, or a zinc finger sequence to thereby create a chimeric enzyme capable of a site- or locus-specific transposition. For instance, in the case of a fusion to a TALE DBD, the enzyme (e.g., without limitation, a chimeric helper) utilizes the specificity of TALE DBD to certain sites within a host genome, which allows using DBDs to target any desired location in the genome. In this way, the chimeric helper in accordance with the present disclosure allows achieving targeted integration of a transgene.

In embodiments, the helper has one or more mutations that confer hyperactivity. In embodiments, the helper is a mammal-derived helper, optionally a helper RNA helper. Thus, in embodiments, the present compositions and methods for gene transfer utilize a dual donor/helper system. Transposable elements are non-viral gene delivery vehicles found ubiquitously in nature. Donor-based vectors have the capacity of stable genomic integration and long-lasting expression of transgene constructs in cells. Generally, dual donor and helper systems work via a cut-and-paste mechanism whereby donor DNA containing a transgene(s) of interest is integrated into chromosomal DNA by a helper enzyme at a repetitive sequence site. Dual donor/helper (or “donor/helper”) plasmid systems insert a transgene flanked by inverted terminal ends (“ends”), such as TTAA (SEQ ID NO: 440) tetranucleotide sites, without leaving a DNA footprint in the human genome. The helper enzyme, in embodiments, is transiently expressed (on the same or a different vector from a vector encoding the donor) and it catalyzes the insertion events from the donor plasmid to the host genome. Genomic insertions primarily target introns but may target other TTAA (SEQ ID NO: 440) sites and integrate into approximately 50% of human genes.

This disclosure describes, in embodiments, a DNA integration system, which is highly active in mammals, and is derived from a mammalian mobile DNA element. In embodiments, this mammal-derived mobile genetic element is engineered to insert donor DNA at specific TTAA insertion “hotspots” that are frequently favored insertion sites for the un-engineered enzyme. In embodiments, this technology exploits a helper RNA encoding enzyme with engineered DNA binding proteins and a donor DNA contained between the ends of a mobile element of the gene to be inserted into the genome. In embodiments, the mammal-derived enzyme is fused to a protein domain at its N-terminus without loss of activity and “engineered” by fusing DNA binding domains (DBD) that can target almost any location in the genome. Excision competent/target binding defective enzymes (Exc+/Int) mutants are described, that when combined with programmable, synthetic DBDs only insert at a TTAAs at a single target site. This enzyme described in this disclosure displays several highly desirable features that are of great advantage for transgene integration. In embodiments, no DNA double strand breaks are introduced into the target genome. Furthermore, upon enzyme-mediated excision containing a gene of interest from its donor DNA, the flanking donor backbone ends are very efficiently rejoined, leaving no double strand break in the donor DNA to signal DNA damage. In embodiments, the enzyme inserts the excised element at high frequency selectively into a TTAA target site. Notably, because excision from the donor site results in the covalent linkage of a TTAA segment to each 5′ donor end, the joining of the 3′ donor ends to staggered positions on the top and bottom strands of the DNA flanking the target TTAA, a simple ligation restores intact duplex DNA, and no DNA synthesis is required for repair. In embodiments, the enzyme delivers a large cargo size as compared to other mobile genetic elements or integrating viral systems to date.

In embodiments, the enzyme is delivered as an RNA instead of as a DNA. Other mobile genetic elements including helpers such as hyperactive PB and SB100X, when delivered as RNA, have significantly less activity when compared to DNA. See Bire, et al. (2013). Exogenous mRNA delivery and bioavailability in gene transfer mediated by piggyBac transposition. BMC Biotechnol, 13, 75; Bire, et al. (2013). Optimization of the piggyBac donor using mRNA and insulators: toward a more reliable gene delivery system. PLoS One, 8 (12), e82559; Wilber, et al. (2006). RNA as a source of helper for Sleeping Beauty-mediated gene insertion and expression in somatic cells and tissues. Mol Ther, 13 (3), 625-630. In embodiments, the enzyme described herein has the same or better activity when delivered as RNA. The use of helper RNA offers several advantages over delivery of a DNA molecule. Wilber, et al. (2006). RNA as a source of helper for Sleeping Beauty-mediated gene insertion and expression in somatic cells and tissues. Mol Ther, 13 (3), 625-630. For instance, without wishing to be bound by theory, there is improved control with respect to the duration of enzyme expression, minimizing persistence in the tissue, and there is potential for transgene re-mobilization and re-insertion following the initial transposition event. Furthermore, in embodiments, the helper-encoding RNA sequence is incapable of integrating into the host genome, thereby eliminating concerns about long-term helper expression and destabilizing effects with respect to the gene of interest. This safety feature, in embodiments, prevents the integration of the enzyme gene into the human genome and circumvents potential oncogenic and mutagenic effects. In embodiments, the present disclosure provides a dual DNA donor and RNA helper system. The donor DNA plasmid contains helper-specific inverted terminal repeats (ITRs) flanking the transgene while the helper-RNA transiently expresses a synthetic enzyme that catalyzes the insertion events from the donor plasmid to the host genome. This two component DNA/RNA system is, in embodiments, co-encapsulated in a single lipid nanoparticle using microfluidic technology and the lipid nanoparticles protect the RNA from extracellular degradation by in vivo injection.

### Helper Enzyme

In embodiments, the present disclosure provides a composition comprising a helper enzyme or a nucleic acid encoding the enzyme, wherein the enzyme comprises an amino acid sequence having at least about 80% sequence identity to SEQ ID NO: 1.

| SEQ ID NO: 1: Amino acid sequence synthetic Eptesicus fuscus (638 amino acids) | |
|---|---|
| transposase. Bolded amino acids filled in from Microcebus marinus genomic | |
| fragments or aligned based on missing nucleotide sequences. | |
| 1 | MDKFSKDIES SDDEFYFENE EKSEKCNSDE SEFSEDASGD DEQIAGPSGT TERKKSLALP |
| 61 | KDLAESTDSD SDIEFIKAKR RRTIVYSSES DGDIGDIIEK SGIRPSESYV SRGKQEKEKW |
| 121 | TSTSVNDKEP SRIPFSTGQL HVGPQVPSGC ATPIDFFQLF FTETLIKNIT DETNEYARHK |
| 181 | ISQKELSQRS TWNNWKDVTI EEMKAFLGVI LNMGVLNHPN LQSYWSMDFE SHIPFFRSVF |
| 241 | KRERFLQIFW MLHLKNDQKS SKDLRTRTEK VNCFLSYLEM KFRERFCPGR EIAVDEAVVG |
| 301 | FKGKIHFITY NPKKPTKWGI RLYVLSDSKC GYVHSFVPYY GGITSETLVR PDLPFTSRIV |
| 361 | LELHERLKNS VPGSQGYHFF TDRYYTSVTL AKELFKEKTH LTGTIMPNRK DNPPVIKHPK |
| 421 | LMKGEIVAFR DENVMLLAWK DKRIVTMLST WDTSETESVE RRVRGGGKEI VLKPKVVTNY |
| 481 | TKFMGGVDIA DHYTGTYCFM RKTLKWWRKL FFWGLEVSVV NSYILYKECQ KRKNEKPITH |
| 541 | VKFIRKLVHD LVGEFRDGTL TSRGRLLSTN LEQRLDGKLH IITPHPNKKH KDCVVCSNRK |
| 601 | IKGGRRETIY ICETCECKPG LHVGECFKKY HTMKNYRD |

In embodiments, the enzyme comprises an amino acid sequence of at least about 80% identity to SEQ ID NO: 1. In embodiments, the enzyme comprises an amino acid sequence of at least about 83% identity to SEQ ID NO: 1. In embodiments, the enzyme comprises an amino acid sequence of at least about 85% identity to SEQ ID NO: 1. In embodiments, the enzyme comprises an amino acid sequence of at least about 88% identity to SEQ ID NO: 1. In embodiments, the enzyme comprises an amino acid sequence of at least about 89% identity to SEQ ID NO: 1. In embodiments, the enzyme comprises an amino acid sequence of at least about 90% identity to SEQ ID NO: 1. In embodiments, the enzyme comprises an amino acid sequence of at least about 93% identity to SEQ ID NO: 1. In embodiments, the enzyme comprises an amino acid sequence of at least about 95% identity to SEQ ID NO: 1. In embodiments, the enzyme comprises an amino acid sequence of at least about 98% identity to SEQ ID NO: 1. In embodiments, the enzyme comprises an amino acid sequence of at least about 99% identity to SEQ ID NO: 1.

In embodiments, the amino acid sequence of the enzyme comprises an aspartic acid or glutamic acid at position 2 relative to SEQ ID NO: 1. In embodiments, the amino acid sequence of the enzyme comprises an aspartic acid at position 2 relative to SEQ ID NO: 1. In embodiments, the amino acid sequence of the enzyme comprises an aspartic acid or glutamic acid at position 41 relative to SEQ ID NO: 1. In embodiments, the amino acid sequence of the enzyme comprises an aspartic acid at position 41 relative to SEQ ID NO: 1. In embodiments, the enzyme comprises a serine, threonine, or tyrosine at position 69 relative to SEQ ID NO: 1. In embodiments, the enzyme comprises a serine at position 69 relative to SEQ ID NO: 1. In embodiments, the amino acid sequence of the enzyme comprises an aspartic acid or glutamic acid at position 70 relative to SEQ ID NO: 1. In embodiments, the amino acid sequence of the enzyme comprises an aspartic acid at position 70 relative to SEQ ID NO: 1. In embodiments, the enzyme comprises an arginine, histidine, or lysine at position 81 relative to SEQ ID NO: 1. In embodiments, the enzyme comprises an arginine at position 81 relative to SEQ ID NO: 1. In embodiments, the amino acid sequence of the enzyme comprises a serine, threonine, or tyrosine at position 87 relative to SEQ ID NO: 1. In embodiments, the amino acid sequence of the enzyme comprises a serine at position 87 relative to SEQ ID NO: 1. In embodiments, the amino acid sequence of the enzyme comprises a serine, threonine, or tyrosine at position 88 relative to SEQ ID NO: 1. In embodiments, the amino acid sequence of the enzyme comprises a serine at position 88 relative to SEQ ID NO: 1. In embodiments, the amino acid sequence of the enzyme comprises a glycine, alanine, isoleucine, leucine, methionine, proline or valine at position 92 relative to SEQ ID NO: 1. In embodiments, the amino acid sequence of the enzyme comprises a glycine at position 92 relative to SEQ ID NO: 1. In embodiments, the amino acid sequence of the enzyme comprises a serine, threonine, or tyrosine at position 101 relative to SEQ ID NO: 1. In embodiments, the amino acid sequence of the enzyme comprises a serine at position 101 relative to SEQ ID NO: 1. In embodiments, the amino acid sequence of the enzyme comprises a serine, threonine, or tyrosine at position 109 relative to SEQ ID NO: 1. In embodiments, the amino acid sequence of the enzyme comprises a tyrosine at position 109 relative to SEQ ID NO: 1. In embodiments, the amino acid sequence of the enzyme comprises an arginine, histidine, or lysine at position 114 relative to SEQ ID NO: 1. In embodiments, the amino acid sequence of the enzyme comprises a lysine at position 114 relative to SEQ ID NO: 1. In embodiments, the amino acid sequence of the enzyme comprises a glutamine or asparagine at position 115 relative to SEQ ID NO: 1. In embodiments, the amino acid sequence of the enzyme comprises a glutamine at position 115 relative to SEQ ID NO: 1. In embodiments, the amino acid sequence of the enzyme comprises an aspartic acid or glutamic acid at position 116 relative to SEQ ID NO: 1. In embodiments, the amino acid sequence of the enzyme comprises a glutamic acid at position 116 relative to SEQ ID NO: 1. In embodiments, the amino acid sequence of the enzyme comprises an aspartic acid or glutamic acid at position 118 relative to SEQ ID NO: 1. In embodiments, the amino acid sequence of the enzyme comprises a glutamic acid at position 118 relative to SEQ ID NO: 1. In embodiments, the amino acid sequence of the enzyme comprises an aspartic acid or glutamic acid at position 185 relative to SEQ ID NO: 1. In embodiments, the amino acid sequence of the enzyme comprises a glutamic acid at position 185 relative to SEQ ID NO: 1. In embodiments, the amino acid sequence of the enzyme comprises an arginine, histidine, or lysine at position 189 relative to SEQ ID NO: 1. In embodiments, the amino acid sequence of the enzyme comprises an arginine at position 189 relative to SEQ ID NO: 1. In embodiments, the amino acid sequence of the enzyme comprises a phenylalanine, threonine, or tryptophan at position 192 relative to SEQ ID NO: 1. In embodiments, the amino acid sequence of the enzyme comprises a tryptophan at position 192 relative to SEQ ID NO: 1. In embodiments, the amino acid sequence of the enzyme comprises a glycine, alanine, isoleucine, leucine, methionine, proline, or valine at position 447 relative to SEQ ID NO: 1. In embodiments, the amino acid sequence of the enzyme comprises a methionine at position 447 relative to SEQ ID NO: 1. In embodiments, the amino acid sequence of the enzyme comprises a serine, threonine, or tyrosine at position 453 relative to SEQ ID NO: 1. In embodiments, the amino acid sequence of the enzyme comprises a threonine at position 453 relative to SEQ ID NO: 1. In embodiments, the amino acid sequence of the enzyme comprises an arginine, histidine, or lysine at position 464 relative to SEQ ID NO: 1. In embodiments, the amino acid sequence of the enzyme comprises an arginine at position 464 relative to SEQ ID NO: 1. In embodiments, the amino acid sequence of the enzyme comprises an arginine, histidine, or lysine at position 492 relative to SEQ ID NO: 1. In embodiments, the amino acid sequence of the enzyme comprises a histidine at position 492 relative to SEQ ID NO: 1.

In embodiments, the enzyme has one or more mutations which confer hyperactivity.

In embodiments, the enzyme has one or more amino acid substitutions generated by by random mutagenesis and/or site directed mutagenesis.

| SEQ ID NO: 2: Nucleotide sequence encoding the helper from the synthetic | |
|---|---|
| Eptesicus fuscus transposase (codon optimized)(1914 nt). | |
| 1 | ATGGACAAGT TTTCCAAGGA CATTGAAAGC TCTGACGATG AATTTTACTT CGAGAACGAG |
| 61 | GAGAAAAGCG AGAAGTGTAA TTCCGATGAG TCCGAGTTTA GCGAGGACGC TAGCGGCGAC |
| 121 | GACGAGCAGA TCGCTGGACC CAGCGGGACC ACGGAGCGCA AAAAGAGCCT GGCTCTGCCT |
| 181 | AAAGACTTGG CCGAGAGTAC CGACAGCGAC TCCGATATCG AGTTCATCAA GGCCAAACGC |
| 241 | AGGCGCACAA TCGTGTACTC TTCCGAGAGC GACGGCGACA TCGGCGATAT TATCGAGAAA |
| 301 | AGCGGGATCC GGCCTTCCGA AAGCTACGTG TCTCGGGGCA AGCAGGAGAA GGAAAAGTGG |
| 361 | ACAAGCACCT CTGTGAACGA CAAAGAGCCT TCCAGAATCC CCTTCAGCAC CGGCCAGCTG |
| 421 | CATGTGGGCC CCCAGGTGCC CAGCGGCTGC GCCACTCCTA TCGACTTCTT CCAGCTGTTT |
| 481 | TTTACTGAGA CCCTGATCAA GAACATCACC GATGAGACAA ATGAGTACGC CAGGCACAAG |
| 541 | ATCTCTCAGA AGGAGCTGAG CCAGCGCAGT ACATGGAACA ACTGGAAGGA CGTGACCATC |
| 601 | GAAGAGATGA AGGCCTTCCT GGGCGTGATC CTGAATATGG GAGTGCTGAA CCATCCTAAT |
| 661 | CTGCAGTCCT ATTGGTCCAT GGATTTCGAG TCCCACATTC CATTCTTCAG GTCCGTGTTC |
| 721 | AAGCGCGAGC GTTTCCTGCA GATCTTCTGG ATGCTGCACC TGAAAAATGA CCAGAAGAGC |
| 781 | TCCAAGGACC TGCGGACACG GACTGAGAAG GTGAATTGTT TCCTGTCCTA CCTGGAGATG |
| 841 | AAATTCAGGG AGAGGTTTTG TCCCGGCCGG GAAATTGCCG TGGATGAGGC CGTGGTGGGC |
| 901 | TTCAAGGGCA AGATCCACTT CATCACCTAC AACCCAAAGA AGCCAACAAA GTGGGGCATC |
| 961 | CGGCTGTATG TCCTGAGTGA CTCCAAGTGT GGCTACGTGC ACAGCTTTGT GCCCTATTAT |
| 1021 | GGCGGCATCA CCTCCGAGAC CCTGGTGAGG CCCGACCTGC CTTTCACCTC TAGAATTGTG |
| 1081 | CTGGAGCTGC ATGAGCGGCT GAAGAACTCT GTGCCTGGCA GCCAGGGCTA CCATTTTTTC |
| 1141 | ACCGACAGGT ACTATACATC CGTTACCCTG GCCAAGGAAC TGTTCAAAGA AAAAACCCAC |
| 1201 | CTGACCGGCA CTATCATGCC CAACCGCAAG GACAACCCCC CTGTGATCAA ACATCCCAAA |
| 1261 | CTGATGAAGG GCGAGATCGT GGCCTTCAGA GACGAGAACG TCATGCTGCT GGCTTGGAAA |
| 1321 | GATAAGCGGA TCGTGACTAT GCTGTCTACA TGGGATACCT CCGAGACAGA GAGCGTTGAA |
| 1381 | CGGCGGGTGA GGGGTGGAGG CAAGGAGATC GTGCTGAAGC CAAAGGTGGT GACCAACTAC |
| 1441 | ACCAAGTTCA TGGGCGGAGT GGATATTGCA GACCATTACA CCGGCACCTA CTGTTTCATG |
| 1501 | CGGAAGACCC TGAAGTGGTG GCGGAAGCTG TTCTTCTGGG GGCTGGAGGT CAGCGTGGTG |
| 1561 | AACTCCTACA TCCTCTACAA GGAGTGCCAG AAGAGGAAGA ACGAGAAACC AATCACACAC |
| 1621 | GTGAAGTTTA TCAGGAAGCT GGTGCACGAC CTGGTGGGAG AGTTCCGCGA CGGCACCCTC |
| 1681 | ACCAGTCGGG GCCGGCTGCT GAGTACAAAC CTGGAGCAGA GGCTGGACGG AAAGCTGCAC |
| 1741 | ATTATCACTC CCCATCCAAA TAAGAAGCAC AAGGACTGCG TGGTCTGCAG CAACCGGAAG |
| 1801 | ATTAAAGGAG GGCGGCGGGA AACCATTTAC ATTTGTGAGA CCTGCGAATG CAAGCCTGGC |
| 1861 | CTGCACGTGG GGGAGTGCTT CAAGAAGTAC CACACAATGA AAAACTACAG GGAT |

In embodiments, the nucleic acid that encodes the enzyme has a nucleotide sequence of SEQ ID NO: 2 or a codon-optimized form thereof.

In embodiments, there is provided a polynucleotide comprising an open reading frame encoding a helper enzyme which is at least 90% identical to SEQ ID NO: 2, or a functional variant thereof, operably linked to a heterologous promoter.

In embodiments, the polynucleotide comprises a polynucleotide sequence of at least about 80% identity to SEQ ID NO: 2. In embodiments, the polynucleotide comprises a polynucleotide sequence of at least about 83% identity to SEQ ID NO: 2. In embodiments, the polynucleotide comprises a polynucleotide sequence of at least about 85% identity to SEQ ID NO: 2. In embodiments, the polynucleotide comprises a polynucleotide sequence of at least about 88% identity to SEQ ID NO: 2. In embodiments, the polynucleotide comprises a polynucleotide sequence of at least about 89% identity to SEQ ID NO: 2. In embodiments, the polynucleotide comprises a polynucleotide sequence of at least about 90% identity to SEQ ID NO: 2. In embodiments, the polynucleotide comprises a polynucleotide sequence of at least about 93% identity to SEQ ID NO: 2. In embodiments, the polynucleotide comprises a polynucleotide sequence of at least about 95% identity to SEQ ID NO: 2. In embodiments, the polynucleotide comprises a polynucleotide sequence of at least about 98% identity to SEQ ID NO: 2. In embodiments, the polynucleotide comprises a polynucleotide sequence of at least about 99% identity to SEQ ID NO: 2.

In embodiments, there is provided a polynucleotide comprising an open reading frame encoding a transposase, the amino acid sequence of which is at least 90% identical to SEQ ID NO: 1, or a functional variant thereof, operably linked to a heterologous promoter.

In embodiments, the enzyme is excision positive. In embodiments, the enzyme is integration deficient. In embodiments, the enzyme has decreased integration activity relative to an enzyme comprising an amino acid sequence of SEQ ID NO: 1 or functional equivalent thereof. In embodiments, the enzyme has increased excision activity relative to an enzyme comprising an amino acid sequence of SEQ ID NO: 1 or functional equivalent thereof.

In embodiments, there is provided a polynucleotide comprising an open reading frame encoding a helper enzyme which is at least 90% identical to SEQ ID NO: 2, or a functional variant thereof, operably linked to a heterologous promoter. In embodiments, there is provided a polynucleotide comprising an open reading frame encoding a transposase, the amino acid sequence of which is at least 90% identical to SEQ ID NO: 1, or a functional variant thereof, operably linked to a heterologous promoter.

In embodiments, the enzyme comprises a targeting element. In embodiments, the enzyme is capable of inserting a donor comprising a transgene in a genomic safe harbor site (GSHS). In embodiments, the binding of a GSHS of a nucleic acid molecule in a mammalian cell is with high target specificity, relative to a control. In embodiments, the control is a composition comprising an enzyme comprising an amino acid sequence of SEQ ID NO: 1 or a nucleic acid comprising a nucleotide sequence of SEQ ID NO: 2 or a codon-optimized form thereof.

| SEQ ID NO: 441: Amino acid sequence of synthetic Eptesicus fuscus (638 amino | |
|---|---|
| acids) transposase showing serine residues (bold) that were mutated to, | |
| without wishing to be bound by theory, increase excision activity (EXC+). | |
| 1 | MDKFSKDIES SDDEFYFENE EKSEKCNSDE SEFSEDASGD DEQIAGPSGT TERKKSLALP |
| 61 | KDLAESTDSD SDIEFIKAKR RRTIVYSSES DGDIGDIIEK SGIRPSESYV SRGKQEKEKW |
| 121 | TSTSVNDKEP SRIPFSTGQL HVGPQVPSGC ATPIDFFQLF FTETLIKNIT DETNEYARHK |
| 181 | ISQKELSQRS TWNNWKDVTI EEMKAFLGVI LNMGVLNHPN LOSYWSMDFE SHIPFFRSVF |
| 241 | KRERFLQIFW MLHLKNDQKS SKDLRTRTEK VNCFLSYLEM KFRERFCPGR EIAVDEAVVG |
| 301 | FKGKIHFITY NPKKPTKWGI RLYVLSDSKC GYVHSFVPYY GGITSETLVR PDLPFTSRIV |
| 361 | LELHERLKNS VPGSQGYHFF TDRYYTSVTL AKELFKEKTH LTGTIMPNRK DNPPVIKHPK |
| 421 | LMKGEIVAFR DENVMLLAWK DKRIVTMLST WDTSETESVE RRVRGGGKEI VLKPKVVTNY |
| 481 | TKFMGGVDIA DHYTGTYCFM RKTLKWWRKL FFWGLEVSVV NSYILYKECQ KRKNEKPITH |
| 541 | VKFIRKLVHD LVGEFRDGTL TSRGRLLSTN LEQRLDGKLH IITPHPNKKH KDCVVCSNRK |
| 601 | IKGGRRETIY ICETCECKPG LHVGECFKKY HTMKNYRD |

In embodiments, the enzyme comprises one or more serine mutations. In embodiments, the enzyme comprises one or more mutations selected from S5X, S11X, S28X, S34X, and S38X, wherein X is any amino acid. In embodiments, the enzyme comprises one or more mutations selected from S5X, S11X, S28X, S34X, S38X, wherein X is A or P. In embodiments, the enzyme comprises one or more mutations selected from S5A, S11A, S28A, S34A, S34P, S38A, and S38P mutations. In embodiments, the enzyme comprises S11A, S28A, S34A, and S38A mutations. In embodiments, the enzyme comprises an amino acid sequence of SEQ ID NO: 441, or an amino acid sequence having at least about 90%, or at least about 93%, or at least about 95%, or at least about 97%, or at least about 98%, or at least about 99% identity thereto.

In embodiments the enzyme comprises a deletion of a plurality of residues at the N-terminus. In embodiments the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125.

In embodiments the enzyme comprises a deletion of a plurality of residues at the N-terminus. In embodiments the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and one or more serine mutations. In embodiments, the enzyme comprises a deletion of a plurality of residues at the N-terminus. In embodiments the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and one or more mutations selected from S5X, S11X, S28X, S34X, and S38X, wherein X is any amino acid. In embodiments, the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and one or more mutations selected from S5X, S11X, S28X, S34X, S38X, wherein X is A or P. In embodiments, the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and one or more mutations selected from S5A, S11A, S28A, S34A, S34P, S38A, and S38P mutations. In embodiments, the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and S11A, S28A, S34A, and S38A mutations. In embodiments, the enzyme comprises a deletion of one of the following: about position 2 to about position 35, about position 2 to about position 36, about position 2 to about position 40, about position 2 to about position 45, about position 2 to about position 47, about position 2 to about position 50, about position 2 to about position 100, about position 2 to about position 110, about position 2 to about position 117, about position 2 to about position 120, about position 2 to about position 122, or about position 2 to about position 125 and an amino acid sequence of SEQ ID NO: 441, or an amino acid sequence having at least about 90%, or at least about 93%, or at least about 95%, or at least about 97%, or at least about 98%, or at least about 99% identity thereto

| SEQ ID NO: 442: Amino acid sequence of synthetic Eptesicus fuscus (603 amino acids) trans- | |
|---|---|
| posase with N-terminus deletions of amino acid 2-36 (N1 EXC+). | |
| 1 | MASGDDEQIA GPSGTTERKK SLALPKDLAE STDSDSDIEF IKAKRRRTIV YSSESDGDIG |
| 61 | DIIEKSGIRP SESYVSRGKQ EKEKWTSTSV NDKEPSRIPF STGQLHVGPQ VPSGCATPID |
| 121 | FFQLFFTETL IKNITDETNE YARHKISQKE LSQRSTWNNW KDVTIEEMKA FLGVILNMGV |
| 181 | LNHPNLQSYW SMDFESHIPF FRSVFKRERF LQIFWMLHLK NDQKSSKDLR TRTEKVNCFL |
| 241 | SYLEMKFRER FCPGREIAVD EAVVGFKGKI HFITYNPKKP TKWGIRLYVL SDSKCGYVHS |
| 301 | FVPYYGGITS ETLVRPDLPF TSRIVLELHE RLKNSVPGSQ GYHFFTDRYY TSVTLAKELF |
| 361 | KEKTHLTGTI MPNRKDNPPV IKHPKLMKGE IVAFRDENVM LLAWKDKRIV TMLSTWDTSE |
| 421 | TESVERRVRG GGKEIVLKPK VVTNYTKFMG GVDIADHYTG TYCFMRKTLK WWRKLFFWGL |
| 481 | EVSVVNSYIL YKECQKRKNE KPITHVKFIR KLVHDLVGEF RDGTLTSRGR LLSTNLEQRL |
| 541 | DGKLHIITPH PNKKHKDCVV CSNRKIKGGR RETIYICETC ECKPGLHVGE CFKKYHTMKN |
| 601 | YRD |
| SEQ ID NO: 443: Nucleotide sequence of synthetic Eptesicus fuscus (1809 bp) transposase | |
| with N-terminus deletions of amino acid 2-36 (N1 EXC+). | |
| 1 | ATGGCTAGCG GCGACGACGA GCAGATCGCT GGACCCAGCG GGACCACGGA GCGCAAAAAG |
| 61 | AGCCTGGCTC TGCCTAAAGA CTTGGCCGAG AGTACCGACA GCGACTCCGA TATCGAGTTC |
| 121 | ATCAAGGCCA AACGCAGGCG CACAATCGTG TACTCTTCCG AGAGCGACGG CGACATCGGC |
| 181 | GATATTATCG AGAAAAGCGG GATCCGGCCT TCCGAAAGCT ACGTGTCTCG GGGCAAGCAG |
| 241 | GAGAAGGAAA AGTGGACAAG CACCTCTGTG AACGACAAAG AGCCTTCCAG AATCCCCTTC |
| 301 | AGCACCGGCC AGCTGCATGT GGGCCCCCAG GTGCCCAGCG GCTGCGCCAC TCCTATCGAC |
| 361 | TTCTTCCAGC TGTTTTTTAC TGAGACCCTG ATCAAGAACA TCACCGATGA GACAAATGAG |
| 421 | TACGCCAGGC ACAAGATCTC TCAGAAGGAG CTGAGCCAGC GCAGTACATG GAACAACTGG |
| 481 | AAGGACGTGA CCATCGAAGA GATGAAGGCC TTCCTGGGCG TGATCCTGAA TATGGGAGTG |
| 541 | CTGAACCATC CTAATCTGCA GTCCTATTGG TCCATGGATT TCGAGTCCCA CATTCCATTC |
| 601 | TTCAGGTCCG TGTTCAAGCG CGAGCGTTTC CTGCAGATCT TCTGGATGCT GCACCTGAAA |
| 661 | AATGACCAGA AGAGCTCCAA GGACCTGCGG ACACGGACTG AGAAGGTGAA TTGTTTCCTG |
| 721 | TCCTACCTGG AGATGAAATT CAGGGAGAGG TTTTGTCCCG GCCGGGAAAT TGCCGTGGAT |
| 781 | GAGGCCGTGG TGGGCTTCAA GGGCAAGATC CACTTCATCA CCTACAACCC AAAGAAGCCA |
| 841 | ACAAAGTGGG GCATCCGGCT GTATGTCCTG AGTGACTCCA AGTGTGGCTA CGTGCACAGC |
| 901 | TTTGTGCCCT ATTATGGCGG CATCACCTCC GAGACCCTGG TGAGGCCCGA CCTGCCTTTC |
| 961 | ACCTCTAGAA TTGTGCTGGA GCTGCATGAG CGGCTGAAGA ACTCTGTGCC TGGCAGCCAG |
| 1021 | GGCTACCATT TTTTCACCGA CAGGTACTAT ACATCCGTTA CCCTGGCCAA GGAACTGTTC |
| 1081 | AAAGAAAAAA CCCACCTGAC CGGCACTATC ATGCCCAACC GCAAGGACAA CCCCCCTGTG |
| 1141 | ATCAAACATC CCAAACTGAT GAAGGGCGAG ATCGTGGCCT TCAGAGACGA GAACGTCATG |
| 1201 | CTGCTGGCTT GGAAAGATAA GCGGATCGTG ACTATGCTGT CTACATGGGA TACCTCCGAG |
| 1261 | ACAGAGAGCG TTGAACGGCG GGTGAGGGGT GGAGGCAAGG AGATCGTGCT GAAGCCAAAG |
| 1321 | GTGGTGACCA ACTACACCAA GTTCATGGGC GGAGTGGATA TTGCAGACCA TTACACCGGC |
| 1381 | ACCTACTGTT TCATGCGGAA GACCCTGAAG TGGTGGCGGA AGCTGTTCTT CTGGGGGCTG |
| 1441 | GAGGTCAGCG TGGTGAACTC CTACATCCTC TACAAGGAGT GCCAGAAGAG GAAGAACGAG |
| 1501 | AAACCAATCA CACACGTGAA GTTTATCAGG AAGCTGGTGC ACGACCTGGT GGGAGAGTTC |
| 1561 | CGCGACGGCA CCCTCACCAG TCGGGGCCGG CTGCTGAGTA CAAACCTGGA GCAGAGGCTG |
| 1621 | GACGGAAAGC TGCACATTAT CACTCCCCAT CCAAATAAGA AGCACAAGGA CTGCGTGGTC |
| 1681 | TGCAGCAACC GGAAGATTAA AGGAGGGCGG CGGGAAACCA TTTACATTTG TGAGACCTGC |
| 1741 | GAATGCAAGC CTGGCCTGCA CGTGGGGGAG TGCTTCAAGA AGTACCACAC AATGAAAAAC |
| 1801 | TACAGGGAT |
| SEQ ID NO: 444: Amino acid sequence of synthetic Eptesicus fuscus (592 amino acids) | |
| transposase with N-terminus deletions of amino acid 2-47 (N2 EXC+). | |
| 1 | MSGTTERKKS LALPKDLAES TDSDSDIEFI KAKRRRTIVY SSESDGDIGD IIEKSGIRPS |
| 61 | ESYVSRGKQE KEKWTSTSVN DKEPSRIPFS TGQLHVGPQV PSGCATPIDF FQLFFTETLI |
| 121 | KNITDETNEY ARHKISQKEL SQRSTWNNWK DVTIEEMKAF LGVILNMGVL NHPNLQSYWS |
| 181 | MDFESHIPFF RSVFKRERFL QIFWMLHLKN DQKSSKDLRT RTEKVNCFLS YLEMKFRERF |
| 241 | CPGREIAVDE AVVGFKGKIH FITYNPKKPT KWGIRLYVLS DSKCGYVHSF VPYYGGITSE |
| 301 | TLVRPDLPFT SRIVLELHER LKNSVPGSQG YHFFTDRYYT SVTLAKELFK EKTHLTGTIM |
| 361 | PNRKDNPPVI KHPKLMKGEI VAFRDENVML LAWKDKRIVT MLSTWDTSET ESVERRVRGG |
| 421 | GKEIVLKPKV VTNYTKFMGG VDIADHYTGT YCFMRKTLKW WRKLFFWGLE VSVVNSYILY |
| 481 | KECQKRKNEK PITHVKFIRK LVHDLVGEFR DGTLTSRGRL LSTNLEQRLD GKLHIITPHP |
| 541 | NKKHKDCVVC SNRKIKGGRR ETIYICETCE CKPGLHVGEC FKKYHTMKNY RD |
| SEQ ID NO: 445: Nucleotide sequence of synthetic Eptesicus fuscus (1776 bp) transposase | |
| with N-terminus deletions of amino acid 2-47 (N2 EXC+). | |
| 1 | ATGAGCGGGA CCACGGAGCG CAAAAAGAGC CTGGCTCTGC CTAAAGACTT GGCCGAGAGT |
| 61 | ACCGACAGCG ACTCCGATAT CGAGTTCATC AAGGCCAAAC GCAGGCGCAC AATCGTGTAC |
| 121 | TCTTCCGAGA GCGACGGCGA CATCGGCGAT ATTATCGAGA AAAGCGGGAT CCGGCCTTCC |
| 181 | GAAAGCTACG TGTCTCGGGG CAAGCAGGAG AAGGAAAAGT GGACAAGCAC CTCTGTGAAC |
| 241 | GACAAAGAGC CTTCCAGAAT CCCCTTCAGC ACCGGCCAGC TGCATGTGGG CCCCCAGGTG |
| 301 | CCCAGCGGCT GCGCCACTCC TATCGACTTC TTCCAGCTGT TTTTTACTGA GACCCTGATC |
| 361 | AAGAACATCA CCGATGAGAC AAATGAGTAC GCCAGGCACA AGATCTCTCA GAAGGAGCTG |
| 421 | AGCCAGCGCA GTACATGGAA CAACTGGAAG GACGTGACCA TCGAAGAGAT GAAGGCCTTC |
| 481 | CTGGGCGTGA TCCTGAATAT GGGAGTGCTG AACCATCCTA ATCTGCAGTC CTATTGGTCC |
| 541 | ATGGATTTCG AGTCCCACAT TCCATTCTTC AGGTCCGTGT TCAAGCGCGA GCGTTTCCTG |
| 601 | CAGATCTTCT GGATGCTGCA CCTGAAAAAT GACCAGAAGA GCTCCAAGGA CCTGCGGACA |
| 661 | CGGACTGAGA AGGTGAATTG TTTCCTGTCC TACCTGGAGA TGAAATTCAG GGAGAGGTTT |
| 721 | TGTCCCGGCC GGGAAATTGC CGTGGATGAG GCCGTGGTGG GCTTCAAGGG CAAGATCCAC |
| 781 | TTCATCACCT ACAACCCAAA GAAGCCAACA AAGTGGGGCA TCCGGCTGTA TGTCCTGAGT |
| 841 | GACTCCAAGT GTGGCTACGT GCACAGCTTT GTGCCCTATT ATGGCGGCAT CACCTCCGAG |
| 901 | ACCCTGGTGA GGCCCGACCT GCCTTTCACC TCTAGAATTG TGCTGGAGCT GCATGAGCGG |
| 961 | CTGAAGAACT CTGTGCCTGG CAGCCAGGGC TACCATTTTT TCACCGACAG GTACTATACA |
| 1021 | TCCGTTACCC TGGCCAAGGA ACTGTTCAAA GAAAAAACCC ACCTGACCGG CACTATCATG |
| 1081 | CCCAACCGCA AGGACAACCC CCCTGTGATC AAACATCCCA AACTGATGAA GGGCGAGATC |
| 1141 | GTGGCCTTCA GAGACGAGAA CGTCATGCTG CTGGCTTGGA AAGATAAGCG GATCGTGACT |
| 1201 | ATGCTGTCTA CATGGGATAC CTCCGAGACA GAGAGCGTTG AACGGCGGGT GAGGGGTGGA |
| 1261 | GGCAAGGAGA TCGTGCTGAA GCCAAAGGTG GTGACCAACT ACACCAAGTT CATGGGCGGA |
| 1321 | GTGGATATTG CAGACCATTA CACCGGCACC TACTGTTTCA TGCGGAAGAC CCTGAAGTGG |
| 1381 | TGGCGGAAGC TGTTCTTCTG GGGGCTGGAG GTCAGCGTGG TGAACTCCTA CATCCTCTAC |
| 1441 | AAGGAGTGCC AGAAGAGGAA GAACGAGAAA CCAATCACAC ACGTGAAGTT TATCAGGAAG |
| 1501 | CTGGTGCACG ACCTGGTGGG AGAGTTCCGC GACGGCACCC TCACCAGTCG GGGCCGGCTG |
| 1561 | CTGAGTACAA ACCTGGAGCA GAGGCTGGAC GGAAAGCTGC ACATTATCAC TCCCCATCCA |
| 1621 | AATAAGAAGC ACAAGGACTG CGTGGTCTGC AGCAACCGGA AGATTAAAGG AGGGCGGCGG |
| 1681 | GAAACCATTT ACATTTGTGA GACCTGCGAA TGCAAGCCTG GCCTGCACGT GGGGGAGTGC |
| 1741 | TTCAAGAAGT ACCACACAAT GAAAAACTAC AGGGAT |
| SEQ ID NO: 446: Amino acid sequence of synthetic Eptesicus fuscus (523 amino acids) | |
| transposase with N-terminus deletions of amino acid 2-117 (N3 EXC+). | |
| 1 | M-EKWTSTSV NDKEPSRIPF STGQLHVGPQ VPSGCATPID FFQLFFTETL IKNITDETNE |
| 61 | YARHKISQKE LSQRSTWNNW KDVTIEEMKA FLGVILNMGV LNHPNLQSYW SMDFESHIPF |
| 121 | FRSVFKRERF LQIFWMLHLK NDQKSSKDLR TRTEKVNCFL SYLEMKFRER FCPGREIAVD |
| 181 | EAVVGFKGKI HFITYNPKKP TKWGIRLYVL SDSKCGYVHS FVPYYGGITS ETLVRPDLPF |
| 241 | TSRIVLELHE RLKNSVPGSQ GYHFFTDRYY TSVTLAKELF KEKTHLTGTI MPNRKDNPPV |
| 301 | IKHPKLMKGE IVAFRDENVM LLAWKDKRIV TMLSTWDTSE TESVERRVRG GGKEIVLKPK |
| 361 | VVTNYTKFMG GVDIADHYTG TYCFMRKTLK WWRKLFFWGL EVSVVNSYIL YKECQKRKNE |
| 421 | KPITHVKFIR KLVHDLVGEF RDGTLTSRGR LLSTNLEQRL DGKLHIITPH PNKKHKDCVV |
| 481 | CSNRKIKGGR RETIYICETC ECKPGLHVGE CFKKYHTMKN YRD |
| SEQ ID NO: 447: Nucleotide sequence of synthetic Eptesicus fuscus (1569 bp) transposase | |
| with N-terminus deletions of amino acid 2-117 (N3 EXC+). | |
| atggaaaagtggacaagcacctctgtgaacgacaaagagccttccagaatccccttcagcaccggccagctgcatgt | |
| gggcccccaggtgcccagcggctgcgccactcctatcgacttcttccagctgttttttactgagaccctgatcaaga | |
| acatcaccgatgagacaaatgagtacgccaggcacaagatctctcagaaggagctgagccagcgcagtacatggaac | |
| aactggaaggacgtgaccatcgaagagatgaaggccttcctgggcgtgatcctgaatatgggagtgctgaaccatcc | |
| taatctgcagtcctattggtccatggatttcgagtcccacattccattcttcaggtccgtgttcaagcgcgagcgtt | |
| tcctgcagatcttctggatgctgcacctgaaaaatgaccagaagagctccaaggacctgcggacacggactgagaag | |
| gtgaattgtttcctgtcctacctggagatgaaattcagggagaggttttgtcccggccgggaaattgccgtggatga | |
| ggccgtggtgggcttcaagggcaagatccacttcatcacctacaacccaaagaagccaacaaagtggggcatccggc | |
| tgtatgtcctgagtgactccaagtgtggctacgtgcacagctttgtgccctattatggcggcatcacctccgagacc | |
| ctggtgaggcccgacctgcctttcacctctagaattgtgctggagctgcatgagcggctgaagaactctgtgcctgg | |
| cagccagggctaccattttttcaccgacaggtactatacatccgttaccctggccaaggaactgttcaaagaaaaaa | |
| cccacctgaccggcactatcatgcccaaccgcaaggacaacccccctgtgatcaaacatcccaaactgatgaagggc | |
| gagatcgtggccttcagagacgagaacgtcatgctgctggcttggaaagataagcggatcgtgactatgctgtctac | |
| atgggatacctccgagacagagagcgttgaacggcgggtgaggggtggaggcaaggagatcgtgctgaagccaaagg | |
| tggtgaccaactacaccaagttcatgggcggagtggatattgcagaccattacaccggcacctactgtttcatgcgg | |
| aagaccctgaagtggtggcggaagctgttcttctgggggctggaggtcagcgtggtgaactcctacatcctctacaa | |
| ggagtgccagaagaggaagaacgagaaaccaatcacacacgtgaagtttatcaggaagctggtgcacgacctggtgg | |
| gagagttccgcgacggcaccctcaccagtcggggccggctgctgagtacaaacctggagcagaggctggacggaaag | |
| ctgcacattatcactccccatccaaataagaagcacaaggactgcgtggtctgcagcaaccggaagattaaaggagg | |
| gcggcgggaaaccatttacatttgtgagacctgcgaatgcaagcctggcctgcacgtgggggagtgcttcaagaagt | |
| accacacaatgaaaaactacagggattaa | |
| SEQ ID NO: 448: Amino acid sequence of synthetic Eptesicus fuscus (520 amino acids) | |
| transposase with N-terminus deletions of amino acid 2-120 (N4 EXC+). | |
| 1 | M-TSTSVNDK EPSRIPFSTG QLHVGPQVPS GCATPIDFFQ LFFTETLIKN ITDETNEYAR |
| 61 | HKISQKELSQ RSTWNNWKDV TIEEMKAFLG VILNMGVINH PNLQSYWSMD FESHIPFFRS |
| 121 | VFKRERFLQI FWMLHLKNDQ KSSKDLRTRT EKVNCFLSYL EMKFRERFCP GREIAVDEAV |
| 181 | VGFKGKIHFI TYNPKKPTKW GIRLYVLSDS KCGYVHSFVP YYGGITSETL VRPDLPFTSR |
| 241 | IVLELHERLK NSVPGSQGYH FFTDRYYTSV TLAKELFKEK THLTGTIMPN RKDNPPVIKH |
| 301 | PKLMKGEIVA FRDENVMLLA WKDKRIVTML STWDTSETES VERRVRGGGK EIVLKPKVVT |
| 361 | NYTKFMGGVD IADHYTGTYC FMRKTLKWWR KLFFWGLEVS VVNSYILYKE CQKRKNEKPI |
| 421 | THVKFIRKLV HDLVGEFRDG TLTSRGRLLS TNLEQRLDGK LHIITPHPNK KHKDCVVCSN |
| 481 | RKIKGGRRET IYICETCECK PGLHVGECFK KYHTMKNYRD |
| SEQ ID NO: 449: Nucleotide sequence of synthetic Eptesicus fuscus (1560 bp) transposase | |
| with N-terminus deletions of amino acid 2-120 (N4 EXC+). | |
| atgacaagcacctctgtgaacgacaaagagccttccagaatccccttcagcaccggccagctgcatgtgggccccca | |
| ggtgcccagcggctgcgccactcctatcgacttcttccagctgttttttactgagaccctgatcaagaacatcaccg | |
| atgagacaaatgagtacgccaggcacaagatctctcagaaggagctgagccagcgcagtacatggaacaactggaag | |
| gacgtgaccatcgaagagatgaaggccttcctgggcgtgatcctgaatatgggagtgctgaaccatcctaatctgca | |
| gtcctattggtccatggatttcgagtcccacattccattcttcaggtccgtgttcaagcgcgagcgtttcctgcaga | |
| tcttctggatgctgcacctgaaaaatgaccagaagagctccaaggacctgcggacacggactgagaaggtgaattgt | |
| ttcctgtcctacctggagatgaaattcagggagaggttttgtcccggccgggaaattgccgtggatgaggccgtggt | |
| gggcttcaagggcaagatccacttcatcacctacaacccaaagaagccaacaaagtggggcatccggctgtatgtcc | |
| tgagtgactccaagtgtggctacgtgcacagctttgtgccctattatggcggcatcacctccgagaccctggtgagg | |
| cccgacctgcctttcacctctagaattgtgctggagctgcatgagcggctgaagaactctgtgcctggcagccaggg | |
| ctaccattttttcaccgacaggtactatacatccgttaccctggccaaggaactgttcaaagaaaaaacccacctga | |
| ccggcactatcatgcccaaccgcaaggacaacccccctgtgatcaaacatcccaaactgatgaagggcgagatcgtg | |
| gccttcagagacgagaacgtcatgctgctggcttggaaagataagcggatcgtgactatgctgtctacatgggatac | |
| ctccgagacagagagcgttgaacggcgggtgaggggtggaggcaaggagatcgtgctgaagccaaaggtggtgacca | |
| actacaccaagttcatgggcggagtggatattgcagaccattacaccggcacctactgtttcatgcggaagaccctg | |
| aagtggtggcggaagctgttcttctgggggctggaggtcagcgtggtgaactcctacatcctctacaaggagtgcca | |
| gaagaggaagaacgagaaaccaatcacacacgtgaagtttatcaggaagctggtgcacgacctggtgggagagttcc | |
| gcgacggcaccctcaccagtcggggccggctgctgagtacaaacctggagcagaggctggacggaaagctgcacatt | |
| atcactccccatccaaataagaagcacaaggactgcgtggtctgcagcaaccggaagattaaaggagggcggcggga | |
| aaccatttacatttgtgagacctgcgaatgcaagcctggcctgcacgtgggggagtgcttcaagaagtaccacacaa | |
| tgaaaaactacagggattaa | |
| SEQ ID NO: 450: Amino acid sequence of synthetic Eptesicus fuscus (518 amino acids) | |
| transposase with N-terminus deletions of amino acid 2-122 (N5 EXC+). | |
| 1 | M-TSVNDKEP SRIPFSTGQL HVGPQVPSGC ATPIDFFQLF FTETLIKNIT DETNEYARHK |
| 61 | ISQKELSQRS TWNNWKDVTI EEMKAFLGVI LNMGVLNHPN LQSYWSMDFE SHIPFFRSVF |
| 121 | KRERFLQIFW MLHLKNDQKS SKDLRTRTEK VNCFLSYLEM KFRERFCPGR EIAVDEAVVG |
| 181 | FKGKIHFITY NPKKPTKWGI RLYVLSDSKC GYVHSFVPYY GGITSETLVR PDLPFTSRIV |
| 241 | LELHERLKNS VPGSQGYHFF TDRYYTSVTL AKELFKEKTH LTGTIMPNRK DNPPVIKHPK |
| 301 | LMKGEIVAFR DENVMLLAWK DKRIVTMLST WDTSETESVE RRVRGGGKEI VLKPKVVTNY |
| 361 | TKFMGGVDIA DHYTGTYCFM RKTLKWWRKL FFWGLEVSVV NSYILYKECQ KRKNEKPITH |
| 421 | VKFIRKLVHD LVGEFRDGTL TSRGRLLSTN LEQRLDGKLH IITPHPNKKH KDCVVCSNRK |
| 481 | IKGGRRETIY ICETCECKPG LHVGECFKKY HTMKNYRD |

SEQ ID NO: 451: Nucleotide sequence of synthetic Eptesicus fuscus (1554 bp) transposase
with N-terminus deletions of amino acid 2-122 (N5 EXC+).
atgacctctgtgaacgacaaagagccttccagaatccccttcagcaccggccagctgcatgtgggcccccaggtgcc
cagcggctgcgccactcctatcgacttcttccagctgttttttactgagaccctgatcaagaacatcaccgatgaga
caaatgagtacgccaggcacaagatctctcagaaggagctgagccagcgcagtacatggaacaactggaaggacgtg
accatcgaagagatgaaggccttcctgggcgtgatcctgaatatgggagtgctgaaccatcctaatctgcagtccta
ttggtccatggatttcgagtcccacattccattcttcaggtccgtgttcaagcgcgagcgtttcctgcagatcttct
ggatgctgcacctgaaaaatgaccagaagagctccaaggacctgcggacacggactgagaaggtgaattgtttcctg
tcctacctggagatgaaattcagggagaggttttgtcccggccgggaaattgccgtggatgaggccgtggtgggctt
caagggcaagatccacttcatcacctacaacccaaagaagccaacaaagtggggcatccggctgtatgtcctgagtg
actccaagtgtggctacgtgcacagctttgtgccctattatggcggcatcacctccgagaccctggtgaggcccgac
ctgcctttcacctctagaattgtgctggagctgcatgagcggctgaagaactctgtgcctggcagccagggctacca
ttttttcaccgacaggtactatacatccgttaccctggccaaggaactgttcaaagaaaaaacccacctgaccggca
ctatcatgcccaaccgcaaggacaacccccctgtgatcaaacatcccaaactgatgaagggcgagatcgtggccttc
agagacgagaacgtcatgctgctggcttggaaagataagcggatcgtgactatgctgtctacatgggatacctccga
gacagagagcgttgaacggcgggtgaggggtggaggcaaggagatcgtgctgaagccaaaggtggtgaccaactaca
ccaagttcatgggcggagtggatattgcagaccattacaccggcacctactgtttcatgcggaagaccctgaagtgg
tggcggaagctgttcttctgggggctggaggtcagcgtggtgaactcctacatcctctacaaggagtgccagaagag
gaagaacgagaaaccaatcacacacgtgaagtttatcaggaagctggtgcacgacctggtgggagagttccgcgacg
gcaccctcaccagtcggggccggctgctgagtacaaacctggagcagaggctggacggaaagctgcacattatcact
ccccatccaaataagaagcacaaggactgcgtggtctgcagcaaccggaagattaaaggagggcggcgggaaaccat
ttacatttgtgagacctgcgaatgcaagcctggcctgcacgtgggggagtgcttcaagaagtaccacacaatgaaaa
actacagggattaa

In embodiments, the present disclosure provides a composition comprising a helper enzyme or a nucleic acid encoding the enzyme, wherein the enzyme comprises an amino acid sequence having at least about 80% sequence identity to SEQ ID NO: 441, 442, 444, 446, 448, or 450.

In embodiments, the enzyme comprises an amino acid sequence of at least about 80% identity to SEQ ID NO: 441, 442, 444, 446, 448, or 450. In embodiments, the enzyme comprises an amino acid sequence of at least about 83% identity to SEQ ID NO: 441, 442, 444, 446, 448, or 450. In embodiments, the enzyme comprises an amino acid sequence of at least about 85% identity to SEQ ID NO: 441, 442, 444, 446, 448, or 450. In embodiments, the enzyme comprises an amino acid sequence of at least about 88% identity to SEQ ID NO: 441, 442, 444, 446, 448, or 450. In embodiments, the enzyme comprises an amino acid sequence of at least about 89% identity to SEQ ID NO: 441, 442, 444, 446, 448, or 450. In embodiments, the enzyme comprises an amino acid sequence of at least about 90% identity to SEQ ID NO: 441, 442, 444, 446, 448, or 450. In embodiments, the enzyme comprises an amino acid sequence of at least about 93% identity to SEQ ID NO: 441, 442, 444, 446, 448, or 450. In embodiments, the enzyme comprises an amino acid sequence of at least about 95% identity to SEQ ID NO: 441, 442, 444, 446, 448, or 450. In embodiments, the enzyme comprises an amino acid sequence of at least about 98% identity to SEQ ID NO: 441, 442, 444, 446, 448, or 450. In embodiments, the enzyme comprises an amino acid sequence of at least about 99% identity to SEQ ID NO: 441, 442, 444, 446, 448, or 450.

In embodiments, the enzyme has one or more mutations which confer hyperactivity.

In embodiments, the enzyme has one or more amino acid substitutions generated by by random mutagenesis and/or site directed mutagenesis.

In embodiments, the nucleic acid that encodes the enzyme has a nucleotide sequence of SEQ ID NO: 443, 445, 447, 449, or 451, or a codon-optimized form thereof.

In embodiments, there is provided a polynucleotide comprising an open reading frame encoding a helper enzyme which is at least 90% identical to SEQ ID NO: 443, 445, 447, 449, or 451, or a functional variant thereof, operably linked to a heterologous promoter.

In embodiments, there is provided a polynucleotide comprising an open reading frame encoding a transposase, the amino acid sequence of which is at least 90% identical to SEQ ID NO: 441, 442, 444, 446, 448, or 450, or a functional variant thereof, operably linked to a heterologous promoter.

In embodiments, the enzyme is excision positive. In embodiments, the enzyme is integration deficient. In embodiments, the enzyme has decreased integration activity relative to an enzyme comprising an amino acid sequence of SEQ ID NO: 441, 442, 444, 446, 448, or 450 or functional equivalent thereof. In embodiments, the enzyme has increased excision activity relative to an enzyme comprising an amino acid sequence of SEQ ID NO: 441, 442, 444, 446, 448, or 450 or functional equivalent thereof.

In embodiments, there is provided a polynucleotide comprising an open reading frame encoding a helper enzyme which is at least 90% identical to SEQ ID NO: 443, 445, 447, 449, or 451, or a functional variant thereof, operably linked to a heterologous promoter.

In embodiments, there is provided a polynucleotide comprising an open reading frame encoding a transposase, the amino acid sequence of which is at least 90% identical to SEQ ID NO: 441, 442, 444, 446, 448, or 450, or a functional variant thereof, operably linked to a heterologous promoter.

In embodiments, the enzyme comprises a targeting element. In embodiments, the enzyme is capable of inserting a donor comprising a transgene in a genomic safe harbor site (GSHS). In embodiments, the binding of a GSHS of a nucleic acid molecule in a mammalian cell is with high target specificity, relative to a control. In embodiments, the control is a composition comprising an enzyme comprising an amino acid sequence of SEQ ID NO: 441, 442, 444, 446, 448, or 450 or a nucleic acid comprising a nucleotide sequence of SEQ ID NO: 443, 445, 447, 449, or 451, or a codon-optimized form thereof.

In embodiments, the targeting element is able to direct a transposition machinery to the GSHS of a nucleic acid molecule in a mammalian cell. In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS is an adeno-associated virus site 1 (AAVS1). In embodiments, the GSHS is a human Rosa26 locus. In embodiments, the GSHS is located on human chromosome 2, 4, 6, 10, 11, 17, 22, or X.

In embodiments, the GSHS is selected from TABLES 1-17. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TA-LER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element is or comprises one or more of a Cas enzyme, which is optionally catalytically inactive and which is optionally associated with a guide RNA (gRNA), transcription activator-like effector (TALE) DNA binding domain (DBD), catalytically inactive Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TniQ subdomain of TnsD) or a variant thereof. In embodiments, the targeting element comprises a TALE DBD. In embodiments, the TALE DBD comprises one or more repeat sequences. In embodiments, the TALE DBD comprises about 14, or about 15, or about, 16, or about 17, or about 18, or about 18.5 repeat sequences. In embodiments, the repeat sequences each independently comprises about 33 or 34 amino acids. In embodiments, the repeat sequences each independently comprises a repeat variable di-residue (RVD) at residue 12 or 13 of the 33 or 34 amino acids, respectively. In embodiments, the RVD recognizes one base pair in a target nucleic acid sequence. In embodiments, the RVD recognizes a C residue in the target nucleic acid sequence and is selected from HD, N (gap), HA, ND, and HI. In embodiments, the RVD recognizes a G residue in the target nucleic acid sequence and is selected from NN, NH, NK, HN, and NA. In embodiments, the RVD recognizes an A residue in the target nucleic acid sequence and is selected from NI and NS. In embodiments, the RVD recognizes a T residue in the target nucleic acid sequence and is selected from NG, HG, H (gap), and IG.

In embodiments, the TALE DBD targets one or more of GSHS sites selected from TABLES 7-12.

In embodiments, the TALE DBD comprises one or more of RVD selected from TABLES 7-12, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

In embodiments, the targeting element comprises a Cas9 enzyme associated with a gRNA. In embodiments, the Cas9 enzyme associated with a gRNA comprises a catalytically inactive dCas9 associated with a gRNA.

In embodiments, the catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

| SEQ ID NO: 5: nucleotide sequence of dead Cas9 DNA binding protein (5004 bp) | |
|---|---|
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 481 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGAC |
| SEQ ID NO: 6: amino acid sequence of dead Cas9 DNA binding protein (1368 amino acids) | |
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVAA |
| 841 | IVPQSFLKDD SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD |

In embodiments, the targeting element comprises a Cas12 enzyme associated with a gRNA. In embodiments, the targeting element comprises a catalytically inactive Cas12 associated with a gRNA, optionally wherein the catalytically inactive Cas12 is dCas12j or dCas12a. In embodiments, the targeting element comprises a TnsC, TnsB, TnsA, TniQ, Cas6, Cas7, Cas8 enzyme associated with a gRNA.

In embodiments, the targeting element comprises a CasX enzyme associated with a gRNA. In embodiments, the targeting element comprises a catalytically inactive CasX associated with a gRNA.

In embodiments, the guide RNA is selected from TABLES 1-6, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations. In embodiments, the guide RNA targets one or more sites selected from TABLES 1-6. In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence. In embodiments, the zinc finger targets one or more sites selected from TABLES 13-17.

In embodiments, the targeting element comprises a nucleic acid binding component of a gene-editing system. In embodiments, the enzyme or variant thereof and the targeting element are connected. In embodiments, the enzyme and the targeting element are fused to one another or linked via a linker to one another. In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the enzyme is directly fused to the N-terminus of the targeting element and, optionally, wherein the targeting element is or comprises dCas9 enzyme.

In embodiments, the E. coli TniQ subdomain of TnsD comprises at least about 90%, or at least about 95%, or at least about 96%, or at least about 97%, or at least about 98%, or at least about 99% identity to an amino acid sequence of SEQ ID NO: 7. In embodiments, the TniQ subdomain of TnsD comprises a truncated TniQ subdomain of TnsD. In embodiments, the TniQ subdomain of TnsD is truncated at its C-terminus. In embodiments, the TniQ subdomain of TnsD is truncated at its N-terminus. In embodiments, the TniQ subdomain of TnsD or variant thereof comprises a zinc finger motif. In embodiments, the zinc finger motif comprises a C3H-type motif (e.g., CCCH).

| SEQ ID NO: 7: amino acid sequence of E. coli TnsD (including the TniQ domain) | |
|---|---|
| (508 amino acids) | |
| 1 | MRNFPVPYSN ELIYSTIARA GVYQGIVSPK QLLDEVYGNR KVVATLGLPS HLGVIARHLH |
| 61 | QTGRYAVQQL IYEHTLFPLY APFVGKERRD EAIRLMEYQA QGAVHLMLGV AASRVKSDNR |
| 121 | FRYCPDCVAL QLNRYGEAFW QRDWYLPALP YCPKHGALVF FDRAVDDHRH QFWALGHTEL |
| 181 | LSDYPKDSLS QLTALAAYIA PLLDAPRAQE LSPSLEQWTL FYQRLAQDLG LTKSKHIRHD |
| 241 | LVAERVRQTF SDEALEKLDL KLAENKDTCW LKSIFRKHRK AFSYLQHSIV WQALLPKLTV |
| 301 | IEALQQASAL TEHSITTRPV SQSVQPNSED LSVKHKDWQQ LVHKYQGIKA ARQSLEGGVL |
| 361 | YAWLYRHDRD WLVHWNQQHQ QERLAPAPRV DWNQRDRIAV RQLLRIIKRL DSSLDHPRAT |
| 421 | SSWLLKQTPN GTSLAKNLQK LPLVALCLKR YSESVEDYQI RRISQAFIKL KQEDVELRRW |
| 481 | RLLRSATLSK ERITEEAQRF LEMVYGEE |

In embodiments, the TniQ subdomain of TnsD binds at or near an attTn7 attachment site. In embodiments, the TniQ subdomain of TnsD binds at or near a region downstream of the glmS gene. GlmS (L-glucosamine-fructose-6-phosphate aminotransferase) is highly conserved and found in a wide variety of organisms from bacteria to humans. In embodiments, the TniQ subdomain of TnsD binding region of glmS encodes the active site region of GlmS. In embodiments, TniQ subdomain of TnsD binds at or near the human homologs of glmS, e.g., gfpt-1 and gfpt-2. In embodiments, TniQ subdomain of TnsD binds the human glmS homologs gfpt-1 and gfpt-2. In embodiments, the transgene is inserted into attTn7.

In embodiments, the TniQ subdomain of TnsD comprises a nucleic acid binding component of a gene-editing system. In embodiments, the enzyme or variant thereof (optionally, wherein the enzyme is a helper enzyme, optionally, wherein the helper enzyme is reconstructed from Eptesicus fuscus) and the TniQ subdomain of TnsD are connected. In embodiments, the enzyme and the TniQ subdomain of TnsD are fused to one another or linked via a linker to one another. In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the enzyme is directly fused to the N-terminus of the TniQ subdomain of TnsD.

In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence. In embodiments, the zinc finger targets one or more sites selected from TABLES 13-17.

In embodiments, the enzyme or variant thereof is able to directly or indirectly cause transposition of a target gene. In embodiments, the enzyme or variant thereof is able to directly or indirectly interact and/or form a complex with one or more proteins or nucleic acids.

### Construct

In embodiments, the composition (e.g., a helper of the present disclosure), system, or method further comprising a nucleic acid encoding a donor comprising a transgene to be integrated. In embodiments, the transgene is defective or substantially absent in a disease state. In embodiments, the transgene comprises a cargo nucleic acid sequence and a first and a second donor end sequences. In embodiments, the cargo nucleic acid sequence is flanked by the first and the second donor end sequences.

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof.

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof, wherein the heterologous polynucleotide is transposable by a helper enzyme having the sequence of SEQ ID NO: 1, or a functional variant thereof.

In embodiments, the donor end sequences are selected from nucleotide sequences of SEQ ID NO: 3 and/or SEQ ID NO: 4, or a nucleotide sequence having at least about 90%, or at least about 93%, or at least about 95%, or at least about 97%, or at least about 98%, or at least about 99% identity thereto.

| SEQ ID NO: 3: Eptesicus fuscus Left ITR (200 bp) (excluding TTAA) | |
|---|---|
| 1 | ccttttgcac tcggatgtcg agtgtgactc gacacggtta gcatcggtag cagctcgtat |
| 61 | gtcgagccac actcgacacg tagtttcacc gaggggggaa gggggatttt tgtctatttt |
| 121 | tccagtatct tttcttgttt tcattagcat gaaaggacaa gtaaaatgta aatgccgtct |
| 181 | caactgatgc caccacctaa |
| SEQ ID NO: 4: Eptesicus fuscus Right ITR (200 bp) (excluding TTAA) | |
| 1 | tgaaaaatta tagagattaa aattactctt tgaatgtatc aataatttga aatataaaaa |
| 61 | aatccaaata aataagtttg tatgaaaaga aactccagtt ttttattcta ctgccgcgct |
| 121 | ttgtaaaatc tggggtattt aaaaaattaa atcccgagta gaataaagga atcgagaaaa |
| 181 | aagcaagcga gtgcaaaggg |

In embodiments, the end sequences include at least one repeat from a nucleotide sequence having at least about 90% identity to the nucleotide sequence of SEQ ID NO: 3. In embodiments, the at least one repeat from the nucleotide sequence having at least about 90%, or at least about 93%, or at least about 95%, or at least about 97%, or at least about 98%, or at least about 99% identity to the nucleotide sequence of SEQ ID NO: 3 is positioned at the 5′ end of the donor. In embodiments, the end sequences can further include at least one repeat from a nucleotide sequence having at least about 90%, or at least about 93%, or at least about 95%, or at least about 97%, or at least about 98%, or at least about 99% identity to the nucleotide sequence of SEQ ID NO: 4. In embodiments, the at least one repeat from the nucleotide sequence having at least about 90% identity to the nucleotide sequence of SEQ ID NO: 4 is positioned at the 3′ end of the donor.

In embodiments, the present disclosure provides a donor construct comprising a heterologous polynucleotide between left and right transposon ends, wherein the left end comprises SEQ ID NO: 3, or a functional variant thereof and the right end comprises SEQ ID NO: 4, or a functional variant thereof. In embodiments, the donor is transposable by a helper enzyme having the sequence of SEQ ID NO: 1, or a functional variant thereof.

In embodiments, the present disclosure provides a donor construct comprising a heterologous polynucleotide between left and right transposon ends, wherein the donor is suitable for transposition by a helper enzyme having the sequence of SEQ ID NO: 1, or a functional variant thereof.

In embodiments, the helper enzyme derived from Eptesicus fuscus, the helper enzyme being suitable for transposition of a heterologous polynucleotide, the heterologous polynucleotide being flanked by two ends elements comprising the polynucleotide sequences of SEQ ID NO: 3, or a functional variant thereof and SEQ ID NO: 4, or a functional variant thereof.

In embodiments, the enzyme or variant thereof is incorporated into a vector or a vector-like particle. In embodiments, the vector or a vector-like particle comprises one or more expression cassettes. In embodiments, the vector or a vector-like particle comprises one expression cassette. In embodiments, the expression cassette further comprises the enzyme or variant thereof, the transgene, the donor end sequences, or a combination thereof.

In embodiments, the enzyme or variant thereof, the transgene, the donor end sequences, or a combination thereof are incorporated into one or more vectors or vector-like particles. In embodiments, the enzyme or variant thereof, the transgene, the donor end sequences, or combination thereof are incorporated into a same vector or vector-like particle. In embodiments, the enzyme or variant thereof, the transgene, the donor end sequences, or combination thereof is incorporated into different vectors or vector-like particles. In embodiments, the vector or vector-like particle is nonviral. In embodiments, the composition comprises DNA, RNA, or both. In embodiments, the enzyme or variant thereof is in the form of RNA.

In embodiments, the donor is under the control of at least one tissue-specific promoter. In embodiments, the at least one tissue-specific promoter is a single promoter. In embodiments, the at least one tissue-specific promoter is under the control of a dual promoter or a tandem promoter.

In embodiments, the transgene to be integrated comprises at least one gene of interest. In embodiments, the transgene to be integrated comprises one gene of interest. In embodiments, the transgene to be integrated comprises two genes of interest. In embodiments, the transgene to be integrated comprises three genes of interest. In embodiments, the transgene to be integrated comprises four genes of interest. In embodiments, the transgene to be integrated comprises five genes of interest. In embodiments, the transgene to be integrated comprises six genes of interest.

In embodiments, the at least one gene of interest comprises peptides for linking genes of interest. In embodiments, the peptides are 2A self-cleaving peptides, or functional variants thereof, wherein the 2A self-cleaving peptide is optionally selected from P2A, E2A, F2A, and T2A, or derivative thereof.

In embodiments, the at least one gene of interest is linked to polynucleotide comprising a sequence comprising a 5′-miRNA, a sense and antisense miRNA pair, and/or a 3′-miRNA.

### Host Cell

In aspects, the present disclosure further provides a host cell comprising the composition in accordance with embodiments of the present disclosure.

### Methods

In certain embodiments, the present disclosure provides a method for inserting a gene into the genome of a cell, comprising contacting a cell with the composition of the present disclosure or host cell of the present disclosure. In embodiments, the method further comprises contacting the cell with a polynucleotide encoding a donor.

In embodiments, the donor comprises a gene encoding a complete polypeptide.

In embodiments, the donor comprises a gene which is defective or substantially absent in a disease state.

In certain embodiments, the present disclosure provides a method for treating a disease or disorder ex vivo, comprising contacting a cell with the composition of the present disclosure or host cell of the present disclosure and administering the cell to a subject in need thereof.

In certain embodiments, the present disclosure provides a method for treating a disease or disorder in vivo, comprising administering the composition of the present disclosure or host cell of the present disclosure to a subject in need thereof.

### Transgene

In embodiments, the transgene is an exogenous wild-type gene that, e.g., corrects a defective function of one or more mutations in a recipient. For instance, in embodiments, the recipient may have a mutation that provides a disease phenotype (e.g., a defective or absent gene product). In embodiments, the donor system or method of the present disclosure provides a correction that restores the gene product and diminishes the disease phenotype.

In embodiments, the transgene is a gene that replaces, inactivates, or provides suicide or helper functions.

In embodiments, the transgene and/or disease to be treated is one or more of:
beta-thalassemia: BCL11a or β-globin or βA-T87Q-globin,
LCA: RPE65,
LHON: ND4,
Achromatopsia: CNGA3 or CNGA3/CNGB3,
Choroideremia: REP1,
PKD: RPK (Red cell PK),
Hemophilia: F8,
ADA-SCID: ADA,
Fabry disease: GLA,
MPS type I: IDUA,
MPS type II: IDS, and
Cystic fibrosis: CFTR transgene.

In embodiments, the donor comprises a gene encoding a complete polypeptide. In embodiments, the donor comprises a gene which is defective or substantially absent in a disease state.

In embodiments, the transfecting of the cell is carried out using electroporation or calcium phosphate precipitation.

In embodiments, the transfecting of the cell is carried out using a lipid vehicle, optionally N-[1-(2,3-dioleoyloxy) propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using a lipid selected from one or more of the following categories: cationic lipids; anionic lipids; neutral lipids; multi-valent charged lipids; and zwitterionic lipids. In embodiments, a cationic lipid may be used to facilitate a charge-charge interaction with nucleic acids. In embodiments, the lipid is a neutral lipid. In embodiments, the neutral lipid is dioleoylphosphatidylethanolamine (DOPE), 1,2-Dioleoyl-sn-glycero-3-phosphocholine (DOPC), or cholesterol. In embodiments, cholesterol is derived from plant sources. In other embodiments, cholesterol is derived from animal, fungal, bacterial, or archaeal sources. In embodiments, the lipid is a cationic lipid. In embodiments, the cationic lipid is N-[1-(2,3-dioleoyloxy) propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP). In embodiments, one or more of the phospholipids 18:0 PC, 18:1 PC, 18:2 PC, DMPC, DSPE, DOPE, 18:2 PE, DMPE, or a combination thereof are used as lipids. In embodiments, the lipid is DOTMA and DOPE, optionally in a ratio of about 1:1. In embodiments, the lipid is DHDOS and DOPE, optionally in a ratio of about 1:1. In embodiments, the lipid is a commercially available product (e.g., LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation) (Life Technologies).

In embodiments, the transfecting of the cell is carried out using a cationic vehicle, optionally LIPOFECTIN or TRANSFECTAM.

In embodiments, the transfecting of the cell is carried out using a lipid nanoparticle or a liposome.

In embodiments, the method is helper virus-free.

Epigenetic regulatory elements can be used to protect a transgene from unwanted epigenetic effects when placed near the transgene on a vector, including the transgene. See Ley et al., PloS One vol. 8,4 e62784. 30 Apr. 2013. For example, MARs were shown to increase genomic integration and integration of a transgene while preventing heterochromatin silencing, as exemplified by the human MAR 1-68. See id.; see also Grandjean et al., Nucleic Acids Res. 2011 August; 39 (15): e104. MARs can also act as insulators and thereby prevent the activation of neighboring cellular genes. Gaussin et al., Gene Ther. 2012 January; 19 (1): 15-24. It has been shown that a piggyBac donor containing human MARs in CHO cells mediated efficient and sustained expression from a few transgene copies, using cell populations generated without an antibiotic selection procedure. See Ley et al. (2013).

In embodiments, the cell is further transfected with a third nucleic acid having at least one chromatin element, wherein the at least one chromatin element is optionally a Matrix Attachment Region (MAR) element. MARs are expression-enhancing, epigenetic regulator elements which are used to enhance and/or facilitate transgene expression, as described, for example, in PCT/IB2010/002337 (WO2011033375), which is incorporated by reference herein in its entirety. A MAR element can be located in cis or trans to the transgene.

In embodiments, the transgene has a size of 100,000 bases or less, e.g., about 100,000 bases, or about 50,000 bases, or about 30,000 bases, or about 10,000 bases, or about 5,000 bases, or about 10,000 to about 100,000 bases, or about 30,000 to about 100,000 bases, or about 50,000 to about 100,000 bases, or about 10,000 to about 50,000 bases, or about 10,000 to about 30,000 bases, or about 30,000 to about 50,000 bases.

In embodiments, the transgene has a size of about 200,000 bases or less, e.g., about 200,000 bases, or about 10,000 to about 200,000 bases, or about 30,000 to about 200,000 bases, or about 50,000 to about 200,000 bases, or about 100,000 to about 200,000 bases, or about 150,000 to about 200,000 bases.

In embodiments, the transgene has a size of about 300,000 bases or less, e.g., about 300,000 bases, or about 10,000 to about 300,000 bases, or about 30,000 to about 300,000 bases, or about 50,000 to about 300,000 bases, or about 100,000 to about 300,000 bases, or about 150,000 to about 300,000 bases.

### Targeting Chimeric Constructs

In aspects, the present disclosure provides for a donor system, e.g., in embodiments, a helper enzyme comprising a targeting element.

In embodiments, the helper enzyme associated with the targeting element, is capable of inserting the donor comprising a transgene, optionally at a TA dinucleotide site or a TTAA (SEQ ID NO: 440) tetranucleotide site in a genomic safe harbor site (GSHS).

In embodiments, the helper enzyme associated with the targeting element has one or more mutations which confer hyperactivity.

In embodiments, the helper enzyme associated with the targeting element has gene cleavage (Exc) and/or gene integration (Int+) activity.

In embodiments, the helper enzyme associated with the targeting element has gene cleavage (Exc) and/or a lack of gene integration (Int−) activity.

In embodiments, the targeting element comprises one or more proteins or nucleic acids that are capable of binding to a nucleic acid.

In embodiments, the targeting element comprises one or more of a gRNA, optionally associated with a Cas enzyme, which is optionally catalytically inactive, transcription activator-like effector (TALE), catalytically inactive Zinc finger, catalytically inactive transcription factor, nickase, a transcriptional activator, a transcriptional repressor, a recombinase, a DNA methyltransferase, a histone methyltransferase, and paternally expressed gene 10 (PEG10).

In embodiments, the targeting element comprises a transcription activator-like effector (TALE) DNA binding domain (DBD).

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H (gap), and IG. In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS is located on human chromosome 2, 4, 6, 10, 11, 17, 22, or X. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a Cas12 enzyme guide RNA complex. In embodiments, the targeting element comprises a nuclease-deficient dCas12 guide RNA complex, optionally dCas12j guide RNA complex or dCas12a guide RNA complex. In embodiments, the targeting element comprises a Cas12k enzyme guide RNA complex. In embodiments, the targeting element comprises a nuclease-deficient dCas12 guide RNA complex, optionally dCas12k guide RNA complex.

In embodiments, a targeting chimeric system or construct, having a DBD fused to the helper enzyme directs binding of the helper to a specific sequence (e.g., transcription activator-like effector proteins (TALE) repeat variable di-residues (RVD) or gRNA) near an enzyme recognition site. The enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS.

In embodiments, TALEs described herein can physically sequester the enzyme to GSHS and promote transposition to nearby TTAA (SEQ ID NO: 440) sequences in close proximity to the RVD TALE nucleotide sequences. GSHS in open chromatin sites are specifically targeted based on the predilection for helpers to insert into open chromatin.

In embodiments, the helper enzyme is capable of targeted genomic integration by transposition is linked to or fused with a TALE DNA binding domain (DBD) or a Cas-based gene-editing system, such as, e.g., Cas9 or a variant thereof.

In embodiments, the targeting element targets the helper enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the enzyme to a chemokine (C—C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 115)
AAACGGATCTGTTTTCGTGGCTCCC;
(SEQ ID NO: 116)
AAACTGTCCCTGGATCTGTTTTCGC;
(SEQ ID NO: 117)
AAACAGCACCGTGTCCCTGGATCTC;
(SEQ ID NO: 118)
AAACCACTGTCCTAGCACCGTGTCC;
(SEQ ID NO: 119)
AAACGAGGCTGTTGGGTCATTTTCC;
(SEQ ID NO: 120);
AAACAGTGGTCAGGCCGGCCAGGCC
(SEQ ID NO: 121)
AAACGCCAGGCCTTCAGTGCTCAGC;
(SEQ ID NO: 122)
AAACTCAGTGCTCAGTGGAAACCAC;
(SEQ ID NO: 123)
AAACCGAAAGGACTCCTGGCTATCC;
(SEQ ID NO: 124)
AAACAGTCTGAGCACTGGAAGCGCC;
(SEQ ID NO: 125)
AAACCTTCCCTAGTCTGAGCACTGC;
(SEQ ID NO: 126)
AAACGGCTCTGAAGGAGGAGGGGCC;
(SEQ ID NO: 127)
AAACGGACTCCTGGCTCTGAAGGAC;
(SEQ ID NO: 128)
AAACAGGGTCAAGCTCGGAAACCAC;
(SEQ ID NO: 129)
AAACCCCCAGCAGATACTCTGCAGC;
(SEQ ID NO: 130)
AAACGCAGATACTCTGCAGGAACGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGTGGAGTGGG;
(SEQ ID NO: 328)
GTGGGGGGTGGAGTGGGGGG;
(SEQ ID NO: 329)
GGGGGGTGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGTGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGGGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAAC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGCGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactogacatggagg (SEQ ID NO: 427) and cctgcaggggagtgagcagc (SEQ ID NO: 428).

In embodiments, guide RNAs (gRNAs) for targeting human genomic safe harbor sites using any of the gRNA-based targeting elements, e.g., without limitation dCas, in areas of open chromatin are as shown in TABLE 1.

TABLE 1
Guide RNAs for targeting human genomic safe harbor sites using any
of the gRNA-based targeting elements in areas of open chromatin.

| GSHS | Identifier | Sequence |
|---|---|---|
| AAVS1 | 14F | ggagccacgaaaacagatcc (SEQ ID NO: 99) |
| AAVS1 | 15F | cgaaaacagatccagggaca (SEQ ID NO: 100) |
| AAVS1 | 16F | agatccagggacacggtgct (SEQ ID NO: 101) |
| AAVS1 | 17F | gacacggtgctaggacagtg (SEQ ID NO: 102) |
| AAVS1 | 18F | gaaaatgacccaacagcctc (SEQ ID NO: 103) |
| AAVS1 | 19F | gcctggccggcctgaccact (SEQ ID NO: 104) |
| AAVS1 | 20F | ctgagcactgaaggcctggc (SEQ ID NO: 105) |
| AAVS1 | 21F | tggtttccactgagcactga (SEQ ID NO: 106) |
| AAVS1 | 22F | gatagccaggagtcctttcg (SEQ ID NO: 107) |
| AAVS1 | 23F | gcgcttccagtgctcagact (SEQ ID NO: 108) |
| AAVS1 | 24F | cagtgctcagactagggaag (SEQ ID NO: 109) |
| AAVS1 | 25F | gcccctcctccttcagagcc (SEQ ID NO: 110) |
| AAVS1 | 26F | tccttcagagccaggagtcc (SEQ ID NO: 111) |
| AAVS1 | 27F | tggtttccgagcttgaccct (SEQ ID NO: 112) |
| AAVS1 | 28F | ctgcagagtatctgctgggg (SEQ ID NO: 113) |
| AAVS1 | 29F | cgttcctgcagagtatctgc (SEQ ID NO: 114) |
| AAVS1 | AAVS1 | tcccctcccagaaagacctg (SEQ ID NO: 131) |
| AAVS1 | gAAVS2 | tgggctccaagcaatcctgg (SEQ ID NO: 132) |
| AAVS1 | gAAVS3 | gtggctcaggaggtacctgg (SEQ ID NO: 133) |
| AAVS1 | gAAVS4 | gagccacgaaaacagatcca (SEQ ID NO: 134) |
| AAVS1 | gAAVS5 | aagtgaacggggaagggagg (SEQ ID NO: 135) |
| AAVS1 | gAAVS6 | gacaaaagccgaagtccagg (SEQ ID NO: 136) |
| AAVS1 | gAAVS7 | gtggttgataaacccacgtg (SEQ ID NO: 137) |
| AAVS1 | gAAVS8 | tgggaacagccacagcaggg (SEQ ID NO: 138) |
| AAVS1 | gAAVS9 | gcaggggaacggggatgcag (SEQ ID NO: 139) |
| AAVS1 | gAAVS10 | gagatggtggacgaggaagg (SEQ ID NO: 140) |
| AAVS1 | gAAVS11 | gagatggctccaggaaatgg (SEQ ID NO: 141) |
| AAVS1 | gAAVS12 | taaggaatctgcctaacagg (SEQ ID NO: 142) |
| AAVS1 | gAAVS13 | tcaggagactaggaaggagg (SEQ ID NO: 143) |
| AAVS1 | gAAVS14 | tataaggtggtcccagctcg (SEQ ID NO: 144) |
| AAVS1 | gAAVS15 | ctggaagatgccatgacagg (SEQ ID NO: 145) |
| AAVS1 | gAAVS16 | gcacagactagagaggtaag (SEQ ID NO: 146) |
| AAVS1 | gAAVS17 | acagactagagaggtaaggg (SEQ ID NO: 147) |
| AAVS1 | gAAVS18 | gagaggtgacccgaatccac (SEQ ID NO: 148) |
| AAVS1 | gAAVS19 | gcacaggccccagaaggaga (SEQ ID NO: 149) |
| AAVS1 | gAAVS20 | ccggagaggacccagacacg (SEQ ID NO: 150) |
| AAVS1 | gAAVS21 | gagaggacccagacacgggg (SEQ ID NO: 151) |
| AAVS1 | gAAVS22 | gcaacacagcagagagcaag (SEQ ID NO: 152) |
| AAVS1 | gAAVS23 | gaagagggagtggaggaaga (SEQ ID NO: 153) |
| AAVS1 | gAAVS24 | aagacggaacctgaaggagg (SEQ ID NO: 154) |
| AAVS1 | gAAVS25 | agaaagcggcacaggcccag (SEQ ID NO: 155) |
| AAVS1 | gAAVS26 | gggaaacagtgggccagagg (SEQ ID NO: 156) |
| AAVS1 | gAAVS27 | gtccggactcaggagagaga (SEQ ID NO: 157) |
| AAVS1 | gAAVS28 | ggcacagcaagggcactcgg (SEQ ID NO: 158) |
| AAVS1 | gAAVS29 | gaagaggggaagtcgaggga (SEQ ID NO: 159) |
| AAVS1 | gAAVS30 | gggaatggtaaggaggcctg (SEQ ID NO: 160) |
| AAVS1 | gAAVS31 | gcagagtggtcagcacagag (SEQ ID NO: 161) |
| AAVS1 | gAAVS32 | gcacagagtggctaagccca (SEQ ID NO: 162) |
| AAVS1 | gAAVS33 | gacggggtgtcagcataggg (SEQ ID NO: 163) |
| AAVS1 | gAAVS34 | gcccagggccaggaacgacg (SEQ ID NO: 164) |
| AAVS1 | gAAVS35 | ggtggagtccagcacggcgc (SEQ ID NO: 165) |
| AAVS1 | gAAVS36 | acaggccgccaggaactcgg (SEQ ID NO: 166) |
| AAVS1 | gAAVS37 | actaggaagtgtgtagcacc (SEQ ID NO: 167) |
| AAVS1 | gAAVS38 | atgaatagcagactgccccg (SEQ ID NO: 168) |
| AAVS1 | gAAVS39 | acacccctaaaagcacagtg (SEQ ID NO: 169) |
| AAVS1 | gAAVS40 | caaggagttccagcaggtgg (SEQ ID NO: 170) |
| AAVS1 | gAAVS41 | aaggagttccagcaggtggg (SEQ ID NO: 171) |
| AAVS1 | gAAVS42 | tggaaagaggagggaagagg (SEQ ID NO: 172) |
| AAVS1 | gAAVS43 | tcgaattcctaactgccccg (SEQ ID NO: 173) |
| AAVS1 | gAAVS44 | gacctgcccagcacaccctg (SEQ ID NO: 174) |
| AAVS1 | gAAVS45 | ggagcagctgcggcagtggg (SEQ ID NO: 175) |
| AAVS1 | gAAVS46 | gggagggagagcttggcagg (SEQ ID NO: 176) |
| AAVS1 | gAAVS47 | gttacgtggccaagaagcag (SEQ ID NO: 177) |
| AAVS1 | gAAVS48 | gctgaacagagaagagctgg (SEQ ID NO: 178) |
| AAVS1 | gAAVS49 | tctgagggtggagggactgg (SEQ ID NO: 179) |
| AAVS1 | gAAVS50 | ggagaggtgagggacttggg (SEQ ID NO: 180) |
| AAVS1 | gAAVS51 | gtgaaccaggcagacaacga (SEQ ID NO: 181) |
| AAVS1 | gAAVS52 | caggtacctcctgagccacg (SEQ ID NO: 182) |
| AAVS1 | gAAVS53 | gggggagtaggggcatgcag (SEQ ID NO: 183) |
| hROSA26 | gHROSA26-1 | gcaaatggccagcaagggtg (SEQ ID NO: 184) |
| hROSA26 | gHROSA26-2 | caaatggccagcaagggtgg (SEQ ID NO: 309) |
| hROSA26 | gHROSA26-3 | gcagaacctgaggatatgga (SEQ ID NO: 310) |
| hROSA26 | gHROSA26-3 | aatacacagaatgaaaatag (SEQ ID NO: 311) |
| hROSA26 | gHROSA26-4 | ctggtgactagaataggcag (SEQ ID NO: 312) |
| hROSA26 | gHROSA26-5 | tggtgactagaataggcagt (SEQ ID NO: 313) |
| hROSA26 | gHROSA26-6 | taaaagaatgtgaaaagatg (SEQ ID NO: 314) |
| hROSA26 | gHROSA26-7 | tcaggagttcaagaccaccc (SEQ ID NO: 315) |
| hROSA26 | gHROSA26-8 | tgtagtcccagttatgcagg (SEQ ID NO: 316) |
| hROSA26 | gHROSA26-9 | gggttcacaccacaaatgca (SEQ ID NO: 317) |
| hROSA26 | gHROSA26-10 | ggcaaatggccagcaagggt (SEQ ID NO: 318) |
| hROSA26 | gHROSA26-11 | agaaaccaatcccaaagcaa (SEQ ID NO: 319) |
| hROSA26 | gHROSA26-12 | gccaaggacaccaaaaccca (SEQ ID NO: 320) |
| hROSA26 | gHROSA26-13 | agtggtgataaggcaacagt (SEQ ID NO: 321) |
| hROSA26 | gHROSA26-14 | cctgagacagaagtattaag (SEQ ID NO: 322) |
| hROSA26 | gHROSA26-15 | aaggtcacacaatgaatagg (SEQ ID NO: 323) |
| hROSA26 | gHROSA26-16 | caccatactagggaagaaga (SEQ ID NO: 324) |
| hROSA26 | gHROSA26-17 | caataccctgcccttagtgg (SEQ ID NO: 327) |
| hROSA26 | gHROSA26-18 | aataccctgcccttagtggg (SEQ ID NO: 325) |
| hROSA26 | gHROSA26-19 | ttagtggggggtggagtggg (SEQ ID NO: 326) |
| hROSA26 | gHROSA26-20 | gtggggggtggagtgggggg (SEQ ID NO: 328) |
| hROSA26 | gHROSA26-21 | ggggggtggagtggggggtg (SEQ ID NO: 329) |
| hROSA26 | gHROSA26-22 | ggggtggagtggggggtggg (SEQ ID NO: 330) |
| hROSA26 | gHROSA26-23 | gggtggagtggggggtgggg (SEQ ID NO: 331) |
| hROSA26 | gHROSA26-24 | gggggtggggaaagacatcg (SEQ ID NO: 332) |
| hROSA26 | gHROSA26-25 | gcaaatggccagcaagggtg (SEQ ID NO: 184) |
| hROSA26 | gHROSA26-26 | caaatggccagcaagggtgg (SEQ ID NO: 309) |
| hROSA26 | gHROSA26-27 | gcagaacctgaggatatgga (SEQ ID NO: 310) |
| hROSA26 | gHROSA26-28 | aatacacagaatgaaaatag (SEQ ID NO: 311) |
| hROSA26 | gHROSA26-29 | ctggtgactagaataggcag (SEQ ID NO: 312) |
| hROSA26 | gHROSA26-30 | tggtgactagaataggcagt (SEQ ID NO: 313) |
| hROSA26 | gHROSA26-31 | taaaagaatgtgaaaagatg (SEQ ID NO: 314) |
| hROSA26 | gHROSA26-32 | tcaggagttcaagaccaccc (SEQ ID NO: 315) |
| hROSA26 | gHROSA26-33 | tgtagtcccagttatgcagg (SEQ ID NO: 316) |
| hROSA26 | gHROSA26-34 | gggttcacaccacaaatgca (SEQ ID NO: 317) |
| hROSA26 | gHROSA26-35 | ggcaaatggccagcaagggt (SEQ ID NO: 318) |
| hROSA26 | gHROSA26-36 | agaaaccaatcccaaagcaa (SEQ ID NO: 319) |
| hROSA26 | gHROSA26-37 | gccaaggacaccaaaaccca (SEQ ID NO: 320) |
| hROSA26 | gHROSA26-38 | agtggtgataaggcaacagt (SEQ ID NO: 321) |
| hROSA26 | gHROSA26-39 | cctgagacagaagtattaag (SEQ ID NO: 322) |
| hROSA26 | gHROSA26-40 | aaggtcacacaatgaatagg (SEQ ID NO: 323) |
| hROSA26 | gHROSA26-41 | caccatactagggaagaaga (SEQ ID NO: 324) |
| hROSA26 | gHROSA26-42 | caataccctgcccttagtgg (SEQ ID NO: 327) |
| hROSA26 | gHROSA26-43 | aataccctgcccttagtggg (SEQ ID NO: 325) |
| hROSA26 | gHROSA26-44 | ttagtggggggtggagtggg (SEQ ID NO: 326) |
| hROSA26 | gHROSA26-45 | gtggggggtggagtgggggg (SEQ ID NO: 328) |
| hROSA26 | gHROSA26-46 | ggggggtggagtggggggtg (SEQ ID NO: 329) |
| hROSA26 | gHROSA26-47 | ggggtggagtggggggtggg (SEQ ID NO: 330) |
| hROSA26 | gHROSA26-48 | gggtggagtggggggtgggg (SEQ ID NO: 331) |
| hROSA26 | gHROSA26-49 | gggggtggggaaagacatcg (SEQ ID NO: 332) |
| hROSA26 | gHROSA26-50 | gcagctgtgaattctgatag (SEQ ID NO: 333) |
| hROSA26 | gHROSA26-51 | gagatcagagaaaccagatg (SEQ ID NO: 334) |
| hROSA26 | gHROSA26-52 | tctatactgattgcagccag (SEQ ID NO: 335) |
| hROSA26 | gHROSA26-1 | gcaaatggccagcaagggtg (SEQ ID NO: 184) |
| hROSA26 | 44F | AATCGAGAAGCGACTCGACA (SEQ ID NO: 185) |
| hROSA26 | 45F | GTCCCTGGGCGTTGCCCTGC (SEQ ID NO: 186) |
| hROSA26 | 46F | CCCTGGGCGTTGCCCTGCAG (SEQ ID NO: 187) |
| hROSA26 | 1nF | ccgtgggaagataaactaat (SEQ ID NO: 188) |
| hROSA26 | 2nF | tcccctgcagggcaacgccc (SEQ ID NO: 189) |
| hROSA26 | 3nF | gtcgagtcgcttctcgatta (SEQ ID NO: 190) |
| hROSA26 | 4nF | ctgctgcctcccgtcttgta (SEQ ID NO: 191) |
| hROSA26 | 5nF | gagtgccgcaatacctttat (SEQ ID NO: 192) |
| hROSA26 | 6nF | ACACTTTGGTGGTGCAGCAA (SEQ ID NO: 193) |
| hROSA26 | 7nF | TCTCAAATGGTATAAAACTC (SEQ ID NO: 194) |
| hROSA26 | 8nF | ccgtgggaagataaactaat (SEQ ID NO: 188) |
| hROSA26 | 9F | aatcccgcccataatcgaga (SEQ ID NO: 195) |
| hROSA26 | 10F | tcccgcccataatcgagaag (SEQ ID NO: 196) |
| hROSA26 | 11F | cccataatcgagaagcgact (SEQ ID NO: 197) |
| hROSA26 | 12F | gagaagcgactcgacatgga (SEQ ID NO: 198) |
| hROSA26 | 13F | gaagcgactcgacatggagg (SEQ ID NO: 199) |
| hROSA26 | 14F | gcgactcgacatggaggcga (SEQ ID NO: 200) |
| hROSA26 | 44F | aaacTGTCGAGTCGCTTCTCGATTc (SEQ ID NO: 201) |
| hROSA26 | 45F | aaacGCAGGGCAACGCCCAGGGACc (SEQ ID NO: 202) |
| hROSA26 | 46F | aaacCTGCAGGGCAACGCCCAGGGc (SEQ ID NO: 203) |
| CCR5 | 1F | acagggttaatgtgaagtcc (SEQ ID NO: 217) |
| CCR5 | 2F | tccccctctacatttaaagt (SEQ ID NO: 218) |
| CCR5 | 3F | catttaaagttggtttaagt (SEQ ID NO: 219) |
| CCR5 | 4F | ttagaaaatataaagaataa (SEQ ID NO: 220) |
| CCR5 | 5 | TAAATGCTTACTGGTTTGAA (SEQ ID NO: 221) |
| CCR5 | 6F | TCCTGGGTCCAGAAAAAGAT (SEQ ID NO: 222) |
| CCR5 | 7F | TTGGGTGGTGAGCATCTGTG (SEQ ID NO: 223) |
| CCR5 | 8F | CGGGGAGAGTGGAGAAAAAG (SEQ ID NO: 224) |
| CCR5 | 9F | GTTAAAACTCTTTAGACAAC (SEQ ID NO: 225) |
| CCR5 | 10F | GAAAATCCCCACTAAGATCC (SEQ ID NO: 226) |
| CCR5 | gCCR5-1 | agtagcagtaatgaagctgg (SEQ ID NO: 237) |
| CCR5 | gCCR5-2 | atacccagacgagaaagctg (SEQ ID NO: 238) |
| CCR5 | gCCR5-3 | tacccagacgagaaagctga (SEQ ID NO: 239) |
| CCR5 | gCCR5-4 | ggtggtgagcatctgtgtgg (SEQ ID NO: 240) |
| CCR5 | gCCR5-5 | aaatgagaagaagaggcaca (SEQ ID NO: 241) |
| CCR5 | gCCR5-6 | cttgtggcctgggagagctg (SEQ ID NO: 242) |
| CCR5 | gCCR5-7 | gctgtagaaggagacagagc (SEQ ID NO: 243) |
| CCR5 | gCCR5-8 | gagctggttgggaagacatg (SEQ ID NO: 244) |
| CCR5 | gCCR5-9 | ctggttgggaagacatgggg (SEQ ID NO: 245) |
| CCR5 | gCCR5-10 | cgtgaggatgggaaggaggg (SEQ ID NO: 246) |
| CCR5 | gCCR5-11 | atgcagagtcagcagaactg (SEQ ID NO: 247) |
| CCR5 | gCCR5-12 | aagacatcaagcacagaagg (SEQ ID NO: 248) |
| CCR5 | gCCR5-13 | tcaagcacagaaggaggagg (SEQ ID NO: 249) |
| CCR5 | gCCR5-14 | aaccgtcaataggcaaaggg (SEQ ID NO: 250) |
| CCR5 | gCCR5-15 | ccgtatttcagactgaatgg (SEQ ID NO: 251) |
| CCR5 | gCCR5-16 | gagaggacaggtgctacagg (SEQ ID NO: 252) |
| CCR5 | gCCR5-17 | aaccaaggaagggcaggagg (SEQ ID NO: 253) |
| CCR5 | gCCR5-18 | gacctctgggtggagacaga (SEQ ID NO: 254) |
| CCR5 | gCCR5-19 | cagatgaccatgacaagcag (SEQ ID NO: 255) |
| CCR5 | gCCR5-20 | aacaccagtgagtagagcgg (SEQ ID NO: 256) |
| CCR5 | gCCR5-21 | aggaccttgaagcacagaga (SEQ ID NO: 257) |
| CCR5 | gCCR5-22 | tacagaggcagactaaccca (SEQ ID NO: 258) |
| CCR5 | gCCR5-23 | acagaggcagactaacccag (SEQ ID NO: 259) |
| CCR5 | gCCR5-24 | taaatgacgtgctagacctg (SEQ ID NO: 260) |
| CCR5 | gCCR5-25 | agtaaccactcaggacaggg (SEQ ID NO: 261) |
| chr2 | gchr2-1 | accacaaaacagaaacacca (SEQ ID NO: 262) |
| chr2 | gchr2-2 | gtttgaagacaagcctgagg (SEQ ID NO: 263) |
| chr4 | gchr4-1 | gctgaaccccaaaagacagg (SEQ ID NO: 264) |
| chr4 | gchr4-2 | gcagctgagacacacaccag (SEQ ID NO: 265) |
| chr4 | gchr4-3 | aggacaccccaaagaagctg (SEQ ID NO: 266) |
| chr4 | gchr4-4 | ggacaccccaaagaagctga (SEQ ID NO: 267) |
| chr6 | gchr6-1 | ccagtgcaatggacagaaga (SEQ ID NO: 268) |
| chr6 | gchr6-2 | agaagagggagcctgcaagt (SEQ ID NO: 269) |
| chr6 | gchr6-3 | gtgtttgggccctagagcga (SEQ ID NO: 270) |
| chr6 | gchr6-4 | catgtgcctggtgcaatgca (SEQ ID NO: 271) |
| chr6 | gchr6-5 | tacaaagaggaagataagtg (SEQ ID NO: 272) |
| chr6 | gchr6-6 | gtcacagaatacaccactag (SEQ ID NO: 273) |
| chr6 | gchr6-7 | gggttaccctggacatggaa (SEQ ID NO: 274) |
| chr6 | gchr6-8 | catggaagggtattcactcg (SEQ ID NO: 275) |
| chr6 | gchr6-9 | agagtggcctagacaggctg (SEQ ID NO: 276) |
| chr6 | gchr6-10 | catgctggacagctcggcag (SEQ ID NO: 277) |
| chr6 | gchr6-11 | agtgaaagaagagaaaattc (SEQ ID NO: 278) |
| chr6 | gchr6-12 | tggtaagtctaagaaaccta (SEQ ID NO: 279) |
| chr6 | gchr6-13 | cccacagcctaaccacccta (SEQ ID NO: 280) |
| chr6 | gchr6-14 | aatatttcaaagccctaggg (SEQ ID NO: 281) |
| chr6 | gchr6-15 | gcactcggaacagggtctgg (SEQ ID NO: 282) |
| chr6 | gchr6-16 | agataggagctccaacagtg (SEQ ID NO: 283) |
| chr6 | gchr6-17 | aagttagagcagccaggaaa (SEQ ID NO: 284) |
| chr6 | gchr6-18 | tagagcagccaggaaaggga (SEQ ID NO: 285) |
| chr6 | gchr6-19 | tgaatacccttccatgtcca (SEQ ID NO: 286) |
| chr6 | gchr6-20 | cctgcattgcaccaggcaca (SEQ ID NO: 287) |
| chr6 | gchr6-21 | tctagggcccaaacacacct (SEQ ID NO: 288) |
| chr6 | gchr6-22 | tccctccatctatcaaaagg (SEQ ID NO: 289) |
| chr10 | gchr10-1 | agccctgagacagaagcagg (SEQ ID NO: 290) |
| chr10 | gchr10-2 | gccctgagacagaagcaggt (SEQ ID NO: 291) |
| chr10 | gchr10-3 | aggagatgcagtgatacgca (SEQ ID NO: 292) |
| chr10 | gchr10-4 | acaataccaagggtatccgg (SEQ ID NO: 293) |
| chr10 | gchr10-5 | tgataaagaaaacaaagtga (SEQ ID NO: 294) |
| chr10 | gchr10-6 | aaagaaaacaaagtgaggga (SEQ ID NO: 295) |
| chr10 | gchr10-7 | gtggcaagtggagaaattga (SEQ ID NO: 296) |
| chr10 | gchr10-8 | caagtggagaaattgaggga (SEQ ID NO: 297) |
| chr10 | gchr10-9 | gtggtgatgattgcagctgg (SEQ ID NO: 298) |
| chr11 | gchr11-1 | ctatgtgcctgacacacagg (SEQ ID NO: 299) |
| chr11 | gchr11-2 | gggttggaccaggaaagagg (SEQ ID NO: 300) |
| chr17 | gchr17-1 | gatgcctggaaaaggaaaga (SEQ ID NO: 301) |
| chr17 | gchr17-2 | tagtatgcacctgcaagagg (SEQ ID NO: 302) |
| chr17 | gchr17-3 | tatgcacctgcaagaggcgg (SEQ ID NO: 303) |
| chr17 | gchr17-4 | aggggaagaagagaagcaga (SEQ ID NO: 304) |
| chr17 | gchr17-5 | gctgaatcaagagacaagcg (SEQ ID NO: 305) |
| chr17 | gchr17-6 | aagcaaataaatctcctggg (SEQ ID NO: 306) |
| chr17 | gchr17-7 | agatgagtgctagagactgg (SEQ ID NO: 307) |
| chr17 | gchr17-8 | ctgatggttgagcacagcag (SEQ ID NO: 308) |

In embodiments, gRNAs for targeting human genomic safe harbor sites using any of the gRNA-based targeting elements, e.g., without limitation, dCas, in areas of open chromatin are shown in TABLES 2-6.

TABLE 2
Guide RNA sequences targeting the genomic safe harbor site, hROSA26.

| HROSA26 GUIDE NO. | DNA SEQUENCE |
|---|---|
| GUIDE 44 | AATCGAGAAGCGACTCGACA (SEQ ID NO: 452) |
| GUIDE 45-C | GTCCCTGGGCGTTGCCCTGC (SEQ ID NO: 453) |
| GUIDE 46-C | CCCTGGGCGTTGCCCTGCAG (SEQ ID NO: 454) |
| SPG GUIDE1-C | GAGTGAGCAGCTGTAAGATT (SEQ ID NO: 455) |
| SPG GUIDE2-C | CAGGGGAGTGAGCAGCTGTA (SEQ ID NO: 456) |
| SPG GUIDE3-C | CCTGCAGGGGAGTGAGCAGC (SEQ ID NO: 457) |
| SPG GUIDE4-C | TGCCCTGCAGGGGAGTGAGC (SEQ ID NO: 458) |
| SPG GUIDE5-C | CGTTGCCCTGCAGGGGAGTG (SEQ ID NO: 459) |
| SPG GUIDE6-C | TGGGCGTTGCCCTGCAGGGG (SEQ ID NO: 460) |
| SPG GUIDE7-C | TTGGTCCCTGGGCGTTGCCC (SEQ ID NO: 461) |
| SPG GUIDE8 | AAGAATCCCGCCCATAATCG (SEQ ID NO: 462) |
| SPG GUIDE9 | AATCCCGCCCATAATCGAGA (SEQ ID NO: 463) |
| SPG GUIDE10 | TCCCGCCCATAATCGAGAAG (SEQ ID NO: 464) |
| SPG GUIDE11 | CCCATAATCGAGAAGCGACT (SEQ ID NO: 465) |
| SPG GUIDE12 | GAGAAGCGACTCGACATGGA (SEQ ID NO: 466) |
| SPG GUIDE13 | GAAGCGACTCGACATGGAGG (SEQ ID NO: 467) |
| SPG GUIDE14 | GCGACTCGACATGGAGGCGA (SEQ ID NO: 468) |
| GUIDE N1 | CCGTGGGAAGATAAACTAAT (SEQ ID NO: 469) |
| GUIDE N2 | TCCCCTGCAGGGCAACGCCC (SEQ ID NO: 470) |
| GUIDE N3-C | GTCGAGTCGCTTCTCGATTA (SEQ ID NO: 471) |
| GUIDE O12 | CGACACCAACTCTAGTCCGT (SEQ ID NO: 472) |
| GUIDE O13 | CAGCTGCTCACTCCCCTGCA (SEQ ID NO: 473) |
| GUIDE O14-C | AGTCGCTTCTCGATTATGGG (SEQ ID NO: 474) |

TABLE 3
Guide RNA sequences targeting the genomic safe harbor site, AAVS1.

| AAVS1 GUIDE NO. | DNA SEQUENCE |
|---|---|
| AAV GUIDE 12 | ACCCTTGGAAGGACCTGGCTGGG (SEQ ID NO: 475) |
| AAV GUIDE 13c | TCCGAGCTTGACCCTTGGAA (SEQ ID NO: 476) |
| AAV GUIDE 14 | GGAGCCACGAAAACAGATCCAGG (SEQ ID NO: 477) |
| AAV GUIDE 14c | TGGTTTCCGAGCTTGACCCT (SEQ ID NO: 478) |
| AAV GUIDE 15 | GGAGCCACGAAAACAGATCCAGG (SEQ ID NO: 479) |
| AAV GUIDE 16 | AGATCCAGGGACACGGTGCTAGG (SEQ ID NO: 480) |
| AAV GUIDE 17 | GACACGGTGCTAGGACAGTGGGG (SEQ ID NO: 481) |
| AAV GUIDE 18 | GAAAATGACCCAACAGCCTCTGG (SEQ ID NO: 482) |
| AAV GUIDE 19 | GCCTGGCCGGCCTGACCACTGGG (SEQ ID NO: 483) |
| AAV GUIDE 20 | CTGAGCACTGAAGGCCTGGCCGG (SEQ ID NO: 484) |
| AAV GUIDE 21 | TGGTTTCCACTGAGCACTGAAGG (SEQ ID NO: 485) |
| AAV GUIDE 22 | GGTGCTTTCCTGAGGACCGATAG (SEQ ID NO: 486) |
| AAV GUIDE 23 | GCGCTTCCAGTGCTCAGACTAGG (SEQ ID NO: 487) |
| AAV GUIDE 24 | CAGTGCTCAGACTAGGGAAGAGG (SEQ ID NO: 488) |
| AAV GUIDE 25 | GCCCCTCCTCCTTCAGAGCCAGG (SEQ ID NO: 489) |
| AAV GUIDE 26 | TCCTTCAGAGCCAGGAGTCCTGG (SEQ ID NO: 490) |
| AAV GUIDE 27 | CCAAGGGTCAAGCTCGGAAACCA (SEQ ID NO: 491) |
| AAV GUIDE 28 | CTGCAGAGTATCTGCTGGGGTGG (SEQ ID NO: 492) |
| AAV GUIDE 29 | CGTTCCTGCAGAGTATCTGCTGG (SEQ ID NO: 493) |
| AAV GUIDE 30c | GTGGGGAAAATGACCCAACA (SEQ ID NO: 494) |
| AAV GUIDE 31 | GAAGGCCTGGCCGGCCTGAC (SEQ ID NO: 495) |
| AAV GUIDE 32c | ACTCCTGGCTCTGAAGGAGG (SEQ ID NO: 496) |
| AAV GUIDE 33c | GGGCTGGGGGCCAGGACTCC (SEQ ID NO: 497) |
| AAV GUIDE 34 | GTCCTTCCAAGGGTCAAGCT (SEQ ID NO: 498) |
| AAV GUIDE 35 | TCAAGCTCGGAAACCACCCC (SEQ ID NO: 499) |

TABLE 4
Guide RNA sequences targeting a chromosome 4 genomic safe harbor
site (hg38 chr4:30,793,039-30,793,980)

| CHR4 GUIDE NO. | DNA SEQUENCE |
|---|---|
| Guide C4-1 | ATTGTCTTCACTAAACCCGTTGG (SEQ ID NO: 500) |
| Guide C4-2 | TAAACCCGTTGGGAATACAATGG (SEQ ID NO: 501) |
| Guide C4-3 | TTGTCTTCACTAAACCCGTTGGG (SEQ ID NO: 502) |
| Guide C4-4 | TGATTCATAGGAGTCTATTAAGG (SEQ ID NO: 503) |
| Guide C4-5 | TTACATATGCTTCGAGTTTGTGG (SEQ ID NO: 504) |
| Guide C4-6 | ACTCTTAAGGTAGGACTAATTGG (SEQ ID NO: 505) |
| Guide C4-7 | TATGTGTGCAATAGCGTTAAAGG (SEQ ID NO: 506) |
| Guide C4-8 | CGTTGGGAATACAATGGCTTAGG (SEQ ID NO: 507) |
| Guide C4-9 | TCACAATGGAACTCTGCCTTTGG (SEQ ID NO: 508) |
| Guide C4-10 | GACCACAAATCAATGCCCAAAGG (SEQ ID NO: 509) |
| Guide C4-11 | CTAAGCCATTGTATTCCCAACGG (SEQ ID NO: 510) |
| Guide C4-12 | AGCATTCTGGAGTGTCACAATGG (SEQ ID NO: 511) |
| Guide C4-13 | CAATAGCCCACTTTAATACTAGG (SEQ ID NO: 512) |
| Guide C4-14 | CTTTATCCAAGTGAATCCTTTGG (SEQ ID NO: 513) |
| Guide C4-15 | GGCATTGATTTGTGGTCATTTGG (SEQ ID NO: 514) |
| Guide C4-16 | TAAGCCATTGTATTCCCAACGGG (SEQ ID NO: 515) |
| Guide C4-17 | AATACAATCACTCTTAAGGTAGG (SEQ ID NO: 516) |
| Guide C4-18 | GAAGTACCTTTCACTATTTTGGG (SEQ ID NO: 517) |
| Guide C4-19 | CAAGCAACAAATGACTTCTAAGG (SEQ ID NO: 518) |
| Guide C4-20 | TTTGAATACAATCACTCTTAAGG (SEQ ID NO: 519) |
| Guide C4A1 | ACAAACGGACTACGTAAACTTGG (SEQ ID NO: 520) |
| Guide C4A2 | ACAAGATGTGAACACGACGATGG (SEQ ID NO: 521) |
| Guide C4A3 | GTTGCACCGTTGATTCCTTCAGG (SEQ ID NO: 522) |
| Guide C4A4 | AGTAATATTGAATTAGGGCGTGG (SEQ ID NO: 523) |
| Guide C4A5 | CCTGATGTTGGCTCGACATTAGG (SEQ ID NO: 524) |
| Guide C4A6 | CTTTGTTGGGTCTTAGCTTAAGG (SEQ ID NO: 525) |
| Guide C4A7 | TCGGAACAGCTCCTTCCTGAAGG (SEQ ID NO: 526) |
| Guide C4A8 | AGTAGTTTCTGAGGTCATGTTGG (SEQ ID NO: 527) |
| Guide C4A9 | CTTGAAAATACGATGATGTGAGG (SEQ ID NO: 528) |
| Guide C4A10 | GCATTAATCTAGAGAGAGGGAGG (SEQ ID NO: 529) |
| Guide C4A11 | GGGTCATGTTAGAATTCATGTGG (SEQ ID NO: 530) |
| Guide C4A12 | TGATGCATTAATCTAGAGAGAGG (SEQ ID NO: 531) |
| Guide C4A13 | ACATCATCGTATTTTCAAGTTGG (SEQ ID NO: 532) |
| Guide C4A14 | CTAGCTGACAAACATGTGAGTGG (SEQ ID NO: 533) |
| Guide C4A15 | AACATGACCCAAGTGAGTCCAGG (SEQ ID NO: 534) |
| Guide C4A16 | GATTCCGTATTTGCTTTGTTGGG (SEQ ID NO: 535) |
| Guide C4A17 | TACGATGATGTGAGGAAATAAGG (SEQ ID NO: 536) |
| Guide C4A18 | GTAATATGTCTAAGTACTGATGG (SEQ ID NO: 537) |
| Guide C4A19 | GTAAAGTGAGCTGGTTCATTAGG (SEQ ID NO: 538) |
| Guide C4A20 | ACTAGAGTCCTTAAGAAGGGGGG (SEQ ID NO: 539) |

CHOPCHOP algorithm

TABLE 5
Guide RNA sequences targeting a chromosome 22 genomic safe harbor
site (hg38 chr22:35,373,429-35,380,000).

| CHR22 GUIDE NO. | DNA SEQUENCE |
|---|---|
| Guide C22-1 | ATAACACGTGAGCCGTCCTAAGG (SEQ ID NO: 912) |
| Guide C22-2 | GGAAGACTTTTCTCTATACGAGG (SEQ ID NO: 540) |
| Guide C22-3 | GCATTCCTTTCATCCATGGCAGG (SEQ ID NO: 541) |
| Guide C22-4 | GACATATGGTTATAAAAATCAGG (SEQ ID NO: 542) |
| Guide C22-5 | GGAGTGCAGTCCCTGACATATGG (SEQ ID NO: 543) |
| Guide C22-6 | GTGGGTTAGGGTGGTTAACTGGG (SEQ ID NO: 544) |
| Guide C22-7 | AGGTGCAAAAAGGTTGCTGTGGG (SEQ ID NO: 545) |
| Guide C22-8 | CGTGACAAGGCAAAGTGGCGTGG (SEQ ID NO: 546) |
| Guide C22-9 | GAAGGACTGCCCCTGACGTCAGG (SEQ ID NO: 547) |
| Guide C22-10 | CTGCCCCTGACGTCAGGAGTTGG (SEQ ID NO: 548) |
| Guide C22-11 | TGTGGGTTAGGGTGGTTAACTGG (SEQ ID NO: 549) |
| Guide C22-12 | ACCCTTTTAGAGTTTTCTGCTGG (SEQ ID NO: 550) |
| Guide C22-13 | AACTTCCTGCCATGGATGAAAGG (SEQ ID NO: 551) |
| Guide C22-14 | GCAAAAAGGTTGCTGTGGGTTGG (SEQ ID NO: 552) |
| Guide C22-15 | AATTTGGGGGTAGATAGGCATGG (SEQ ID NO: 553) |
| Guide C22-16 | AGAAAACTCTAAAAGGGTATAGG (SEQ ID NO: 554) |
| Guide C22-17 | ATTAGCATTCCTTTCATCCATGG (SEQ ID NO: 555) |
| Guide C22-18 | CCCAGCAGAAAACTCTAAAAGGG (SEQ ID NO: 556) |
| Guide C22-19 | CAGGTGCAAAAAGGTTGCTGTGG (SEQ ID NO: 557) |
| Guide C22-20 | GCAAGAGATGAAATTCCATATGG (SEQ ID NO: 558) |
| Guide C22A1 | GGGCTGTTCTAACGAAGTCTGGG (SEQ ID NO: 559) |
| Guide C22A2 | TGTCCATTCAGCGACCCTAGAGG (SEQ ID NO: 560) |
| Guide C22A3 | GGCTGTTCTAACGAAGTCTGGGG (SEQ ID NO: 561) |
| Guide C22A4 | GTCCATTCAGCGACCCTAGAGGG (SEQ ID NO: 562) |
| Guide C22A5 | GGGGCTGTTCTAACGAAGTCTGG (SEQ ID NO: 563) |
| Guide C22A6 | GGCTGAATCAGCATGCGAAAGGG (SEQ ID NO: 564) |
| Guide C22A7 | TTCCAATGGGGGGCATAGCCTGG (SEQ ID NO: 565) |
| Guide C22A8 | TACCCTCTAGGGTCGCTGAATGG (SEQ ID NO: 566) |
| Guide C22A9 | ATCCTCTTGGGCCTTATAAGAGG (SEQ ID NO: 567) |
| Guide C22A10 | GGCCAGGCTATGCCCCCCATTGG (SEQ ID NO: 568) |
| Guide C22A11 | CTAGAGGACCAGAACAACTCTGG (SEQ ID NO: 569) |
| Guide C22A12 | TCCCTCTTATAAGGCCCAAGAGG (SEQ ID NO: 570) |
| Guide C22A13 | AGGCTGAATCAGCATGCGAAAGG (SEQ ID NO: 571) |
| Guide C22A14 | GGACCAGAACAACTCTGGCCTGG (SEQ ID NO: 572) |
| Guide C22A15 | GGGCTTTTATTTGGCCCAGCAGG (SEQ ID NO: 573) |
| Guide C22A16 | GTCGCTGAATGGACAGACTCTGG (SEQ ID NO: 574) |
| Guide C22A17 | CTCATGAGTTTTACCCTCTAGGG (SEQ ID NO: 575) |
| Guide C22A18 | TCCTCTTGGGCCTTATAAGAGGG (SEQ ID NO: 576) |
| Guide C22A19 | TCTTGGGCCTTATAAGAGGGAGG (SEQ ID NO: 577) |
| Guide C22A20 | TAGAACAGCCCCCCACACAGTGG (SEQ ID NO: 578) |

TABLE 6
Guide RNA sequences targeting chromosome X (HPRT) (hg38
chrX:134,475,807-134,476,794).

| CHRX GUIDE NO. | DNA SEQUENCE |
|---|---|
| Guide CX-1 | GTTACGTTATGACTAATCTTTGG (SEQ ID NO: 579) |
| Guide CX-2 | TACGTTATGACTAATCTTTGGGG (SEQ ID NO: 580) |
| Guide CX-3 | GGAAGTAGTGTTATGATGTATGG (SEQ ID NO: 581) |
| Guide CX-4 | GTTATGATGTATGGGCATAAAGG (SEQ ID NO: 582) |
| Guide CX-5 | GAAGTAGTGTTATGATGTATGGG (SEQ ID NO: 583) |
| Guide CX-6 | ATAGCTGCTGGCAGTATAACTGG (SEQ ID NO: 584) |
| Guide CX-7 | GCATCACAACATTGACACTGTGG (SEQ ID NO: 585) |
| Guide CX-8 | AAGGCGAGTTTCTACAAAGATGG (SEQ ID NO: 586) |
| Guide CX-9 | TTACGTTATGACTAATCTTTGGG (SEQ ID NO: 587) |
| Guide CX-10 | CAAGACTGATTAAGACTGATGGG (SEQ ID NO: 588) |
| Guide CX-11 | AGCAGCAATGTATTAAAGGCTGG (SEQ ID NO: 589) |
| Guide CX-12 | CTACAGGATTGATGTAAACATGG (SEQ ID NO: 590) |
| Guide CX-13 | TGGGCATAAAGGGTTTTAATGGG (SEQ ID NO: 591) |
| Guide CX-14 | ACATCAATCCTGTAGGTGATTGG (SEQ ID NO: 592) |
| Guide CX-15 | ATTCTAGTCATTATAGCTGCTGG (SEQ ID NO: 593) |
| Guide CX-16 | CATCAATCCTGTAGGTGATTGGG (SEQ ID NO: 594) |
| Guide CX-17 | GTTATAAGATCAATTCTGAGTGG (SEQ ID NO: 595) |
| Guide CX-18 | GGCAGACTGTGGATCAAAAGTGG (SEQ ID NO: 596) |
| Guide CX-19 | ATGGCTGCCCAATCACCTACAGG (SEQ ID NO: 597) |
| Guide CX-20 | TCAAAGCATGTACTTAGAGTTGG (SEQ ID NO: 598) |

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises Cas12 or a variant thereof, e.g., without limitation, Cas12a (e.g., dCas12a), or Cas12j (e.g., dCas12j), or Cas12k (e.g., dCas12k). In embodiments, the targeting element comprises a Cas12 enzyme guide RNA complex. In embodiments, comprises a nuclease-deficient dCas12 guide RNA complex, optionally dCas12j guide RNA complex or dCas12a guide RNA complex.

In embodiments, the targeting element is selected from a zinc finger (ZF), transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the helper enzyme of the present disclosure is capable of inserting a donor DNA at a TA dinucleotide site or a TTAA tetranucleotide site in a genomic safe harbor site (GSHS) of a nucleic acid molecule. The helper enzyme of the present disclosure is suitable for causing insertion of the donor DNA in a GSHS when contacted with a biological cell.

In embodiments, the targeting element is suitable for directing the helper enzyme of the present disclosure to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9, CasX, or Cas12 OR dCas9, dCasX, or dCas12, or variants thereof) DBDs cause the the helper enzyme of the present disclosure to bind specifically to human GSHS. In embodiments, the TALEs or Cas DBDs sequester the helper to GSHS and promote transposition to nearby TA dinucleotide or a TTAA tetranucleotide sites which can be located in proximity to the repeat variable di-residues (RVD)

TALE or gRNA nucleotide sequences. The GSHS regions are located in open chromatin sites that are susceptible to helper activity. Accordingly, the helper enzyme of the present disclosure does not only operate based on its ability to recognize TA or TTAA sites, but it also directs a donor DNA (having a transgene) to specific locations in proximity to a TALE or Cas DBD. The helper enzyme of the present disclosure in accordance with embodiments of the present disclosure has negligible risk of genotoxicity and exhibits superior features as compared to existing gene therapies. In embodiments, the helper enzyme of the present disclosure is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies.

In embodiments, TALE or Cas DBDs are customizable, such as a TALE or Cas DBDs is selected for targeting a specific genomic location. In embodiments, the genomic location is in proximity to a TA dinucleotide site or a TTAA (SEQ ID NO: 440) tetranucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as Fokl nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nat. Biotechnol. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14 (1): 49-55. The following table, for example, shows such code:

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat. Biotechnol. 2011; 29:143-148. Guidelines for selection of potential target sites and for use of particular TALE repeat domains (harboring NH residues at the hypervariable positions) for recognition of G bases have been proposed. See Streubel et al. Nat. Biotechnol. 2012; 30:593-595.

Accordingly, in embodiments, the TALE DBD comprises one or more repeat sequences. In embodiments, the TALE DBD comprises about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H (gap), and IG.

In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor; and human Rosa26 locus. In embodiments, the GSHS is located on human chromosome 2, 4, 6, 10, 11, 17, 22 or X.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32) TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53) TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26) TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of

(SEQ ID NO: 355)
NH NH HD HD NH NH HD HD NG NH NI HD HD NI HD NG
NH NH,
(SEQ ID NO: 356)
NH NI NI NH NH HD HD NG NH NH HD HD NH NH HD HD
NG NH,
(SEQ ID NO: 357)
NH NI NH HD NI HD NG NH NI NI NH NH HD HD NG NH
NH HD,
(SEQ ID NO: 358)
HD HD NI HD NG NH NI NH HD NI HD NG NH NI NI NH
NH HD,
(SEQ ID NO: 359)
NH NH NG NG NG HD HD NI HD NG NH NI NH HD NI HD
NG NH,
(SEQ ID NO: 360)
NH NH NH NH NI NI NI NI NG NH NI HD HD HD NI NI
HD NI,
(SEQ ID NO: 361)
NI NH NH NI HD NI NH NG NH NH NH NH NI NI NI NI
NG NH,
(SEQ ID NO: 362)
HD HD NI NH NH NH NI HD NI HD NH NH NG NH HD NG
NI NH,
(SEQ ID NO: 363)
HD NI NH NI NH HD HD NI NH NH NI NH NG HD HD NG
NH NH,
(SEQ ID NO: 364)
HD HD NG NG HD NI NH NI NH HD HD NI NH NH NI NH
NG HD,
(SEQ ID NO: 365)
HD HD NG HD HD NG NG HD NI NH NI NH HD HD NI NH
NH NI,
(SEQ ID NO: 366)
HD HD NI NH HD HD HD HD NG HD HD NG HD HD NG NG
HD NI,
(SEQ ID NO: 367)
HD HD NH NI NH HD NG NG NH NI HD HD HD NG NG NH
NH NI,
(SEQ ID NO: 368)
NH NH NG NG NG HD HD NH NI NH HD NG NG NH NI HD
HD HD,
(SEQ ID NO: 369)
NH NH NH NH NG NH NH NG NG NG HD HD NH NI NH HD
NG NG,
(SEQ ID NO: 370)
HD NG NH HD NG NH NH NH NH NG NH NH NG NG NG HD
HD NH,
(SEQ ID NO: 371)
NH HD NI NH NI NH NG NI NG HD NG NH HD NG NH NH
NH NH,
(SEQ ID NO: 372)
HD HD NI NI NG HD HD HD HD NG HD NI NH NG,
(SEQ ID NO: 373)
HD NI NH NG NH HD NG HD NI NH NG NH NH NI NI,
(SEQ ID NO: 374)
NH NI NI NI HD NI NG HD HD NH NH HD NH NI HD NG
HD NI,
(SEQ ID NO: 375)
HD NH HD HD HD HD NG HD NI NI NI NG HD NG NG NI
HD NI,
(SEQ ID NO: 376)
HD NI NI NI NG HD NG NG NI HD NI NH HD NG NH HD
NG HD,
(SEQ ID NO: 377)
HD NG NG NI HD NI NH HD NG NH HD NG HD NI HD NG
HD HD,
(SEQ ID NO: 378)
NI HD NI NH HD NG NH HD NG HD NI HD NG HD HD HD
HD NG,
(SEQ ID NO: 379)
NH HD NG HD NI HD NG HD HD HD HD NG NH HD NI NH
NH NH,
(SEQ ID NO: 380)
HD HD HD HD NG NH HD NI NH NH NH HD NI NI HD NH
HD HD,
(SEQ ID NO: 381)
NH HD NI NH NH NH HD NI NI HD NH HD HD HD NI NH
NH NH,
(SEQ ID NO: 382)
HD NG HD NH NI NG NG NI NG NH NH NH HD NH NH NH
NI NG,
(SEQ ID NO: 383)
HD NH HD NG NG HD NG HD NH NI NG NG NI NG NH NH
NH HD,
(SEQ ID NO: 384)
NH NG HD NH NI NH NG HD NH HD NG NG HD NG HD NH
NI NG,
(SEQ ID NO: 385)
HD HD NI NG NH NG HD NH NI NH NG HD NH HD NG NG
HD NG,
(SEQ ID NO: 386)
HD NH HD HD NG HD HD NI NG NH NG HD NH NI NH NG
HD NH,
(SEQ ID NO: 387)
HD NH NG HD NI NG HD NH HD HD NG HD HD NI NG NH
NG HD,
(SEQ ID NO: 388)
NH NI NG HD NG HD NH NG HD NI NG HD NH HD HD NG
HD HD,
(SEQ ID NO: 389)
NH HD NG NG HD NI NH HD NG NG HD HD NG NI,
(SEQ ID NO: 390)
HD NG NK NG NH NI NG HD NI NG NH HD HD NI,
(SEQ ID NO: 391)
NI HD NI NN NG NN NN NG NI HD NI HD NI HD HD NG,
(SEQ ID NO: 392)
HD HD NI HD HD HD HD HD HD NI HD NG NI NI NN,
(SEQ ID NO: 393)
HD NI NG NG NN NN HD HD NN NN NN HD NI HD,
(SEQ ID NO: 394)
NN HD NG NG NN NI NI HD HD HD NI NN NN NI NN NI,
(SEQ ID NO: 395)
NI HD NI HD HD HD NN NI NG HD HD NI HD NG NN NN
NN,
(SEQ ID NO: 396)
NN HD NG NN HD NI NG HD NI NI HD HD HD HD,
(SEQ ID NO: 397)
NN NN HD NI HD NN NI NI NI HD NI HD HD HD NG HD
HD,
(SEQ ID NO: 398)
NN NN NG NN NN HD NG HD NI NG NN HD HD NG NN,
(SEQ ID NO: 399)
NN NI NG NG NG NN HD NI HD NI NN HD NG HD NI NG,
(SEQ ID NO: 400)
NI NI NH HD NG HD NG NH NI NH NH NI NH HD,
(SEQ ID NO: 401)
HD HD HD NG NI NK HD NG NH NG HD HD HD HD,
(SEQ ID NO: 402)
NH HD HD NG NI NH HD NI NG NH HD NG NI NH,
(SEQ ID NO: 403)
NI NG NH NH NH HD NG NG HD NI HD NH NH NI NG,
(SEQ ID NO: 404)
NH NI NI NI HD NG NI NG NH HD HD NG NH HD,
(SEQ ID NO: 405)
NH HD NI HD HD NI NG NG NH HD NG HD HD HD,
(SEQ ID NO: 406)
NH NI HD NI NG NH HD NI NI HD NG HD NI NH,
(SEQ ID NO: 407)
NI HD NI HD HD NI HD NG NI NH NH NH NH NG,
(SEQ ID NO: 408)
NH NG HD NG NH HD NG NI NH NI HD NI NH NH,
(SEQ ID NO: 409)
NH NH HD HD NG NI NH NI HD NI NH NH HD NG NH,
(SEQ ID NO: 410)
NH NI NH NH HD NI NG NG HD NG NG NI NG HD NH,
(SEQ ID NO: 411)
NN HD HD NG NN NN NI NI NI HD NN NG NG HD HD,
(SEQ ID NO: 412)
NN NG NN HD NG HD NG NN NI HD NI NI NG NI,
(SEQ ID NO: 413)
NN NG NG NG NG NN HD NI NN HD HD NG HD HD,
(SEQ ID NO: 414)
NI HD NI NN HD NG NN NG NN NN NI NI HD NN NG,
(SEQ ID NO: 415)
HD NI NI NN NI HD HD NN NI NN HD NI HD NG NN HD
NG NN,
(SEQ ID NO: 416)
HD NG NI NG HD HD HD NI NI NI NI HD NG HD NG,
(SEQ ID NO: 417)
NH NI NI NI NI NI HD NG NI NG NH NG NI NG,
(SEQ ID NO: 418)
NI NH NH HD NI NH NH HD NG NH NH NG NG NH NI,
(SEQ ID NO: 419)
HD NI NI NG NI HD NI NI HD HD NI HD NN HD,
(SEQ ID NO: 420)
NI NG NN NI HD NN NN NI HD NG HD NI NI HD NG,
(SEQ ID NO: 421)
HD NI HD NI NI HD NI NG NG NG NN NG NI NI,
and
(SEQ ID NO: 422)
NI NG NG NG HD HD NI NN NG NN HD NI HD NI.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
(SEQ ID NO: 355)
NH NH HD HD NH NH HD HD NG NH NI HD HD NI HD NG
NH NH;
(SEQ ID NO: 24)
TGAAGGCCTGGCCGGCCTG
and
(SEQ ID NO: 356)
NH NI NI NH NH HD HD NG NH NH HD HD NH NH HD HD
NG NH;
(SEQ ID NO: 25)
TGAGCACTGAAGGCCTGGC
and
(SEQ ID NO: 357)
NH NI NH HD NI HD NG NH NI NI NH NH HD HD NG NH
NH HD;
(SEQ ID NO: 26)
TCCACTGAGCACTGAAGGC
and
(SEQ ID NO: 358)
HD HD NI HD NG NH NI NH HD NI HD NG NH NI NI NH
NH HD;
(SEQ ID NO: 27)
TGGTTTCCACTGAGCACTG
and
(SEQ ID NO: 359)
NH NH NG NG NG HD HD NI HD NG NH NI NH HD NI HD
NG NH;
(SEQ ID NO: 28)
TGGGGAAAATGACCCAACA
and
(SEQ ID NO: 360)
NH NH NH NH NI NI NI NI NG NH NI HD HD HD NI NI
HD NI;
(SEQ ID NO: 29)
TAGGACAGTGGGGAAAATG
and
(SEQ ID NO: 361)
NI NH NH NI HD NI NH NG NH NH NH NH NI NI NI NI
NG NH;
(SEQ ID NO: 30)
TCCAGGGACACGGTGCTAG
and
(SEQ ID NO: 362)
HD HD NI NH NH NH NI HD NI HD NH NH NG NH HD NG
NI NH;
(SEQ ID NO: 31)
TCAGAGCCAGGAGTCCTGG
and
(SEQ ID NO: 363)
HD NI NH NI NH HD HD NI NH NH NI NH NG HD HD NG
NH NH;
(SEQ ID NO: 32)
TCCTTCAGAGCCAGGAGTC
and
(SEQ ID NO: 364)
HD HD NG NG HD NI NH NI NH HD HD NI NH NH NI NH
NG HD;
(SEQ ID NO: 33)
TCCTCCTTCAGAGCCAGGA
and
(SEQ ID NO: 365)
HD HD NG HD HD NG NG HD NI NH NI NH HD HD NI NH
NH NI;
(SEQ ID NO: 34)
TCCAGCCCCTCCTCCTTCA
and
(SEQ ID NO: 366)
HD HD NI NH HD HD HD HD NG HD HD NG HD HD NG NG
HD NI;
(SEQ ID NO: 35)
TCCGAGCTTGACCCTTGGA
and
(SEQ ID NO: 367)
HD HD NH NI NH HD NG NG NH NI HD HD HD NG NG NH
NH NI;
(SEQ ID NO: 36)
TGGTTTCCGAGCTTGACCC
and
(SEQ ID NO: 368)
NH NH NG NG NG HD HD NH NI NH HD NG NG NH NI HD
HD HD;
(SEQ ID NO: 37)
TGGGGTGGTTTCCGAGCTT
and
(SEQ ID NO: 369)
NH NH NH NH NG NH NH NG NG NG HD HD NH NI NH HD
NG NG;
(SEQ ID NO: 38)
TCTGCTGGGGTGGTTTCCG
and
(SEQ ID NO: 370)
HD NG NH HD NG NH NH NH NH NG NH NH NG NG NG HD
HD NH;
(SEQ ID NO: 39)
TGCAGAGTATCTGCTGGGG
and
(SEQ ID NO: 371)
NH HD NI NH NI NH NG NI NG HD NG NH HD NG NH NH
NH NH;
(SEQ ID NO: 40)
CCAATCCCCTCAGT
and
(SEQ ID NO: 372)
HD HD NI NI NG HD HD HD HD NG HD NI NH NG;
(SEQ ID NO: 41)
CAGTGCTCAGTGGAA
and
(SEQ ID NO: 373)
HD NI NH NG NH HD NG HD NI NH NG NH NH NI NI;
(SEQ ID NO: 42)
GAAACATCCGGCGACTCA
and
(SEQ ID NO: 374)
NH NI NI NI HD NI NG HD HD NH NH HD NH NI HD NG
HD NI;
(SEQ ID NO: 43)
TCGCCCCTCAAATCTTACA
and
(SEQ ID NO: 375)
HD NH HD HD HD HD NG HD NI NI NI NG HD NG NG NI
HD NI;
(SEQ ID NO: 44)
TCAAATCTTACAGCTGCTC
and
(SEQ ID NO: 376)
HD NI NI NI NG HD NG NG NI HD NI NH HD NG NH HD
NG HD;
(SEQ ID NO: 45)
TCTTACAGCTGCTCACTCC
and
(SEQ ID NO: 377)
HD NG NG NI HD NI NH HD NG NH HD NG HD NI HD NG
HD HD;
(SEQ ID NO: 46)
TACAGCTGCTCACTCCCCT
and
(SEQ ID NO: 378)
NI HD NI NH HD NG NH HD NG HD NI HD NG HD HD HD
HD NG;
(SEQ ID NO: 47)
TGCTCACTCCCCTGCAGGG
and
(SEQ ID NO: 379)
NH HD NG HD NI HD NG HD HD HD HD NG NH HD NI NH
NH NH;
(SEQ ID NO: 48)
TCCCCTGCAGGGCAACGCC
and
(SEQ ID NO: 380)
HD HD HD HD NG NH HD NI NH NH NH HD NI NI HD NH
HD HD;
(SEQ ID NO: 49)
TGCAGGGCAACGCCCAGGG
and
(SEQ ID NO: 381)
NH HD NI NH NH NH HD NI NI HD NH HD HD HD NI NH
NH NH;
(SEQ ID NO: 50)
TCTCGATTATGGGGGGAT
and
(SEQ ID NO: 382)
HD NG HD NH NI NG NG NI NG NH NH NH HD NH NH NH
NI NG;
(SEQ ID NO: 51)
TCGCTTCTCGATTATGGGC
and
(SEQ ID NO: 383)
HD NH HD NG NG HD NG HD NH NI NG NG NI NG NH NH
NH HD;
(SEQ ID NO: 52)
TGTCGAGTCGCTTCTCGAT
and
(SEQ ID NO: 384)
NH NG HD NH NI NH NG HD NH HD NG NG HD NG HD NH
NI NG;
(SEQ ID NO: 53)
TCCATGTCGAGTCGCTTCT
and
(SEQ ID NO: 385)
HD HD NI NG NH NG HD NH NI NH NG HD NH HD NG NG
HD NG;
(SEQ ID NO: 54)
TCGCCTCCATGTCGAGTCG
and
(SEQ ID NO: 386)
HD NH HD HD NG HD HD NI NG NH NG HD NH NI NH NG
HD NH;
(SEQ ID NO: 55)
TCGTCATCGCCTCCATGTC
and
(SEQ ID NO: 387)
HD NH NG HD NI NG HD NH HD HD NG HD HD NI NG NH
NG HD;
(SEQ ID NO: 56)
TGATCTCGTCATCGCCTCC
and
(SEQ ID NO: 388)
NH NI NG HD NG HD NH NG HD NI NG HD NH HD HD NG
HD HD;
(SEQ ID NO: 57)
GCTTCAGCTTCCTA
and
(SEQ ID NO: 389)
NH HD NG NG HD NI NH HD NG NG HD HD NG NI;
(SEQ ID NO: 58)
CTGTGATCATGCCA
and
(SEQ ID NO: 390)
HD NG NK NG NH NI NG HD NI NG NH HD HD NI;
(SEQ ID NO: 59)
ACAGTGGTACACACCT
and
(SEQ ID NO: 391)
NI HD NI NN NG NN NN NG NI HD NI HD NI HD HD NG;
(SEQ ID NO: 60)
CCACCCCCCACTAAG
and
(SEQ ID NO: 392)
HD HD NI HD HD HD HD HD HD NI HD NG NI NI NN;
(SEQ ID NO: 61)
CATTGGCCGGGCAC
and
(SEQ ID NO: 393)
HD NI NG NG NN NN HD HD NN NN NN HD NI HD;
(SEQ ID NO: 62)
GCTTGAACCCAGGAGA
and
(SEQ ID NO: 394)
NN HD NG NG NN NI NI HD HD HD NI NN NN NI NN NI;
(SEQ ID NO: 63)
ACACCCGATCCACTGGG
and
(SEQ ID NO: 395)
NI HD NI HD HD HD NN NI NG HD HD NI HD NG NN NN
NN;
(SEQ ID NO: 64)
GCTGCATCAACCCC
and
(SEQ ID NO: 396)
NN HD NG NN HD NI NG HD NI NI HD HD HD HD;
(SEQ ID NO: 65)
GCCACAAACAGAAATA
and
(SEQ ID NO: 397)
NN NN HD NI HD NN NI NI NI HD NI HD HD HD NG HD
HD;
(SEQ ID NO: 66)
GGTGGCTCATGCCTG
and
(SEQ ID NO: 398)
NN NN NG NN NN HD NG HD NI NG NN HD HD NG NN;
(SEQ ID NO: 67)
GATTTGCACAGCTCAT
and
(SEQ ID NO: 399)
NN NI NG NG NG NN HD NI HD NI NN HD NG HD NI NG;
(SEQ ID NO: 68)
AAGCTCTGAGGAGCA
and
(SEQ ID NO: 400)
NI NI NH HD NG HD NG NH NI NH NH NI NH HD;
(SEQ ID NO: 69)
CCCTAGCTGTCCC
and
(SEQ ID NO: 401)
HD HD HD NG NI NK HD NG NH NG HD HD HD HD;
(SEQ ID NO: 70)
GCCTAGCATGCTAG
and
(SEQ ID NO: 402)
NH HD HD NG NI NH HD NI NG NH HD NG NI NH;
(SEQ ID NO: 71)
ATGGGCTTCACGGAT
and
(SEQ ID NO: 403)
NI NG NH NH NH HD NG NG HD NI HD NH NH NI NG;
(SEQ ID NO: 72)
GAAACTATGCCTGC
and
(SEQ ID NO: 404)
NH NI NI NI HD NG NI NG NH HD HD NG NH HD;
(SEQ ID NO: 73)
GCACCATTGCTCCC
and
(SEQ ID NO: 405)
NH HD NI HD HD NI NG NG NH HD NG HD HD HD;
(SEQ ID NO: 74)
GACATGCAACTCAG
and
(SEQ ID NO: 406)
NH NI HD NI NG NH HD NI NI HD NG HD NI NH;
(SEQ ID NO: 75)
ACACCACTAGGGGT
and
(SEQ ID NO: 407)
NI HD NI HD HD NI HD NG NI NH NH NH NH NG;
(SEQ ID NO: 76)
GTCTGCTAGACAGG
and
(SEQ ID NO: 408)
NH NG HD NG NH HD NG NI NH NI HD NI NH NH;
(SEQ ID NO: 77)
GGCCTAGACAGGCTG
and
(SEQ ID NO: 409)
NH NH HD HD NG NI NH NI HD NI NH NH HD NG NH;
(SEQ ID NO: 78)
GAGGCATTCTTATCG
and
(SEQ ID NO: 410)
NH NI NH NH HD NI NG NG HD NG NG NI NG HD NH;
(SEQ ID NO: 79)
GCCTGGAAACGTTCC
and
(SEQ ID NO: 411)
NN HD HD NG NN NN NI NI NI HD NN NG NG HD HD;
(SEQ ID NO: 80)
GTGCTCTGACAATA
and
(SEQ ID NO: 412)
NN NG NN HD NG HD NG NN NI HD NI NI NG NI;
(SEQ ID NO: 81)
GTTTTGCAGCCTCC
and
(SEQ ID NO: 413)
NN NG NG NG NG NN HD NI NN HD HD NG HD HD;
(SEQ ID NO: 82)
ACAGCTGTGGAACGT
and
(SEQ ID NO: 414)
NI HD NI NN HD NG NN NG NN NN NI NI HD NN NG;
(SEQ ID NO: 83)
GGCTCTCTTCCTCCT
and
(SEQ ID NO: 415)
HD NI NI NN NI HD HD NN NI NN HD NI HD NG NN HD
NG NN;
(SEQ ID NO: 84)
CTATCCCAAAACTCT
and
(SEQ ID NO: 416)
HD NG NI NG HD HD HD NI NI NI NI HD NG HD NG;
(SEQ ID NO: 85)
GAAAAACTATGTAT
and
(SEQ ID NO: 417)
NH NI NI NI NI NI HD NG NI NG NH NG NI NG;
(SEQ ID NO: 86)
AGGCAGGCTGGTTGA
and
(SEQ ID NO: 418)
NI NH NH HD NI NH NH HD NG NH NH NG NG NH NI;
(SEQ ID NO: 87)
CAATACAACCACGC
and
(SEQ ID NO: 419)
HD NI NI NG NI HD NI NI HD HD NI HD NN HD;
(SEQ ID NO: 88)
ATGACGGACTCAACT
and
(SEQ ID NO: 420)
NI NG NN NI HD NN NN NI HD NG HD NI NI HD NG;
and
(SEQ ID NO: 89)
CACAACATTTGTAA
and
(SEQ ID NO: 421)
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site or TTAA (SEQ ID NO: 440) tetranucleotide site.

Illustrative DNA binding codes for targeting human genomic safe harbor in areas of open chromatin via TALES, encompassed by various embodiments are provided in TABLE 7.

TABLE 7
DNA binding codes for targeting human genomic safe harbor in areas of open chromatin
via TALEs.

| GSHS | ID | Sequence | TALE (DNA binding code) |
|---|---|---|---|
| AAVS1 | 1 | tggccggcctgaccactgg (SEQ ID | NH NH HD HD NH NH HD HD NG NH NI HD |
| | | NO: 23) | HD NI HD NG NH NH (SEQ ID NO: 355) |
| AAVS1 | 2 | tgaaggcctggccggcctg (SEQ ID | NH NI NI NH NH HD HD NG NH NH HD HD |
| | | NO: 24) | NH NH HD HD NG NH (SEQ ID NO: 356) |
| AAVS1 | 3 | tgagcactgaaggcctggc (SEQ | NH NI NH HD NI HD NG NH NI NI NH NH |
| | | ID NO: 25) | HD HD NG NH NH HD (SEQ ID NO: 357) |
| AAVS1 | 4 | tccactgagcactgaaggc (SEQ ID | HD HD NI HD NG NH NI NH HD NI HD NG |
| | | NO: 26) | NH NI NI NH NH HD (SEQ ID NO: 358) |
| AAVS1 | 5 | tggtttccactgagcactg (SEQ ID | NH NH NG NG NG HD HD NI HD NG NH NI |
| | | NO: 27) | NH HD NI HD NG NH (SEQ ID NO: 359) |
| AAVS1 | 6 | tggggaaaatgacccaaca (SEQ | NH NH NH NH NI NI NI NI NG NH NI HD HD |
| | | ID NO: 28) | HD NI NI HD NI (SEQ ID NO: 360) |
| AAVS1 | 7 | taggacagtggggaaaatg (SEQ | NI NH NH NI HD NI NH NG NH NH NH NH |
| | | ID NO: 29) | NI NI NI NI NG NH (SEQ ID NO: 361) |
| AAVS1 | 8 | tccagggacacggtgctag (SEQ | HD HD NI NH NH NH NI HD NI HD NH NH |
| | | ID NO: 30) | NG NH HD NG NI NH (SEQ ID NO: 362) |
| AAVS1 | 9 | tcagagccaggagtcctgg (SEQ | HD NI NH NI NH HD HD NI NH NH NI NH |
| | | ID NO: 31) | NG HD HD NG NH NH (SEQ ID NO: 363) |
| AAVS1 | 10 | tccttcagagccaggagtc (SEQ ID | HD HD NG NG HD NI NH NI NH HD HD NI |
| | | NO: 32) | NH NH NI NH NG HD (SEQ ID NO: 364) |
| AAVS1 | 11 | tcctccttcagagccagga (SEQ ID | HD HD NG HD HD NG NG HD NI NH NI NH |
| | | NO: 33) | HD HD NI NH NH NI (SEQ ID NO: 365) |
| AAVS1 | 12 | tccagcccctcctccttca (SEQ ID | HD HD NI NH HD HD HD HD NG HD HD NG |
| | | NO: 34) | HD HD NG NG HD NI (SEQ ID NO: 366) |
| AAVS1 | 13 | tccgagcttgacccttgga (SEQ ID | HD HD NH NI NH HD NG NG NH NI HD HD |
| | | NO: 35) | HD NG NG NH NH NI (SEQ ID NO: 367) |
| AAVS1 | 14 | tggtttccgagcttgaccc (SEQ ID | NH NH NG NG NG HD HD NH NI NH HD NG |
| | | NO: 36) | NG NH NI HD HD HD (SEQ ID NO: 368) |
| AAVS1 | 15 | tggggtggtttccgagctt (SEQ ID | NH NH NH NH NG NH NH NG NG NG HD |
| | | NO: 37) | HD NH NI NH HD NG NG (SEQ ID NO: 369) |
| AAVS1 | 16 | tctgctggggtggtttccg (SEQ ID | HD NG NH HD NG NH NH NH NH NG NH |
| | | NO: 38) | NH NG NG NG HD HD NH (SEQ ID NO: |
| | | | 370) |
| AAVS1 | 17 | tgcagagtatctgctgggg (SEQ ID | NH HD NI NH NI NH NG NI NG HD NG NH |
| | | NO: 39) | HD NG NH NH NH NH (SEQ ID NO: 371) |
| AAVS1 | AVS1 | CCAATCCCCTCAGT (SEQ | HD HD NI NI NG HD HD HD HD NG HD NI |
| | | ID NO: 40) | NH NG (SEQ ID NO: 372) |
| AAVS1 | AVS2 | CAGTGCTCAGTGGAA (SEQ | HD NI NH NG NH HD NG HD NI NH NG NH |
| | | ID NO: 41) | NH NI NI (SEQ ID NO: 373) |
| AAVS1 | AVS3 | GAAACATCCGGCGACTCA | NH NI NI NI HD NI NG HD HD NH NH HD |
| | | (SEQ ID NO: 42) | NH NI HD NG HD NI (SEQ ID NO: 374) |
| hROSA26 | 1F | tcgcccctcaaatcttaca (SEQ ID | HD NH HD HD HD HD NG HD NI NI NI NG |
| | | NO: 43) | HD NG NG NI HD NI (SEQ ID NO: 375) |
| hROSA26 | 2F | tcaaatcttacagctgctc (SEQ ID | HD NI NI NI NG HD NG NG NI HD NI NH HD |
| | | NO: 44) | NG NH HD NG HD (SEQ ID NO: 376) |
| hROSA26 | 3F | tcttacagctgctcactcc (SEQ ID | HD NG NG NI HD NI NH HD NG NH HD NG |
| | | NO: 45) | HD NI HD NG HD HD (SEQ ID NO: 377) |
| hROSA26 | 4F | tacagctgctcactcccct (SEQ ID | NI HD NI NH HD NG NH HD NG HD NI HD |
| | | NO: 46) | NG HD HD HD HD NG (SEQ ID NO: 378) |
| hROSA26 | 5F | tgctcactcccctgcaggg (SEQ ID | NH HD NG HD NI HD NG HD HD HD HD NG |
| | | NO: 47) | NH HD NI NH NH NH (SEQ ID NO: 379) |
| hROSA26 | 6F | tcccctgcagggcaacgcc (SEQ | HD HD HD HD NG NH HD NI NH NH NH HD |
| | | ID NO: 48) | NI NI HD NH HD HD (SEQ ID NO: 380) |
| hROSA26 | 7F | tgcagggcaacgcccaggg (SEQ | NH HD NI NH NH NH HD NI NI HD NH HD |
| | | ID NO: 49) | HD HD NI NH NH NH (SEQ ID NO: 381) |
| hROSA26 | 8R | tctcgattatggggggat (SEQ ID | HD NG HD NH NI NG NG NI NG NH NH NH |
| | | NO: 50) | HD NH NH NH NI NG (SEQ ID NO: 382) |
| hROSA26 | 9R | tcgcttctcgattatgggc (SEQ ID | HD NH HD NG NG HD NG HD NH NI NG |
| | | NO: 51) | NG NI NG NH NH NH HD (SEQ ID NO: 383) |
| hROSA26 | 10R | tgtcgagtcgcttctcgat (SEQ ID | NH NG HD NH NI NH NG HD NH HD NG NG |
| | | NO: 52) | HD NG HD NH NI NG (SEQ ID NO: 384) |
| hROSA26 | 11R | tccatgtcgagtcgcttct (SEQ ID | HD HD NI NG NH NG HD NH NI NH NG HD |
| | | NO: 53) | NH HD NG NG HD NG (SEQ ID NO: 385) |
| hROSA26 | 12R | tcgcctccatgtcgagtcg (SEQ ID | HD NH HD HD NG HD HD NI NG NH NG HD |
| | | NO: 54) | NH NI NH NG HD NH (SEQ ID NO: 386) |
| hROSA26 | 13R | tcgtcatcgcctccatgtc (SEQ ID | HD NH NG HD NI NG HD NH HD HD NG HD |
| | | NO: 55) | HD NI NG NH NG HD (SEQ ID NO: 387) |
| hROSA26 | 14R | tgatctcgtcatcgcctcc (SEQ ID | NH NI NG HD NG HD NH NG HD NI NG HD |
| | | NO: 56) | NH HD HD NG HD HD (SEQ ID NO: 388) |
| hROSA26 | ROSA1 | GCTTCAGCTTCCTA (SEQ | NH HD NG NG HD NI NH HD NG NG HD HD |
| | | ID NO: 57) | NG NI (SEQ ID NO: 389) |
| hROSA26 | ROSA2 | CTGTGATCATGCCA (SEQ | HD NG NK NG NH NI NG HD NI NG NH HD |
| | | ID NO: 58) | HD NI (SEQ ID NO: 390) |
| hROSA26 | TALER2 | ACAGTGGTACACACCT | NI HD NI NN NG NN NN NG NI HD NI HD NI |
| | | (SEQ ID NO: 59) | HD HD NG (SEQ ID NO: 391) |
| hROSA26 | TALER3 | CCACCCCCCACTAAG (SEQ | HD HD NI HD HD HD HD HD HD NI HD NG |
| | | ID NO: 60) | NI NI NN (SEQ ID NO: 392) |
| hROSA26 | TALER4 | CATTGGCCGGGCAC (SEQ | HD NI NG NG NN NN HD HD NN NN NN HD |
| | | ID NO: 61) | NI HD (SEQ ID NO: 393) |
| hROSA26 | TALER5 | GCTTGAACCCAGGAGA | NN HD NG NG NN NI NI HD HD HD NI NN |
| | | (SEQ ID NO: 62) | NN NI NN NI (SEQ ID NO: 394) |
| CCR5 | TALC3 | ACACCCGATCCACTGGG | NI HD NI HD HD HD NN NI NG HD HD NI |
| | | (SEQ ID NO: 63) | HD NG NN NN NN (SEQ ID NO: 395) |
| CCR5 | TALC4 | GCTGCATCAACCCC (SEQ | NN HD NG NN HD NI NG HD NI NI HD HD |
| | | ID NO: 64) | HD HD (SEQ ID NO: 396) |
| CCR5 | TALC5 | GCCACAAACAGAAATA | NN NN HD NI HD NN NI NI NI HD NI HD HD |
| | | (SEQ ID NO: 65) | HD NG HD HD (SEQ ID NO: 397) |
| CCR5 | TALC7 | GGTGGCTCATGCCTG | NN NN NG NN NN HD NG HD NI NG NN HD |
| | | (SEQ ID NO: 66) | HD NG NN (SEQ ID NO: 398) |
| CCR5 | TALC8 | GATTTGCACAGCTCAT | NN NI NG NG NG NN HD NI HD NI NN HD |
| | | (SEQ ID NO: 67) | NG HD NI NG (SEQ ID NO: 399) |
| Chr 2 | SHCHR2-1 | AAGCTCTGAGGAGCA (SEQ | NI NI NH HD NG HD NG NH NI NH NH NI |
| | | ID NO: 68) | NH HD (SEQ ID NO: 400) |
| Chr 2 | SHCHR2-2 | CCCTAGCTGTCCC (SEQ ID | HD HD HD NG NI NK HD NG NH NG HD HD |
| | | NO: 69) | HD HD (SEQ ID NO: 401) |
| Chr 2 | SHCHR2-3 | GCCTAGCATGCTAG (SEQ | NH HD HD NG NI NH HD NI NG NH HD NG |
| | | ID NO: 70) | NI NH (SEQ ID NO: 402) |
| Chr 2 | SHCHR2-4 | ATGGGCTTCACGGAT (SEQ | NI NG NH NH NH HD NG NG HD NI HD NH |
| | | ID NO: 71) | NH NI NG (SEQ ID NO: 403) |
| Chr 4 | SHCHR4-1 | GAAACTATGCCTGC (SEQ | NH NI NI NI HD NG NI NG NH HD HD NG |
| | | ID NO: 72) | NH HD (SEQ ID NO: 404) |
| Chr 4 | SHCHR4-2 | GCACCATTGCTCCC (SEQ | NH HD NI HD HD NI NG NG NH HD NG HD |
| | | ID NO: 73) | HD HD (SEQ ID NO: 405) |
| Chr 4 | SHCHR4-3 | GACATGCAACTCAG (SEQ | NH NI HD NI NG NH HD NI NI HD NG HD NI |
| | | ID NO: 74) | NH (SEQ ID NO: 406) |
| Chr 6 | SHCHR6-1 | ACACCACTAGGGGT (SEQ | NI HD NI HD HD NI HD NG NI NH NH NH |
| | | ID NO: 75) | NH NG (SEQ ID NO: 407) |
| Chr 6 | SHCHR6-2 | GTCTGCTAGACAGG (SEQ | NH NG HD NG NH HD NG NI NH NI HD NI |
| | | ID NO: 76) | NH NH (SEQ ID NO: 408) |
| Chr 6 | SHCHR6-3 | GGCCTAGACAGGCTG | NH NH HD HD NG NI NH NI HD NI NH NH |
| | | (SEQ ID NO: 77) | HD NG NH (SEQ ID NO: 409) |
| Chr 6 | SHCHR6-4 | GAGGCATTCTTATCG (SEQ | NH NI NH NH HD NI NG NG HD NG NG NI |
| | | ID NO: 78) | NG HD NH (SEQ ID NO: 410) |
| Chr 10 | SHCHR10-1 | GCCTGGAAACGTTCC (SEQ | NN HD HD NG NN NN NI NI NI HD NN NG |
| | | ID NO: 79) | NG HD HD (SEQ ID NO: 411) |
| Chr 10 | SHCHR10-2 | GTGCTCTGACAATA (SEQ | NN NG NN HD NG HD NG NN NI HD NI NI |
| | | ID NO: 80) | NG NI (SEQ ID NO: 412) |
| Chr 10 | SHCHR10-3 | GTTTTGCAGCCTCC (SEQ | NN NG NG NG NG NN HD NI NN HD HD |
| | | ID NO: 81) | NG HD HD (SEQ ID NO: 413) |
| Chr 10 | SHCHR10-4 | ACAGCTGTGGAACGT (SEQ | NI HD NI NN HD NG NN NG NN NN NI NI |
| | | ID NO: 82) | HD NN NG (SEQ ID NO: 414) |
| Chr 10 | SHCHR10-5 | GGCTCTCTTCCTCCT (SEQ | HD NI NI NN NI HD HD NN NI NN HD NI HD |
| | | ID NO: 83) | NG NN HD NG NN (SEQ ID NO: 415) |
| Chr 11 | SHCHR11-1 | CTATCCCAAAACTCT (SEQ | HD NG NI NG HD HD HD NI NI NI NI HD NG |
| | | ID NO: 84) | HD NG (SEQ ID NO: 416) |
| Chr 11 | SHCHR11-2 | GAAAAACTATGTAT (SEQ ID | NH NI NI NI NI NI HD NG NI NG NH NG NI |
| | | NO: 85) | NG (SEQ ID NO: 417) |
| Chr 11 | SHCHR11-3 | AGGCAGGCTGGTTGA | NI NH NH HD NI NH NH HD NG NH NH NG |
| | | (SEQ ID NO: 86) | NG NH NI (SEQ ID NO: 418) |
| Chr 17 | SHCHR17-1 | CAATACAACCACGC (SEQ | HD NI NI NG NI HD NI NI HD HD NI HD NN |
| | | ID NO: 87) | HD (SEQ ID NO: 419) |
| Chr 17 | SHCHR17-2 | ATGACGGACTCAACT (SEQ | NI NG NN NI HD NN NN NI HD NG HD NI NI |
| | | ID NO: 88) | HD NG (SEQ ID NO: 420) |
| Chr 17 | SHCHR17-3 | CACAACATTTGTAA (SEQ ID | HD NI HD NI NI HD NI NG NG NG NN NG |
| | | NO: 89) | NI NI (SEQ ID NO: 421) |
| Chr 17 | SHCHR17-4 | ATTTCCAGTGCACA (SEQ | NI NG NG NG HD HD NI NN NG NN HD NI |
| | | ID NO: 90) | HD NI (SEQ ID NO: 422) |

Further illustrative DNA binding codes for targeting human genomic safe harbor in areas of open chromatin via TALEs, encompassed by embodiments are provided in TABLES 8-12. In embodiments, the helper enzyme of the present disclosure is capable of inserting a donor DNA at a TA dinucleotide site. In embodiments, the helper enzyme of the present disclosure is capable of inserting a donor DNA at a TTAA (SEQ ID NO: 440) tetranucleotide site.

TABLE 8
TALE sequences targeting the genomic safe harbor site, hROSA26.

| NAME | DNA SEQUENCE | RVD AMINO ACID CODE |
|---|---|---|
| R1 | TCGCCCCTCAAATCTTACAG | HD NH HD HD HD HD NG HD NI NI NI NG HD NG NG NI HD NI NH |
| | (SEQ ID NO: 599) | (SEQ ID NO: 613) |
| R2 | TCAAATCTTACAGCTGCTCA | HD NI NI NI NG HD NG NG NI HD NI NH HD NG NH HD NG HD NI |
| | (SEQ ID NO: 600) | (SEQ ID NO: 614) |
| R3 | TCTTACAGCTGCTCACTCCC | HD NG NG NI HD NI NH HD NG NH HD NG HD NI HD NG HD HD |
| | (SEQ ID NO: 601) | HD (SEQ ID NO: 615) |
| R4 | TACAGCTGCTCACTCCCCTG | NI HD NI NH HD NG NH HD NG HD NI HD NG HD HD HD HD NG |
| | (SEQ ID NO: 602) | NH (SEQ ID NO: 616) |
| R5 | TGCTCACTCCCCTGCAGGGC | NH HD NG HD NI HD NG HD HD HD HD NG NH HD NI NH NH NH |
| | (SEQ ID NO: 603) | HD (SEQ ID NO: 617) |
| R6 | TCCCCTGCAGGGCAACGCCC | HD HD HD HD NG NH HD NI NH NH NH HD NI NI HD NH HD HD |
| | (SEQ ID NO: 604) | HD (SEQ ID NO: 618) |
| R7 | TGCAGGGCAACGCCCAGGGA | NH HD NI NH NH NH HD NI NI HD NH HD HD HD NI NH NH NH NI |
| | (SEQ ID NO: 605) | (SEQ ID NO: 619) |
| R8 | TCTCGATTATGGGGGGGATT | HD NG HD NH NI NG NG NI NG NH NH NH HD NH NH NH NI NG |
| | (SEQ ID NO: 606) | NG (SEQ ID NO: 620) |
| R9 | TCGCTTCTCGATTATGGGCG | HD NH HD NG NG HD NG HD NH NI NG NG NI NG NH NH NH HD |
| | (SEQ ID NO: 607) | NH (SEQ ID NO: 621) |
| R10 | TGTCGAGTCGCTTCTCGATT | NH NG HD NH NI NH NG HD NH HD NG NG HD NG HD NH NI NG |
| | (SEQ ID NO: 608) | NG (SEQ ID NO: 622) |
| R11 | TCCATGTCGAGTCGCTTCTC | HD HD NI NG NH NG HD NH NI NH NG HD NH HD NG NG HD NG |
| | (SEQ ID NO: 609) | HD (SEQ ID NO: 623) |
| R12 | TCGCCTCCATGTCGAGTCGC | HD NH HD HD NG HD HD NI NG NH NG HD NH NI NH NG HD NH |
| | (SEQ ID NO: 610) | HD (SEQ ID NO: 624) |
| R13 | TCGTCATCGCCTCCATGTCG | HD NH NG HD NI NG HD NH HD HD NG HD HD NI NG NH NG HD |
| | (SEQ ID NO: 611) | NH (SEQ ID NO: 625) |
| R14 | TGATCTCGTCATCGCCTCCA | NH NI NG HD NG HD NH NG HD NI NG HD NH HD HD NG HD HD |
| | (SEQ ID NO: 612) | NI (SEQ ID NO: 626) |

TABLE 9
TALE sequences targeting the genomic safe harbor site, AAVS1.

| NAME | DNA SEQUENCE | RVD AMINO ACID CODE |
|---|---|---|
| AAV1c | TGGCCGGCCTGACCACTGGG (SEQ ID | NH NH HD HD NH NH HD HD NG NH NI HD HD NI HD NG |
| | NO: 627) | NH NH NH (SEQ ID NO: 644) |
| AAV2c | TGAAGGCCTGGCCGGCCTGA (SEQ ID | NH NI NI NH NH HD HD NG NH NH HD HD NH NH HD HD |
| | NO: 628) | NG NH NI (SEQ ID NO: 645) |
| AAV3c | TGAGCACTGAAGGCCTGGCC (SEQ ID | NH NI NH HD NI HD NG NH NI NI NH NH HD HD NG NH NH |
| | NO: 629) | HD HD (SEQ ID NO: 646) |
| AAV4c | TCCACTGAGCACTGAAGGCC (SEQ ID | HD HD NI HD NG NH NI NH HD NI HD NG NH NI NI NH NH |
| | NO: 630) | HD HD (SEQ ID NO: 647) |
| AAV5c | TGGTTTCCACTGAGCACTGA (SEQ ID | NH NH NG NG NG HD HD NI HD NG NH NI NH HD NI HD NG |
| | NO: 631) | NH NI (SEQ ID NO: 648) |
| AAV6 | TGGGGAAAATGACCCAACAG (SEQ ID | NH NH NH NH NI NI NI NI NG NH NI HD HD HD NI NI HD |
| | NO: 632) | NI NH (SEQ ID NO: 649) |
| AAV7 | TAGGACAGTGGGGAAAATGA (SEQ ID | NI NH NH NI HD NI NH NG NH NH NH NH NI NI NI NI NG |
| | NO: 633) | NH NI (SEQ ID NO: 650) |
| AAV8 | TCCAGGGACACGGTGCTAGG (SEQ ID | HD HD NI NH NH NH NI HD NI HD NH NH NG NH HD NG NI |
| | NO: 634) | NH NH (SEQ ID NO: 651) |
| AAV9 | TCAGAGCCAGGAGTCCTGGC (SEQ ID | HD NI NH NI NH HD HD NI NH NH NI NH NG HD HD NG NH |
| | NO: 635) | NH HD (SEQ ID NO: 652) |
| AAV10 | TCCTTCAGAGCCAGGAGTCC (SEQ ID | HD HD NG NG HD NI NH NI NH HD HD NI NH NH NI NH NG |
| | NO: 636) | HD HD (SEQ ID NO: 653) |
| AAV11 | TCCTCCTTCAGAGCCAGGAG (SEQ ID | HD HD NG HD HD NG NG HD NI NH NI NH HD HD NI NH NH |
| | NO: 637) | NI NH (SEQ ID NO: 654) |
| AAV12 | TCCAGCCCCTCCTCCTTCAG (SEQ ID | HD HD NI NH HD HD HD HD NG HD HD NG HD HD NG NG |
| | NO: 638) | HD NI NH (SEQ ID NO: 655) |
| AAV13c | TCCGAGCTTGACCCTTGGAA (SEQ ID | HD HD NH NI NH HD NG NG NH NI HD HD HD NG NG NH |
| | NO: 639) | NH NI NI (SEQ ID NO: 656) |
| AAV14c | TGGTTTCCGAGCTTGACCCT (SEQ ID | NH NH NG NG NG HD HD NH NI NH HD NG NG NH NI HD |
| | NO: 640) | HD HD NG (SEQ ID NO: 657) |
| AAV15c | TGGGGTGGTTTCCGAGCTTG (SEQ ID | NH NH NH NH NG NH NH NG NG NG HD HD NH NI NH HD |
| | NO: 641) | NG NG NH (SEQ ID NO: 658) |
| AAV16c | TCTGCTGGGGTGGTTTCCGA (SEQ ID | HD NG NH HD NG NH NH NH NH NG NH NH NG NG NG HD |
| | NO: 642) | HD NH NI (SEQ ID NO: 659) |
| AAV17c | TGCAGAGTATCTGCTGGGGT (SEQ ID | NH HD NI NH NI NH NG NI NG HD NG NH HD NG NH NH NH |
| | NO: 643) | NH NG (SEQ ID NO: 660) |

TABLE 10
TALE sequences targeting a chromosome 4 genomic safe harbor site (hg38 chr4:30,793,039-
30,793,980).

| NAME | DNA SEQUENCE | RVD AMINO ACID CODE |
|---|---|---|
| TALE4-R001 | TCTTCCTAGTATTAAAGT (SEQ ID NO: 661) | HD NG NG HD HD NG NI NH NG NI NG NG |
| | | NI NI NI NH NG (SEQ ID NO: 681) |
| TALE4-R002 | TCCTTAATATTACCAGT (SEQ ID NO: 662) | HD HD NG NG NI NI NG NI NG NG NI HD HD |
| | | NI NH NG (SEQ ID NO: 682) |
| TALE4-F003 | TACCAAGCTGAAATGACACAAAAGT (SEQ ID | NI HD HD NI NI NH HD NG NH NI NI NI NG |
| | NO: 663) | NH NI HD NI HD NI NI NI NI NH NG (SEQ ID |
| | | NO: 683) |
| TALE4-F004 | TGGCTGTGTCACATACCAGCAGAAT (SEQ ID | NH NH HD NG NH NG NH NG HD NI HD NI |
| | NO: 664) | NG NI HD HD NI NH HD NI NH NI NI NG (SEQ |
| | | ID NO: 684) |
| TALE4-F005 | TGTTAATTTGAATACAATCACT (SEQ ID NO: | NH NG NG NI NI NG NG NG NH NI NI NG NI |
| | 665) | HD NI NI NG HD NI HD NG (SEQ ID NO: 685) |
| TALE4-F006 | TGTGTCACATACCAGCAGAAT (SEQ ID | NH NG NH NG HD NI HD NI NG NI HD HD NI |
| | NO: 666) | NH HD NI NH NI NI NG (SEQ ID NO: 686) |
| TALE4-R007 | TGGTAACTACTAATTT (SEQ ID NO: 667) | NH NH NG NI NI HD NG NI HD NG NI NI NG |
| | | NG NG (SEQ ID NO: 687) |
| TALE4-F008 | TGTCACATACCAGCAGAAT (SEQ ID NO: 668) | NH NG HD NI HD NI NG NI HD HD NI NH HD |
| | | NI NH NI NI NG (SEQ ID NO: 688) |
| TALE4-R009 | TGTGACACAGCCATCAACAAT (SEQ ID | NH NG NH NI HD NI HD NI NH HD HD NI NG |
| | NO: 669) | HD NI NI HD NI NI NG (SEQ ID NO: 689) |
| TALE4-F010 | TCCTTTGATGAACAGT (SEQ ID NO: 670) | HD HD NG NG NG NH NI NG NH NI NI HD NI |
| | | NH NG (SEQ ID NO: 690) |
| TALE4-F011 | TGTGTGCAATAGCGTTAAAGGAACTACAT | NH NG NH NG NH HD NI NI NG NI NH HD NH |
| | (SEQ ID NO: 671) | NG NG NI NI NI NH NH NI NI HD NG NI HD NI |
| | | NG (SEQ ID NO: 691) |
| TALE4-F012 | TCTTTCAATAGCCCACT (SEQ ID NO: 672) | HD NG NG NG HD NI NI NG NI NH HD HD HD |
| | | NI HD NG (SEQ ID NO: 692) |
| TALE4-R013 | TCTCAAATGACAAGAGCACAGT (SEQ ID | HD NG HD NI NI NI NG NH NI HD NI NI NH NI |
| | NO: 673) | NH HD NI HD NI NH NG (SEQ ID NO: 693) |
| TALE4-F014 | TACCAGTTAATTAGCACT (SEQ ID NO: 674) | NI HD HD NI NH NG NG NI NI NG NG NI NH |
| | | HD NI HD NG (SEQ ID NO: 694) |
| TALE4-F015 | TGTTGTGACCTAAGCCAT (SEQ ID NO: 675) | NH NG NG NH NG NH NI HD HD NG NI NI NH |
| | | HD HD NI NG (SEQ ID NO: 695) |
| TALE4-R016 | TCTCATGTTTTAAAGTCAAGAAT (SEQ ID | HD NG HD NI NG NH NG NG NG NG NI NI NI |
| | NO: 676) | NH NG HD NI NI NH NI NI NG (SEQ ID NO: |
| | | 696) |
| TALE4-F017 | TCCTGAATTCAGAACAGAT (SEQ ID NO: 677) | HD HD NG NH NI NI NG NG HD NI NH NI NI |
| | | HD NI NH NI NG (SEQ ID NO: 697) |
| TALE4-F018 | TAGCATGATGTTTCATGTTGTGACCT (SEQ | NI NH HD NI NG NH NI NG NH NG NG NG |
| | ID NO: 678) | HD NI NG NH NG NG NH NG NH NI HD HD |
| | | NG (SEQ ID NO: 698) |
| TALE4-F019 | TGTTTCATGTTGTGACCTAAGCCAT (SEQ ID | NH NG NG NG HD NI NG NH NG NG NH NG |
| | NO: 679) | NH NI HD HD NG NI NI NH HD HD NI NG |
| | | (SEQ ID NO: 699) |
| TALE4-F020 | TACAACAGTCTATTTCAT (SEQ ID NO: 680) | NI HD NI NI HD NI NH NG HD NG NI NG NG |
| | | NG HD NI NG (SEQ ID NO: 700) |

TABLE 11
TALE sequences targeting a chromosome 22 genomic safe harbor site (hg38 chr22:35,373,429-
35,380,000).

| NAME | DNA SEQUENCE | RVD AMINO ACID CODE |
|---|---|---|
| TALE22F- | TCTTCCTAGTCTCTTCTCTACCCAGT (SEQ | HD NG NG HD HD NG NI NH NG HD NG HD |
| R001 | ID NO: 701) | NG NG HD NG HD NG NI HD HD HD NI NH |
| | | NG (SEQ ID NO: 721) |
| TALE22- | TACACTCCAGCCTGGGAAACAGAGT (SEQ | NI HD NI HD NG HD HD NI NH HD HD NG NH |
| F002 | ID NO: 702) | NH NH NI NI NI HD NI NH NI NH NG (SEQ ID |
| | | NO: 722) |
| TALE22- | TCTTTTCCTTAGGACGGCT (SEQ ID | HD NG NG NG NG HD HD NG NG NI NH NH |
| F003 | NO: 703) | NI HD NH NH HD NG (SEQ ID NO: 723) |
| TALE22- | TCGCTCAGGCCTGTCAT (SEQ ID NO: 704) | HD NH HD NG HD NI NH NH HD HD NG NH |
| F004 | | NG HD NI NG (SEQ ID NO: 724) |
| TALE22- | TCCATATGGAAGACTT (SEQ ID NO: 705) | HD HD NI NG NI NG NH NH NI NI NH NI HD |
| F005 | | NG NG (SEQ ID NO: 725) |
| TALE22- | TACCCAGTTAACCACCCT (SEQ ID NO: 706) | NI HD HD HD NI NH NG NG NI NI HD HD NI |
| F006 | | HD HD HD NG (SEQ ID NO: 726) |
| TALE22- | TGGCGCATGCCTGTAATCCCAGCTACT (SEQ | NH NH HD NH HD NI NG NH HD HD NG NH |
| F007 | ID NO: 707) | NG NI NI NG HD HD HD NI NH HD NG NI HD |
| | | NG (SEQ ID NO: 727) |
| TALE22- | TATACGAGGAGAAAATTAGCATTCCT (SEQ | NI NG NI HD NH NI NH NH NI NH NI NI NI NI |
| F008 | ID NO: 708) | NG NG NI NH HD NI NG NG HD HD NG (SEQ |
| | | ID NO: 728) |
| TALE22- | TCTGCCTCCCAGGTTCACGCAAT (SEQ ID | HD NG NH HD HD NG HD HD HD NI NH NH |
| R009 | NO: 709) | NG NG HD NI HD NH HD NI NI NG (SEQ ID |
| | | NO: 729) |
| TALE22- | TGCCTTGTCACGTTTTCACAGT (SEQ ID | NH HD HD NG NG NH NG HD NI HD NH NG |
| F010 | NO: 710) | NG NG NG HD NI HD NI NH NG (SEQ ID NO: |
| | | 730) |
| TALE22- | TGTCACCTTCTGTATGTGCAACCAT (SEQ | NH NG HD NI HD HD NG NG HD NG NH NG |
| F001A | ID NO: 711) | NI NG NH NG NH HD NI NI HD HD NI NG |
| | | (SEQ ID NO: 731) |
| TALE22- | TCTGTATGTGCAACCAT (SEQ ID NO: 712) | HD NG NH NG NI NG NH NG NH HD NI NI HD |
| F002A | | HD NI NG (SEQ ID NO: 732) |
| TALE22- | TAGTCAAGCAACAGGAT (SEQ ID NO: 713) | NI NH NG HD NI NI NH HD NI NI HD NI NH |
| R03A | | NH NI NG (SEQ ID NO: 733) |
| TALE22- | TCCAAGATAATTCCCCAT (SEQ ID NO: 714) | HD HD NI NI NH NI NG NI NI NG NG HD HD |
| F004A | | HD HD NI NG (SEQ ID NO: 734) |
| TALE22- | TCTGCAAGATCCTTTT (SEQ ID NO: 715) | HD NG NH HD NI NI NH NI NG HD HD NG NG |
| F005A | | NG NG (SEQ ID NO: 735) |
| TALE22- | TGCTATGTAAGGTAGCAAAAAGGTAACCT | NH HD NG NI NG NH NG NI NI NH NH NG NI |
| F006A | (SEQ ID NO: 716) | NH HD NI NI NI NI NI NH NH NG NI NI HD HD |
| | | NG (SEQ ID NO: 736) |
| TALE22- | TCTCTCTCCTCCTGCT (SEQ ID NO: 717) | HD NG HD NG HD NG HD HD NG HD HD NG |
| R007A | | NH HD NG (SEQ ID NO: 737) |
| TALE22- | TCCAAATGCTATTCTCTCT (SEQ ID | HD HD NI NI NI NG NH HD NG NI NG NG HD |
| R008A | NO: 718) | NG HD NG HD NG (SEQ ID NO: 738) |
| TALE22- | TGCTGATTCAGCCTCCT (SEQ ID NO: 719) | NH HD NG NH NI NG NG HD NI NH HD HD |
| R009A | | NG HD HD NG (SEQ ID NO: 739) |
| TALE22- | TAGAACAGCCCCCCACACAGT (SEQ ID | NI NH NI NI HD NI NH HD HD HD HD HD HD |
| F010A | NO: 720) | NI HD NI HD NI NH NG (SEQ ID NO: 740) |

TABLE 12
TALE sequences targeting chromosome X (HPRT) (hg38 chrX:134,475,808-135,476,794).

| NAME | DNA SEQUENCE | RVD AMINO ACID CODE |
|---|---|---|
| TALE F002 | TTTAGCAGATGCATCAGC (SEQ ID | NG NG NI NH HD NI NH NI NG NH HD NI NG HD NI NH |
| | NO: 741) | HD (SEQ ID NO: 765) |
| TALE F003 | TGACCAGGGGCATGTCCTGG (SEQ | NH NI HD HD NI NH NH NH NH HD NI NG NH NG HD HD |
| | ID NO: 742) | NG NH NH (SEQ ID NO: 766) |
| TALE F004 | TGGTCCACCTACCTGAAAATG (SEQ | HD NI NI NH NH NI NH NG NG HD NG NH NH HD NG NH |
| | ID NO: 743) | NH NH NG HD (SEQ ID NO: 767) |
| TALE F007 | TGTCCCACAGGTATTACGGGC (SEQ | NH NG HD HD HD NI HD NI NH NH NG NI NG NG NI HD |
| | ID NO: 744) | NH NH NH HD (SEQ ID NO: 768) |
| TALE F008 | TACGGGCCAACCTGACAATAC (SEQ | NI HD NH NH NH HD HD NI NI HD HD NG NH NI HD NI |
| | ID NO: 745) | NI NG NI HD (SEQ ID NO: 769) |
| TALE F009 | TGAGCTTTGGGGACTGAAAGA (SEQ | NH NI NH HD NG NG NG NH NH NH NH NI HD NG NH NI |
| | ID NO: 746) | NI NI NH NI (SEQ ID NO: 770) |
| TALE R002 | CTGGCATAATCTTTTCCCCCA (SEQ | NH NH NH NH NH NI NI NI NI NH NI NG NG NI NG NH |
| | ID NO: 747) | HD HD NI NH (SEQ ID NO: 771) |
| TALE R003 | CCAGCCTCCTGGCCATGTGCA (SEQ | NH HD NI HD NI NG NH NH HD HD NI NH NH NI NH NH |
| | ID NO: 748) | HD NG NH NH (SEQ ID NO: 772) |
| TALE R004 | GGCCATGTGCACAGGGGCTGA (SEQ | HD NI NH HD HD HD HD NG NH NG NH HD NI HD NI NG |
| | ID NO: 749) | NH NH HD HD (SEQ ID NO: 773) |
| TALE R005 | CTGATATGTGAAGGTTTAGCA (SEQ | NH HD NG NI NI NI HD HD NG NG HD NI HD NI NG NI |
| | ID NO: 750) | NG HD NI NH (SEQ ID NO: 774) |
| TALE R007 | TGACCAGGCGTGGTGGCTCAC (SEQ | NH NI HD HD NI NH NH HD NH NG NH NH NG NH NH |
| | ID NO: 751) | HD NG HD NI HD (SEQ ID NO: 775) |
| TALE F020* | TATAGACATTTTCACT (SEQ ID | NI NG NI NH NI HD NI NG NG NG NG HD NI HD NG |
| | NO: 752) | (SEQ ID NO: 776) |
| TALE F021* | TCTACATTTAACTATCAACCT (SEQ | HD NG NI HD NI NG NG NG NI NI HD NG NI NG HD NI |
| | ID NO: 753) | NI HD HD NG (SEQ ID NO: 777) |
| TALE F030* | TCGTGCAAACGTTTGAT (SEQ ID | HD NH NG NH HD NI NI NI HD NH NG NG NG NH NI NG |
| | NO: 754) | (SEQ ID NO: 778) |
| TALE F031* | TACATCAATCCTGTAGGT* (SEQ ID | NI HD NI NG HD NI NI NG HD HD NG NH NG NI NH NH |
| | NO: 755) | NG (SEQ ID NO: 779) |
| TALE F034* | TCTATTTTAGTGACCCAAGT (SEQ | HD NG NI NG NG NG NG NI NH NG NH NI HD HD HD NI |
| | ID NO: 756) | NI NH NG (SEQ ID NO: 780) |
| TALE F036* | TAGAGTCAAAGCATGTACT (SEQ | NI NH NI NH NG HD NI NI NI NH HD NI NG NH NG NI |
| | ID NO: 757) | HD NG (SEQ ID NO: 781) |
| TALE F037* | TCCTACCCATAAGCTCCT (SEQ ID | HD HD NG NI HD HD HD NI NG NI NI NH HD NG HD HD |
| | NO: 758) | NG (SEQ ID NO: 782) |
| TALE F040* | TCCCCATCCCCATCAGT (SEQ ID | HD HD HD HD NI NG HD HD HD HD NI NG HD NI NH NG |
| | NO: 759) | (SEQ ID NO: 783) |
| TALE | TCTTTAATTCAAGCAAGACTTTAACAAGT | HD NG NG NG NI NI NG NG HD NI NI NH HD NI NI NH |
| R022* | (SEQ ID NO: 760) | NI HD NG NG NG NI NI HD NI NI NH NG (SEQ ID |
| | | NO: 784) |
| TALE | TGCAGTCCCCTTTCTT (SEQ ID | NH HD NI NH NG HD HD HD HD NG NG NG HD NG NG |
| R033* | NO: 761) | (SEQ ID NO: 785) |
| TALE | TCTGCACAAATCCCCAAAGAT (SEQ | HD NG NH HD NI HD NI NI NI NG HD HD HD HD NI NI |
| R035* | ID NO: 762) | NI NH NI NG (SEQ ID NO: 786) |
| TALE | TACATGCTTTGACTCT (SEQ ID | NI HD NI NG NH HD NG NG NG NH NI HD NG HD NG |
| R038* | NO: 763) | (SEQ ID NO: 787) |
| TALE | TGGCCAGTTATACTGCCAGCAGCTATAAT | NH NH HD HD NI NH NG NG NI NG NI HD NG NH HD HD |
| R039* | (SEQ ID NO: 764) | NI NH HD NI NH HD NG NI NG NI NI NG (SEQ ID NO: |
| | | 788) |

*TALEs near hotspots with 85 and 51 hits.

In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence.

In embodiments, the zinc finger targets one or more sites selected from TABLES 13-17.

TABLE 13
Zinc finger sequences targeting the genomic safe harbor site, hROSA26.

| hROS | | | | |
|---|---|---|---|---|
| A26 | | | | |
| TTAA | NAME | TARGET | SCORE | ZFP AMINO ACID CODE |
| 5′ | ZnF3a | TGG GAA GAT | 58.64 | LEPGEKPYKCPECGKSFSQNSTLTEHQRTHTGEKPYKCPECGKSF |
| | | AAA CTA (SEQ | | SQRANLRAHQRTHTGEKPYKCPECGKSFSTSGNLVRHQRTHTGEK |
| | | ID NO: 789) | | PYKCPECGKSFSQSSNLVRHQRTHTGEKPYKCPECGKSFSRSDHL |
| | | | | TTHQRTHTGKKTS (SEQ ID NO: 914) |
| 5′ | ZnF5a | ACT CCC CTG | 56.25 | LEPGEKPYKCPECGKSFSDSGNLRVHQRTHTGEKPYKCPECGKSF |
| | | CAG GGC AAC | | SDPGHLVRHQRTHTGEKPYKCPECGKSFSRADNLTEHQRTHTGEK |
| | | (SEQ ID NO: | | PYKCPECGKSFSRNDALTEHQRTHTGEKPYKCPECGKSFSSKKHL |
| | | 790) | | AEHQRTHTGEKPYKCPECGKSFSTHLDLIRHQRTHTGKKTS (SEQ |
| | | | | ID NO: 802) |
| 5′ | ZnF5b | CCC CTG CAG | 56.25 | LEPGEKPYKCPECGKSFSDCRDLARHQRTHTGEKPYKCPECGKSF |
| | | GGC AAC GCC | | SDSGNLRVHQRTHTGEKPYKCPECGKSFSDPGHLVRHQRTHTGE |
| | | (SEQ ID NO: | | KPYKCPECGKSFSRADNLTEHQRTHTGEKPYKCPECGKSFSRNDA |
| | | 791) | | LTEHQRTHTGEKPYKCPECGKSFSSKKHLAEHQRTHTGKKTS |
| | | | | (SEQ ID NO: 803) |
| 5′ | ZnF5c | CTG CAG GGC | 60.58 | LEPGEKPYKCPECGKSFSRADNLTEHQRTHTGEKPYKCPECGKSF |
| | | AAC GCC CAG | | SDCRDLARHQRTHTGEKPYKCPECGKSFSDSGNLRVHQRTHTGEK |
| | | (SEQ ID NO: | | PYKCPECGKSFSDPGHLVRHQRTHTGEKPYKCPECGKSFSRADNL |
| | | 792) | | TEHQRTHTGEKPYKCPECGKSFSRNDALTEHQRTHTGKKTS (SEQ |
| | | | | ID NO: 804) |
| 5′ | ZnF5d | CAG GGC AAC | 58.08 | LEPGEKPYKCPECGKSFSQRAHLERHQRTHTGEKPYKCPECGKSF |
| | | GCC CAG GGA | | SRADNLTEHQRTHTGEKPYKCPECGKSFSDCRDLARHQRTHTGEK |
| | | (SEQ ID NO: | | PYKCPECGKSFSDSGNLRVHQRTHTGEKPYKCPECGKSFSDPGHL |
| | | 793) | | VRHQRTHTGEKPYKCPECGKSFSRADNLTEHQRTHTGKKTS (SEQ |
| | | | | ID NO: 805) |
| 5′ | ZnF5e | GGC AAC GCC | 57.32 | LEPGEKPYKCPECGKSFSTSHSLTEHQRTHTGEKPYKCPECGKSF |
| | | CAG GGA CCA | | SQRAHLERHQRTHTGEKPYKCPECGKSFSRADNLTEHQRTHTGEK |
| | | (SEQ ID NO: | | PYKCPECGKSFSDCRDLARHQRTHTGEKPYKCPECGKSFSDSGNL |
| | | 794 | | RVHQRTHTGEKPYKCPECGKSFSDPGHLVRHQRTHTGKKTS (SEQ |
| | | | | ID NO: 806) |
| 5′ | ZnF5f | AAC GCC CAG | 54.99 | LEPGEKPYKCPECGKSFSHRTTLTNHQRTHTGEKPYKCPECGKSF |
| | | GGA CCA AGT | | STSHSLTEHQRTHTGEKPYKCPECGKSFSQRAHLERHQRTHTGEK |
| | | (SEQ ID NO: | | PYKCPECGKSFSRADNLTEHQRTHTGEKPYKCPECGKSFSDCRDL |
| | | 795) | | ARHQRTHTGEKPYKCPECGKSFSDSGNLRVHQRTHTGKKTS (SEQ |
| | | | | ID NO: 807) |
| 5′ | ZnF5g | GCC CAG GGA | 55.31 | LEPGEKPYKCPECGKSFSREDNLHTHQRTHTGEKPYKCPECGKSF |
| | | CCA AGT TAG | | SHRTTLTNHQRTHTGEKPYKCPECGKSFSTSHSLTEHQRTHTGEK |
| | | (SEQ ID NO: | | PYKCPECGKSFSQRAHLERHQRTHTGEKPYKCPECGKSFSRADNL |
| | | 796) | | TEHQRTHTGEKPYKCPECGKSFSDCRDLARHQRTHTGKKTS (SEQ |
| | | | | ID NO: 808) |
| 5′ | ZnF5h | CAG GGA CCA | 50.76 | LEPGEKPYKCPECGKSFSSKKHLAEHQRTHTGEKPYKCPECGKSF |
| | | AGT TAG CCC | | SREDNLHTHQRTHTGEKPYKCPECGKSFSHRTTLTNHQRTHTGEK |
| | | (SEQ ID NO: | | PYKCPECGKSFSTSHSLTEHQRTHTGEKPYKCPECGKSFSQRAHL |
| | | 797) | | ERHQRTHTGEKPYKCPECGKSFSRADNLTEHQRTHTGKKTS (SEQ |
| | | | | ID NO: 809) |
| 3 | ZnF12a | GCC TAG GCA | 59.09 | LEPGEKPYKCPECGKSFSQSSNLVRHQRTHTGEKPYKCPECGKSF |
| | | AAA GAA (SEQ | | SQRANLRAHQRTHTGEKPYKCPECGKSFSQSGDLRRHQRTHTGE |
| | | ID NO: 798) | | KPYKCPECGKSFSREDNLHTHQRTHTGEKPYKCPECGKSFSDCRD |
| | | | | LARHQRTHTGKKTS (SEQ ID NO: 810) |
| 3 | ZnF13a | CGC GAG GAG | 57.19 | LEPGEKPYKCPECGKSFSRSDHLTNHQRTHTGEKPYKCPECGKSF |
| | | GAA AGG AGG | | SRSDHLTNHQRTHTGEKPYKCPECGKSFSQSSNLVRHQRTHTGEK |
| | | (SEQ ID NO: | | PYKCPECGKSFSRSDNLVRHQRTHTGEKPYKCPECGKSFSRSDNL |
| | | 799) | | VRHQRTHTGEKPYKCPECGKSFSHTGHLLEHQRTHTGKKTS (SEQ |
| | | | | ID NO: 811) |
| 3′ | ZnF13b | GAG GAG GAA | 57.80 | LEPGEKPYKCPECGKSFSRSDNLVRHQRTHTGEKPYKCPECGKSF |
| | | AGG AGG GAG | | SRSDHLTNHQRTHTGEKPYKCPECGKSFSRSDHLTNHQRTHTGEK |
| | | (SEQ ID NO: | | PYKCPECGKSFSQSSNLVRHQRTHTGEKPYKCPECGKSFSRSDNL |
| | | 800) | | VRHQRTHTGEKPYKCPECGKSFSRSDNLVRHQRTHTGKKTS (SEQ |
| | | | | ID NO: 812) |
| 3′ | ZnF13c | GAG GAA AGG | 57.61 | LEPGEKPYKCPECGKSFSDPGHLVRHQRTHTGEKPYKCPECGKSF |
| | | AGG GAG GGC | | SRSDNLVRHQRTHTGEKPYKCPECGKSFSRSDHLTNHQRTHTGEK |
| | | (SEQ ID NO: | | PYKCPECGKSFSRSDHLTNHQRTHTGEKPYKCPECGKSFSQSSNL |
| | | 801) | | VRHQRTHTGEKPYKCPECGKSFSRSDNLVRHQRTHTGKKTS (SEQ |
| | | | | ID NO: 813) |

No Sequences have Target site overlap (TSO)
Available on the world wide web at scripps.edu/barbas/zfdesign/searchsequence.php

TABLE 14
Zinc finger sequences targeting the genomic safe harbor site, AAVS1.

| AAVS1 | | | | |
|---|---|---|---|---|
| TTAA | NAME | TARGET | SCORE | ZFP AMINO ACID CODE |
| 5′ | ZnF11a | TAG GAC AGT GGG GAA AAT | 57.08 | LEPGEKPYKCPECGKSFSDCRDLARHQRTHTGEK |
| | | GAC CCA ACA GCC (SEQ ID | | PYKCPECGKSFSSPADLTRHQRTHTGEKPYKCPE |
| | | NO: 814) | | CGKSFSTSHSLTEHQRTHTGEKPYKCPECGKSFS |
| | | | | DPGNLVRHQRTHTGEKPYKCPECGKSFSTTGNLT |
| | | | | VHQRTHTGEKPYKCPECGKSFSQSSNLVRHQRT |
| | | | | HTGEKPYKCPECGKSFSRSDKLVRHQRTHTGEKP |
| | | | | YKCPECGKSFSHRTTLTNHQRTHTGEKPYKCPEC |
| | | | | GKSFSDPGNLVRHQRTHTGEKPYKCPECGKSFS |
| | | | | REDNLHTHQRTHTGKKTS (SEQ ID NO: 829) |
| 5′ | ZnF10a | AGA GGG AGC CAC GAA AAC | 56.91 | LEPGEKPYKCPECGKSFSQLAHLRAHQRTHTGEK |
| | | AGA (SEQ ID NO: 815) | | PYKCPECGKSFSDSGNLRVHQRTHTGEKPYKCPE |
| | | | | CGKSFSQSSNLVRHQRTHTGEKPYKCPECGKSFS |
| | | | | SKKALTEHQRTHTGEKPYKCPECGKSFSERSHLR |
| | | | | EHQRTHTGEKPYKCPECGKSFSRSDKLVRHQRT |
| | | | | HTGEKPYKCPECGKSFSQLAHLRAHQRTHTGKKT |
| | | | | S (SEQ ID NO: 830) |
| 3′ | ZnF12b | GCA GAT AGC CAG GAG (SEQ | 59.97 | LEPGEKPYKCPECGKSFSRSDNLVRHQRTHTGEK |
| | | ID NO: 816) | | PYKCPECGKSFSRADNLTEHQRTHTGEKPYKCPE |
| | | | | CGKSFSERSHLREHQRTHTGEKPYKCPECGKSFS |
| | | | | TSGNLVRHQRTHTGEKPYKCPECGKSFSQSGDL |
| | | | | RRHQRTHTGKKTS (SEQ ID NO: 831) |
| 3′ | ZnF13b | AGA TAG CCA GGA GTC CTT | 56.80 | LEPGEKPYKCPECGKSFSTTGALTEHQRTHTGEK |
| | | (SEQ ID NO: 817) | | PYKCPECGKSFSDPGALVRHQRTHTGEKPYKCPE |
| | | | | CGKSFSQRAHLERHQRTHTGEKPYKCPECGKSF |
| | | | | STSHSLTEHQRTHTGEKPYKCPECGKSFSREDNL |
| | | | | HTHQRTHTGEKPYKCPECGKSFSQLAHLRAHQRT |
| | | | | HTGKKTS (SEQ ID NO: 832) |
| 5′ | ZnF14a | CCC AGT GGT CAG GCC GGC | 61.78 | LEPGEKPYKCPECGKSFSDCRDLARHQRTHTGEK |
| | | CAG GCC (SEQ ID NO: 818) | | PYKCPECGKSFSRADNLTEHQRTHTGEKPYKCPE |
| | | | | CGKSFSDPGHLVRHQRTHTGEKPYKCPECGKSF |
| | | | | SDCRDLARHQRTHTGEKPYKCPECGKSFSRADNL |
| | | | | TEHQRTHTGEKPYKCPECGKSFSTSGHLVRHQRT |
| | | | | HTGEKPYKCPECGKSFSHRTTLTNHQRTHTGEKP |
| | | | | YKCPECGKSFSSKKHLAEHQRTHTGKKTS (SEQ |
| | | | | ID NO: 833) |
| 5′ | ZnF15a | GGC CGG CCA GGC CTT CAG | 58.15 | LEPGEKPYKCPECGKSFSRADNLTEHQRTHTGEK |
| | | (SEQ ID NO: 819) | | PYKCPECGKSFSTTGALTEHQRTHTGEKPYKCPE |
| | | | | CGKSFSDPGHLVRHQRTHTGEKPYKCPECGKSF |
| | | | | STSHSLTEHQRTHTGEKPYKCPECGKSFSRSDKL |
| | | | | TEHQRTHTGEKPYKCPECGKSFSDPGHLVRHQR |
| | | | | THTGKKTS (SEQ ID NO: 834) |
| 5′ | ZnF16a | AGT GCT CAG TGG AAA CCA | 58.65 | LEPGEKPYKCPECGKSFSDPGNLVRHQRTHTGEK |
| | | CGA AAG GAC (SEQ ID | | PYKCPECGKSFSRKDNLKNHQRTHTGEKPYKCPE |
| | | NO: 820) | | CGKSFSQSGHLTEHQRTHTGEKPYKCPECGKSFS |
| | | | | TSHSLTEHQRTHTGEKPYKCPECGKSFSQRANLR |
| | | | | AHQRTHTGEKPYKCPECGKSFSRSDHLTTHQRTH |
| | | | | TGEKPYKCPECGKSFSRADNLTEHQRTHTGEKPY |
| | | | | KCPECGKSFSTSGELVRHQRTHTGEKPYKCPECG |
| | | | | KSFSHRTTLTNHQRTHTGKKTS (SEQ ID NO: |
| | | | | 835) |
| 5′ | ZnF17a | TGG CCC CCA GCC CCT CCT | 60.89 | LEPGEKPYKCPECGKSFSDCRDLARHQRTHTGEK |
| | | GCC (SEQ ID NO: 821) | | PYKCPECGKSFSTKNSLTEHQRTHTGEKPYKCPE |
| | | | | CGKSFSTKNSLTEHQRTHTGEKPYKCPECGKSFS |
| | | | | DCRDLARHQRTHTGEKPYKCPECGKSFSTSHSLT |
| | | | | EHQRTHTGEKPYKCPECGKSFSSKKHLAEHQRTH |
| | | | | TGEKPYKCPECGKSFSRSDHLTTHQRTHTGKKTS |
| | | | | (SEQ ID NO: 836) |
| 5′ | ZnF18a | AGA GCC AGG AGT CCT GGC | 57.23 | LEPGEKPYKCPECGKSFSSKKHLAEHQRTHTGEK |
| | | CCC CAG CCC (SEQ ID | | PYKCPECGKSFSRADNLTEHQRTHTGEKPYKCPE |
| | | NO: 822) | | CGKSFSSKKHLAEHQRTHTGEKPYKCPECGKSFS |
| | | | | DPGHLVRHQRTHTGEKPYKCPECGKSFSTKNSLT |
| | | | | EHQRTHTGEKPYKCPECGKSFSHRTTLTNHQRTH |
| | | | | TGEKPYKCPECGKSFSRSDHLTNHQRTHTGEKPY |
| | | | | KCPECGKSFSDCRDLARHQRTHTGEKPYKCPEC |
| | | | | GKSFSQLAHLRAHQRTHTGKKTS (SEQ ID NO: |
| | | | | 837) |
| 3′ | ZnF19a | GCA GGA GGG GCT GGG GGC | 59.93 | LEPGEKPYKCPECGKSFSDPGNLVRHQRTHTGEK |
| | | CAG GAC (SEQ ID NO: 823) | | PYKCPECGKSFSRADNLTEHQRTHTGEKPYKCPE |
| | | | | CGKSFSDPGHLVRHQRTHTGEKPYKCPECGKSF |
| | | | | SRSDKLVRHQRTHTGEKPYKCPECGKSFSTSGEL |
| | | | | VRHQRTHTGEKPYKCPECGKSFSRSDKLVRHQR |
| | | | | THTGEKPYKCPECGKSFSQRAHLERHQRTHTGEK |
| | | | | PYKCPECGKSFSQSGDLRRHQRTHTGKKTS |
| | | | | (SEQ ID NO: 838) |
| 3′ | ZnF20b | ATA GCC CTG GGC CCA CGG | 59.53 | LEPGEKPYKCPECGKSFSSRRTCRAHQRTHTGEK |
| | | CTT CGT (SEQ ID NO: 824) | | PYKCPECGKSFSTTGALTEHQRTHTGEKPYKCPE |
| | | | | CGKSFSRSDKLTEHQRTHTGEKPYKCPECGKSFS |
| | | | | TSHSLTEHQRTHTGEKPYKCPECGKSFSDPGHLV |
| | | | | RHQRTHTGEKPYKCPECGKSFSRNDALTEHQRT |
| | | | | HTGEKPYKCPECGKSFSDCRDLARHQRTHTGEK |
| | | | | PYKCPECGKSFSQKSSLIAHQRTHTGKKT (SEQ |
| | | | | ID NO: 839) |
| 3′ | ZnF21b | GAA GGA CCT GGC TGG (SEQ | 55.22 | LEPGEKPYKCPECGKSFSRSDHLTTHQRTHTGEK |
| | | ID NO: 825) | | PYKCPECGKSFSDPGHLVRHQRTHTGEKPYKCPE |
| | | | | CGKSFSTKNSLTEHQRTHTGEKPYKCPECGKSFS |
| | | | | QRAHLERHQRTHTGEKPYKCPECGKSFSQSSNLV |
| | | | | RHQRTHTGKKTS (SEQ ID NO: 840) |
| 5′ | ZnF22a | GCA GGA ACG AAG CCG TGG | 56.47 | LEPGEKPYKCPECGKSFSDPGHLVRHQRTHTGEK |
| | | GCC CAG GGC (SEQ ID | | PYKCPECGKSFSRADNLTEHQRTHTGEKPYKCPE |
| | | NO: 826) | | CGKSFSDCRDLARHQRTHTGEKPYKCPECGKSF |
| | | | | SRSDHLTTHQRTHTGEKPYKCPECGKSFSRNDTL |
| | | | | TEHQRTHTGEKPYKCPECGKSFSRKDNLKNHQRT |
| | | | | HTGEKPYKCPECGKSFSRTDTLRDHQRTHTGEKP |
| | | | | YKCPECGKSFSQRAHLERHQRTHTGEKPYKCPE |
| | | | | CGKSFSQSGDLRRHQRTHTGKKTS (SEQ ID |
| | | | | NO: 841) |
| 5′ | ZnF23a | GGA AAC CAC CCC AGC AGA | 52.63 | LEPGEKPYKCPECGKSFSQLAHLRAHQRTHTGEK |
| | | (SEQ ID NO: 827) | | PYKCPECGKSFSERSHLREHQRTHTGEKPYKCPE |
| | | | | CGKSFSSKKHLAEHQRTHTGEKPYKCPECGKSFS |
| | | | | SKKALTEHQRTHTGEKPYKCPECGKSFSDSGNLR |
| | | | | VHQRTHTGEKPYKCPECGKSFSQRAHLERHQRT |
| | | | | HTGKKTS (SEQ ID NO: 842) |
| 5′ | ZnF24a | AAG GGT CAA GCT CGG AAA | 55.09 | LEPGEKPYKCPECGKSFSQKSSLIAHQRTHTGEK |
| | | CCA CCC CAG CAG ATA | | PYKCPECGKSFSRADNLTEHQRTHTGEKPYKCPE |
| | | (SEQ ID NO: 828) | | CGKSFSRADNLTEHQRTHTGEKPYKCPECGKSFS |
| | | | | SKKHLAEHQRTHTGEKPYKCPECGKSFSTSHSLT |
| | | | | EHQRTHTGEKPYKCPECGKSFSQRANLRAHQRT |
| | | | | HTGEKPYKCPECGKSFSRSDKLTEHQRTHTGEKP |
| | | | | YKCPECGKSFSTSGELVRHQRTHTGEKPYKCPEC |
| | | | | GKSFSQSGNLTEHQRTHTGEKPYKCPECGKSFST |
| | | | | SGHLVRHQRTHTGEKPYKCPECGKSFSRKDNLKN |
| | | | | HQRTHTGKKTS (SEQ ID NO: 843) |

No Sequences have Target site overlap (TSO)
Available on the world wide web at scripps.edu/barbas/zfdesign/searchsequence.php

TABLE 15
Zinc finger sequences targeting a chromosome 4 genomic safe harbor site (hg38 chr4:30,793,039-
30,793,980).

| Chr4 | | | | |
|---|---|---|---|---|
| TTAA | NAME | TARGET | SCORE | ZFP AMINO ACID CODE |
| 5′ | ZnF31F | CTTTGATGAACAGTCACA (SEQ | 58.41 | LEPGEKPYKCPECGKSFSSPADLTRHQRTHTGEK |
| | | ID NO: 844) | | PYKCPECGKSFSDPGALVRHQRTHTGEKPYKCPE |
| | | | | CGKSFSSPADLTRHQRTHTGEKPYKCPECGKSFS |
| | | | | QAGHLASHQRTHTGEKPYKCPECGKSFSQAGHL |
| | | | | ASHQRTHTGEKPYKCPECGKSFSTTGALTEHQRT |
| | | | | HTGKKTS (SEQ ID NO: 853) |
| 5′ | ZnF32F | CTTCCAATTAGTCCTACC (SEQ | 55.84 | LEPGEKPYKCPECGKSFSDKKDLTRHQRTHTGEK |
| | | ID NO: 845) | | PYKCPECGKSFSTKNSLTEHQRTHTGEKPYKCPE |
| | | | | CGKSFSHRTTLTNHQRTHTGEKPYKCPECGKSFS |
| | | | | HKNALQNHQRTHTGEKPYKCPECGKSFSTSHSLT |
| | | | | EHQRTHTGEKPYKCPECGKSFSTTGALTEHQRTH |
| | | | | TGKKTS (SEQ ID NO: 854) |
| 5′ | ZnF33F | ATACTAGGAAGAAATACAATA | 57.27 | LEPGEKPYKCPECGKSFSQKSSLIAHQRTHTGEK |
| | | (SEQ ID NO: 846) | | PYKCPECGKSFSSPADLTRHQRTHTGEKPYKCPE |
| | | | | CGKSFSTTGNLTVHQRTHTGEKPYKCPECGKSFS |
| | | | | QLAHLRAHQRTHTGEKPYKCPECGKSFSQRAHLE |
| | | | | RHQRTHTGEKPYKCPECGKSFSQNSTLTEHQRTH |
| | | | | TGEKPYKCPECGKSFSQKSSLIAHQRTHTGKKTS |
| | | | | (SEQ ID NO: 855) |
| 5′ | ZnF34F | GCTCTTGTCATTTGAGAT (SEQ | 57.38 | LEPGEKPYKCPECGKSFSTSGNLVRHQRTHTGEK |
| | | ID NO: 847) | | PYKCPECGKSFSQAGHLASHQRTHTGEKPYKCPE |
| | | | | CGKSFSHKNALQNHQRTHTGEKPYKCPECGKSF |
| | | | | SDPGALVRHQRTHTGEKPYKCPECGKSFSTTGAL |
| | | | | TEHQRTHTGEKPYKCPECGKSFSTSGELVRHQRT |
| | | | | HTGKKTS (SEQ ID NO: 856) |
| 5′ | ZnF35F | CCAAGCTGAAATGACACAAAA | 58.23 | LEPGEKPYKCPECGKSFSRKDNLKNHQRTHTGEK |
| | | GTTAAAACAAAG (SEQ ID NO: | | PYKCPECGKSFSSPADLTRHQRTHTGEKPYKCPE |
| | | 848) | | CGKSFSQRANLRAHQRTHTGEKPYKCPECGKSF |
| | | | | STSGSLVRHQRTHTGEKPYKCPECGKSFSQRANL |
| | | | | RAHQRTHTGEKPYKCPECGKSFSSPADLTRHQRT |
| | | | | HTGEKPYKCPECGKSFSDPGNLVRHQRTHTGEK |
| | | | | PYKCPECGKSFSTTGNLTVHQRTHTGEKPYKCPE |
| | | | | CGKSFSQAGHLASHQRTHTGEKPYKCPECGKSF |
| | | | | SERSHLREHQRTHTGEKPYKCPECGKSFSTSHSL |
| | | | | TEHQRTHTGKKTS (SEQ ID NO: 857) |
| 5′ | ZnF36F | CTTATACCAGTTAATTAGCAC | 49.93 | LEPGEKPYKCPECGKSFSSKKALTEHQRTHTGEK |
| | | (SEQ ID NO: 849) | | PYKCPECGKSFSREDNLHTHQRTHTGEKPYKCPE |
| | | | | CGKSFSTTGNLTVHQRTHTGEKPYKCPECGKSFS |
| | | | | TSGSLVRHQRTHTGEKPYKCPECGKSFSTSHSLT |
| | | | | EHQRTHTGEKPYKCPECGKSFSQKSSLIAHQRTH |
| | | | | TGEKPYKCPECGKSFSTTGALTEHQRTHTGKKTS |
| | | | | (SEQ ID NO: 858) |
| 3′ | ZnF37R | AACGCTATTGCACACATAGTTA | 57.67 | LEPGEKPYKCPECGKSFSSPADLTRHQRTHTGEK |
| | | CA (SEQ ID NO: 850) | | PYKCPECGKSFSTSGSLVRHQRTHTGEKPYKCPE |
| | | | | CGKSFSQKSSLIAHQRTHTGEKPYKCPECGKSFS |
| | | | | SKKALTEHQRTHTGEKPYKCPECGKSFSQSGDLR |
| | | | | RHQRTHTGEKPYKCPECGKSFSHKNALQNHQRT |
| | | | | HTGEKPYKCPECGKSFSTSGELVRHQRTHTGEKP |
| | | | | YKCPECGKSFSDSGNLRVHQRTHTGKKTS (SEQ |
| | | | | ID NO: 859) |
| 3′ | ZnF38R | TGAATTCAGGAACAAAGTATA | 53.21 | LEPGEKPYKCPECGKSFSQKSSLIAHQRTHTGEK |
| | | (SEQ ID NO: 851) | | PYKCPECGKSFSHRTTLTNHQRTHTGEKPYKCPE |
| | | | | CGKSFSQSGNLTEHQRTHTGEKPYKCPECGKSFS |
| | | | | QSSNLVRHQRTHTGEKPYKCPECGKSFSRADNLT |
| | | | | EHQRTHTGEKPYKCPECGKSFSHKNALQNHQRT |
| | | | | HTGEKPYKCPECGKSFSQAGHLASHQRTHTGKKT |
| | | | | S (SEQ ID NO: 860) |
| 3′ | ZnF39R | GCTGGTATGTGACACAGCCAT | 50.63 | LEPGEKPYKCPECGKSFSQSGNLTEHQRTHTGEK |
| | | CAACAA (SEQ ID NO: 852) | | PYKCPECGKSFSQSGNLTEHQRTHTGEKPYKCPE |
| | | | | CGKSFSTSGNLTEHQRTHTGEKPYKCPECGKSFS |
| | | | | ERSHLREHQRTHTGEKPYKCPECGKSFSSKKALT |
| | | | | EHQRTHTGEKPYKCPECGKSFSQAGHLASHQRT |
| | | | | HTGEKPYKCPECGKSFSRRDELNVHQRTHTGEKP |
| | | | | YKCPECGKSFSTSGHLVRHQRTHTGEKPYKCPEC |
| | | | | GKSFSTSGELVRHQRTHTGKKTS (SEQ ID NO: |
| | | | | 861) |

No Sequences have Target site overlap (TSO)
Available on the world wide web at scripps.edu/barbas/zfdesign/searchsequence.php

TABLE 16
Zinc finger sequences targeting a chromosome 22 genomic safe harbor site (hg38
chr22:35,373,429-35,380,000).

| Chr22 | | | | |
|---|---|---|---|---|
| TTAA | NAME | TARGET | SCORE | ZFP |
| 5′ | ZnF1a | CTTCCTGAAAGCAAGA | 57.34 | LEPGEKPYKCPECGKSFSTTGNLTVHQRTHTGEKPYKCPECGKS |
| | | GATGAAAT (SEQ ID | | FSQAGHLASHQRTHTGEKPYKCPECGKSFSQLAHLRAHQRTHTG |
| | | NO: 862) | | EKPYKCPECGKSFSRKDNLKNHQRTHTGEKPYKCPECGKSFSER |
| | | | | SHLREHQRTHTGEKPYKCPECGKSFSQSSNLVRHQRTHTGEKPY |
| | | | | KCPECGKSFSTKNSLTEHQRTHTGEKPYKCPECGKSFSTTGALTE |
| | | | | HQRTHTGKKTS (SEQ ID NO: 879) |
| 5′ | ZnF1b | CTGAAAGCAAGAGATG | 58.92 | LEPGEKPYKCPECGKSFSTSHSLTEHQRTHTGEKPYKCPECGKSF |
| | | AAATTCCA (SEQ ID | | SHKNALQNHQRTHTGEKPYKCPECGKSFSQSSNLVRHQRTHTGE |
| | | NO: 863) | | KPYKCPECGKSFSTSGNLVRHQRTHTGEKPYKCPECGKSFSQLA |
| | | | | HLRAHQRTHTGEKPYKCPECGKSFSQSGDLRRHQRTHTGEKPYK |
| | | | | CPECGKSFSQRANLRAHQRTHTGEKPYKCPECGKSFSRNDALTE |
| | | | | HQRTHTGKKTS (SEQ ID NO: 880) |
| 5′ | ZnF2a | ATACGAGGAGAAAATT | 51.25 | LEPGEKPYKCPECGKSFSTSGNLTEHQRTHTGEKPYKCPECGKS |
| | | AGCAT (SEQ ID NO: | | FSREDNLHTHQRTHTGEKPYKCPECGKSFSTTGNLTVHQRTHTG |
| | | 864) | | EKPYKCPECGKSFSQSSNLVRHQRTHTGEKPYKCPECGKSFSQR |
| | | | | AHLERHQRTHTGEKPYKCPECGKSFSQSGHLTEHQRTHTGEKPY |
| | | | | KCPECGKSFSQKSSLIAHQRTHTGKKTS (SEQ ID NO: 881) |
| 5′ | ZnF3a | CATCCATGGCAGGAA | 58.67 | LEPGEKPYKCPECGKSFSRNDALTEHQRTHTGEKPYKCPECGKS |
| | | GTTGAAGCCAAAATAA | | FSTTGNLTVHQRTHTGEKPYKCPECGKSFSQKSSLIAHQRTHTGE |
| | | ATCTG (SEQ ID NO: | | KPYKCPECGKSFSQRANLRAHQRTHTGEKPYKCPECGKSFSDCR |
| | | 865) | | DLARHQRTHTGEKPYKCPECGKSFSQSSNLVRHQRTHTGEKPYK |
| | | | | CPECGKSFSTSGSLVRHQRTHTGEKPYKCPECGKSFSQSSNLVR |
| | | | | HQRTHTGEKPYKCPECGKSFSRADNLTEHQRTHTGEKPYKCPEC |
| | | | | GKSFSRSDHLTTHQRTHTGEKPYKCPECGKSFSTSHSLTEHQRT |
| | | | | HTGEKPYKCPECGKSFSTSGNLTEHQRTHTGKKTS (SEQ ID NO: |
| | | | | 882 |
| 5′ | ZnF3b | ATGGCAGGAAGTTGAA | 54.14 | LEPGEKPYKCPECGKSFSQRANLRAHQRTHTGEKPYKCPECGKS |
| | | GCCAAAATAAA (SEQ | | FSTTGNLTVHQRTHTGEKPYKCPECGKSFSQSGNLTEHQRTHTG |
| | | ID NO: 866) | | EKPYKCPECGKSFSERSHLREHQRTHTGEKPYKCPECGKSFSQA |
| | | | | GHLASHQRTHTGEKPYKCPECGKSFSHRTTLTNHQRTHTGEKPY |
| | | | | KCPECGKSFSQRAHLERHQRTHTGEKPYKCPECGKSFSQSGDLR |
| | | | | RHQRTHTGEKPYKCPECGKSFSRRDELNVHQRTHTGKKTS (SEQ |
| | | | | ID NO: 883) |
| 3′ | ZnF5aR | GAAAAGAAGACTCAAG | 55.40 | LEPGEKPYKCPECGKSFSSKKALTEHQRTHTGEKPYKCPECGKSF |
| | | GAAACAGAGCCAAACA | | SQRANLRAHQRTHTGEKPYKCPECGKSFSDCRDLARHQRTHTGE |
| | | C (SEQ ID NO: 867) | | KPYKCPECGKSFSQLAHLRAHQRTHTGEKPYKCPECGKSFSDSG |
| | | | | NLRVHQRTHTGEKPYKCPECGKSFSQRAHLERHQRTHTGEKPYK |
| | | | | CPECGKSFSQSGNLTEHQRTHTGEKPYKCPECGKSFSTHLDLIRH |
| | | | | QRTHTGEKPYKCPECGKSFSRKDNLKNHQRTHTGEKPYKCPECG |
| | | | | KSFSRKDNLKNHQRTHTGEKPYKCPECGKSFSQSSNLVRHQRTH |
| | | | | TGKKTS (SEQ ID NO: 884) |
| 3′ | ZnF5bR | AGGAAACAGAGCCAAA | 54.66 | LEPGEKPYKCPECGKSFSSPADLTRHQRTHTGEKPYKCPECGKS |
| | | CACTTACA (SEQ ID | | FSTTGALTEHQRTHTGEKPYKCPECGKSFSSPADLTRHQRTHTGE |
| | | NO: 868) | | KPYKCPECGKSFSQSGNLTEHQRTHTGEKPYKCPECGKSFSERS |
| | | | | HLREHQRTHTGEKPYKCPECGKSFSRADNLTEHQRTHTGEKPYK |
| | | | | CPECGKSFSQRANLRAHQRTHTGEKPYKCPECGKSFSRSDHLTN |
| | | | | HQRTHTGKKTS (SEQ ID NO: 885) |
| 3′ | ZnF6aR | ATGCAGATTTGGACAC | 58.57 | LEPGEKPYKCPECGKSFSRSDKLVRHQRTHTGEKPYKCPECGKS |
| | | AGAGTAGTAAACTGTG | | FSSRRTCRAHQRTHTGEKPYKCPECGKSFSRSDHLTTHQRTHTG |
| | | AAAACGTGACAAGGCA | | EKPYKCPECGKSFSRKDNLKNHQRTHTGEKPYKCPECGKSFSQS |
| | | AAGTGGCGTGGG | | GDLRRHQRTHTGEKPYKCPECGKSFSRKDNLKNHQRTHTGEKPY |
| | | (SEQ ID NO: 869) | | KCPECGKSFSDPGNLVRHQRTHTGEKPYKCPECGKSFSSRRTCR |
| | | | | AHQRTHTGEKPYKCPECGKSFSQRANLRAHQRTHTGEKPYKCPE |
| | | | | CGKSFSQAGHLASHQRTHTGEKPYKCPECGKSFSRNDALTEHQR |
| | | | | THTGEKPYKCPECGKSFSQRANLRAHQRTHTGEKPYKCPECGKS |
| | | | | FSHRTTLTNHQRTHTGEKPYKCPECGKSFSHRTTLTNHQRTHTGE |
| | | | | KPYKCPECGKSFSRADNLTEHQRTHTGEKPYKCPECGKSFSSPA |
| | | | | DLTRHQRTHTGEKPYKCPECGKSFSRSDHLTTHQRTHTGEKPYK |
| | | | | CPECGKSFSHKNALQNHQRTHTGEKPYKCPECGKSFSRADNLTE |
| | | | | HQRTHTGEKPYKCPECGKSFSRRDELNVHQRTHTGKKTS (SEQ |
| | | | | ID NO: 886) |
| 3′ | ZnF6bR | GGACACAGAGTAGTAA | 55.80 | LEPGEKPYKCPECGKSFSDSGNLRVHQRTHTGEKPYKCPECGKS |
| | | AC (SEQ ID NO: 870) | | FSQSSSLVRHQRTHTGEKPYKCPECGKSFSQSSSLVRHQRTHTG |
| | | | | EKPYKCPECGKSFSQLAHLRAHQRTHTGEKPYKCPECGKSFSSK |
| | | | | KALTEHQRTHTGEKPYKCPECGKSFSQRAHLERHQRTHTGKKTS |
| | | | | (SEQ ID NO: 887) |
| 5′ | ZnF10F | AAAGCTAGCAGCATGG | 57.55 | LEPGEKPYKCPECGKSFSQSGDLRRHQRTHTGEKPYKCPECGKS |
| | | CA (SEQ ID NO: 871) | | FSRRDELNVHQRTHTGEKPYKCPECGKSFSERSHLREHQRTHTG |
| | | | | EKPYKCPECGKSFSERSHLREHQRTHTGEKPYKCPECGKSFSTS |
| | | | | GELVRHQRTHTGEKPYKCPECGKSFSQRANLRAHQRTHTGKKTS |
| | | | | (SEQ ID NO: 888) |
| 5′ | ZnF11F | CCTCTTATAAGGCCCA | 52.55 | LEPGEKPYKCPECGKSFSQKSSLIAHQRTHTGEKPYKCPECGKSF |
| | | AGAGGATA (SEQ ID | | SRSDHLTNHQRTHTGEKPYKCPECGKSFSRKDNLKNHQRTHTGE |
| | | NO: 872) | | KPYKCPECGKSFSSKKHLAEHQRTHTGEKPYKCPECGKSFSRSD |
| | | | | HLTNHQRTHTGEKPYKCPECGKSFSQKSSLIAHQRTHTGEKPYKC |
| | | | | PECGKSFSTTGALTEHQRTHTGEKPYKCPECGKSFSTKNSLTEHQ |
| | | | | RTHTGKKTS (SEQ ID NO: 889) |
| 5′ | ZnF12F | CAACATCCTTGACTTA | 55.00 | LEPGEKPYKCPECGKSFSSKKALTEHQRTHTGEKPYKCPECGKSF |
| | | ATCAC (SEQ ID NO: | | STTGNLTVHQRTHTGEKPYKCPECGKSFSTTGALTEHQRTHTGEK |
| | | 873) | | PYKCPECGKSFSQAGHLASHQRTHTGEKPYKCPECGKSFSTKNS |
| | | | | LTEHQRTHTGEKPYKCPECGKSFSTSGNLTEHQRTHTGEKPYKC |
| | | | | PECGKSFSQSGNLTEHQRTHTGKKTS (SEQ ID NO: 890) |
| 5′ | ZnF13F | GGTAGCAAAAAGGTAA | 46.33 | LEPGEKPYKCPECGKSFSDKKDLTRHQRTHTGEKPYKCPECGKS |
| | | CC | | FSQSSSLVRHQRTHTGEKPYKCPECGKSFSRKDNLKNHQRTHTG |
| | | (SEQ ID NO: 874) | | EKPYKCPECGKSFSQRANLRAHQRTHTGEKPYKCPECGKSFSER |
| | | | | SHLREHQRTHTGEKPYKCPECGKSFSTSGHLVRHQRTHTGKKTS |
| | | | | (SEQ ID NO: 891) |
| 3′ | ZnF14R | TGGGGTGCAAGAGGC | 61.28 | LEPGEKPYKCPECGKSFSDPGALVRHQRTHTGEKPYKCPECGKS |
| | | CAGGCCAGAGTTGTTC | | FSRNDALTEHQRTHTGEKPYKCPECGKSFSTSGSLVRHQRTHTG |
| | | TGGTC (SEQ ID NO: | | EKPYKCPECGKSFSTSGSLVRHQRTHTGEKPYKCPECGKSFSQL |
| | | 875) | | AHLRAHQRTHTGEKPYKCPECGKSFSDCRDLARHQRTHTGEKPY |
| | | | | KCPECGKSFSRADNLTEHQRTHTGEKPYKCPECGKSFSDPGHLV |
| | | | | RHQRTHTGEKPYKCPECGKSFSQLAHLRAHQRTHTGEKPYKCPE |
| | | | | CGKSFSQSGDLRRHQRTHTGEKPYKCPECGKSFSTSGHLVRHQR |
| | | | | THTGEKPYKCPECGKSFSRSDHLTTHQRTHTGKKTS (SEQ ID |
| | | | | NO: 892) |
| 3′ | ZnF15R | CGCATGCTGATTCAGC | 58.41 | LEPGEKPYKCPECGKSFSDPGNLVRHQRTHTGEKPYKCPECGKS |
| | | CTCCTGAC (SEQ ID | | FSTKNSLTEHQRTHTGEKPYKCPECGKSFSTKNSLTEHQRTHTGE |
| | | NO: 876) | | KPYKCPECGKSFSRADNLTEHQRTHTGEKPYKCPECGKSFSHKN |
| | | | | ALQNHQRTHTGEKPYKCPECGKSFSRNDALTEHQRTHTGEKPYK |
| | | | | CPECGKSFSRRDELNVHQRTHTGEKPYKCPECGKSFSHTGHLLE |
| | | | | HQRTHTGKKTS (SEQ ID NO: 893) |
| 3′ | ZnF14R | AGTCAAGCAACAGGAT | 50.89 | LEPGEKPYKCPECGKSFSQAGHLASHQRTHTGEKPYKCPECGKS |
| | | GA (SEQ ID NO: 877) | | FSQRAHLERHQRTHTGEKPYKCPECGKSFSSPADLTRHQRTHTG |
| | | | | EKPYKCPECGKSFSQSGDLRRHQRTHTGEKPYKCPECGKSFSQS |
| | | | | GNLTEHQRTHTGEKPYKCPECGKSFSHRTTLTNHQRTHTGKKTS |
| | | | | (SEQ ID NO: 894) |
| 3 | ZnF15R | GTCAAGCAACAGGATG | 59.22 | LEPGEKPYKCPECGKSFSHKNALQNHQRTHTGEKPYKCPECGKS |
| | | ATCCAAATGCTATT | | FSTSGELVRHQRTHTGEKPYKCPECGKSFSTTGNLTVHQRTHTG |
| | | (SEQ ID NO: 878) | | EKPYKCPECGKSFSTSHSLTEHQRTHTGEKPYKCPECGKSFSTS |
| | | | | GNLVRHQRTHTGEKPYKCPECGKSFSTSGNLVRHQRTHTGEKPY |
| | | | | KCPECGKSFSRADNLTEHQRTHTGEKPYKCPECGKSFSQSGNLT |
| | | | | EHQRTHTGEKPYKCPECGKSFSRKDNLKNHQRTHTGEKPYKCPE |
| | | | | CGKSFSDPGALVRHQRTHTGKKTS (SEQ ID NO: 895) |

No Sequences have Target site overlap (TSO)
Available on the world wide web at scripps.edu/barbas/zfdesign/searchsequence.php

TABLE 17
Zinc finger sequences targeting chromosome X (HPRT) (hg38 chrX:134,475,809-134,476,794)..

| ChrX | | | | |
|---|---|---|---|---|
| TTAA | NAME | TARGET | SCORE | ZFP AMINO ACID CODE |
| 5′ | ZnF41F | GTAGAAACTCGCCTTATG (SEQ | 54.04 | LEPGEKPYKCPECGKSFSRRDELNVHQRTHTGEK |
| | | ID NO: 896) | | PYKCPECGKSFSTTGALTEHQRTHTGEKPYKCPE |
| | | | | CGKSFSHTGHLLEHQRTHTGEKPYKCPECGKSFS |
| | | | | THLDLIRHQRTHTGEKPYKCPECGKSFSQSSNLV |
| | | | | RHQRTHTGEKPYKCPECGKSFSQSSSLVRHQRT |
| | | | | HTGKKTS (SEQ ID NO: 904) |
| 5′ | ZnF42F | TGAATGAGTCCTGTCCATCTT | 55.08 | LEPGEKPYKCPECGKSFSTTGALTEHQRTHTGEK |
| | | (SEQ ID NO: 897) | | PYKCPECGKSFSTSGNLTEHQRTHTGEKPYKCPE |
| | | | | CGKSFSDPGALVRHQRTHTGEKPYKCPECGKSFS |
| | | | | TKNSLTEHQRTHTGEKPYKCPECGKSFSHRTTLT |
| | | | | NHQRTHTGEKPYKCPECGKSFSRRDELNVHQRT |
| | | | | HTGEKPYKCPECGKSFSQAGHLASHQRTHTGKKT |
| | | | | S (SEQ ID NO: 905) |
| 5′ | ZnF43F | AAGATTAGAACAAATGTCCAG | 60.20 | LEPGEKPYKCPECGKSFSRADNLTEHQRTHTGEK |
| | | (SEQ ID NO: 898) | | PYKCPECGKSFSDPGALVRHQRTHTGEKPYKCPE |
| | | | | CGKSFSTTGNLTVHQRTHTGEKPYKCPECGKSFS |
| | | | | SPADLTRHQRTHTGEKPYKCPECGKSFSQLAHLR |
| | | | | AHQRTHTGEKPYKCPECGKSFSHKNALQNHQRT |
| | | | | HTGEKPYKCPECGKSFSRKDNLKNHQRTHTGKKT |
| | | | | S (SEQ ID NO: 906) |
| 3′ | ZnF44R | ACTCTAAGCAGCAATGTA (SEQ | 59.94 | LEPGEKPYKCPECGKSFSQSSSLVRHQRTHTGEK |
| | | ID NO: 899) | | PYKCPECGKSFSTTGNLTVHQRTHTGEKPYKCPE |
| | | | | CGKSFSERSHLREHQRTHTGEKPYKCPECGKSFS |
| | | | | ERSHLREHQRTHTGEKPYKCPECGKSFSQNSTLT |
| | | | | EHQRTHTGEKPYKCPECGKSFSTHLDLIRHQRTH |
| | | | | TGKKTS (SEQ ID NO: 907) |
| 5′ | ZnF45R | TGGGATAGTGAAAATGTC (SEQ | 57.10 | LEPGEKPYKCPECGKSFSDPGALVRHQRTHTGEK |
| | | ID NO: 900) | | PYKCPECGKSFSTTGNLTVHQRTHTGEKPYKCPE |
| | | | | CGKSFSQSSNLVRHQRTHTGEKPYKCPECGKSFS |
| | | | | HRTTLTNHQRTHTGEKPYKCPECGKSFSTSGNLV |
| | | | | RHQRTHTGEKPYKCPECGKSFSRSDHLTTHQRTH |
| | | | | TGKKTS (SEQ ID NO: 908) |
| 5′ | ZnF46R | AAAACTTGGGTCACTAAAATAGA | 61.20 | LEPGEKPYKCPECGKSFSTSGNLVRHQRTHTGEK |
| | | TGAT (SEQ ID NO: 901) | | PYKCPECGKSFSTSGNLVRHQRTHTGEKPYKCPE |
| | | | | CGKSFSQKSSLIAHQRTHTGEKPYKCPECGKSFS |
| | | | | QRANLRAHQRTHTGEKPYKCPECGKSFSTHLDLI |
| | | | | RHQRTHTGEKPYKCPECGKSFSDPGALVRHQRT |
| | | | | HTGEKPYKCPECGKSFSRSDHLTTHQRTHTGEKP |
| | | | | YKCPECGKSFSTHLDLIRHQRTHTGEKPYKCPEC |
| | | | | GKSFSQRANLRAHQRTHTGKKTS (SEQ ID NO: |
| | | | | 909) |
| 5′ | ZnF47R | AAACATGGAAAAGGTCAAAAAC | 43.59 | LEPGEKPYKCPECGKSFSRSDKLVRHQRTHTGEK |
| | | TTGGG (SEQ ID NO: 902) | | PYKCPECGKSFSTTGALTEHQRTHTGEKPYKCPE |
| | | | | CGKSFSQRANLRAHQRTHTGEKPYKCPECGKSF |
| | | | | SQSGNLTEHQRTHTGEKPYKCPECGKSFSTSGHL |
| | | | | VRHQRTHTGEKPYKCPECGKSFSQRANLRAHQR |
| | | | | THTGEKPYKCPECGKSFSQRAHLERHQRTHTGEK |
| | | | | PYKCPECGKSFSTSGNLTEHQRTHTGEKPYKCPE |
| | | | | CGKSFSQRANLRAHQRTHTGKKTS (SEQ ID NO: |
| | | | | 910) |
| 3′ | ZnF48R | AATGACTAGAATGAAGTCCTACT | 59.44 | LEPGEKPYKCPECGKSFSRNDALTEHQRTHTGEK |
| | | G (SEQ ID NO: 903) | | PYKCPECGKSFSQNSTLTEHQRTHTGEKPYKCPE |
| | | | | CGKSFSDPGALVRHQRTHTGEKPYKCPECGKSFS |
| | | | | QSSNLVRHQRTHTGEKPYKCPECGKSFSTTGNLT |
| | | | | VHQRTHTGEKPYKCPECGKSFSREDNLHTHQRT |
| | | | | HTGEKPYKCPECGKSFSDPGNLVRHQRTHTGEK |
| | | | | PYKCPECGKSFSTTGNLTVHQRTHTGKKTS (SEQ |
| | | | | ID NO: 911) |

No Sequences have Target site overlap (TSO)
Available on the world wide web at scripps. edu/barbas/zfdesign/searchsequence. php

In embodiments, the present disclosure relates to a system having nucleic acids encoding the enzyme (e.g., without limitation, the helper enzyme) and the donor DNA, respectively.

### Linkers

In embodiments, the targeting element comprises a nucleic acid binding component of a gene-editing system. In embodiments, the helper enzyme the targeting element are connected. Without wishing to be bound by a particular theory, the targeting element may refer to a nucleic acid binding component of the gene-editing system. In embodiments, the helper enzyme and the targeting element are connected. For example, in embodiments, the helper enzyme and the targeting element are fused to one another or linked via a linker (e.g., original linker AKLAGGAPAVGGGPKAADKFAATGGS (SEQ ID NO: 913) to one another.

In embodiments, the linker is a flexible linker. In embodiments, the flexible linker is substantially comprised of glycine and serine residues, optionally wherein the flexible linker comprises (Gly₄Ser)ₙ, where n is an integer from 1 to 12. In embodiments, the flexible linker is of about 20, or about 30, or about 40, or about 50, or about 60 amino acid residues. In embodiments, the flexible linker is about 50, or about 100, or about 150, or about 200 amino acid residues in length. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

### Inteins

Inteins (INTervening protEINS) are mobile genetic elements that are protein domains, found in nature, with the capability to carry out the process of protein splicing. See Sarmiento & Camarero (2019) Curr. Protein Pept. Sci., 20 (5), 408-424, which is incorporated by reference herein in its entirety. Protein spicing is a post-translation biochemical modification which results in the cleavage and formation of peptide bonds between precursor polypeptide segments flanking the intein. Id. Inteins apply standard enzymatic strategies to excise themselves post-translationally from a precursor protein via protein splicing. Nanda A, Nasker S S, Mehra A, Panda S, Nayak S. Inteins in Science: Evolution to Application. Microorganisms. 2020; 8 (12): 2004. An intein can splice its flanking N- and C-terminal domains to become a mature protein and excise itself from a sequence. For example, split inteins have been used to control the delivery of heterologous genes into transgenic organisms. See Wood & Camarero (2014) J. Biol. Chem. 289 (21): 14512-14519. This approach relies on splitting the target protein into two segments, which are then post-translationally reconstituted in vivo by protein trans-splicing (PTS). See Aboye & Camarero (2012) J. Biol. Chem. 287, 27026-27032. More recently, an intein-mediated split-Cas9 system has been developed to incorporate Cas9 into cells and reconstitute nuclease activity efficiently. Truong et al., Nucleic Acids Res. 2015, 43 (13), 6450-6458. The protein splicing excises the internal region of the precursor protein, which is then followed by the ligation of the N-extein and C-extein fragments, resulting in two polypeptides—the excised intein and the new polypeptide produced by joining the C- and N-exteins. Sarmiento & Camarero (2019) Curr. Protein Pept. Sci., 20 (5), 408-424.

In embodiments, intein-mediated incorporation of DNA binders such as, without limitation, dCas9, dCasX, dCas12j, TALEs, or ZnF, allows creation of a split-enzyme system such as, without limitation, split helper system, that permits reconstitution of the full-length enzyme, e.g., helper, from two smaller fragments. This allows avoiding the need to express DNA binders at the N- or C-terminus of an enzyme, e.g., helper. In this approach, the two portions of an enzyme, e.g., helper, are fused to the intein and, after co-expression, the intein allows producing a full-length enzyme, e.g., helper, by post-translation modification. Thus, in embodiments, a nucleic acid encoding the enzyme capable of targeted genomic integration by transposition comprises an intein. In embodiments, the nucleic acid encodes the enzyme in the form of first and second portions with the intein encoded between the first and second portions, such that the first and second portions are fused into a functional enzyme upon post-translational excision of the intein from the enzyme.

In embodiments, an intein is a suitable ligand-dependent intein, for example, an intein selected from those described in U.S. Pat. No. 9,200,045; Mootz et al., J. Am. Chem. Soc. 2002; 124, 9044-9045; Mootz et al., J. Am. Chem. Soc. 2003; 125, 10561-10569; Buskirk et al., PNAS 2004; 101, 10505-10510; Skretas & Wood. Protein Sci. 2005; 14, 523-532; Schwartz, et al., Nat. Chem. Biol. 2007; 3, 50-54; Peck et al., Chem. Biol. 2011; 18 (5), 619-630; the entire contents of each of which are hereby incorporated by reference herein.

In embodiments the intein is NpuN (Intein-N) (SEQ ID NO: 423) and/or NpuC (Intein-C) (SEQ ID NO: 424), or a variant thereof, e.g., a sequence having at least about 90%, or at least about 93%, or at least about 95%, or at least about 97%, or at least about 98%, or at least about 99% identity thereto.

SEQ ID NO: 423: nucleotide sequence of NpuN (Intein-N)
GGCGGATCTGGCGGTAGTGCTGAGTATTGTCTGAGTTACGAAACGGAAATACTCACGGTTGAGTATGGGCTTCTTCC
AATTGGCAAAATCGTTGAAAAGCGCATAGAGTGTACGGTGTATTCCGTCGATAACAACGGTAATATCTACACCCAGC
CGGTAGCTCAGTGGCACGACCGAGGCGAACAGGAAGTGTTCGAGTATTGCTTGGAAGATGGCTCCCTTATCCGCGCC
ACTAAAGACCATAAGTTTATGACGGTTGACGGGCAGATGCTGCCTATAGACGAAATATTTGAGAGAGAGCTGGACTT
GATGAGAGTCGATAATCTGCCAAAT
SEQ ID NO: 424: nucleotide sequence of NpuC (Intein-C)
GGCGGATCTGGCGGTAGTGGGGGTTCCGGATCCATAAAGATAGCTACTAGGAAATATCTTGGCAAACAAAACGTCTA
TGACATAGGAGTTGAGCGAGATCACAATTTTGCTTTGAAGAATGGGTTCATCGCGTCTAATTGCTTCAACGCTAGCG
GCGGGTCAGGAGGCTCTGGTGGAAGC

### Dimerization Enhancers

In embodiments, a nucleic acid encoding the enzyme capable of targeted genomic integration by transposition comprises a dimerization enhancer. In embodiments, the nucleic acid encodes the enzyme in the form of first and second portions with the dimerization enhancer encoded between the first and second portions, such that the first and second portions are fused into a functional enzyme upon post-translational excision of the dimerization enhancer from the enzyme. In embodiments, the dimerization enhancer is suitable for linking the helper enzyme and the targeting element. In embodiments, the dimerization enhancer is selected from: a protein comprising a SRC Homology 3 Domain (or SH3 domain), biotin, avidin, or a rapamycin binder, optionally, wherein the rapamycin binder is FKBP12 or mTOR, or a variant thereof.

### Nucleic Acids of the Disclosure

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof.

In embodiments, there is provided a donor construct comprising a heterologous polynucleotide between left and right transposon ends, wherein the left end comprises SEQ ID NO: 3, or a functional variant thereof and the right end comprises SEQ ID NO: 4, or a functional variant thereof, wherein the heterologous polynucleotide is transposable by a helper enzyme having the sequence of SEQ ID NO: 1, or a functional variant thereof.

In embodiments, there is provided a polynucleotide comprising an open reading frame encoding a helper enzyme which is at least 90% identical to SEQ ID NO: 2, or a functional variant thereof, operably linked to a heterologous promoter.

In embodiments, there is provided a polynucleotide comprising an open reading frame encoding a transposase, the amino acid sequence of which is at least 90% identical to SEQ ID NO: 1, or a functional variant thereof, operably linked to a heterologous promoter.

In embodiments, a nucleic acid encoding the enzyme (e.g., without limitation, the helper enzyme) is RNA. In embodiments, a nucleic acid encoding the transgene is DNA.

In embodiments, the enzyme (e.g., without limitation, the helper enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine), and optionally a poly-A tail of about 30, or about 50, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a 5′-UTR comprising a sequence that increases RNA stability in vivo, a 3′-UTR comprising a sequence that increases RNA stability in vivo, and/or a 3′ poly(A) tail.

In embodiments, the enzyme (e.g., without limitation, a helper) is incorporated into a vector or a vector-like particle. In embodiments, the vector is a non-viral vector.

In embodiments, a nucleic acid encoding the enzyme in accordance with embodiments of the present disclosure, is DNA.

In various embodiments, a construct comprising a donor is any suitable genetic construct, such as a nucleic acid construct, a plasmid, or a vector. In various embodiments, the construct is DNA, which is referred to herein as a donor DNA. In embodiments, sequences of a nucleic acid encoding the donor is codon optimized to provide improved mRNA stability and protein expression in mammalian systems.

In embodiments, the enzyme and the donor are included in different vectors. In embodiments, the enzyme and the donor are included in the same vector.

In various embodiments, a nucleic acid encoding the enzyme capable of targeted genomic integration by transposition (e.g., without limitation, the helper enzyme) is RNA (e.g., helper RNA), and a nucleic acid encoding a donor is DNA.

As would be appreciated in the art, a donor often includes an open reading frame that encodes a transgene at the middle of donor and terminal repeat sequences at the 5′ and 3′ end of the donor. The translated helper (e.g., without limitation, the helper enzyme) binds to the 5′ and 3′ sequence of the donor and carries out the transposition function.

In embodiments, a donor is used interchangeably with transposable elements, which are used to refer to polynucleotides capable of inserting copies of themselves into other polynucleotides. The term donor is well known to those skilled in the art and includes classes of donors that can be distinguished on the basis of sequence organization, for example inverted terminal sequences at each end, and/or directly repeated long terminal repeats (LTRs) at the ends. In embodiments, the donor as described herein may be described as a piggyBac like element, e.g., a donor element that is characterized by its traceless excision, which recognizes TTAA (SEQ ID NO: 440) sequence and restores the sequence at the insert site back to the original TTAA (SEQ ID NO: 440) sequence after removal of the donor.

In embodiments, the donor is flanked by one or more end sequences or terminal ends. In embodiments, the donor is or comprises a gene encoding a complete polypeptide. In embodiments, the donor is or comprises a gene which is defective or substantially absent in a disease state.

In embodiments, a transgene is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the donor (transgene cassette) to reduce transcriptional silencing and position effects imparted by chromosomal sequences. As an additional effect, the insulators can eliminate functional interactions of the transgene enhancer and promoter sequences with neighboring chromosomal sequences. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facioscapulohumeral muscular dystrophy (FSHD). In embodiments, the sequences of the HS4 insulator and the D4Z4 insulator are as described in Rival-Gervier et al. Mol Ther. 2013 August; 21 (8): 1536-50, which is incorporated herein by reference in its entirety.

In embodiments, the transgene is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: (1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat. Biotechnol. 2011; 29:73-8; Bejerano et al. Science 2004; 304:1321-5.

Furthermore, the use of GSHS locations can allow stable transgene expression across multiple cell types. One such site, chemokine C—C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz et al., Viruses 2010; 2:1069-105. A clinical trial has demonstrated safety and efficacy of disrupting CCR5 via targetable nucleases. Tebas et al., HIV. N Engl J Med 2014; 370:901-10.

In embodiments, the donor is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107 (7): 2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from donors, from yeast episomes, from insertion elements, from yeast chromosomal elements, and vectors from combinations thereof. The present constructs may contain control regions that regulate as well as engender expression.

In embodiments, the construct comprising the enzyme and/or transgene is codon optimized. Transgene codon optimization is used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the enzyme and/or transgene includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facioscapulohumeral muscular dystrophy (FSHD). In embodiments, the sequences of the HS4 insulator and the D4Z4 insulator are as described in Rival-Gervier et al. Mol Ther. 2013 August; 21 (8): 1536-50, which is incorporated herein by reference in its entirety. In embodiments, the gene of the construct comprising the enzyme and/or transgene is capable of transposition in the presence of a helper. In embodiments, the non-viral vector in accordance with embodiments of the present disclosure comprises a nucleic acid construct encoding a helper. The helper (e.g., without limitation, the helper enzyme of the present disclosure) is an RNA helper plasmid. In embodiments, the non-viral vector further comprises a nucleic acid construct encoding a DNA helper plasmid. In embodiments, the helper is an in vitro-transcribed mRNA helper. The helper (e.g., without limitation, the helper enzyme of the present disclosure) is capable of excising and/or transposing the gene from the construct comprising the enzyme and/or transgene to site- or locus-specific genomic regions.

In embodiments, the enzyme (e.g., without limitation, the helper enzyme) and the donor are included in the same vector. In embodiments, the enzyme is disposed on the same (cis) or different vector (trans) than a donor with a transgene. Accordingly, in embodiments, the enzyme and the donor encompassing a transgene are in cis configuration such that they are included in the same vector. In embodiments, the enzyme and the donor encompassing a transgene are in trans configuration such that they are included in different vectors. The vector is any non-viral vector in accordance with the present disclosure.

In aspects, a nucleic acid encoding the donor system of the present disclosure capable of targeted genomic integration by transposition (e.g., a helper) in accordance with embodiments of the present disclosure is provided. The nucleic acid is or comprises DNA or RNA. In embodiments, the nucleic acid encoding the enzyme is DNA. In embodiments, the nucleic acid encoding the enzyme capable of targeted genomic integration by transposition (e.g., a helper of the present disclosure) is RNA such as, e.g., helper RNA. In embodiments, the helper is incorporated into a vector. In embodiments, the vector is a non-viral vector.

In embodiments, a nucleic acid encoding the transgene in accordance with embodiments of the present disclosure is provided. The nucleic acid is or comprises DNA or RNA. In embodiments, the nucleic acid encoding the transgene is DNA. In embodiments, the nucleic acid encoding the transgene is RNA such as, e.g., helper RNA. In embodiments, the transgene is incorporated into a vector. In embodiments, the vector is a non-viral vector.

In embodiments, the present enzyme can be in the form or an RNA or DNA and have one or two N-terminus nuclear localization signal (NLS) to shuttle the protein more efficiently into the nucleus. For example, in embodiments, the present enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K (K/R) X (K/R). In an embodiment, the NLS comprises the consensus sequence (K/R) (K/R) X10-12 (K/R) 3/5l, where (K/R) 3/5 represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions, or deletions (e.g., about 1, or about 2, or about 3, or about 4, or about 5, or about 10 substitutions, additions, or deletions). In aspects, a host cell comprising the nucleic acid in accordance with embodiments of the present disclosure is provided.

### Lipids and LNP Delivery

In embodiments, a composition or a nucleic acid in accordance with embodiments of the present disclosure is provided wherein the composition is in the form of a lipid nanoparticle (LNP). In embodiments, the composition is encapsulated in an LNP.

In embodiments, a nucleic acid encoding the enzyme and a nucleic acid encoding the transgene are contained within the same lipid nanoparticle (LNP). In embodiments, the nucleic acid encoding the enzyme and the nucleic acid encoding the donor are a mixture incorporated into or associated with the same LNP. In embodiments, the polynucleotide encoding the helper enzyme and the polynucleotide encoding the donor are in the form of the same LNP, optionally in a co-formulation.

In embodiments, the LNP is selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-8 carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly (lactic-co-glycolic acid) (PLGA), and N-Acetylgalactosamine (GalNAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In aspects, the cell in accordance with the present disclosure is prepared via an in vivo genetic modification method. In embodiments, a genetic modification in accordance with the present disclosure is performed via an ex vivo method.

In aspects, the cell in accordance with the present disclosure is prepared by contacting a cell with an enzyme capable of targeted genomic integration by transposition (e.g., without limitation, the helper enzyme) in vivo. In embodiments, the cell is contacted with the enzyme ex vivo.

In embodiments, the present method provides high specific targeting as compared to a method that does not use the helper enzyme with a target selector.

### Therapeutic Applications

In embodiments, the transgene of interest in accordance with embodiments of the present disclosure can encode various genes.

In embodiments, the helper enzyme and the donor are included in the same pharmaceutical composition.

In embodiments, the helper enzyme and the donor are included in different pharmaceutical compositions. In embodiments, the helper enzyme and the donor are co-transfected.

In embodiments the helper enzyme and the donor are transfected separately.

In embodiments, a transfected cell for gene therapy is provided, wherein the transfected cell is generated using the helper enzyme in accordance with embodiments of the present disclosure.

In embodiments, a method of delivering a cell therapy is provided, comprising administering to a patient in need thereof the transfected cell generated using the helper enzyme in accordance with embodiments of the present disclosure.

In embodiments, a method of treating a disease or condition using a cell therapy, comprising administering to a patient in need thereof the transfected cell generated using the helper enzyme in accordance with embodiments of the present disclosure.

In embodiments, the disease or condition may comprise cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs syndrome.

In embodiments, the disease or condition is or comprises an infectious disease. In embodiments, the infectious disease is a coronavirus infection, optionally selected from infection with SAR-COV, MERS-CoV, and SARS-CoV-2, or variants thereof.

In embodiments, the infectious disease is or comprises a disease comprising a viral infection, a parasitic infection, or a bacterial infection. In embodiments, the viral infection is caused by a virus of family Flaviviridae, a virus of family Picornaviridae, a virus of family Orthomyxoviridae, a virus of family Coronaviridae, a virus of family Retroviridae, a virus of family Paramyxoviridae, a virus of family Bunyaviridae, or a virus of family Reoviridae.

In embodiments, the virus of family Coronaviridae comprises a betacoronavirus or an alphacoronavirus, optionally wherein the betacoronavirus is selected from SARS-CoV-2, SARS-CoV, MERS-CoV, HCoV-HKU1, and HCoV-OC43, or the alphacoronavirus is selected from a HCoV-NL63 and HCoV-229E. In embodiments, the infectious disease comprises a coronavirus infection 2019 (COVID-19).

In embodiments, the method requires a single administration. In embodiments, the method requires a plurality of administrations.

### Isolated Cell

In aspects of the present disclosure, an isolated cell is provided that comprises the transfected cell in accordance with embodiments of the present disclosure, e.g., transfected with a helper and/or donor.

In aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell(s) to the patient. For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient, or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly [1,3-bis(carboxyphenoxy) propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79 (3-4): 141-152.

In embodiments, there is provided a method of transforming a cell using the construct comprising the ends and/or transgene described herein in the presence of a helper (e.g., without limitation, the helper enzyme) to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a chimpanzee, an elephant, a dog, a rabbit, and the like. When the organism is an insect, the organism may include, but is not limited to, a fruit fly, an ant, a mosquito, a bollworm, and the like.

### Definitions

The following definitions are used in connection with the disclosure disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of′ or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### Selected Sequences

In embodiments, the present disclosure provides for any of the sequence provided herein, including the below, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

| SEQ ID NO: 1: Amino acid sequence of a helper from Eptesicus fuscus (638 amino acids) | |
|---|---|
| 1 | MDKFSKDIES SDDEFYFENE EKSEKCNSDE SEFSEDASGD DEQIAGPSGT TERKKSLALP |
| 61 | KDLAESTDSD SDIEFIKAKR RRTIVYSSES DGDIGDIIEK SGIRPSESYV SRGKQEKEKW |
| 121 | TSTSVNDKEP SRIPFSTGQL HVGPQVPSGC ATPIDFFQLF FTETLIKNIT DETNEYARHK |
| 181 | ISQKELSQRS TWNNWKDVTI EEMKAFLGVI LNMGVLNHPN LQSYWSMDFE SHIPFFRSVF |
| 241 | KRERFLQIFW MLHLKNDQKS SKDLRTRTEK VNCFLSYLEM KFRERFCPGR EIAVDEAVVG |
| 301 | FKGKIHFITY NPKKPTKWGI RLYVLSDSKC GYVHSFVPYY GGITSETLVR PDLPFTSRIV |
| 361 | LELHERLKNS VPGSQGYHFF TDRYYTSVTL AKELFKEKTH LTGTIMPNRK DNPPVIKHPK |
| 421 | LMKGEIVAFR DENVMLLAWK DKRIVTMLST WDTSETESVE RRVRGGGKEI VLKPKVVTNY |
| 481 | TKFMGGVDIA DHYTGTYCFM RKTLKWWRKL FFWGLEVSVV NSYILYKECQ KRKNEKPITH |
| 541 | VKFIRKLVHD LVGEFRDGTL TSRGRLLSTN LEQRLDGKLH IITPHPNKKH KDCVVCSNRK |
| 601 | IKGGRRETIY ICETCECKPG LHVGECFKKY HTMKNYRD |
| SEQ ID NO: 2: Nucleotide sequence encoding the helper from Eptesicus fuscus (1869 nt) | |
| 1 | ATGGACAAGT TTTCCAAGGA CATTGAAAGC TCTGACGATG AATTTTACTT CGAGAACGAG |
| 61 | GAGAAAAGCG AGAAGTGTAA TTCCGATGAG TCCGAGTTTA GCGAGGACGC TAGCGGCGAC |
| 121 | GACGAGCAGA TCGCTGGACC CAGCGGGACC ACGGAGCGCA AAAAGAGCCT GGCTCTGCCT |
| 181 | AAAGACTTGG CCGAGAGTAC CGACAGCGAC TCCGATATCG AGTTCATCAA GGCCAAACGC |
| 241 | AGGCGCACAA TCGTGTACTC TTCCGAGAGC GACGGCGACA TCGGCGATAT TATCGAGAAA |
| 301 | AGCGGGATCC GGCCTTCCGA AAGCTACGTG TCTCGGGGCA AGCAGGAGAA GGAAAAGTGG |
| 361 | ACAAGCACCT CTGTGAACGA CAAAGAGCCT TCCAGAATCC CCTTCAGCAC CGGCCAGCTG |
| 421 | CATGTGGGCC CCCAGGTGCC CAGCGGCTGC GCCACTCCTA TCGACTTCTT CCAGCTGTTT |
| 661 | TTTACTGAGA CCCTGATCAA GAACATCACC GATGAGACAA ATGAGTACGC CAGGCACAAG |
| 541 | ATCTCTCAGA AGGAGCTGAG CCAGCGCAGT ACATGGAACA ACTGGAAGGA CGTGACCATC |
| 601 | GAAGAGATGA AGGCCTTCCT GGGCGTGATC CTGAATATGG GAGTGCTGAA CCATCCTAAT |
| 661 | CTGCAGTCCT ATTGGTCCAT GGATTTCGAG TCCCACATTC CATTCTTCAG GTCCGTGTTC |
| 721 | AAGCGCGAGC GTTTCCTGCA GATCTTCTGG ATGCTGCACC TGAAAAATGA CCAGAAGAGC |
| 781 | TCCAAGGACC TGCGGACACG GACTGAGAAG GTGAATTGTT TCCTGTCCTA CCTGGAGATG |
| 841 | AAATTCAGGG AGAGGTTTTG TCCCGGCCGG GAAATTGCCG TGGATGAGGC CGTGGTGGGC |
| 901 | TTCAAGGGCA AGATCCACTT CATCACCTAC AACCCAAAGA AGCCAACAAA GTGGGGCATC |
| 961 | CGGCTGTATG TCCTGAGTGA CTCCAAGTGT GGCTACGTGC ACAGCTTIGT GCCCTATTAT |
| 1021 | GGCGGCATCA CCTCCGAGAC CCTGGTGAGG CCCGACCTGC CTTTCACCTC TAGAATTGTG |
| 1081 | CTGGAGCTGC ATGAGCGGCT GAAGAACTCT GTGCCTGGCA GCCAGGGCTA CCATTTTTTC |
| 1141 | ACCGACAGGT ACTATACATC CGTTACCCTG GCCAAGGAAC TGTTCAAAGA AAAAACCCAC |
| 1201 | CTGACCGGCA CTATCATGCC CAACCGCAAG GACAACCCCC CTGTGATCAA ACATCCCAAA |
| 1261 | CTGATGAAGG GCGAGATCGT GGCCTTCAGA GACGAGAACG TCATGCTGCT GGCTTGGAAA |
| 1321 | GATAAGCGGA TCGTGACTAT GCTGTCTACA TGGGATACCT CCGAGACAGA GAGCGTTGAA |
| 1381 | CGGCGGGTGA GGGGTGGAGG CAAGGAGATC GTGCTGAAGC CAAAGGTGGT GACCAACTAC |
| 1441 | ACCAAGTTCA TGGGGGGAGT GGATATTGCA GACCATTACA CCGGCACCTA CTGTTTCATG |
| 1501 | CGGAAGACCC TGAAGTGGTG GCGGAAGCTG TTCTTCTGGG GGCTGGAGGT CAGCGTGGTG |
| 1561 | AACTCCTACA TCCTCTACAA GGAGTGCCAG AAGAGGAAGA ACGAGAAACC AATCACACAC |
| 1621 | GTGAAGTTTA TCAGGAAGCT GGTGCACGAC CTGGTGGGAG AGTTCCGCGA CGGCACCCTC |
| 1681 | ACCAGTCGGG GCCGGCTGCT GAGTACAAAC CTGGAGCAGA GGCTGGACGG AAAGCTGCAC |
| 1741 | ATTATCACTC CCCATCCAAA TAAGAAGCAC AAGGACTGCG TGGTCTGCAG CAACCGGAAG |
| 1801 | ATTAAAGGAG GGGGGGGGA AACCATTTAC ATTTGTGAGA CCTGCGAATG CAAGCCTGGC |
| 1861 | CTGCACGTG |
| SEQ ID NO: 3: Eptesicus fuscus Left ITR (200 bp) (excluding TTAA) | |
| 1 | ccttttgcac toggatgtcg agtgtgactc gacacggtta gcatcggtag cagctcgtat |
| 61 | gtcgagccac actcgacacg tagtttcacc gaggggggaa gggggatttt tgtctatttt |
| 121 | tccagtatct tttcttgttt tcattagcat gaaaggacaa gtaaaatgta aatgccgtct |
| 181 | caactgatgc caccacctaa |
| SEQ ID NO: 4: Eptesicus fuscus Right ITR (200 bp) (excluding TTAA) | |
| 1 | tgaaaaatta tagagattaa aattactctt tgaatgtatc aataatttga aatataaaaa |
| 61 | aatccaaata aataagtttg tatgaaaaga aactccagtt ttttattcta ctgccgcgct |
| 121 | ttgtaaaatc tggggtattt aaaaaattaa atcccgagta gaataaagga atcgagaaaa |
| 181 | aagcaagcga gtgcaaaggg |
| SEQ ID NO: 5: Nucleotide sequence of dead Cas9 DNA binding protein (5004 bp) | |
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 661 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGAC |
| SEQ ID NO: 6: Amino acid sequence of dead Cas9 DNA binding protein (1368 amino acids) | |
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVAA |
| 841 | IVPQSFLKDD SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD |
| SEQ ID NO: 7: Amino acid sequence of E. coli TniQ subdomain of TnsD (508 amino acids) | |
| 1 | MRNFPVPYSN ELIYSTIARA GVYQGIVSPK QLLDEVYGNR KVVATLGLPS HLGVIARHLH |
| 61 | QTGRYAVQQL IYEHTLFPLY APFVGKERRD EAIRLMEYQA QGAVHLMLGV AASRVKSDNR |
| 121 | FRYCPDCVAL QLNRYGEAFW QRDWYLPALP YCPKHGALVF FDRAVDDHRH QFWALGHTEL |
| 181 | LSDYPKDSLS QLTALAAYIA PLLDAPRAQE LSPSLEQWTL FYQRLAQDLG LTKSKHIRHD |
| 241 | LVAERVRQTF SDEALEKLDL KLAENKDTCW LKSIFRKHRK AFSYLQHSIV WQALLPKLTV |
| 301 | IEALQQASAL TEHSITTRPV SQSVQPNSED LSVKHKDWQQ LVHKYQGIKA ARQSLEGGVL |
| 361 | YAWLYRHDRD WLVHWNQQHQ QERLAPAPRV DWNQRDRIAV RQLLRIIKRL DSSLDHPRAT |
| 421 | SSWLLKQTPN GTSLAKNLQK LPLVALCLKR YSESVEDYQI RRISQAFIKL KQEDVELRRW |
| 481 | RLLRSATLSK ERITEEAQRF LEMVYGEE |
| SEQ ID NO: 441: Amino acid sequence of synthetic Eptesicus fuscus (638 amino acids) trans- | |
| posase showing serine residues (bold) that were mutated to, without wishing to be bound by | |
| theory, increase excision activity (EXC+). | |
| 1 | MDKFSKDIES SDDEFYFENE EKSEKCNSDE SEFSEDASGD DEQIAGPSGT TERKKSLALP |
| 61 | KDLAESTDSD SDIEFIKAKR RRTIVYSSES DGDIGDIIEK SGIRPSESYV SRGKQEKEKW |
| 121 | TSTSVNDKEP SRIPFSTGQL HVGPQVPSGC ATPIDFFQLF FTETLIKNIT DETNEYARHK |
| 181 | ISQKELSQRS TWNNWKDVTI EEMKAFLGVI LNMGVLNHPN LQSYWSMDFE SHIPFFRSVF |
| 241 | KRERFLQIFW MLHLKNDQKS SKDLRTRTEK VNCFLSYLEM KFRERFCPGR EIAVDEAVVG |
| 301 | FKGKIHFITY NPKKPTKWGI RLYVLSDSKC GYVHSFVPYY GGITSETLVR PDLPFTSRIV |
| 361 | LELHERLKNS VPGSQGYHFF TDRYYTSVTL AKELFKEKTH LTGTIMPNRK DNPPVIKHPK |
| 421 | LMKGEIVAFR DENVMLLAWK DKRIVTMLST WDTSETESVE RRVRGGGKEI VLKPKVVTNY |
| 481 | TKFMGGVDIA DHYTGTYCFM RKTLKWWRKL FFWGLEVSVV NSYILYKECQ KRKNEKPITH |
| 541 | VKFIRKLVHD LVGEFRDGTL TSRGRLLSTN LEQRLDGKLH IITPHPNKKH KDCVVCSNRK |
| 601 | IKGGRRETIY ICETCECKPG LHVGECFKKY HTMKNYRD |
| SEQ ID NO: 442: Amino acid sequence of synthetic Eptesicus fuscus (603 amino acids) trans- | |
| posase with N-terminus deletions of amino acid 2-36 (N1 EXC+). | |
| 1 | MASGDDEQIA GPSGTTERKK SLALPKDLAE STDSDSDIEF IKAKRRRTIV YSSESDGDIG |
| 61 | DIIEKSGIRP SESYVSRGKQ EKEKWTSTSV NDKEPSRIPF STGQLHVGPQ VPSGCATPID |
| 121 | FFQLFFTETL IKNITDETNE YARHKISQKE LSQRSTWNNW KDVTIEEMKA FLGVILNMGV |
| 181 | LNHPNLQSYW SMDFESHIPF FRSVFKRERF LQIFWMLHLK NDQKSSKDLR TRTEKVNCFL |
| 241 | SYLEMKFRER FCPGREIAVD EAVVGFKGKI HFITYNPKKP TKWGIRLYVL SDSKCGYVHS |
| 301 | FVPYYGGITS ETLVRPDLPF TSRIVLELHE RLKNSVPGSQ GYHFFTDRYY TSVTLAKELF |
| 361 | KEKTHLTGTI MPNRKDNPPV IKHPKLMKGE IVAFRDENVM LLAWKDKRIV TMLSTWDTSE |
| 421 | TESVERRVRG GGKEIVLKPK VVTNYTKFMG GVDIADHYTG TYCFMRKTLK WWRKLFFWGL |
| 481 | EVSVVNSYIL YKECQKRKNE KPITHVKFIR KLVHDLVGEF RDGTLTSRGR LLSTNLEQRL |
| 541 | DGKLHIITPH PNKKHKDCVV CSNRKIKGGR RETIYICETC ECKPGLHVGE CFKKYHTMKN |
| 601 | YRD |
| SEQ ID NO: 443: Nucleotide sequence of synthetic Eptesicus fuscus (1809 bp) transposase | |
| with N-terminus deletions of amino acid 2-36 (N1 EXC+). | |
| 1 | ATGGCTAGCG GCGACGACGA GCAGATCGCT GGACCCAGCG GGACCACGGA GCGCAAAAAG |
| 61 | AGCCTGGCTC TGCCTAAAGA CTTGGCCGAG AGTACCGACA GCGACTCCGA TATCGAGTTC |
| 121 | ATCAAGGCCA AACGCAGGCG CACAATCGTG TACTCTTCCG AGAGCGACGG CGACATCGGC |
| 181 | GATATTATCG AGAAAAGCGG GATCCGGCCT TCCGAAAGCT ACGTGTCTCG GGGCAAGCAG |
| 241 | GAGAAGGAAA AGTGGACAAG CACCTCTGTG AACGACAAAG AGCCTTCCAG AATCCCCTTC |
| 301 | AGCACCGGCC AGCTGCATGT GGGCCCCCAG GTGCCCAGCG GCTGCGCCAC TCCTATCGAC |
| 361 | TTCTTCCAGC TGTTTTTTAC TGAGACCCTG ATCAAGAACA TCACCGATGA GACAAATGAG |
| 421 | TACGCCAGGC ACAAGATCTC TCAGAAGGAG CTGAGCCAGC GCAGTACATG GAACAACTGG |
| 481 | AAGGACGTGA CCATCGAAGA GATGAAGGCC TTCCTGGGCG TGATCCTGAA TATGGGAGTG |
| 541 | CTGAACCATC CTAATCTGCA GTCCTATTGG TCCATGGATT TCGAGTCCCA CATTCCATTC |
| 601 | TTCAGGTCCG TGTTCAAGCG CGAGCGTTTC CTGCAGATCT TCTGGATGCT GCACCTGAAA |
| 661 | AATGACCAGA AGAGCTCCAA GGACCTGCGG ACACGGACTG AGAAGGTGAA TTGTTTCCTG |
| 721 | TCCTACCTGG AGATGAAATT CAGGGAGAGG TTTTGTCCCG GCCGGGAAAT TGCCGTGGAT |
| 781 | GAGGCCGTGG TGGGCTTCAA GGGCAAGATC CACTTCATCA CCTACAACCC AAAGAAGCCA |
| 841 | ACAAAGTGGG GCATCCGGCT GTATGTCCTG AGTGACTCCA AGTGTGGCTA CGTGCACAGC |
| 901 | TTTGTGCCCT ATTATGGCGG CATCACCTCC GAGACCCTGG TGAGGCCCGA CCTGCCTTTC |
| 961 | ACCTCTAGAA TTGTGCTGGA GCTGCATGAG CGGCTGAAGA ACTCTGTGCC TGGCAGCCAG |
| 1021 | GGCTACCATT TTTTCACCGA CAGGTACTAT ACATCCGTTA CCCTGGCCAA GGAACTGTTC |
| 1081 | AAAGAAAAAA CCCACCTGAC CGGCACTATC ATGCCCAACC GCAAGGACAA CCCCCCTGTG |
| 1141 | ATCAAACATC CCAAACTGAT GAAGGGCGAG ATCGTGGCCT TCAGAGACGA GAACGTCATG |
| 1201 | CTGCTGGCTT GGAAAGATAA GCGGATCGTG ACTATGCTGT CTACATGGGA TACCTCCGAG |
| 1261 | ACAGAGAGCG TTGAACGGCG GGTGAGGGGT GGAGGCAAGG AGATCGTGCT GAAGCCAAAG |
| 1321 | GTGGTGACCA ACTACACCAA GTTCATGGGC GGAGTGGATA TTGCAGACCA TTACACCGGC |
| 1381 | ACCTACTGTT TCATGCGGAA GACCCTGAAG TGGTGGCGGA AGCTGTTCTT CTGGGGGCTG |
| 1441 | GAGGTCAGCG TGGTGAACTC CTACATCCTC TACAAGGAGT GCCAGAAGAG GAAGAACGAG |
| 1501 | AAACCAATCA CACACGTGAA GTTTATCAGG AAGCTGGTGC ACGACCTGGT GGGAGAGTTC |
| 1561 | CGCGACGGCA CCCTCACCAG TCGGGGCCGG CTGCTGAGTA CAAACCTGGA GCAGAGGCTG |
| 1621 | GACGGAAAGC TGCACATTAT CACTCCCCAT CCAAATAAGA AGCACAAGGA CTGCGTGGTC |
| 1681 | TGCAGCAACC GGAAGATTAA AGGAGGGGGG CGGGAAACCA TTTACATTTG TGAGACCTGC |
| 1741 | GAATGCAAGC CTGGCCTGCA CGTGGGGGAG TGCTTCAAGA AGTACCACAC AATGAAAAAC |
| 1801 | TACAGGGAT |
| SEQ ID NO: 444: Amino acid sequence of synthetic Eptesicus fuscus (592 amino acids) tran- | |
| sposase with N-terminus deletions of amino acid 2-47 (N2 EXC+). | |
| 1 | MSGTTERKKS LALPKDLAES TDSDSDIEFI KAKRRRTIVY SSESDGDIGD IIEKSGIRPS |
| 61 | ESYVSRGKQE KEKWTSTSVN DKEPSRIPFS TGQLHVGPQV PSGCATPIDF FQLFFTETLI |
| 121 | KNITDETNEY ARHKISQKEL SQRSTWNNWK DVTIEEMKAF LGVILNMGVL NHPNLQSYWS |
| 181 | MDFESHIPFF RSVFKRERFL QIFWMLHLKN DQKSSKDLRT RTEKVNCFLS YLEMKFRERF |
| 241 | CPGREIAVDE AVVGFKGKIH FITYNPKKPT KWGIRLYVLS DSKCGYVHSF VPYYGGITSE |
| 301 | TLVRPDLPFT SRIVLELHER LKNSVPGSQG YHFFTDRYYT SVTLAKELFK EKTHLTGTIM |
| 361 | PNRKDNPPVI KHPKLMKGEI VAFRDENVML LAWKDKRIVT MLSTWDTSET ESVERRVRGG |
| 421 | GKEIVLKPKV VTNYTKFMGG VDIADHYTGT YCFMRKTLKW WRKLFFWGLE VSVVNSYILY |
| 481 | KECQKRKNEK PITHVKFIRK LVHDLVGEFR DGTLTSRGRL LSTNLEQRLD GKLHIITPHP |
| 541 | NKKHKDCVVC SNRKIKGGRR ETIYICETCE CKPGLHVGEC FKKYHTMKNY RD |
| SEQ ID NO: 445: Nucleotide sequence of synthetic Eptesicus fuscus (1776 bp) transposase | |
| with N-terminus deletions of amino acid 2-47 (N2 EXC+). | |
| 1 | ATGAGCGGGA CCACGGAGCG CAAAAAGAGC CTGGCTCTGC CTAAAGACTT GGCCGAGAGT |
| 61 | ACCGACAGCG ACTCCGATAT CGAGTTCATC AAGGCCAAAC GCAGGCGCAC AATCGTGTAC |
| 121 | TCTTCCGAGA GCGACGGCGA CATCGGCGAT ATTATCGAGA AAAGCGGGAT CCGGCCTTCC |
| 181 | GAAAGCTACG TGTCTCGGGG CAAGCAGGAG AAGGAAAAGT GGACAAGCAC CTCTGTGAAC |
| 241 | GACAAAGAGC CTTCCAGAAT CCCCTTCAGC ACCGGCCAGC TGCATGTGGG CCCCCAGGTG |
| 301 | CCCAGCGGCT GCGCCACTCC TATCGACTTC TTCCAGCTGT TTTTTACTGA GACCCTGATC |
| 361 | AAGAACATCA CCGATGAGAC AAATGAGTAC GCCAGGCACA AGATCTCTCA GAAGGAGCTG |
| 421 | AGCCAGCGCA GTACATGGAA CAACTGGAAG GACGTGACCA TCGAAGAGAT GAAGGCCTTC |
| 481 | CTGGGCGTGA TCCTGAATAT GGGAGTGCTG AACCATCCTA ATCTGCAGTC CTATTGGTCC |
| 541 | ATGGATTTCG AGTCCCACAT TCCATTCTTC AGGTCCGTGT TCAAGCGCGA GCGTTTCCTG |
| 601 | CAGATCTTCT GGATGCTGCA CCTGAAAAAT GACCAGAAGA GCTCCAAGGA CCTGCGGACA |
| 661 | CGGACTGAGA AGGTGAATTG TTTCCTGTCC TACCTGGAGA TGAAATTCAG GGAGAGGTTT |
| 721 | TGTCCCGGCC GGGAAATTGC CGTGGATGAG GCCGTGGTGG GCTTCAAGGG CAAGATCCAC |
| 781 | TTCATCACCT ACAACCCAAA GAAGCCAACA AAGTGGGGCA TCCGGCTGTA TGTCCTGAGT |
| 841 | GACTCCAAGT GTGGCTACGT GCACAGCTTT GTGCCCTATT ATGGCGGCAT CACCTCCGAG |
| 901 | ACCCTGGTGA GGCCCGACCT GCCTTTCACC TCTAGAATTG TGCTGGAGCT GCATGAGCGG |
| 961 | CTGAAGAACT CTGTGCCTGG CAGCCAGGGC TACCATTTTT TCACCGACAG GTACTATACA |
| 1021 | TCCGTTACCC TGGCCAAGGA ACTGTTCAAA GAAAAAACCC ACCTGACCGG CACTATCATG |
| 1081 | CCCAACCGCA AGGACAACCC CCCTGTGATC AAACATCCCA AACTGATGAA GGGCGAGATC |
| 1141 | GTGGCCTTCA GAGACGAGAA CGTCATGCTG CTGGCTTGGA AAGATAAGCG GATCGTGACT |
| 1201 | ATGCTGTCTA CATGGGATAC CTCCGAGACA GAGAGCGTTG AACGGCGGGT GAGGGGTGGA |
| 1261 | GGCAAGGAGA TCGTGCTGAA GCCAAAGGTG GTGACCAACT ACACCAAGTT CATGGGCGGA |
| 1321 | GTGGATATTG CAGACCATTA CACCGGCACC TACTGTTTCA TGCGGAAGAC CCTGAAGTGG |
| 1381 | TGGCGGAAGC TGTTCTTCTG GGGGCTGGAG GTCAGCGTGG TGAACTCCTA CATCCTCTAC |
| 1441 | AAGGAGTGCC AGAAGAGGAA GAACGAGAAA CCAATCACAC ACGTGAAGTT TATCAGGAAG |
| 1501 | CTGGTGCACG ACCTGGTGGG AGAGTTCCGC GACGGCACCC TCACCAGTCG GGGCCGGCTG |
| 1561 | CTGAGTACAA ACCTGGAGCA GAGGCTGGAC GGAAAGCTGC ACATTATCAC TCCCCATCCA |
| 1621 | AATAAGAAGC ACAAGGACTG CGTGGTCTGC AGCAACCGGA AGATTAAAGG AGGGCGGCGG |
| 1681 | GAAACCATTT ACATTTGTGA GACCTGCGAA TGCAAGCCTG GCCTGCACGT GGGGGAGTGC |
| 1741 | TTCAAGAAGT ACCACACAAT GAAAAACTAC AGGGAT |
| SEQ ID NO: 446: Amino acid sequence of synthetic Eptesicus fuscus (523 amino acids) trans- | |
| posase with N-terminus deletions of amino acid 2-117 (N3 EXC+). | |
| 1 | M-KWTSTSV NDKEPSRIPF STGQLHVGPQ VPSGCATPID FFQLFFTETL IKNITDETNE |
| 61 | YARHKISQKE LSQRSTWNNW KDVTIEEMKA FLGVILNMGV LNHPNLQSYW SMDFESHIPF |
| 121 | FRSVFKRERF LQIFWMLHLK NDQKSSKDLR TRTEKVNCFL SYLEMKFRER FCPGREIAVD |
| 181 | EAVVGFKGKI HFITYNPKKP TKWGIRLYVL SDSKCGYVHS FVPYYGGITS ETLVRPDLPF |
| 241 | TSRIVLELHE RLKNSVPGSQ GYHFFTDRYY TSVTLAKELF KEKTHLTGTI MPNRKDNPPV |
| 301 | IKHPKLMKGE IVAFRDENVM LLAWKDKRIV TMLSTWDTSE TESVERRVRG GGKEIVLKPK |
| 361 | VVTNYTKFMG GVDIADHYTG TYCFMRKTLK WWRKLFFWGL EVSVVNSYIL YKECQKRKNE |
| 421 | KPITHVKFIR KLVHDLVGEF RDGTLTSRGR LLSTNLEQRL DGKLHIITPH PNKKHKDCVV |
| 481 | CSNRKIKGGR RETIYICETC ECKPGLHVGE CFKKYHTMKN YRD |
| SEQ ID NO: 447: Nucleotide sequence of synthetic Eptesicus fuscus (1569 bp) transposase | |
| with N-terminus deletions of amino acid 2-117 (N3 EXC+). | |
| atggaaaagtggacaagcacctctgtgaacgacaaagagccttccagaatccccttcagcaccggccagctgcatgt | |
| gggcccccaggtgcccagcggctgcgccactcctatcgacttcttccagctgttttttactgagaccctgatcaaga | |
| acatcaccgatgagacaaatgagtacgccaggcacaagatctctcagaaggagctgagccagcgcagtacatggaac | |
| aactggaaggacgtgaccatcgaagagatgaaggccttcctgggcgtgatcctgaatatgggagtgctgaaccatcc | |
| taatctgcagtcctattggtccatggatttcgagtcccacattccattcttcaggtccgtgttcaagcgcgagcgtt | |
| tcctgcagatcttctggatgctgcacctgaaaaatgaccagaagagctccaaggacctgcggacacggactgagaag | |
| gtgaattgtttcctgtcctacctggagatgaaattcagggagaggttttgtcccggccgggaaattgccgtggatga | |
| ggccgtggtgggcttcaagggcaagatccacttcatcacctacaacccaaagaagccaacaaagtggggcatccggc | |
| tgtatgtcctgagtgactccaagtgtggctacgtgcacagctttgtgccctattatggcggcatcacctccgagacc | |
| ctggtgaggcccgacctgcctttcacctctagaattgtgctggagctgcatgagcggctgaagaactctgtgcctgg | |
| cagccagggctaccattttttcaccgacaggtactatacatccgttaccctggccaaggaactgttcaaagaaaaaa | |
| cccacctgaccggcactatcatgcccaaccgcaaggacaacccccctgtgatcaaacatcccaaactgatgaagggc | |
| gagatcgtggccttcagagacgagaacgtcatgctgctggcttggaaagataagcggatcgtgactatgctgtctac | |
| atgggatacctccgagacagagagcgttgaacggcgggtgaggggtggaggcaaggagatcgtgctgaagccaaagg | |
| tggtgaccaactacaccaagttcatgggcggagtggatattgcagaccattacaccggcacctactgtttcatgcgg | |
| aagaccctgaagtggtggcggaagctgttcttctgggggctggaggtcagcgtggtgaactcctacatcctctacaa | |
| ggagtgccagaagaggaagaacgagaaaccaatcacacacgtgaagtttatcaggaagctggtgcacgacctggtgg | |
| gagagttccgcgacggcaccctcaccagtcggggccggctgctgagtacaaacctggagcagaggctggacggaaag | |
| ctgcacattatcactccccatccaaataagaagcacaaggactgcgtggtctgcagcaaccggaagattaaaggagg | |
| gcggcgggaaaccatttacatttgtgagacctgcgaatgcaagcctggcctgcacgtgggggagtgcttcaagaagt | |
| accacacaatgaaaaactacagggattaa | |
| SEQ ID NO: 448: Amino acid sequence of synthetic Eptesicus fuscus (520 amino acids) trans- | |
| posase with N-terminus deletions of amino acid 2-120 (N4 EXC+). | |
| 1 | M-TSTSVNDK EPSRIPFSTG QLHVGPQVPS GCATPIDFFQ LFFTETLIKN ITDETNEYAR |
| 61 | HKISQKELSQ RSTWNNWKDV TIEEMKAFLG VILNMGVINH PNLQSYWSMD FESHIPFFRS |
| 121 | VFKRERFLQI FWMLHLKNDQ KSSKDLRTRT EKVNCFLSYL EMKFRERFCP GREIAVDEAV |
| 181 | VGFKGKIHFI TYNPKKPTKW GIRLYVLSDS KCGYVHSFVP YYGGITSETL VRPDLPFTSR |
| 241 | IVLELHERLK NSVPGSQGYH FFTDRYYTSV TLAKELFKEK THLTGTIMPN RKDNPPVIKH |
| 301 | PKLMKGEIVA FRDENVMLLA WKDKRIVTML STWDTSETES VERRVRGGGK EIVLKPKVVT |
| 361 | NYTKFMGGVD IADHYTGTYC FMRKTLKWWR KLFFWGLEVS VVNSYILYKE CQKRKNEKPI |
| 421 | THVKFIRKLV HDLVGEFRDG TLTSRGRLLS TNLEQRLDGK LHIITPHPNK KHKDCVVCSN |
| 481 | RKIKGGRRET IYICETCECK PGLHVGECFK KYHTMKNYRD |
| SEQ ID NO: 449: Nucleotide sequence of synthetic Eptesicus fuscus (1560 bp) transposase | |
| with N-terminus deletions of amino acid 2-120 (N4 EXC+). | |
| atgacaagcacctctgtgaacgacaaagagccttccagaatccccttcagcaccggccagctgcatgtgggccccca | |
| ggtgcccagcggctgcgccactcctatcgacttcttccagctgttttttactgagaccctgatcaagaacatcaccg | |
| atgagacaaatgagtacgccaggcacaagatctctcagaaggagctgagccagcgcagtacatggaacaactggaag | |
| gacgtgaccatcgaagagatgaaggccttcctgggcgtgatcctgaatatgggagtgctgaaccatcctaatctgca | |
| gtcctattggtccatggatttcgagtcccacattccattcttcaggtccgtgttcaagcgcgagcgtttcctgcaga | |
| tcttctggatgctgcacctgaaaaatgaccagaagagctccaaggacctgcggacacggactgagaaggtgaattgt | |
| ttcctgtcctacctggagatgaaattcagggagaggttttgtcccggccgggaaattgccgtggatgaggccgtggt | |
| gggcttcaagggcaagatccacttcatcacctacaacccaaagaagccaacaaagtggggcatccggctgtatgtcc | |
| tgagtgactccaagtgtggctacgtgcacagctttgtgccctattatggcggcatcacctccgagaccctggtgagg | |
| cccgacctgcctttcacctctagaattgtgctggagctgcatgagcggctgaagaactctgtgcctggcagccaggg | |
| ctaccattttttcaccgacaggtactatacatccgttaccctggccaaggaactgttcaaagaaaaaacccacctga | |
| ccggcactatcatgcccaaccgcaaggacaacccccctgtgatcaaacatcccaaactgatgaagggcgagatcgtg | |
| gccttcagagacgagaacgtcatgctgctggcttggaaagataagcggatcgtgactatgctgtctacatgggatac | |
| ctccgagacagagagcgttgaacggcgggtgaggggtggaggcaaggagatcgtgctgaagccaaaggtggtgacca | |
| actacaccaagttcatgggcggagtggatattgcagaccattacaccggcacctactgtttcatgcggaagaccctg | |
| aagtggtggcggaagctgttcttctgggggctggaggtcagcgtggtgaactcctacatcctctacaaggagtgcca | |
| gaagaggaagaacgagaaaccaatcacacacgtgaagtttatcaggaagctggtgcacgacctggtgggagagttcc | |
| gcgacggcaccctcaccagtcggggccggctgctgagtacaaacctggagcagaggctggacggaaagctgcacatt | |
| atcactccccatccaaataagaagcacaaggactgcgtggtctgcagcaaccggaagattaaaggagggcggcggga | |
| aaccatttacatttgtgagacctgcgaatgcaagcctggcctgcacgtgggggagtgcttcaagaagtaccacacaa | |
| tgaaaaactacagggattaa | |
| SEQ ID NO: 450: Amino acid sequence of synthetic Eptesicus fuscus (518 amino acids) trans- | |
| posase with N-terminus deletions of amino acid 2-122 (N5 EXC+). | |
| 1 | M-TSVNDKEP SRIPFSTGQL HVGPQVPSGC ATPIDFFQLF FTETLIKNIT DETNEYARHK |
| 61 | ISQKELSQRS TWNNWKDVTI EEMKAFLGVI LNMGVLNHPN LQSYWSMDFE SHIPFFRSVF |
| 121 | KRERFLQIFW MLHLKNDQKS SKDLRTRTEK VNCFLSYLEM KFRERFCPGR EIAVDEAVVG |
| 181 | FKGKIHFITY NPKKPTKWGI RLYVLSDSKC GYVHSFVPYY GGITSETLVR PDLPFTSRIV |
| 241 | LELHERLKNS VPGSQGYHFF TDRYYTSVTL AKELFKEKTH LTGTIMPNRK DNPPVIKHPK |
| 301 | LMKGEIVAFR DENVMLLAWK DKRIVTMLST WDTSETESVE RRVRGGGKEI VLKPKVVTNY |
| 361 | TKFMGGVDIA DHYTGTYCFM RKTLKWWRKL FFWGLEVSVV NSYILYKECQ KRKNEKPITH |
| 421 | VKFIRKLVHD LVGEFRDGTL TSRGRLLSTN LEQRLDGKLH IITPHPNKKH KDCVVCSNRK |
| 481 | IKGGRRETIY ICETCECKPG LHVGECFKKY HTMKNYRD |

SEQ ID NO: 451: Nucleotide sequence of synthetic Eptesicus fuscus (1554 bp) transposase
with N-terminus deletions of amino acid 2-122 (N5 EXC+).
atgacctctgtgaacgacaaagagccttccagaatccccttcagcaccggccagctgcatgtgggcccccaggtgcc
cagcggctgcgccactcctatcgacttcttccagctgttttttactgagaccctgatcaagaacatcaccgatgaga
caaatgagtacgccaggcacaagatctctcagaaggagctgagccagcgcagtacatggaacaactggaaggacgtg
accatcgaagagatgaaggccttcctgggcgtgatcctgaatatgggagtgctgaaccatcctaatctgcagtccta
ttggtccatggatttcgagtcccacattccattcttcaggtccgtgttcaagcgcgagcgtttcctgcagatcttct
ggatgctgcacctgaaaaatgaccagaagagctccaaggacctgcggacacggactgagaaggtgaattgtttcctg
tcctacctggagatgaaattcagggagaggttttgtcccggccgggaaattgccgtggatgaggccgtggtgggctt
caagggcaagatccacttcatcacctacaacccaaagaagccaacaaagtggggcatccggctgtatgtcctgagtg
actccaagtgtggctacgtgcacagctttgtgccctattatggcggcatcacctccgagaccctggtgaggcccgac
ctgcctttcacctctagaattgtgctggagctgcatgagcggctgaagaactctgtgcctggcagccagggctacca
ttttttcaccgacaggtactatacatccgttaccctggccaaggaactgttcaaagaaaaaacccacctgaccggca
ctatcatgcccaaccgcaaggacaacccccctgtgatcaaacatcccaaactgatgaagggcgagatcgtggccttc
agagacgagaacgtcatgctgctggcttggaaagataagcggatcgtgactatgctgtctacatgggatacctccga
gacagagagcgttgaacggcgggtgaggggtggaggcaaggagatcgtgctgaagccaaaggtggtgaccaactaca
ccaagttcatgggcggagtggatattgcagaccattacaccggcacctactgtttcatgcggaagaccctgaagtgg
tggcggaagctgttcttctgggggctggaggtcagcgtggtgaactcctacatcctctacaaggagtgccagaagag
gaagaacgagaaaccaatcacacacgtgaagtttatcaggaagctggtgcacgacctggtgggagagttccgcgacg
gcaccctcaccagtcggggccggctgctgagtacaaacctggagcagaggctggacggaaagctgcacattatcact
ccccatccaaataagaagcacaaggactgcgtggtctgcagcaaccggaagattaaaggagggcggcgggaaaccat
ttacatttgtgagacctgcgaatgcaagcctggcctgcacgtgggggagtgcttcaagaagtaccacacaatgaaaa
actacagggattaa

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1—Design of Transposon System

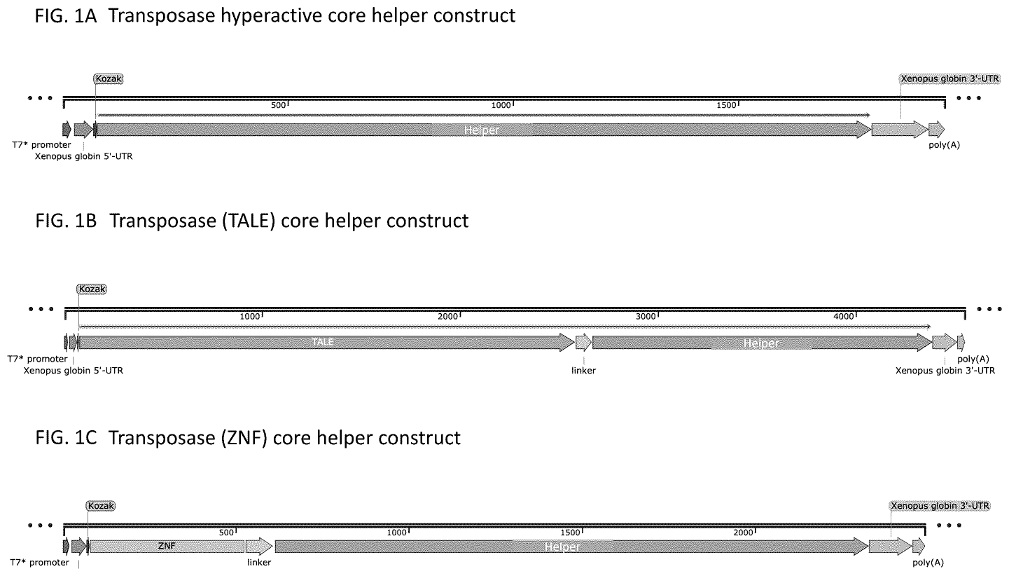

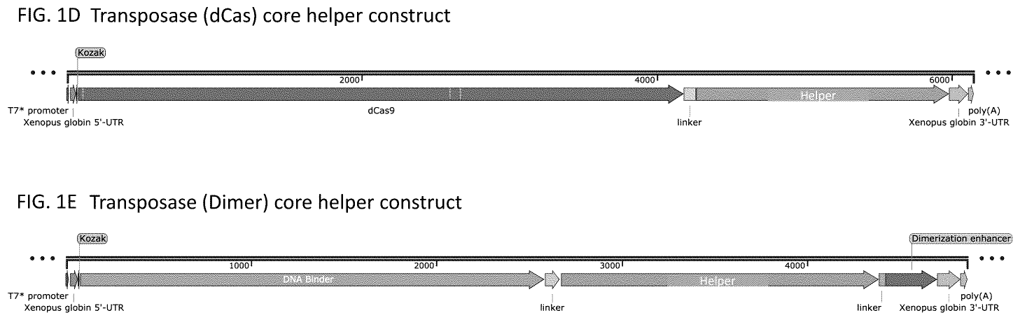

FIG. 1A-FIG. 1E depict five illustrative bioengineered RNA helper constructs that are contained in a replication backbone (e.g., plasmid or miniplasmid) with a T7 promoter (cap dependent), beta-globin 5′-UTR, and a helper enzyme (SEQ ID NO: 1, SEQ ID NO: 2) from Eptesicus fuscus followed by a beta-globin 3′-UTR, and a poly-alanine tail (FIG. 1A). TALEs (FIG. 1B, TABLE 7-TABLE 12), ZnF (FIG. 1C, TABLE 13-TABLE 17), or a dead Cas9 (dCas9) binding protein (FIG. 1D, SEQ ID NO: 5, SEQ ID NO: 6) with guide RNAs (TABLE 1-TABLE 6) were linked to the N-terminus to target the specific TTAA sites at hROSA 26, AAVS1, chromosome 4, chromosome 22, and chromosome X loci. FIG. 1E depicts a construct with a dimerization enhancer. The dimerization enhancer may be selected from, without limitation, SH3, biotin, avidin, and rapamycin binders. The dimerization enhancer can be replaced with an intein.

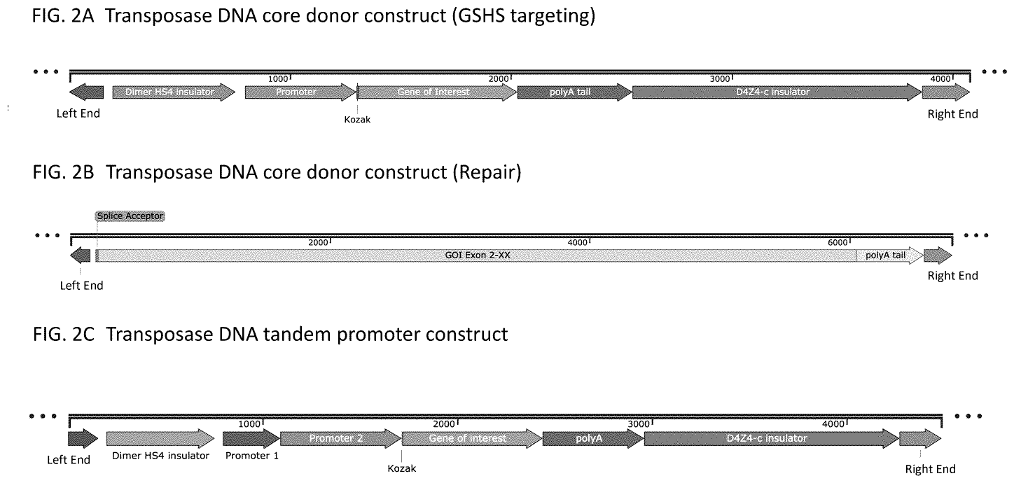

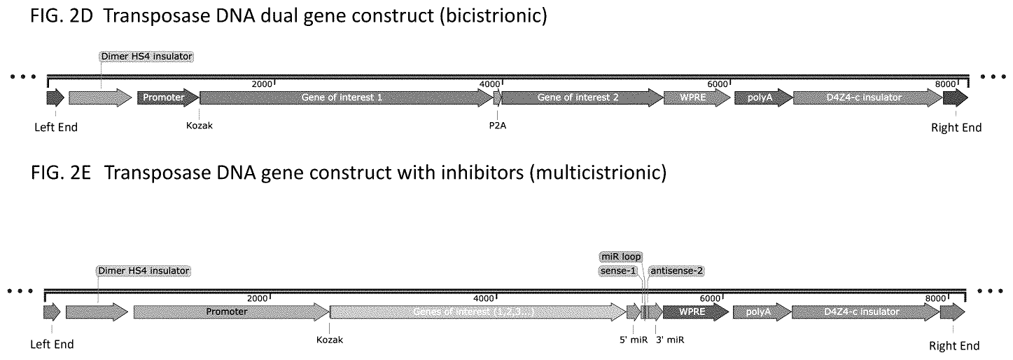

FIG. 2A depicts an illustrative core donor construct that is contained in a replication backbone (e.g., plasmid or miniplasmid) with a promoter driving a gene of interest (GOI) with a polyA tail flanked by two insulators and ITRs. The inverted terminal repeat (ITR) recognition sequences are included at the 5′-(SEQ ID NO: 3) and 3′-ends (SEQ ID NO: 4). This construct is used for targeting genomic safe harbor sites (GSHS) or other loci.

FIG. 2B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. The inverted terminal repeat (ITR) recognition sequences are included at the 5′-(SEQ ID NO: 3) and 3′-ends (SEQ ID NO: 4). This construct is used for targeting endogenous genes in the first intron (or other introns) to repair downstream mutations.

FIG. 2C depicts an illustrative core donor construct that is contained in a replication backbone (e.g., plasmid or miniplasmid) with tandem promoters to affect expression in different tissues (e.g., without limitation, liver specific promoter, cardiac specific promoter, retinal specific promoter, basal lung cell promoter) and a gene(s) of interest (GOI) followed by a polyA tail and flanked by ITRs. The inverted terminal repeat (ITR) recognition sequences are included at the 5′-(SEQ ID NO: 3) and 3′-ends (SEQ ID NO: 4). This construct is used to differentially promote expression of genes in different organs, tissues or cell types.

FIG. 2D depicts an illustrative core donor construct that is contained in a replication backbone (e.g., plasmid or miniplasmid) with two or more genes of interest (GOI) linked by 2A “self-cleaving” peptides and followed by WPRE and a polyA tail. The construct is flanked by ITRs. The inverted terminal repeat (ITR) recognition sequences are included at the 5′-(SEQ ID NO: 3) and 3′-ends (SEQ ID NO: 4). This construct is used for delivering multiple genes or genetic factors.

FIG. 2E depicts an illustrative core donor construct that is contained in a replication backbone (e.g., plasmid or miniplasmid) with a promoter(s) driving the expression of two or more genes as in FIG. 2D and linked to a sequence consisting of a 5′-miRNA, a sense and antisense miRNA pair, and completed with the 3′-miRNA. The construct is followed by WPRE and flanked by ITRs. The inverted terminal repeat (ITR) recognition sequences are included at the 5′-(SEQ ID NO: 3) and 3′-ends (SEQ ID NO: 4). This construct combines protein replacement and miRNA to inhibit other related protein expression. The sense and anti-sense miRNA pair regulate the sense miRNAs, probably via modulating the chromatin architectures of the resided genomic loci. See Brown, T., Howe, F. S., Murray, S. C., Wouters, M., Lorenz, P., Seward, E., . . . . Mellor, J. (2018). Antisense transcription-dependent chromatin signature modulates sense transcript dynamics. Mol Syst Biol, 14 (2), e8007; Murray, S. C., Haenni, S., Howe, F. S., Fischl, H., Chocian, K., Nair, A., & Mellor, J. (2015). Sense and antisense transcription are associated with distinct chromatin architectures across genes. Nucleic Acids Res, 43 (16), 7823-7837.

### Example 2—Identification of Excision Positive and Integration Negative Mutants

Random mutagenesis and/or site directed mutagenesis were performed on the Eptesicus fuscus helper enzyme of SEQ ID NO: 1. The variants were screened using integration and excision assays. The excision assay was a PCR-based assay to test for excision of the donor DNA. A HEK293 cell line that expresses GFP at a known genomic site were transfected with helper plasmid alone to excise the donor GFP DNA at the genomic locus by recognizing the end sequences. For the integration assay, HEK293 cells were plated in 12-well size plates the day before transfection. The day of the transfection the media was exchanged 1 hour and 30 min before the transfection was performed. A 3:1 ratio of X-tremeGENE™ 9 DNA Transfection Reagent protocol reagent was used to co-transfect a donor plasmid containing GFP and a helper plasmid in duplicate using 600 ng of DNA each. Forty-eight (48) hrs after transfection, the cells were analyzed by flow cytometry to count the percentage of GFP expressing cells to measure transient transfection efficiency. The cells were gated to distinguish them from debris and 20,000 cells were counted. The cultures were grown for 15-20 days without antibiotic. Cells were passaged 2 to 3 times per week. Flow cytometry was used to count the percentage of GFP expressing cells to measure integration efficiency at 2 weeks. The final integration efficiency were calculated by dividing the 2-week percentage of GFP cells by the percentage of GFP cell at 48 hr.

The excision assay were performed by measuring the percentage of GFP cells in a cell line with a known GFP donor integration. The cells were grown to 80% confluency and analyzed by flow cytometry to count the percentage of GFP expressing cells as a baseline measurement. This percentage was used as the standard (i.e., 100%). X-tremeGENE™ 9 DNA Transfection Reagent protocol reagent were used to transfect helper plasmid in duplicate using 600 ng of DNA. The cells were gated to distinguish them from debris and 20,000 cells were counted. Forty-eight (48) hrs after transfection, the cells were analyzed by flow cytometry to count the percentage of GFP expressing cells. The cells were gated to distinguish them from debris and 20,000 cells were counted. The final integration efficiency were calculated by the baseline percentage of GFP cells by the percentage of GFP cells at 48 hr.

Excision positive (EXC+) and integration deficient (INT−) mutants were identified using the method described above.

### Example 3—Identification of Deletion Mutants and Fusion Protein Mutants

Multiple deletion mutations were generated using known methods. Some deletion mutants were deleted at the N-terminus at varying number of residues relative to SEQ ID NO: 1. Some deletion mutants were deleted at the C-terminus at varying number of residues relative to SEQ ID NO: 1. Some deletion mutants were deleted in between the N-terminus and the C-terminus at varying number of residues relative to SEQ ID NO: 1. Some deletion mutants were deleted at the N-terminus and at the C-terminus. Some deletion mutants were deleted at the N-terminus, at the C-terminus, and in between the N-terminus and the C-terminus relative to SEQ ID NO: 1. Integration and excision activity were tested on the mutants. Mutants with high excision activity and low integration activity were selected as lead candidates for further optimization (e.g., without limitation, additional rounds of screening and/or addition of fusion proteins as described below).

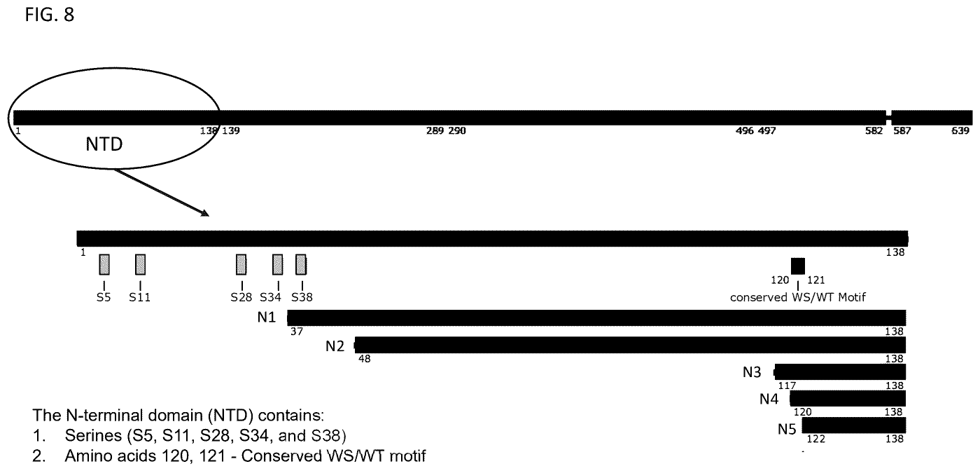

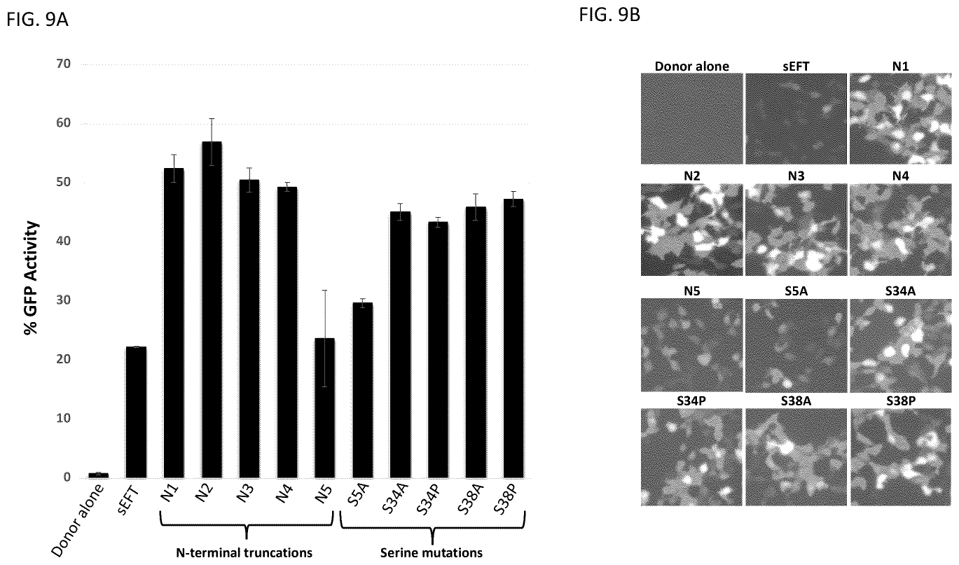

FIG. 8 represents a graphical representation of the structure of synthetic Eptisicus fuscus and Microcebus murinus transposase (sETF). The N-terminal domain (NTD) is circled and magnified to show the serine residues that are putative phosphorylation sites for transposases, without wishing to be bound by theory. The indicated serines are, without wishing to be bound by theory, target sites for phosphorylation by cellular Casein kinases. The N terminal domain was subject to series of truncations to release N-terminal mediated suppression of transposition activity. Conserved serine residues are shown in the N-terminus [S5, S11, S28, S34 and S38] which were mutated to alanine to, without wishing to be bound by theory, make it hyperactive (FIG. 8). The series of N-terminal deletions were made and named as N1 (A2-36) (SEQ ID NO: 442 and 443), N2 (A2-47) (SEQ ID NO: 444 and 445), N3 (A2-117) (SEQ ID NO: 446 and 447), N4 (A2-120) (SEQ ID NO: 448 and 449) and N5 (A2-122) (SEQ ID NO: 450 and 451). Alanine substitutions at these sites increased enzyme activity (FIG. 9B). Similarly, N-terminus deletions (N1-N4) before the conserved WS/WT motif also increased enzyme activity (FIG. 9B).

FIGS. 9A and 9B show the excision activity of sEFT as measured flow cytometry GFP expression in FIG. 9A and direct visualization of the transposed cells in FIG. 9B. The excision GFP reporter construct is a plasmid DNA construct where the GFP gene is separated by transposon with appropriate ends that are recognized by transposase. Without transposition activity, this construct does not produce any effective GFP protein due to disruption of the open reading frame. However, when excision happens by the transposase, the transposon is removed and the entire GFP protein is produced which results in green color.

Excision reporter construct was co-transfected with different helper variants in non-fluorescent HEK293T cells. The extent of excision activity for any construct was estimated by reconstructing the GFP reporter, which resulted in green fluorescence. The donor alone constructs [MLT-DO, BBT-DO] were used as negative controls (FIG. 9A). On the second day post transfection, cells underwent flow cytometric analysis to estimate the relative percentages of GFP producing cells. The mean fluorescence activity is presented by error bars with Standard Error of Mean [SEM] (B) (FIG. 9B).

All variants except for the deletion of amino acid residues 2-122 (N5) (SEQ ID NO. 450 and SEQ ID NO. 451) showed hyperactive enzyme activity (EXC+) (FIG. 9B). The deletion of amino acid residues 2-47 (N2) (SEQ ID NO. 444 and SEQ ID NO. 445) showed the most excision activity (FIG. 9B).

The helper enzyme from Eptesicus fuscus will also be subjected to fusion with protein binding domains (e.g., without limitation, TALEs, TniQ subdomain of TnsD, dCas9, and dCas12j) as described throughout the present application. Fusion proteins mutants will be generated using known methods and the mutants will be screened for integration and excision activity. Mutants that show optimized activity will be selected as candidates for additional rounds of optimization (e.g., without limitation, additional rounds of screening and/or addition of fusion proteins as described herein).

### Example 4—Construction of Targeting Elements Directed to TTAA Sites in hROSA26, AAVS1, Chromosome 4, Chromosome 22, and Chromosome X Targeted by guideRNAs, TALES, and ZnF

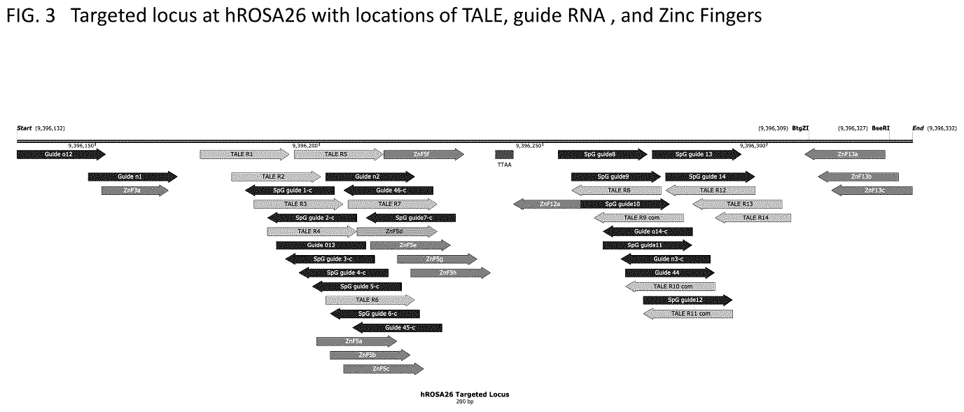

FIG. 3 depicts the TTAA site in hROSA26 (hg38 chr3: 9,396, 133-9,396,332) that is targeted by guideRNAs (TABLE 2), TALES (TABLE 8), and ZnF (TABLE 13).

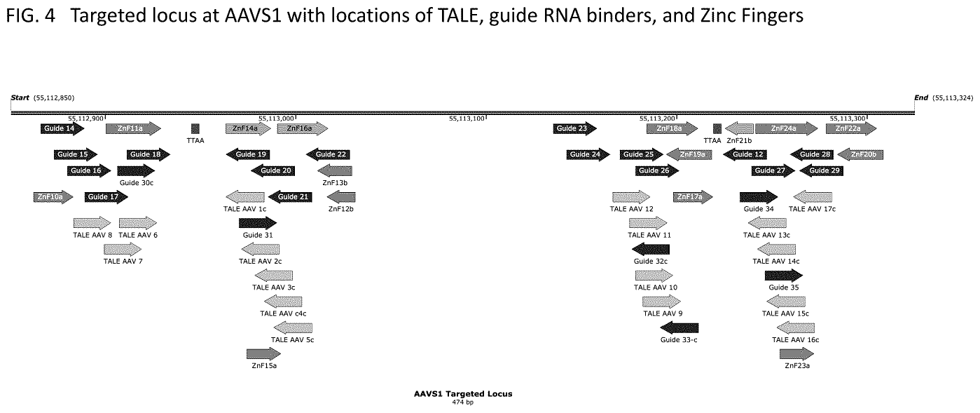

FIG. 4 depicts two TTAA sites in AAVS1 (hg38 chr19: 55, 112,851-55, 113,324) that are targeted by guideRNAs (TABLE 3) or TALES (TABLE 9), and ZnF (TABLE 14).

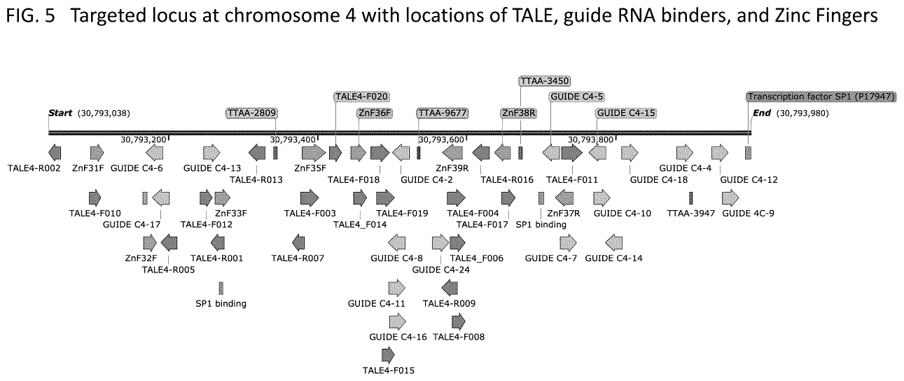

FIG. 5 depicts two TTAA sites in Chromosome 4 (hg38 chr4: 30,793,039-30,793,980) that are targeted by guideRNAs (TABLE 4) or TALES (TABLE 10), and ZnF (TABLE 15).

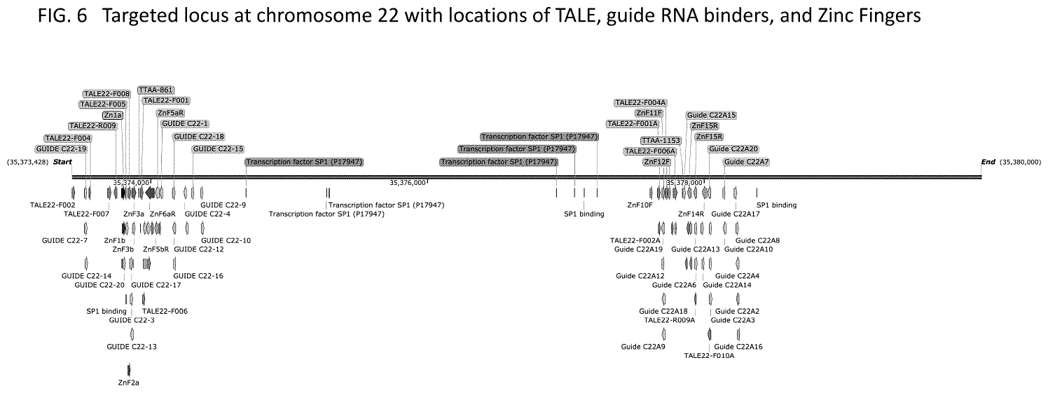

FIG. 6 depicts two TTAA sites in Chromosome 22 (hg38 chr22: 35,373,429-35,380,000) that are targeted by guideRNAs (TABLE 5) or TALES (TABLE 11), and ZnF (TABLE 16).

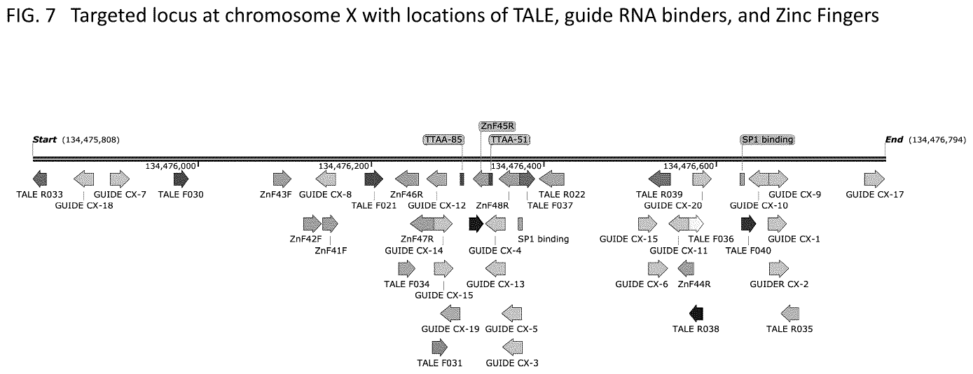

FIG. 7 depicts two TTAA sites in Chromosome X (hg38 chrX: 134,475,809-134,476,794) that are targeted by guideRNAs (TABLE 6) or TALES (TABLE 12), and ZnF (TABLE 17).

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising a helper enzyme or a nucleic acid encoding the enzyme, wherein the enzyme comprises an amino acid sequence having at least about 80% sequence identity to SEQ ID NO: 1, SEQ ID NO: 441, SEQ ID NO: 442, SEQ ID NO: 444, SEQ ID NO: 446, SEQ ID NO: 448, or SEQ ID NO: 450.

2. The composition of claim 1, wherein the enzyme comprises an amino acid sequence of at least about 90% identity to SEQ ID NO: 1, SEQ ID NO: 441, SEQ ID NO: 442, SEQ ID NO: 444, SEQ ID NO: 446, SEQ ID NO: 448, or SEQ ID NO: 450.

3. The composition of claim 1, wherein the enzyme comprises an amino acid sequence of at least about 93% identity to SEQ ID NO: 1, SEQ ID NO: 441, SEQ ID NO: 442, SEQ ID NO: 444, SEQ ID NO: 446, SEQ ID NO: 448, or SEQ ID NO: 450.

4. The composition of claim 1, wherein the enzyme comprises an amino acid sequence of at least about 95% identity to SEQ ID NO: 1, SEQ ID NO: 441, SEQ ID NO: 442, SEQ ID NO: 444, SEQ ID NO: 446, SEQ ID NO: 448, or SEQ ID NO: 450.

5. The composition of claim 1, wherein the enzyme comprises an amino acid sequence of at least about 98% identity to SEQ ID NO: 1, SEQ ID NO: 441, SEQ ID NO: 442, SEQ ID NO: 444, SEQ ID NO: 446, SEQ ID NO: 448, or SEQ ID NO: 450.

6. The composition of claim 1, wherein the enzyme comprises an amino acid sequence of at least about 99% identity to SEQ ID NO: 1, SEQ ID NO: 441, SEQ ID NO: 442, SEQ ID NO: 444, SEQ ID NO: 446, SEQ ID NO: 448, or SEQ ID NO: 450.

7. The composition of any one of claims 1-6, wherein the enzyme has one or more mutations which confer hyperactivity.

8. The composition of any one of claims 1-7, wherein the enzyme has one or more amino acid substitutions.

9. The composition of any one of claims 1-8, wherein the enzyme is a hyperactive variant of SEQ ID NO: 1, SEQ ID NO: 441, SEQ ID NO: 442, SEQ ID NO: 444, SEQ ID NO: 446, SEQ ID NO: 448, or SEQ ID NO: 450.

10. The composition of any one of claims 1-6, wherein the nucleic acid that encodes the enzyme has a nucleotide sequence of at least about 80% identical to SEQ ID NO: 2, SEQ ID NO: 443, SEQ ID NO: 445, SEQ ID NO: 447, SEQ ID NO: 449, or SEQ ID NO: 451, or a codon-optimized form thereof.

11. The composition of claim 10, wherein the nucleic acid that encodes the enzyme has a nucleotide sequence of at least about 90% identical to SEQ ID NO: 2, SEQ ID NO: 443, SEQ ID NO: 445, SEQ ID NO: 447, SEQ ID NO: 449, or SEQ ID NO: 451, or a codon-optimized form thereof.

12. The composition of claim 10, wherein the nucleic acid that encodes the enzyme has a nucleotide sequence of at least about 93% identical to SEQ ID NO: 2, SEQ ID NO: 443, SEQ ID NO: 445, SEQ ID NO: 447, SEQ ID NO: 449, or SEQ ID NO: 451, or a codon-optimized form thereof.

13. The composition of any one of claims 1-12, wherein the enzyme has increased activity relative to an enzyme comprising an amino acid sequence of SEQ ID NO: 1 or functional equivalent thereof.

14. The composition of any one of claims 1-13, wherein the enzyme is excision positive.

15. The composition of any one of claims 1-14, wherein the enzyme is integration deficient.

16. The composition of any one of claims 14-15, wherein the enzyme has decreased integration activity relative to an enzyme comprising an amino acid sequence of SEQ ID NO: 1 or functional equivalent thereof.

17. The composition of any one of claims 14-16, wherein the enzyme has increased excision activity relative to an enzyme comprising an amino acid sequence of SEQ ID NO: 1 or functional equivalent thereof.

18. The composition of any one of claims 1-17, wherein the enzyme comprises a targeting element.

19. The composition of any one of claims 1-18, wherein the enzyme is capable of inserting a donor comprising a transgene in a genomic safe harbor site (GSHS).

20. The composition of claim 19, wherein the binding of a GSHS of a nucleic acid molecule in a mammalian cell is with high target specificity, relative to a control.

21. The composition of claim 20, wherein the control is a composition comprising an enzyme comprising an amino acid sequence of SEQ ID NO: 1 or a nucleic acid comprising a nucleotide sequence of SEQ ID NO: 2 or a codon-optimized form thereof.

22. The composition of any one of claims 18-21, wherein the targeting element is able to direct a transposition machinery to the GSHS of a nucleic acid molecule in a mammalian cell.

23. The composition of any one of claims 18-22, wherein the GSHS is in an open chromatin location in a chromosome.

24. The composition of any one of claims 18-23, wherein the GSHS is selected from adeno-associated virus site 1 (AAVS1), chemokine (C—C motif) receptor 5 (CCR5) gene, HIV-1 coreceptor, and human Rosa26 locus.

25. The composition of any one of claims 18-24, wherein the GSHS is an adeno-associated virus site 1 (AAVS1).

26. The composition of any one of claims 18-25, wherein the GSHS is a human Rosa26 locus.

27. The composition of any one of claims 18-26, wherein the GSHS is located on human chromosome 2, 4, 6, 10, 11, 17, 22, or X.

28. The composition of any one of claims 18-27, wherein the GSHS is selected from TABLES 1-17.

29. The composition of any one of claims 18-28, wherein the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

30. The composition of any one of claims 18-29, wherein the targeting element is or comprises one or more of a Cas enzyme, which is optionally catalytically inactive and which is optionally associated with a guide RNA (gRNA), transcription activator-like effector (TALE) DNA binding domain (DBD), catalytically inactive Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TniQ subdomain of TnsD) or a variant thereof.

31. The composition of claim 30, wherein the targeting element comprises a TALE DBD.

32. The composition of claim 31, wherein the TALE DBD comprises one or more repeat sequences.

33. The composition of claim 32, wherein the TALE DBD comprises about 14, or about 15, or about, 16, or about 17, or about 18, or about 18.5 repeat sequences.

34. The composition of claim 32 or claim 33, wherein the repeat sequences each independently comprises about 33 or 34 amino acids.

35. The composition of claim 34, wherein the repeat sequences each independently comprises a repeat variable di-residue (RVD) at residue 12 or 13 of the 33 or 34 amino acids, respectively.

36. The composition of claim 35, wherein the RVD recognizes one base pair in a target nucleic acid sequence.

37. The composition of claim 34 or claim 35, wherein the RVD recognizes a C residue in the target nucleic acid sequence and is selected from HD, N (gap), HA, ND, and HI.

38. The composition of claim 34 or claim 35, wherein the RVD recognizes a G residue in the target nucleic acid sequence and is selected from NN, NH, NK, HN, and NA.

39. The composition of claim 34 or claim 35, wherein the RVD recognizes an A residue in the target nucleic acid sequence and is selected from NI and NS.

40. The composition of claim 34 or claim 35, wherein the RVD recognizes a T residue in the target nucleic acid sequence and is selected from NG, HG, H (gap), and IG.

41. The composition of claim 30-40, wherein the TALE DBD targets one or more of GSHS sites selected from TABLES 7-12.

42. The composition of any one of claims 30-41, wherein the TALE DBD comprises one or more of RVD selected from TABLES 7-12, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

43. The composition of claim 30, wherein the targeting element comprises a Cas9 enzyme associated with a gRNA or a CasX enzyme associated with a gRNA.

44. The composition of claim 43, wherein the Cas9 enzyme associated with a gRNA comprises a catalytically inactive dCas9 associated with a gRNA or a inactive dCasX associated with a gRNA.

45. The composition of claim 44, wherein catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

46. The composition of any one of claim 30 or 43-45, wherein the targeting element comprises a Cas12 enzyme associated with a gRNA.

47. The composition of claim 46, wherein the targeting element comprises a catalytically inactive Cas12 associated with a gRNA, optionally wherein the catalytically inactive Cas12 is dCas12j or dCas12a.

48. The composition of any one of claim 30 or 43-45, wherein the targeting element comprises a TnsC, TnsB, TnsA, TniQ, Cas6, Cas7, Cas8 enzyme associated with a gRNA.

49. The composition of any one of claim 30 or 43-45, wherein the targeting element comprises a TniQ subdomain of TnsD.

50. The composition of claim 30 or 43-47, wherein the guide RNA is selected from TABLES 1-6, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

51. The composition of claim 30 or 43-47, wherein the guide RNA targets one or more sites selected from TABLES 1-6.

52. The composition of claim 30, wherein the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence.

53. The composition of claim 30, wherein the zinc finger targets one or more sites selected from TABLES 13-17.

54. The composition of any one of claims 30-53, wherein the targeting element comprises a nucleic acid binding component of a gene-editing system.

55. The composition of any one of claims 30-54, wherein the enzyme or variant thereof and the targeting element are connected.

56. The composition of claim 55, wherein the enzyme and the targeting element are fused to one another or linked via a linker to one another.

57. The composition of claim 56, wherein the linker is a flexible linker.

58. The composition of claim 57, wherein the flexible linker is substantially comprised of glycine and serine residues, optionally wherein the flexible linker comprises (Gly₄Ser)ₙ, where n is an integer from 1-12.

59. The composition of claim 58, wherein the flexible linker is of about 20, or about 30, or about 40, or about 50, or about 60 amino acid residues.

60. The composition of claim 59, wherein the enzyme is directly fused to the N-terminus of the targeting element and, optionally, wherein the targeting element is or comprises dCas9 enzyme.

61. The composition of any one of claims 1-60, wherein the enzyme or variant thereof is able to directly or indirectly cause transposition of a target gene.

62. The composition of any one of claims 1-61, wherein the enzyme or variant thereof is able to directly or indirectly interact and/or form a complex with one or more proteins or nucleic acids.

63. The composition of any one of the preceding claims, wherein a nucleic acid encoding the enzyme capable of targeted genomic integration by transposition comprises an intein, optionally NpuN (Intein-N) (SEQ ID NO: 423) and/or NpuC (Intein-C) (SEQ ID NO: 424), or a variant thereof.

64. The composition of claim 63, wherein the nucleic acid encodes the enzyme in the form of first and second portions with the intein encoded between the first and second portions, such that the first and second portions are fused into a functional enzyme upon post-translational excision of the intein from the enzyme.

65. The composition of claim 63 or claim 64, wherein the intein is suitable for linking the helper enzyme and the targeting element.

66. The composition of any one of the preceding claims, wherein a nucleic acid encoding the enzyme capable of targeted genomic integration by transposition comprises a dimerization enhancer.

67. The composition of claim 66, wherein the nucleic acid encodes the enzyme in the form of first and second portions with the dimerization enhancer encoded between the first and second portions, such that the first and second portions are fused into a functional enzyme upon post-translational excision of the dimerization enhancer from the enzyme.

68. The composition of claim 66 or claim 67, wherein the dimerization enhancer is suitable for linking the helper enzyme and the targeting element.

69. The composition of any one of claims 66-68, wherein the dimerization enhancer is selected from: a protein comprising a SH3 domain, biotin, avidin, or a rapamycin binder, optionally, wherein the rapamycin binder is FKBP12 or mTOR, or a variant thereof.

70. The composition of any one of claims 1-69, further comprising a nucleic acid encoding a donor comprising a transgene to be integrated, optionally wherein the transgene is defective or substantially absent in a disease state.

71. The composition of claim 70, wherein the transgene comprises a cargo nucleic acid sequence and a first and a second donor end sequences.

72. The composition of claim 71, wherein the cargo nucleic acid sequence is flanked by the first and the second donor end sequences.

73. The composition of claim 71 or claim 72, wherein the donor end sequences are selected from nucleotide sequences of SEQ ID NO: 3 and/or SEQ ID NO: 4, or a nucleotide sequence having at least about 90% identity thereto.

74. The composition of any one of claims 71-73, wherein the end sequences include at least one repeat from a nucleotide sequence having at least about 90% identity to the nucleotide sequence of SEQ ID NO: 3.

75. The composition of claim 74, wherein the at least one repeat from the nucleotide sequence having at least about 90% identity to the nucleotide sequence of SEQ ID NO: 3 is positioned at the 5′ end of the donor.

76. The composition of any one of claims 71-75, wherein the end sequences can further include at least one repeat from a nucleotide sequence having at least about 90% identity to the nucleotide sequence of SEQ ID NO: 4.

77. The composition of any one of claims 72-76, wherein the at least one repeat from the nucleotide sequence having at least about 90% identity to the nucleotide sequence of SEQ ID NO: 4 is positioned at the 3′ end of the donor.

78. The composition of any one of claims 1-77, wherein the donor construct comprising a heterologous polynucleotide between left and right transposon ends, wherein the left end comprises SEQ ID NO: 3, or a functional variant thereof and the right end comprises SEQ ID NO: 4, or a functional variant thereof.

79. The composition of any one of claims 1-78, wherein the polynucleotide comprising an open reading frame encoding a transposase, the amino acid sequence of which is at least 90% identical to SEQ ID NO: 1, SEQ ID NO: 441, SEQ ID NO: 442, SEQ ID NO: 444, SEQ ID NO: 446, SEQ ID NO: 448, or SEQ ID NO: 450, or a functional variant thereof, operably linked to a heterologous promoter.

80. The composition of claim 79, wherein the enzyme or variant thereof is incorporated into a vector or a vector-like particle, wherein the vector or a vector-like particle comprises one or more expression cassettes, and/or wherein the vector or a vector-like particle comprises one expression cassette.

81. The composition of claim 80, wherein the expression cassette further comprises the enzyme or variant thereof, the transgene, the donor end sequences, or a combination thereof.

82. The composition of claim 81, wherein the enzyme or variant thereof, the transgene, the donor end sequences, or a combination thereof are incorporated into one or more vectors or vector-like particles.

83. The composition of claim 81, wherein the enzyme or variant thereof, the transgene, the donor end sequences, or combination thereof are incorporated into a same vector or vector-like particle.

84. The composition of claim 81, wherein the enzyme or variant thereof, the transgene, the donor end sequences, or combination thereof is incorporated into different vectors or vector-like particles.

85. The composition of any one of claims 78-84, wherein the vector or vector-like particle is nonviral.

86. The composition of any one of claims 70-85, wherein the donor is under the control of at least one tissue-specific promoter.

87. The composition of claim 86, wherein the at least one tissue-specific promoter is a single promoter.

88. The composition of claim 86, wherein the at least one tissue-specific promoter is under the control of a dual promoter or a tandem promoter.

89. The composition of any one of claims 70-88, wherein the transgene to be integrated comprises at least one gene of interest.

90. The composition of any one of claims 70-89, wherein the transgene to be integrated comprises one gene of interest.

91. The composition of any one of claims 70-89, wherein the transgene to be integrated comprises two or more genes of interest.

92. The composition of any one of claims 70-91, wherein the at least one gene of interest comprises peptides for linking genes of interest.

93. The composition of claim 92, wherein the peptides are 2A self-cleaving peptides, or functional variants thereof, wherein the 2A self-cleaving peptide is optionally selected from P2A, E2A, F2A, and T2A, or derivative thereof.

94. The composition of any one of claims 70-93, wherein the at least one gene of interest is linked to polynucleotide comprising a sequence comprising a 5′-miRNA, a sense and antisense miRNA pair, and/or a 3′-miRNA.

95. The composition of any one of claims 1-94, wherein the composition comprises DNA, RNA, or both.

96. The composition of any one of claims 1-95, wherein the enzyme or variant thereof is in the form of RNA.

97. A host cell comprising the composition any one of claims 1-96.

98. The composition of any one of claims 1-96, wherein the composition is encapsulated in a lipid nanoparticle (LNP).

99. The composition of any one of claims 1-96, wherein the polynucleotide encoding the enzyme or variant thereof and the polynucleotide encoding the donor are in the form of the same LNP, optionally in a co-formulation.

100. The composition of claim 98 or claim 99, wherein the LNP comprises one or more lipids selected from 1,2-dioleoyl-3-trimethylammonium propane (DOTAP), a cationic cholesterol derivative mixed with dimethylaminoethane-carbamoyl (DC—Chol), phosphatidylcholine (PC), triolein (glyceryl trioleate), and 1,2-distearoyl-sn-glycero-3-phosphoethanolamine-N-8 carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

101. A method for inserting a gene into the genome of a cell, comprising contacting a cell with the composition of any one of claim 1-96 or 98-100 or host cell of claim 97.

102. The method of claim 101, further comprising contacting the cell with a polynucleotide encoding a donor DNA.

103. The method of claim 101 or claim 102, wherein the donor comprises a gene encoding a complete polypeptide.

104. The method of any one of claims 101-103, wherein the donor comprises a gene which is defective or substantially absent in a disease state.

105. A method for treating a disease or disorder ex vivo, comprising contacting a cell with the composition of any one of claim 1-96 or 98-100 or host cell of claim 97 and administering the cell to a subject in need thereof.

106. A method for treating a disease or disorder in vivo, comprising administering the composition of any one of claim 1-96 or 98-100 or host cell of claim 97 to a subject in need thereof.

107. A donor construct comprising a heterologous polynucleotide between left and right transposon ends, wherein the left end comprises SEQ ID NO: 3, or a functional variant thereof and the right end comprises SEQ ID NO: 4, or a functional variant thereof.

108. The donor construct of claim 107, wherein the donor is transposable by a helper enzyme having the sequence of SEQ ID NO: 1, SEQ ID NO: 441, SEQ ID NO: 442, SEQ ID NO: 444, SEQ ID NO: 446, SEQ ID NO: 448, or SEQ ID NO: 450, or a functional variant thereof.

109. A donor construct comprising a heterologous polynucleotide between left and right transposon ends, wherein the donor is suitable for transposition by a helper enzyme having the sequence of SEQ ID NO: 1, SEQ ID NO: 441, SEQ ID NO: 442, SEQ ID NO: 444, SEQ ID NO: 446, SEQ ID NO: 448, or SEQ ID NO: 450, or a functional variant thereof.

110. A helper enzyme derived from Eptesicus fuscus, the helper enzyme being suitable for transposition of a heterologous polynucleotide, the heterologous polynucleotide being flanked by two ends elements comprising the polynucleotide sequences of SEQ ID NO: 3, or a functional variant thereof and SEQ ID NO: 4, or a functional variant thereof.

111. A helper enzyme derived from Eptesicus fuscus, the helper enzyme having the sequence of SEQ ID NO: 1.

112. A helper enzyme derived from Eptesicus fuscus, the helper enzyme having the sequence of SEQ ID NO: 441.

113. A helper enzyme derived from Eptesicus fuscus, the helper enzyme having the sequence of SEQ ID NO: 442.

114. A helper enzyme derived from Eptesicus fuscus, the helper enzyme having the sequence of SEQ ID NO: 444.

115. A helper enzyme derived from Eptesicus fuscus, the helper enzyme having the sequence of SEQ ID NO: 446.

116. A helper enzyme derived from Eptesicus fuscus, the helper enzyme having the sequence of SEQ ID NO: 448.

117. A helper enzyme derived from Eptesicus fuscus, the helper enzyme having the sequence of SEQ ID NO: 450.

118. A helper enzyme derived from Eptesicus fuscus, the helper enzyme having the sequence of SEQ ID NO: 1.
